SEQUENCE LISTING (254 sequences)

SEQ ID NO: 1 - DNA, 2179 bp, Escherichia coli
  gene 1..2179: note = E. coli essential gene folP-glmM
  source 1..2179: mol_type = genomic DNA
  CDS 1..849: note = folP
atgaaactctttgcccagggtacttcactggaccttagccatcctcacgtaatggggatcctcaacgtcacgcctgattccttttcggatggtggcacgcataactcgctgatagatgcggtgaaacatgcgaatctgatgatcaatgctggcgcgacgatcattgacgttggtggcgagtccacgcgcccaggggcggcggaagttagcgttgaagaagagttgcaacgtgttattcctgtggttgaggcaattgctcaacgcttcgaagtctggatctcggtcgatacatccaaaccagaagtcatccgtgagtcagcgaaagttggcgctcacattattaatgatatccgctccctttccgaacctggcgctctggaggcggctgcagaaaccggtttaccggtttgtctgatgcatatgcagggaaatccaaaaaccatgcaggaagctccgaagtatgacgatgtctttgcagaagtgaatcgctactttattgagcaaatagcacgttgcgagcaggcgggtatcgcaaaagagaaattgttgctcgaccccggattcggtttcggtaaaaatctctcccataactattcattactggcgcgcctggctgaatttcaccatttcaacctgccgctgttggtgggtatgtcacgaaaatcgatgattgggcagctgctgaacgtggggccgtccgagcgcctgagcggtagtctggcctgtgcggtcattgccgcaatgcaaggcgcgcacatcattcgtgttcatgacgtcaaagaaaccgtagaagcgatgcgggtggtggaagccactctgtctgcaaaggaaaacaaacgctatgagtaatcgtaaatatttcggtaccgatgggattcgtggtcgtgtaggggatgcgccgatcacacctgattttgtgcttaagctgggttgggccgcgggtaaagtgctggcgcgccacggctcccgtaagatcattattggtaaagacacgcgtatttctggctatatgctggagtcagcactggaagcgggtctggcggcagcgggcctttccgcactcttcactggcccgatgccaacaccggccgtggcttatctgacgcgtaccttccgcgcagaggccggaattgtgatatctgcatcgcataacccgttctacgataatggcattaaattcttctctatcgacggcaccaaactgccggatgcggtagaagaggccatcgaagcggaaatggaaaaggagatcagctgcgttgattcggcagaactgggtaaagccagccgtatcgttgatgccgcgggtcgctatatcgagttttgcaaagccacgttcccgaacgaacttagcctcagtgaactgaagattgtggtggattgtgcaaacggtgcgacttatcacatcgcgccgaacgtgctgcgcgaactgggggcgaacgttatcgctatcggttgtgagccaaacggtgtaaacatcaatgccgaagtgggggctaccgacgttcgcgcgctccaggctcgtgtgctggctgaaaaagcggatctcggtattgccttcgacggcgatggcgatcgcgtgattatggttgaccatgaaggcaataaagtcgatggcgatcagatcatgtatatcatcgcgcgtgaaggtcttcgtcagggccagctgcgtggtggcgctgtgggtacattgatgagcaacatggggcttgaactggcgctgaaacagttaggaattccatttgcgcgcgcgaaagtgggtgaccgctacgtactggaaaaaatgcaggagaaaggctggcgtatcggtgcagagaattccggtcatgtgatcctgctggataaaactactaccggtgacggcatcgttgctggcttgcaggtgctggcggctatggcacgtaaccatatgagcctgcacgacctttgcagcggcatgaaaatgttcccgcagattctggttaacgtacgttacaccgcaggtagcggcgatccacttgagcatgagtcagttaaagccgtgaccgcagaggttgaagctgcgctgggcaaccgtggacgcgtgttgctgcgtaaatccggcaccgaaccgttaattcgcgtgatggtggaaggcgaagacgaagcgcaggtgactgaatttgcacaccgcatcgccgatgcagtaaaagccgtttaa

SEQ ID NO: 2 - protein, 282 aa, Escherichia coli
  source 1..282: note = The sequence is a translation of CDS folP of SEQ ID NO. 1 (nt position 1..859 of SEQ ID NO. 1)
MKLFAQGTSLDLSHPHVMGILNVTPDSFSDGGTHNSLIDAVKHANLMINAGATIIDVGGESTRPGAAEVSVEEELQRVIPVVEAIAQRFEVWISVDTSKPEVIRESAKVGAHIINDIRSLSEPGALEAAAETGLPVCLMHMQGNPKTMQEAPKYDDVFAEVNRYFIEQIARCEQAGIAKEKLLLDPGFGFGKNLSHNYSLLARLAEFHHFNLPLLVGMSRKSMIGQLLNVGPSERLSGSLACAVIAAMQGAHIIRVHDVKETVEAMRVVEATLSAKENKRYE

SEQ ID NO: 3 - DNA, 1338 bp, Escherichia coli
  source 1..1338: mol_type = genomic DNA
  CDS 1..1338: note = E. coli essential gene glmM
atgagtaatcgtaaatatttcggtaccgatgggattcgtggtcgtgtaggggatgcgccgatcacacctgattttgtgcttaagctgggttgggccgcgggtaaagtgctggcgcgccacggctcccgtaagattattattggtaaagacacgcgtatttctggctatatgctggagtcagcactggaagcgggtctggcggcagcgggcctttccgcactcttcactggcccgatgccaacaccggccgtggcttatctgacgcgtaccttccgcgcagaggccggaattgtgatatctgcatcgcataacccgttctacgataatggcattaaattcttctctatcgacggcaccaaactgccggatgcggtagaagaggccatcgaagcggaaatggaaaaggagatcagctgcgttgattcggcagaactgggtaaagccagccgtatcgttgatgccgcgggtcgctatatcgagttttgcaaagccacgttcccgaacgaacttagcctcagtgaactgaagattgtggtggattgtgcaaacggtgcgacttatcacatcgcgccgaacgtgctgcgcgaactgggggcgaacgttatcgctatcggttgtgagccaaacggtgtaaacatcaatgccgaagtgggggctaccgacgttcgcgcgctccaggctcgtgtgctggctgaaaaagcggatctcggtattgccttcgacggcgatggcgatcgcgtgattatggttgaccatgaaggcaataaagtcgatggcgatcagatcatgtatatcatcgcgcgtgaaggtcttcgtcagggccagctgcgtggtggcgctgtgggtacattgatgagcaacatggggcttgaactggcgctgaaacagttaggaattccatttgcgcgcgcgaaagtgggtgaccgctacgtactggaaaaaatgcaggagaaaggctggcgtatcggtgcagagaattccggtcatgtgatcctgctggataaaactactaccggtgacggcatcgttgctggcttgcaggtgctggcggcgatggcacgtaaccatatgagcctgcacgacctttgcagcggcatgaaaatgttcccgcagattctggttaacgtacgttacaccgcaggtagcggcgatccacttgagcatgagtcagttaaagccgtgaccgcagaggttgaagctgcgctgggcaaccgtggacgcgtgttgctgcgtaaatccggcaccgaaccgttaattcgcgtgatggtggaaggcgaagacgaagcgcaggtgactgaatttgcacaccgcatcgccgatgcagtaaaagccgtttaa

SEQ ID NO: 4 - protein, 445 aa, Escherichia coli
  source 1..445: note = The sequence is a translation of SEQ ID NO.3
MSNRKYFGTDGIRGRVGDAPITPDFVLKLGWAAGKVLARHGSRKIIIGKDTRISGYMLESALEAGLAAAGLSALFTGPMPTPAVAYLTRTFRAEAGIVISASHNPFYDNGIKFFSIDGTKLPDAVEEAIEAEMEKEISCVDSAELGKASRIVDAAGRYIEFCKATFPNELSLSELKIVVDCANGATYHIAPNVLRELGANVIAIGCEPNGVNINAEVGATDVRALQARVLAEKADLGIAFDGDGDRVIMVDHEGNKVDGDQIMYIIAREGLRQGQLRGGAVGTLMSNMGLELALKQLGIPFARAKVGDRYVLEKMQEKGWRIGAENSGHVILLDKTTTGDGIVAGLQVLAAMARNHMSLHDLCSGMKMFPQILVNVRYTAGSGDPLEHESVKAVTAEVEAALGNRGRVLLRKSGTEPLIRVMVEGEDEAQVTEFAHRIADAVKAV

SEQ ID NO: 5 - DNA, 858 bp, Escherichia coli
  source 1..858: mol_type = genomic DNA
  CDS 1..858: note = E. coli essential gene murI
atggctaccaaactgcaggacgggaatacaccttgtctggcagctacaccttctgaaccacgtcccaccgtgctggtgtttgactccggcgtcggtgggttgtcggtctatgacgagatccggcatctcttaccggatctccattacatttatgctttcgataacgtcgctttcccgtatggcgaaaaaagcgaagcgtttattgttgagcgagtggtggcaattgtcaccgcggtgcaagaacgttatccccttgcgctggctgtggtcgcttgcaacactgccagtaccgtttcacttcctgcattacgcgaaaagttcgacttcccggttgttggtgtcgtgccggcgattaaacctgctgcacgtctgacggcaaatggcattgtcggattactggcaacccgcggaacagttaaacgttcttatactcatgagctgatcgcgcgtttcgctaatgaatgccagatagaaatgctgggctcggcagagatggttgagttggctgaagcgaagctacatggcgaagatgtttctctggatgcactaaaacgtatcctacgcccgtggttaagaatgaaagagccgccagataccgttgtattgggttgcacccatttccctctactacaagaagaactgttacaagtgctgccagagggaacccggctggtggattctggcgcagcgattgctcgccgaacggcctggttgttagaacatgaagccccggatgcaaaatctgccgatgcgaatattgccttttgtatggcaatgacgccaggagctgaacaattattgcccgttttacagcgttacggcttcgaaacgctcgaaaaactggcagttttaggctga

SEQ ID NO: 6 - protein, 285 aa, Escherichia coli
  source 1..285: note = The sequence is a translation of SEQ ID NO.5
MATKLQDGNTPCLAATPSEPRPTVLVFDSGVGGLSVYDEIRHLLPDLHYIYAFDNVAFPYGEKSEAFIVERVVAIVTAVQERYPLALAVVACNTASTVSLPALREKFDFPVVGVVPAIKPAARLTANGIVGLLATRGTVKRSYTHELIARFANECQIEMLGSAEMVELAEAKLHGEDVSLDALKRILRPWLRMKEPPDTVVLGCTHFPLLQEELLQVLPEGTRLVDSGAAIARRTAWLLEHEAPDAKSADANIAFCMAMTPGAEQLLPVLQRYGFETLEKLAVLG

SEQ ID NO: 7 - DNA, 1104 bp, Escherichia coli
  source 1..1104: mol_type = genomic DNA
  CDS 1..1104: note = E. coli essential gene asd
atgaaaaatgttggttttatcggctggcgcggtatggtcggctccgttctcatgcaacgcatggttgaagagcgcgacttcgacgccattcgccctgtctacttttctacttctcagcttggtcaggctgcgccgtcttttggcggaaccactggcacacttcaggatgcctttgatctggaggcgctaaaggccctcgatatcattgtgacctgtcagggcggcgattataccaacgaaatctatccaaagcttcgtgaaagcggatggcagggttactggattgacgcagcatcgtctctgcgcatgaaagatgacgccatcatcattcttgaccccgtcaatcaggacgtcattaccgacggattaaataatggcatcaggacttttgttggcggtaactgtaccgtaagcctgatgttgatgtcgttgggtggtttattcgccaatgatcttgttgattgggtgtccgttgcaacctaccaggccgcttccggcggtggtgcgcgacatatgcgtgagttattaacccagatgggccatctgtatggccatgtagcagatgaactcgcgaccccgtcctctgctattctcgatatcgaacgcaaagtcacaaccttaacccgtagcggtgagctgccggtggataactttggcgtgccgctggcgggtagcctgattccgtggatcgacaaacagctcgataacggtcagagccgcgaagagtggaaagggcaggcggaaaccaacaagatcctcaacacatcttccgtaattccggtagatggtttatgtgtgcgtgtcggggcattgcgctgccacagccaggcattcactattaaattgaaaaaagatgtgtcgattccgaccgtggaagaactgctggctgcgcacaatccgtgggcgaaagtcgttccgaacgatcgggaaatcactatgcgtgagctaaccccagctgccgttaccggcacgctgaccacgccggtaggccgcctgcgtaagctgaatatgggaccagagttcctgtcagcctttaccgtgggcgaccagctgctgtggggggccgcggagccgctgcgtcggatgcttcgtcaactggcgtaa

SEQ ID NO: 8 - protein, 367 aa, Escherichia coli
  source 1..367: note = The sequence is a translation of SEQ ID NO.7
MKNVGFIGWRGMVGSVLMQRMVEERDFDAIRPVYFSTSQLGQAAPSFGGTTGTLQDAFDLEALKALDIIVTCQGGDYTNEIYPKLRESGWQGYWIDAASSLRMKDDAIIILDPVNQDVITDGLNNGIRTFVGGNCTVSLMLMSLGGLFANDLVDWVSVATYQAASGGGARHMRELLTQMGHLYGHVADELATPSSAILDIERKVTTLTRSGELPVDNFGVPLAGSLIPWIDKQLDNGQSREEWKGQAETNKILNTSSVIPVDGLCVRVGALRCHSQAFTIKLKKDVSIPTVEELLAAHNPWAKVVPNDREITMRELTPAAVTGTLTTPVGRLRKLNMGPEFLSAFTVGDQLLWGAAEPLRRMLRQLA

SEQ ID NO: 9 - DNA, 1104 bp, Escherichia coli; identical to SEQ ID NO: 7
  source 1..1104: mol_type = genomic DNA
  CDS 1..1104: note = E. coli essential gene thyA

SEQ ID NO: 10 - protein, 367 aa, Escherichia coli; identical to SEQ ID NO: 8
  source 1..367: note = The sequence is a translation of SEQ ID NO.9

SEQ ID NO: 11 - DNA, 1842 bp, Escherichia coli
  source 1..1842: mol_type = genomic DNA
  CDS 1..1842: note = E. coli essential gene rpoD
atggagcaaaacccgcagtcacagctgaaacttcttgtcacccgtggtaaggagcaaggctatctgacctatgccgaggtcaatgaccatctgccggaagatatcgtcgattcagatcagatcgaagacatcatccaaatgatcaacgacatgggcattcaggtgatggaagaagcaccggatgccgatgatctgatgctggctgaaaacaccgcggacgaagatgctgccgaagccgccgcgcaggtgctttccagcgtggaatctgaaatcgggcgcacgactgacccggtacgcatgtacatgcgtgaaatgggcaccgttgaactgttgacccgcgaaggcgaaattgacatcgctaagcgtattgaagacgggatcaaccaggttcaatgctccgttgctgaatatccggaagcgatcacctatctgctggaacagtacgatcgtgttgaagcagaagaagcgcgtctgtccgatctgatcaccggctttgttgacccgaacgcagaagaagatctggcacctaccgccactcacgtcggttctgagctttcccaggaagatctggacgatgacgaagatgaagacgaagaagatggcgatgacgacagcgccgatgatgacaacagcatcgacccggaactggctcgcgaaaaatttgcggaactacgcgctcagtacgttgtaacgcgtgacaccatcaaagcgaaaggtcgcagtcacgctaccgctcaggaagagatcctgaaactgtctgaagtattcaaacagttccgcctggtgccgaagcagtttgactacctggtcaacagcatgcgcgtcatgatggaccgcgttcgtacgcaagaacgtctgatcatgaagctctgcgttgagcagtgcaaaatgccgaagaaaaacttcattaccctgtttaccggcaacgaaaccagcgatacctggttcaacgcggcaattgcgatgaacaagccgtggtcggaaaaactgcacgatgtctctgaagaagtgcatcgcgccctgcaaaaactgcagcagattgaagaagaaaccggcctgaccatcgagcaggttaaagatatcaaccgtcgtatgtccatcggtgaagcgaaagcccgccgtgcgaagaaagagatggttgaagcgaacttacgtctggttatttctatcgctaagaaatacaccaaccgtggcttgcagttccttgacctgattcaggaaggcaacatcggtctgatgaaagcggttgataaattcgaataccgccgtggttacaagttctccacctacgcaacctggtggatccgtcaggcgatcacccgctctatcgcggatcaggcgcgcaccatccgtattccggtgcatatgattgagaccatcaacaagctcaaccgtatttctcgccagatgctgcaagagatgggccgtgaaccgacgccggaagaactggctgaacgtatgctgatgccggaagacaagatccgcaaagtgctgaagatcgccaaagagccaatctccatggaaacgccgatcggtgatgatgaagattcgcatctgggggatttcatcgaggataccaccctcgagctgccgctggattctgcgaccaccgaaagcctgcgtgcggcaacgcacgacgtgctggctggcctgaccgcgcgtgaagcaaaagttctgcgtatgcgtttcggtatcgatatgaacaccgaccacacgctggaagaagtgggtaaacagttcgacgttacccgcgaacgtatccgtcagatcgaagcgaaggcgctgcgcaaactgcgtcacccgagccgttctgaagtgctgcgtagcttcctggacgattaa

SEQ ID NO: 12 - protein, 613 aa, Escherichia coli
  source 1..613: note = The sequence is a translation of SEQ ID NO. 11
MEQNPQSQLKLLVTRGKEQGYLTYAEVNDHLPEDIVDSDQIEDIIQMINDMGIQVMEEAPDADDLMLAENTADEDAAEAAAQVLSSVESEIGRTTDPVRMYMREMGTVELLTREGEIDIAKRIEDGINQVQCSVAEYPEAITYLLEQYDRVEAEEARLSDLITGFVDPNAEEDLAPTATHVGSELSQEDLDDDEDEDEEDGDDDSADDDNSIDPELAREKFAELRAQYVVTRDTIKAKGRSHATAQEEILKLSEVFKQFRLVPKQFDYLVNSMRVMMDRVRTQERLIMKLCVEQCKMPKKNFITLFTGNETSDTWFNAAIAMNKPWSEKLHDVSEEVHRALQKLQQIEEETGLTIEQVKDINRRMSIGEAKARRAKKEMVEANLRLVISIAKKYTNRGLQFLDLIQEGNIGLMKAVDKFEYRRGYKFSTYATWWIRQAITRSIADQARTIRIPVHMIETINKLNRISRQMLQEMGREPTPEELAERMLMPEDKIRKVLKIAKEPISMETPIGDDEDSHLGDFIEDTTLELPLDSATTESLRAATHDVLAGLTAREAKVLRMRFGIDMNTDHTLEEVGKQFDVTRERIRQIEAKALRKLRHPSRSEVLRSFLDD

SEQ ID NO: 13 - DNA, 546 bp, Escherichia coli
  source 1..546: mol_type = genomic DNA
  CDS 1..546: note = E. coli essential gene nusG
atgtctgaagctcctaaaaagcgctggtacgtcgttcaggcgttttccggttttgaaggccgcgtagcaacgtcgctgcgtgagcatatcaaattacacaacatggaagatttgtttggtgaagtcatggtaccaaccgaagaagtggttgaaatccgtggcggtcagcgtcgcaaaagcgaacgtaaattcttccctggctacgtcctcgttcagatggtgatgaacgacgcgagctggcacctggtgcgcagcgtaccgcgtgtgatgggcttcatcggcggtacttccgatcgtcctgcgccaatcagcgataaagaagtcgatgcgattatgaaccgcctgcagcaggttggtgataagccgcgtccgaaaacgctgtttgaaccgggtgaaatggtccgtgttaatgatggtccgttcgctgacttcaacggtgttgttgaagaagtggattacgagaaatctcgtctgaaagtgtctgtttctatcttcggtcgtgcgaccccggtagagctggacttcagccaggttgaaaaagcctaa

SEQ ID NO: 14 - protein, 181 aa, Escherichia coli
  source 1..181: note = The sequence is a translation of SEQ ID NO. 13
MSEAPKKRWYVVQAFSGFEGRVATSLREHIKLHNMEDLFGEVMVPTEEVVEIRGGQRRKSERKFFPGYVLVQMVMNDASWHLVRSVPRVMGFIGGTSDRPAPISDKEVDAIMNRLQQVGDKPRPKTLFEPGEMVRVNDGPFADFNGVVEEVDYEKSRLKVSVSIFGRATPVELDFSQVEKA

SEQ ID NO: 15 - DNA, 216 bp, Escherichia coli
  source 1..216: mol_type = genomic DNA
  CDS 1..216: note = E. coli essential gene rpsU
atgccggtaattaaagtacgtgaaaacgagccgttcgacgtagctctgcgtcgcttcaagcgttcctgcgaaaaagcaggtgttctggcggaagttcgtcgtcgtgagttctatgaaaaaccgactaccgaacgtaagcgcgctaaagcttctgcagtgaaacgtcacgcgaagaaactggctcgcgaaaacgcacgccgcactcgtctgtactaa

SEQ ID NO: 16 - protein, 71 aa, Escherichia coli
  source 1..71: note = The sequence is a translation of SEQ ID NO. 15
MPVIKVRENEPFDVALRRFKRSCEKAGVLAEVRRREFYEKPTTERKRAKASAVKRHAKKLARENARRTRLY

SEQ ID NO: 17 - DNA, 216 bp, Escherichia coli; identical to SEQ ID NO: 15
  source 1..216: mol_type = genomic DNA
  CDS 1..216: note = E. coli essential gene accD

SEQ ID NO: 18 - protein, 71 aa, Escherichia coli; identical to SEQ ID NO: 16
  source 1..71: note = The sequence is a translation of SEQ ID NO. 17

SEQ ID NO: 19 - DNA, 216 bp, Escherichia coli; identical to SEQ ID NO: 15
  source 1..216: mol_type = genomic DNA
  CDS 1..216: note = E coli essential gene degS

SEQ ID NO: 20 - protein, 71 aa, Escherichia coli; identical to SEQ ID NO: 16
  source 1..71: note = The sequence is a translation of SEQ ID NO. 19

SEQ ID NO: 21 - DNA, 531 bp, Escherichia coli
  source 1..531: mol_type = genomic DNA
  CDS 1..531: note = E. coli essential gene fldA
atggctatcactggcatctttttcggcagcgacaccggtaataccgaaaatatcgcaaaaatgattcaaaaacagcttggtaaagacgttgccgatgtccatgacattgcaaaaagcagcaaagaagatctggaagcttatgacattctgctgctgggcatcccaacctggtattacggcgaagcgcagtgtgactgggatgacttcttcccgactctcgaagagattgatttcaatggcaaactggttgcgctgtttggttgtggtgaccaggaagattacgccgaatatttctgcgacgcattgggcaccatccgcgacatcattgaaccgcgcggtgcaaccatcgttggtcactggccaactgcgggctatcatttcgaagcatcaaaaggtctggcagatgacgaccactttgtcggtctggctatcgacgaagaccgtcagccggaactgaccgctgaacgtgtagaaaaatgggttaaacagatttctgaagagttgcatctcgacgaaattctcaatgcctga

SEQ ID NO: 22 - protein, 176 aa, Escherichia coli
  source 1..176: note = The sequence is a translation of SEQ ID NO. 21
MAITGIFFGSDTGNTENIAKMIQKQLGKDVADVHDIAKSSKEDLEAYDILLLGIPTWYYGEAQCDWDDFFPTLEEIDFNGKLVALFGCGDQEDYAEYFCDALGTIRDIIEPRGATIVGHWPTAGYHFEASKGLADDDHFVGLAIDEDRQPELTAERVEKWVKQISEELHLDEILNA

SEQ ID NO: 23 - DNA, 960 bp, Escherichia coli
  source 1..960: mol_type = genomic DNA
  CDS 1..960: note = E. coli essential gene ftsN
gtggcacaacgagattatgtacgccgcagccaaccggcaccttcgcggcgaaaaaagagcacctcacggaaaaagcaacgaaatctgcctgcggtttctcccgctatggtcgctattgctgccgccgttcttgtgacctttatcggtggtctgtacttcattacgcatcacaagaaagaagagtccgagacgctgcaaagccagaaagtgaccggaaacggactaccaccaaaaccagaagaacgctggcgctacattaaagagctggaaagtcgccagccgggagtgcgtgcgcccacagaaccttctgccggtggtgaagtgaaaacgccggagcaactgacaccagaacaacgtcagcttcttgaacaaatgcaggctgatatgcgccagcagccaacgcagctggttgaagttccgtggaatgaacagacgccggaacagcgtcagcaaacgctacagcgccaacgtcaggcgcagcaattagcggaacagcaacgcctggctcagcagtcgcgtacgactgaacaaagctggcagcagcagacgcgtacgtcgcaagccgcaccggtacaggcgcaaccgcgccagtctaaaccggcttcttctcagcagccgtaccaggatctgctgcaaactcctgcgcacacgactgcgcagtcgaagccgcagcaagctgcgccagttgctcgtgccgctgacgcgccaaaaccgacggcggagaaaaaagacgaacgccgctggatggtgcagtgcggttcgttcagaggcgcggaacaggcagagacggtacgtgctcagctggcgttcgaaggctttgactcgaaaatcaccaccaacaatggctggaatcgtgtggtcattggcccggtgaaaggcaaagagaacgcagacagcaccctcaatcggttgaagatggcgggccatacaaactgcattcggctcgccgccgggggttga

SEQ ID NO: 24 - protein, 319 aa, Escherichia coli
  source 1..319: note = The sequence is a translation of SEQ ID NO. 23
VAQRDYVRRSQPAPSRRKKSTSRKKQRNLPAVSPAMVAIAAAVLVTFIGGLYFITHHKKEESETLQSQKVTGNGLPPKPEERWRYIKELESRQPGVRAPTEPSAGGEVKTPEQLTPEQRQLLEQMQADMRQQPTQLVEVPWNEQTPEQRQQTLQRQRQAQQLAEQQRLAQQSRTTEQSWQQQTRTSQAAPVQAQPRQSKPASSQQPYQDLLQTPAHTTAQSKPQQAAPVARAADAPKPTAEKKDERRWMVQCGSFRGAEQAETVRAQLAFEGFDSKITTNNGWNRVVIGPVKGKENADSTLNRLKMAGHTNCIRLAAGG

SEQ ID NO: 25 - DNA, 1935 bp, Escherichia coli
  source 1..1935: mol_type = genomic DNA
  CDS 1..1935: note = E. coli essential gene hflB
atggcgaaaaacctaatactctggctggtcattgccgttgtgctgatgtcagtattccagagctttgggcccagcgagtctaatggccgtaaggtggattactctaccttcctacaagaggtcaataacgaccaggttcgtgaagcgcgtatcaacggacgtgaaatcaacgttaccaagaaagatagtaaccgttataccacttacattccggttcaggatccgaaattactggataacctgttgaccaagaacgtcaaggttgtcggtgaaccgcctgaagaaccaagcctgctggcttctatcttcatctcctggttcccgatgctgttgctgattggtgtctggatcttcttcatgcgtcaaatgcagggcggcggtggcaaaggtgccatgtcgtttggtaagagcaaagcgcgcatgctgacggaagatcagatcaaaacgacctttgctgacgttgcgggctgcgacgaagcaaaagaagaagttgctgaactggttgagtatctgcgcgagccgagccgcttccagaaactcggcggtaagatcccgaaaggcgtcttgatggtcggtcctccgggtaccggtaaaacgctgctggcgaaagcgattgcaggcgaagcgaaagttccgttctttactatctccggttctgacttcgtagaaatgttcgtcggtgtgggtgcatcccgtgttcgtgacatgttcgaacaggcgaagaaagcggcaccgtgcatcatctttatcgatgaaatcgacgccgtaggccgccagcgtggcgctggtctgggcggtggtcacgatgaacgtgaacagactctgaaccagatgctggttgagatggatggcttcgaaggtaacgaaggtatcatcgttatcgccgcgactaaccgtccggacgttctcgacccggccctgctgcgtcctggccgtttcgaccgtcaggttgtggtcggcttgccagatgttcgcggtcgtgagcagatcctgaaagttcacatgcgtcgcgtaccattggcacccgatatcgacgcggcaatcattgcccgtggtactcctggtttctccggtgctgacctggcgaacctggtgaacgaagcggcactgttcgctgctcgtggcaacaaacgcgttgtgtcgatggttgagttcgagaaagcgaaagacaaaatcatgatgggtgcggaacgtcgctccatggtgatgacggaagcgcagaaagaatcgacggcttaccacgaagcgggtcatgcgattatcggtcgcctggtgccggaacacgatccggtgcacaaagtgacgattatcccacgcggtcgtgcgctgggtgtgactttcttcttgcctgagggcgacgcaatcagcgccagccgtcagaaactggaaagccagatttctacgctgtacggtggtcgtctggcagaagagatcatctacgggccggaacatgtatctaccggtgcgtccaacgatattaaagttgcgaccaacctggcacgtaacatggtgactcagtggggcttctctgagaaattgggtccactgctgtacgcggaagaagaaggtgaagtgttcctcggccgtagcgtagcgaaagcgaaacatatgtccgatgaaactgcacgtatcatcgaccaggaagtgaaagcactgattgagcgtaactataatcgtgcgcgtcagcttctgaccgacaatatggatattctgcatgcgatgaaagatgctctcatgaaatatgagactatcgacgcaccgcagattgatgacctgatggcacgtcgcgatgtacgtccgccagcgggctgggaagaaccaggcgcttctaacaattctggcgacaatggtagtccaaaggctcctcgtccggttgatgaaccgcgtacgccgaacccgggtaacaccatgtcagagcagttaggcgacaagtaa

SEQ ID NO: 26 - protein, 644 aa, Escherichia coli
  source 1..644: note = The sequence is a translation of SEQ ID NO. 25
MAKNLILWLVIAVVLMSVFQSFGPSESNGRKVDYSTFLQEVNNDQVREARINGREINVTKKDSNRYTTYIPVQDPKLLDNLLTKNVKVVGEPPEEPSLLASIFISWFPMLLLIGVWIFFMRQMQGGGGKGAMSFGKSKARMLTEDQIKTTFADVAGCDEAKEEVAELVEYLREPSRFQKLGGKIPKGVLMVGPPGTGKTLLAKAIAGEAKVPFFTISGSDFVEMFVGVGASRVRDMFEQAKKAAPCIIFIDEIDAVGRQRGAGLGGGHDEREQTLNQMLVEMDGFEGNEGIIVIAATNRPDVLDPALLRPGRFDRQVVVGLPDVRGREQILKVHMRRVPLAPDIDAAIIARGTPGFSGADLANLVNEAALFAARGNKRVVSMVEFEKAKDKIMMGAERRSMVMTEAQKESTAYHEAGHAIIGRLVPEHDPVHKVTIIPRGRALGVTFFLPEGDAISASRQKLESQISTLYGGRLAEEIIYGPEHVSTGASNDIKVATNLARNMVTQWGFSEKLGPLLYAEEEGEVFLGRSVAKAKHMSDETARIIDQEVKALIERNYNRARQLLTDNMDILHAMKDALMKYETIDAPQIDDLMARRDVRPPAGWEEPGASNNSGDNGSPKAPRPVDEPRTPNPGNTMSEQLGDK

SEQ ID NO: 27 - DNA, 612 bp, Escherichia coli
  source 1..612: mol_type = genomic DNA
  CDS 1..612: note = E. coli essential gene lolA
atgaaaaaaattgccatcacctgtgcattactctcaagcttagtagcaagcagcgtttgggctgatgccgcaagcgatctgaaaagccgcctggataaagtcagcagcttccacgccagcttcacacaaaaagtgactgacggtagcggcgcggcggtgcaggaaggtcagggcgatctgtgggtgaaacgtccaaacttattcaactggcatatgacacaacctgatgaaagcattctggtttctgacggtaaaacactgtggttctataacccgttcgttgagcaagctacggcaacctggctgaaagatgccaccggtaatacgccgtttatgctgattgcccgcaaccagtccagcgactggcagcagtacaatatcaaacagaatggcgatgactttgtcctgacgccgaaagccagcaatggcaatctgaagcagttcaccattaacgtgggacgtgatggcacaatccatcagtttagcgcggtggagcaggacgatcagcgcagcagttatcaactgaaatcccagcaaaatggggctgtggatgcagcgaaatttaccttcaccccgccgcaaggcgtcacggtagatgatcaacgtaagtag

SEQ ID NO: 28 - protein, 203 aa, Escherichia coli
  source 1..203: note = The sequence is a translation of SEQ ID NO. 27
MKKIAITCALLSSLVASSVWADAASDLKSRLDKVSSFHASFTQKVTDGSGAAVQEGQGDLWVKRPNLFNWHMTQPDESILVSDGKTLWFYNPFVEQATATWLKDATGNTPFMLIARNQSSDWQQYNIKQNGDDFVLTPKASNGNLKQFTINVGRDGTIHQFSAVEQDDQRSSYQLKSQQNGAVDAAKFTFTPPQGVTVDDQRK

SEQ ID NO: 29 - DNA, 1083 bp, Escherichia coli
  source 1..1083: mol_type = genomic DNA
  CDS 1..1083: note = E. coli essential gene mraY
atgttagtttggctggccgaacatttggtcaaatattattccggctttaacgtcttttcctatctgacgtttcgcgccatcgtcagcctgctgaccgcgctgttcatctcattgtggatgggcccgcgtatgattgctcatttgcaaaaactttcctttggtcaggtggtgcgtaacgacggtcctgaatcacacttcagcaagcgcggtacgccgaccatgggcgggattatgatcctgacggcgattgtgatctccgtactgctgtgggcttacccgtccaatccgtacgtctggtgcgtgttggtggtgctggtaggttacggtgttattggctttgttgatgattatcgcaaagtggtgcgtaaagacaccaaagggttgatcgctcgttggaagtatttctggatgtcggtcattgcgctgggtgtcgccttcgccctgtaccttgccggcaaagacacgcccgcaacgcagctggtggtcccattctttaaagatgtgatgccgcagctggggctgttctacattctgctggcttacttcgtcattgtgggtactggcaacgcggtaaacctgaccgatggtctcgacggcctggcaattatgccgaccgtatttgtcgccggtggttttgcgctggtggcgtgggcgaccggcaatatgaactttgccagctacttgcatataccgtatctgcgacacgccggggaactggttattgtctgtaccgcgatagtcggggcaggactgggcttcctgtggtttaacacctatccggcgcaggtctttatgggcgatgtaggttcgctggcgttaggtggtgcgttaggcattatcgccgtactgctacgtcaggaattcctgctggtgattatggggggcgtgttcgtggtagaaacgctttctgtcatcctgcaggtcggctcctttaaactgcgcggacaacgtattttccgcatggcaccgattcatcaccactatgaactgaaaggctggccggaaccgcgcgtcattgtgcgtttctggattatttcgctgatgctggttctgattggtctggcaacgctgaaggtacgttaa

SEQ ID NO: 30 - protein, 360 aa, Escherichia coli
  source 1..360: note = The sequence is a translation of SEQ ID NO. 29
MLVWLAEHLVKYYSGFNVFSYLTFRAIVSLLTALFISLWMGPRMIAHLQKLSFGQVVRNDGPESHFSKRGTPTMGGIMILTAIVISVLLWAYPSNPYVWCVLVVLVGYGVIGFVDDYRKVVRKDTKGLIARWKYFWMSVIALGVAFALYLAGKDTPATQLVVPFFKDVMPQLGLFYILLAYFVIVGTGNAVNLTDGLDGLAIMPTVFVAGGFALVAWATGNMNFASYLHIPYLRHAGELVIVCTAIVGAGLGFLWFNTYPAQVFMGDVGSLALGGALGIIAVLLRQEFLLVIMGGVFVVETLSVILQVGSFKLRGQRIFRMAPIHHHYELKGWPEPRVIVRFWIISLMLVLIGLATLKVR

SEQ ID NO: 31 - DNA, 489 bp, Escherichia coli
  source 1..489: mol_type = genomic DNA
  CDS 1..489: note = E. coli essential gene mreD
gtggcgagctatcgtagccagggacgctgggtaatctggctctctttcctcattgcgctgttgctgcaaatcatgccctggccggataacctgattgttttccggccaaactgggtgttactcatcttgttgtattggatcctggccttgcctcatcgcgtaaatgtgggcacaggttttgtgatgggtgccatactggatctgatcagcggctcgacgcttggcgtacgcgtattggcgatgagcatcattgcttacctggtggcgctgaaataccagcttttccgcaacctcgcattatggcagcaggcgctggtcgtcatgttgctttcgctggtggtggatattattgttttctgggcagagtttttagtgattaacgtctctttcagaccggaagtgttctggagtagtgtagtcaatggggtgctctggccgtggattttcttgctgatgcgcaaagtccgtcagcagtttgcagtgcaataa

SEQ ID NO: 32 - protein, 162 aa, Escherichia coli
  source 1..162: note = The sequence is a translation of SEQ ID NO. 31
VASYRSQGRWVIWLSFLIALLLQIMPWPDNLIVFRPNWVLLILLYWILALPHRVNVGTGFVMGAILDLISGSTLGVRVLAMSIIAYLVALKYQLFRNLALWQQALVVMLLSLVVDIIVFWAEFLVINVSFRPEVFWSSVVNGVLWPWIFLLMRKVRQQFAVQ

SEQ ID NO: 33 - DNA, 1260 bp, Escherichia coli
  source 1..1260: mol_type = genomic DNA
  CDS 1..1260: note = E. coli essential gene murA
atggataaatttcgtgttcaggggccaacgaagctccagggcgaagtcacaatttccggcgctaaaaatgctgctctgcctatcctttttgccgctctactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcactggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtctgaaaggcgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgtgctgccggatcgtatcgaaaccggtacattcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggactggattcatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgagtaa

SEQ ID NO: 34 - protein, 419 aa, Escherichia coli
  source 1..419: note = The sequence is a translation of SEQ ID NO. 33
MDKFRVQGPTKLQGEVTISGAKNAALPILFAALLAEEPVEIQNVPKLKDVDTSMKLLSQLGAKVERNGSVHIDARDVNVFCAPYDLVKTMRASIWALGPLVARFGQGQVSLPGGCTIGARPVDLHISGLEQLGATIKLEEGYVKASVDGRLKGAHIVMDKVSVGATVTIMCAATLAEGTTIIENAAREPEIVDTANFLITLGAKISGQGTDRIVIEGVERLGGGVYRVLPDRIETGTFLVAAAISRGKIICRNAQPDTLDAVLAKLRDAGADIEVGEDWISLDMHGKRPKAVNVRTAPHPAFPTDMQAQFTLLNLVAEGTGFITETVFENRFMHVPELSRMGAHAEIESNTVICHGVEKLSGAQVMATDLRASASLVLAGCIAEGTTVVDRIYHIDRGYERIEDKLRALGANIERVKGE

SEQ ID NO: 35 - DNA, 1029 bp, Escherichia coli
  source 1..1029: mol_type = genomic DNA
  CDS 1..1029: note = E. coli essential gene murB
atgaaccactccttaaaaccctggaacacatttggcattgatcataatgctcagcacattgtatgtgccgaagacgaacaacaattactcaatgcctggcagcatgcaaccgcagaaggacaacccgttcttattctgggtgaaggaagtaatgtactttttctggaagactatcgcggcacggtgatcatcaaccggatcaaaggtatcgaaattcatgatgaacctgatgcgtggtatttacatgtaggagccggagaaaactggcatcgcctggtaaaatacactttgcaggaaggtatgcctggtctggaaaatctggcattaattcctggttgtgtcggctcatcacctatccagaatattggtgcttatggcgtagaattacagcgagtttgcgcttatgttgattgtgttgaactggcgacaggcaagcaagtgcgcttaactgccaaagagtgccgttttggctatcgcgacagtatttttaaacatgaataccaggatcgcttcgccattgtagccgtaggtctgcgtctgccaaaagagtggcaacctgtactaacgtatggtgacttaactcgtctggatcctacaacagtaacgccacagcaagtatttaatgcggtgtgtcatatgcgcaccaccaaactccctgatccaaaagtgaatggcaatgccggtagtttcttcaaaaaccctgttgtatctgccgaaacggctgaagcattactgtcacaatttccaacagcaccaaattacccccaggcggatggttcagtaaaactggcagcaggttggcttattgatcagtgccagctaaaagggatgcaaatgggtggggctgcggtgcaccgtcaacaggcgttagttctcattaatgaagacaatgcaaaaagcgaagatgtggtgcaactggcacaccatgtaagacaaaaagtgggtgaaaaatttaatgtctggcttgagcctgaagtccgctttattggtgcatcaggtgaagtgagcgcagtggagacaatttcatga

SEQ ID NO: 36 - protein, 342 aa, Escherichia coli
  source 1..342: note = The sequence is a translation of SEQ ID NO. 35
MNHSLKPWNTFGIDHNAQHIVCAEDEQQLLNAWQHATAEGQPVLILGEGSNVLFLEDYRGTVIINRIKGIEIHDEPDAWYLHVGAGENWHRLVKYTLQEGMPGLENLALIPGCVGSSPIQNIGAYGVELQRVCAYVDCVELATGKQVRLTAKECRFGYRDSIFKHEYQDRFAIVAVGLRLPKEWQPVLTYGDLTRLDPTTVTPQQVFNAVCHMRTTKLPDPKVNGNAGSFFKNPVVSAETAEALLSQFPTAPNYPQADGSVKLAAGWLIDQCQLKGMQMGGAAVHRQQALVLINEDNAKSEDVVQLAHHVRQKVGEKFNVWLEPEVRFIGASGEVSAVETIS

SEQ ID NO: 37 - DNA, 1359 bp, Escherichia coli
  source 1..1359: mol_type = genomic DNA
  CDS 1..1359: note = E. coli essential gene murF
atgattagcgtaacccttagccaacttaccgacattctcaacggtgaactgcaaggtgcagatatcacccttgatgctgtaaccactgatacccgaaaactgacgccgggctgcctgtttgttgccctgaaaggcgaacgttttgatgcccacgattttgccgaccaggcgaaagctggcggcgcaggcgcactactggttagccgtccgctggacatcgacctgccgcagttaatcgtcaaggatacgcgtctggcgtttggtgaactggctgcatgggttcgccagcaagttccggcgcgcgtggttgctctgacggggtcctccggcaaaacctccgttaaagagatgacggcggcgattttaagccagtgcggcaacacgctttatacggcaggcaatctcaacaacgacatcggtgtaccgatgacgctgttgcgcttaacgccggaatacgattacgcagttattgaacttggcgcgaaccatcagggcgaaatagcctggactgtgagtctgactcgcccggaagctgcgctggtcaacaacctggcagcggcgcatctggaaggttttggctcgcttgcgggtgtcgcgaaagcgaaaggtgaaatctttagcggcctgccggaaaacggtatcgccattatgaacgccgacaacaacgactggctgaactggcagagcgtaattggctcacgcaaagtgtggcgtttctcacccaatgccgccaacagcgatttcaccgccaccaatatccatgtgacctcgcacggtacggaatttaccctacaaaccccaaccggtagcgtcgatgttctgctgccgttgccggggcgtcacaatattgcgaatgcgctggcagccgctgcgctctccatgtccgtgggcgcaacgcttgatgctatcaaagcggggctggcaaatctgaaagctgttccaggccgtctgttccccatccaactggcagaaaaccagttgctgctcgacgactcctacaacgccaatgtcggttcaatgactgcagcagtccaggtactggctgaaatgccgggctaccgcgtgctggtggtgggcgatatggcggaactgggcgctgaaagcgaagcctgccatgtacaggtgggcgaggcggcaaaagctgctggtattgaccgcgtgttaagcgtgggtaaacaaagccatgctatcagcaccgccagcggcgttggcgaacattttgctgataaaactgcgttaattacgcgtcttaaatcactgattgctgagcaacaggtaattacgattttagttaagggttcacgtagtgccgccatggaagaggtagtacgcgctttacaggagaatgggacatgttag

SEQ ID NO: 38 - protein, 452 aa, Escherichia coli
  source 1..452: note = The sequence is a translation of SEQ ID NO. 37
MISVTLSQLTDILNGELQGADITLDAVTTDTRKLTPGCLFVALKGERFDAHDFADQAKAGGAGALLVSRPLDIDLPQLIVKDTRLAFGELAAWVRQQVPARVVALTGSSGKTSVKEMTAAILSQCGNTLYTAGNLNNDIGVPMTLLRLTPEYDYAVIELGANHQGEIAWTVSLTRPEAALVNNLAAAHLEGFGSLAGVAKAKGEIFSGLPENGIAIMNADNNDWLNWQSVIGSRKVWRFSPNAANSDFTATNIHVTSHGTEFTLQTPTGSVDVLLPLPGRHNIANALAAAALSMSVGATLDAIKAGLANLKAVPGRLFPIQLAENQLLLDDSYNANVGSMTAAVQVLAEMPGYRVLVVGDMAELGAESEACHVQVGEAAKAAGIDRVLSVGKQSHAISTASGVGEHFADKTALITRLKSLIAEQQVITILVKGSRSAAMEEVVRALQENGTC

SEQ ID NO: 39 - DNA, 642 bp, Escherichia coli
  source 1..642: mol_type = genomic DNA
  CDS 1..642: note = E. coli essential gene nadD
atgaaatctttacaggctctgtttggcggcacctttgatccggtgcactatggtcatctaaaacccgtggaaacgctggcgaatttgattggtctgacgcgggtcacaatcatccctaataatgttcctccgcatcgtccccagccggaagcgaacagcgtgcagcgtaaacacatgcttgaactggcgattgccgacaagccattatttactcttgatgaacgcgagctaaagcgcaatgccccctcttacactgcgcaaacactgaaagagtggcggcaggaacaaggaccggacgtgccgctggcgtttattattggtcaggattcattgctgacctttccgacctggtacgaatacgaaacgatactcgacaatgcacatttgatcgtctgtcggcgtccaggttacccacttgaaatggcgcaaccgcaataccagcaatggctggaagatcatttgacacataacccggaagatcttcaccttcagcctgccggtaaaatttatctggctgaaacgccgtggtttaacatctcggcgaccatcatccgcgaacgtttgcaaaacggtgaatcgtgtgaggatttattgccggaaccggtattgacttacattaaccaacaaggcttgtatcgctga

SEQ ID NO: 40 - protein, 213 aa, Escherichia coli
  source 1..213: note = The sequence is a translation of SEQ ID NO. 39
MKSLQALFGGTFDPVHYGHLKPVETLANLIGLTRVTIIPNNVPPHRPQPEANSVQRKHMLELAIADKPLFTLDERELKRNAPSYTAQTLKEWRQEQGPDVPLAFIIGQDSLLTFPTWYEYETILDNAHLIVCRRPGYPLEMAQPQYQQWLEDHLTHNPEDLHLQPAGKIYLAETPWFNISATIIRERLQNGESCEDLLPEPVLTYINQQGLYR

SEQ ID NO: 41 - DNA, 333 bp, Escherichia coli
  source 1..333: mol_type = genomic DNA
  CDS 1..333: note = E. coli essential gene rplV
atggaaactatcgctaaacatcgccatgctcgttcttctgctcagaaggttcgccttgttgctgacctgattcgcggtaagaaagtgtcgcaggctctggatattttgacctacaccaacaagaaagcggctgtactggtcaagaaagttctggaatctgccattgctaacgctgaacacaacgatggcgctgacattgacgatctgaaagttacgaaaattttcgtagacgaaggcccgagcatgaagcgcattatgccgcgtgcaaaaggtcgtgcagatcgcatcctgaagcgcaccagccacatcactgtggttgtgtccgatcgctga

SEQ ID NO: 42 - protein, 110 aa, Escherichia coli [sequence omitted]

SEQ ID NO: 43 - DNA, 471 bp, Escherichia coli
  source 1..471: mol_type = genomic DNA
  CDS 1..471: note = E. coli essential gene rpsG
atgccacgtcgtcgcgtcattggtcagcgtaaaattctgccggatccgaagttcggatcagaactgctggctaaatttgtaaatatcctgatggtagatggtaaaaaatctactgctgaatctatcgtatacagcgcgctggagaccctggctcagcgctctggtaaatctgaactggaagcattcgaagtagctctcgaaaacgtgcgcccgactgtagaagttaagtctcgccgcgttggtggttctacttatcaggtaccagttgaagtccgtccggttcgtcgtaatgctctggcaatgcgttggatcgttgaagctgctcgtaaacgcggtgataaatccatggctctgcgcctggcgaacgaactttctgatgctgcagaaaacaaaggtactgcagttaagaaacgtgaagacgttcaccgtatggccgaagccaacaaggcgttcgcacactaccgttggtaa

SEQ ID NO: 44 - protein, 156 aa, Escherichia coli
  source 1..156: note = The sequence is a translation of SEQ ID NO. 43
MPRRRVIGQRKILPDPKFGSELLAKFVNILMVDGKKSTAESIVYSALETLAQRSGKSELEAFEVALENVRPTVEVKSRRVGGSTYQVPVEVRPVRRNALAMRWIVEAARKRGDKSMALRLANELSDAAENKGTAVKKREDVHRMAEANKAFAHYRW

SEQ ID NO: 45 - DNA, 2475 bp, Saccharomyces cerevisiae
  source 1..2475: mol_type = genomic DNA
  CDS 1..2475: note = S. cerevisiae essential gene FOL1 YNL256W SGDID:S000005200
atgtcaaagctattttctactgtcaattctgcaagacatagtgtaccactaggcggcatgagagattatgtgcacattaagaaactagagatgaatacagttcttgggcctgattcctggaatcaattaatgcctcagaaatgtctactaagcttagatatgggtacagattttagtaaatctgcggctacggatgatttgaaatattctctaaattatgcagttatttctcgtgatttgacgaatttcgtcagcaaaaaaaagaattggggttctgtttctaatttggctaaatctgtgtctcaatttgttatggacaaatattctggtgtcgagtgtctgaatttagaagtgcaggcggatacaacgcatattagaagtgaccacatatcttgtattattcaacaagaaagagggaatccagaatcacaggaatttgacgttgttaggatatctgagttaaaaatgttgactttgattggtgttttcacctttgagagacttaagaaacagtatgtaactttggatataaagttgccttggccaaagaaagccgaattgccaccgccagtgcaaagcataattgataacgttgtcaagtttgtggaggaatcaaatttcaagactgtggaagctcttgtagaatctgtgtcagctgttattgcccataacgagtattttcaaaagtttccagattcgcctttggtggtgaaggttttgaaattaaacgcaatcacagccacagaaggtgttggtgtaagctgtattagagagcccagggagattgcgatggtaaatattccatatctttcctccatacatgaatcgtctgatattaagttccaattgtcttcatcacaaaacactcctattgagggtaaaaatacatggaaaagagcgtttttagcgtttggttcaaacattggggaccgtttcaaacacattcaaatggcgttgcaattattatcaagggaaaaaacggttaaattacggaatatttcgtctatttttgaaagtgaaccaatgtatttcaaagatcaaacccctttcatgaatgggtgtgttgaggtggagacattactgaccccaagcgaattattaaaattgtgtaaaaaaattgaatatgaagagttgcaaagagtcaagcattttgataatggtccgagaacaatagatctggatattgttatgtttttgaatagcgccggagaagatattatagtaaatgaaccggatttgaatataccgcatcctagaatgctggagaggactttcgttcttgagccgttatgtgaattaatatcccccgttcaccttcatcctgtgacagcggaacccattgtagaccatttaaaacagttatacgacaaacagcatgatgaagataccttatggaaattagttccattgccttatcgtagtggtgtggagcctagatttttgaaattcaagaccgctacaaaacttgacgaatttactggagaaacaaacagaattactgtttcacctacatatatcatggctatcttcaacgctacaccagattcattttccgatggaggtgagcattttgcggacattgaaagtcaattgaatgatatcattaaattgtgtaaagacgcattatatttgcatgagagcgtcatcatcgacgttggagggtgttctaccaggcctaactctattcaggcgtctgaggaagaagaaatacgcaggtctatcccattaattaaggccattagagaaagcactgagttaccgcaagataaagtcatactatccattgatacttatcgttccaatgtcgctaaagaagcgattaaagttggagtggatattattaatgatatttcgggaggtttatttgacagcaacatgtttgccgtaattgcagagaacccagaaatttgttatattttatcacacacacgtggtgatatttcaacgatgaataggctggcgcattacgaaaattttgcattgggtgattctattcagcaagaatttgttcataataccgacattcagcagctagacgacttgaaagacaaaacagtgttaatcaggaatgttggtcaagaaattggcgaaaggtatatcaaagcgattgataatggagtaaagcgctggcaaattctaatcgaccctggacttggttttgctaagacctggaagcaaaacttacaaattattagacatatccccattttaaagaactactcattcaccatgaactcaaacaattcgcaagtgtatgttaacctcagaaatatgcccgttttattgggtccatcgcgcaaaaaattcattggacatatcacaaaagatgtggatgcgaagcaaagagactttgctactggagcggtggtagcgtcgtgtattggtttcggcagcgacatggttagggtccatgacgttaaaaattgttcgaagagcattaaattagcagatgctatttataaaggtttggaataa

SEQ ID NO: 46 - protein, 824 aa, Saccharomyces cerevisiae
  source 1..824: note = The sequence is a translation of SEQ ID NO. 45
MSKLFSTVNSARHSVPLGGMRDYVHIKKLEMNTVLGPDSWNQLMPQKCLLSLDMGTDFSKSAATDDLKYSLNYAVISRDLTNFVSKKKNWGSVSNLAKSVSQFVMDKYSGVECLNLEVQADTTHIRSDHISCIIQQERGNPESQEFDVVRISELKMLTLIGVFTFERLKKQYVTLDIKLPWPKKAELPPPVQSIIDNVVKFVEESNFKTVEALVESVSAVIAHNEYFQKFPDSPLVVKVLKLNAITATEGVGVSCIREPREIAMVNIPYLSSIHESSDIKFQLSSSQNTPIEGKNTWKRAFLAFGSNIGDRFKHIQMALQLLSREKTVKLRNISSIFESEPMYFKDQTPFMNGCVEVETLLTPSELLKLCKKIEYEELQRVKHFDNGPRTIDLDIVMFLNSAGEDIIVNEPDLNIPHPRMLERTFVLEPLCELISPVHLHPVTAEPIVDHLKQLYDKQHDEDTLWKLVPLPYRSGVEPRFLKFKTATKLDEFTGETNRITVSPTYIMAIFNATPDSFSDGGEHFADIESQLNDIIKLCKDALYLHESVIIDVGGCSTRPNSIQASEEEEIRRSIPLIKAIRESTELPQDKVILSIDTYRSNVAKEAIKVGVDIINDISGGLFDSNMFAVIAENPEICYILSHTRGDISTMNRLAHYENFALGDSIQQEFVHNTDIQQLDDLKDKTVLIRNVGQEIGERYIKAIDNGVKRWQILIDPGLGFAKTWKQNLQIIRHIPILKNYSFTMNSNNSQVYVNLRNMPVLLGPSRKKFIGHITKDVDAKQRDFATGAVVASCIGFGSDMVRVHDVKNCSKSIKLADAIYKGLE

SEQ ID NO: 47 - DNA, 669 bp, Saccharomyces cerevisiae
  source 1..669: mol_type = genomic DNA
  CDS 1..669: note = S. cerevisiae essential gene MED7 YOL135C SGDID:S000005495
atgtccaatgatcctggtaatgaggttagctccttgtacccacctccacccccatacgtgaagtttttcacacagtccaatctggaaaaattaccaaaatacaaagaaaagaaggccgcaagtgcgaagcagacggctccaaataacagtaacggcggcagtgaagaggagattacatgtgcgctagattacctaatcccaccccccatgcccaaaaatcagcagtaccgagcgtttgggagtatatggcaggtgaaggaccaactgcccgatttagagagcatgggactcacccaactttataagaaatccacagaaaacgaaagcacaaattatcaatacaaaatccaagagctacgcaaactattgaaatccctcctgctaaattacttggaactcatcggcgtattgagcataaatccggatatgtatgagcgcaaagtggagaacataaggaccattcttgtcaatattcaccacctacttaatgagtacagaccccatcagtcccgagagtcccttattatgctattggaagaacaattagaatacaagcgcggggaaattcgcgagatcgagcaagtatgcaaacaggtacacgacaaactcaccagcatacaggacaccttaagaaccggatcacaatctcctccatcgtcgtcccaatga

SEQ ID NO: 48 - protein, 222 aa, Saccharomyces cerevisiae
  source 1..222: note = The sequence is a translation of SEQ ID NO. 47
MSNDPGNEVSSLYPPPPPYVKFFTQSNLEKLPKYKEKKAASAKQTAPNNSNGGSEEEITCALDYLIPPPMPKNQQYRAFGSIWQVKDQLPDLESMGLTQLYKKSTENESTNYQYKIQELRKLLKSLLLNYLELIGVLSINPDMYERKVENIRTILVNIHHLLNEYRPHQSRESLIMLLEEQLEYKRGEIREIEQVCKQVHDKLTSIQDTLRTGSQSPPSSSQ

SEQ ID NO: 49 - DNA, 723 bp, Saccharomyces cerevisiae
  source 1..723: mol_type = genomic DNA
  CDS 1..723: note = S. cerevisiae essential gene RRP40 YOL142W SGDID:S000005502
atgtctacgttcatattccctggtgatagctttcctgtagaccctactacacctgttaaactgggccctggcatatattgtgaccccaatactcaagaaatacgacctgttaatacaggtgttttgcatgtttccgctaagggtaagagtggtgttcagaccgcatatatagactattctagtaagagatacattccatctgtaaacgattttgtaatcggtgtcattatagggacattttcagatagctataaggtttcgttgcaaaatttctcctccagtgtttcactatcgtatatggcttttcccaatgcttcaaagaaaaacaggccaactttgcaagtgggagatctagtgtatgcgagagtttgtaccgcagaaaaggaactagaagccgaaatagaatgttttgactcaactacaggacgcgatgctggtttcgggatattggaagatggtatgatcattgacgtgaatttgaatttcgcacgccagttgcttttcaataatgacttcccgttattaaaagtgttggctgcacataccaagtttgaagtcgccattggtctcaatgggaagatctgggttaagtgcgaggaattatctaacactttagcttgttatagaaccataatggagtgttgtcaaaaaaacgacacggcagcgttcaaggatatagcaaaaagacagtttaaagaaatacttacggtcaaggaggagtag

SEQ ID NO: 50 - protein, 240 aa, Saccharomyces cerevisiae
  source 1..240: note = The sequence is a translation of SEQ ID NO. 49
MSTFIFPGDSFPVDPTTPVKLGPGIYCDPNTQEIRPVNTGVLHVSAKGKSGVQTAYIDYSSKRYIPSVNDFVIGVIIGTFSDSYKVSLQNFSSSVSLSYMAFPNASKKNRPTLQVGDLVYARVCTAEKELEAEIECFDSTTGRDAGFGILEDGMIIDVNLNFARQLLFNNDFPLLKVLAAHTKFEVAIGLNGKIWVKCEELSNTLACYRTIMECCQKNDTAAFKDIAKRQFKEILTVKEE

SEQ ID NO: 51 - DNA, 1455 bp, Saccharomyces cerevisiae
  source 1..1455: mol_type = genomic DNA
  CDS 1..1455: note = S. cerevisiae essential gene NOP8 YOL144W SGDID:S000005504
atggatagtgtaattcaaaaaagaatttttgtcggaaatattttccataatgcagatgattgttattcagaactactagatcgatttggaaaatttggtgattgccaagattttcagtttgaaaaacacaaccactttgcatttatcgatataaggtttaatgatgaggcagattttaacaagctaagaaagagctttaataatgttaagtttaaagggaacattttgaaggttgatgaagcaaagccgaattgggaaagcacctgggcggtacaacatgcaaaggaccttaaagaagatattatactaaatgctaaaatgaaaaaaaagaattggcaacattataagaaaatggaaaatgtagcgaaaagttggaaagatcataaggaagttattgctggcagaatgagggaggcgccgagaaaaaggagtcaattgaggaacattacattcaggatcaatgtcaatggttcattgaaagtttacaaatgctataagactaaattatggggttatgaaaggaacaaagaattgaatgaccttgtgtacaagtttacaaataatttctggaaaaacggatacaaccacattgtcgatagattggattacagtcgtgctgttaaaactgttcgattcaaaaatgggctgaaacagttaacagtgtcaaaagatgaaaacgtttgcagtggagagatggacagcgatgagaatatgtcagaagaggagaaggagaaaaataatgttattcttaatgacctattaaaggactttgacttcgataagccaatgacgttgaatgattcggacgaggaacttctgacagagcaacgtaaaggagaggaagaagaagaagaagaagaagaaaaagaagtaaacgctcccgaatacgagaatgttaataagacgaaggaccagagcaccctaccacaagaaaaaccagaggaaaggaaagaacaggacgagggcgacgggcaggaggataatgaatttattccgactttcaccaaggaaattggtcaaggtacaataagcaacactgagacgttaagaaacctattcaaccccaacgaggcagaacctgtatcacaattcaaattgattgaagattctgataatgatattgatcatgcgaaagatgttgatgtgaaccaactggaggaagaagtaagtaaatcatctgacaccctaggtttgacatcggctcccgtaccacatgtatctagagataaagataacaaaaatttcttgtttttccctcatttgcaatcgccatttttagtaggacaaacacagttgagtaaagtaagggctcctggaagagaaacaatgctatcgaactgggatgaggagttctgggctaacagaggtaattggacccgtgatatgaggcgcaaaatgaaagatgcattgaagcaccgtaagaggaaacaatcaaagagcgggcttcttctatag

SEQ ID NO: 52 - protein, 484 aa, Saccharomyces cerevisiae
  source 1..484: note = The sequence is a translation of SEQ ID NO. 51
MDSVIQKRIFVGNIFHNADDCYSELLDRFGKFGDCQDFQFEKHNHFAFIDIRFNDEADFNKLRKSFNNVKFKGNILKVDEAKPNWESTWAVQHAKDLKEDIILNAKMKKKNWQHYKKMENVAKSWKDHKEVIAGRMREAPRKRSQLRNITFRINVNGSLKVYKCYKTKLWGYERNKELNDLVYKFTNNFWKNGYNHIVDRLDYSRAVKTVRFKNGLKQLTVSKDENVCSGEMDSDENMSEEEKEKNNVILNDLLKDFDFDKPMTLNDSDEELLTEQRKGEEEEEEEEEKEVNAPEYENVNKTKDQSTLPQEKPEERKEQDEGDGQEDNEFIPTFTKEIGQGTISNTETLRNLFNPNEAEPVSQFKLIEDSDNDIDHAKDVDVNQLEEEVSKSSDTLGLTSAPVPHVSRDKDNKNFLFFPHLQSPFLVGQTQLSKVRAPGRETMLSNWDEEFWANRGNWTRDMRRKMKDALKHRKRKQSKSGLLL

SEQ ID NO: 53 - DNA, 1665 bp, Saccharomyces cerevisiae
  source 1..1665: mol_type = genomic DNA
  CDS 1..1665: note = S. cerevisiae essential gene PGI1 YBR196C SGDID:S000000400
atgtccaataactcattcactaacttcaaactggccactgaattgccagcctggtctaagttgcaaaaaatttatgaatctcaaggtaagactttgtctgtcaagcaagaattccaaaaagatgccaagcgttttgaaaaattgaacaagactttcaccaactatgatggttccaaaatcttgttcgactactcaaagaacttggtcaacgatgaaatcattgctgcattgattgaactggccaaggaggctaacgtcaccggtttgagagatgctatgttcaaaggtgaacacatcaactccactgaagatcgtgctgtctaccacgtcgcattgagaaacagagctaacaagccaatgtacgttgatggtgtcaacgttgctccagaagtcgactctgtcttgaagcacatgaaggagttctctgaacaagttcgttctggtgaatggaagggttataccggtaagaagatcaccgatgttgttaacatcggtattggtggttccgatttgggtccagtcatggtcactgaggctttgaagcactacgctggtgtcttggatgtccacttcgtttccaacattgacggtactcacattgctgaaaccttgaaggttgttgacccagaaactactttgtttttgattgcttccaagactttcactaccgctgaaactatcactaacgctaacactgccaagaactggttcttgtcgaagacaggtaatgatccatctcacattgctaagcatttcgctgctttgtccactaacgaaaccgaagttgccaagttcggtattgacaccaaaaacatgtttggtttcgaaagttgggtcggtggtcgttactctgtctggtcggctattggtttgtctgttgccttgtacattggctatgacaactttgaggctttcttgaagggtgctgaagccgtcgacaaccacttcacccaaaccccattggaagacaacattccattgttgggtggtttgttgtctgtctggtacaacaacttctttggtgctcaaacccatttggttgctccattcgaccaatacttgcacagattcccagcctacttgcaacaattgtcaatggaatctaacggtaagtctgttaccagaggtaacgtgtttactgactactctactggttctatcttgtttggtgaaccagctaccaacgctcaacactctttcttccaattggttcaccaaggtaccaagttgattccatctgatttcatcttagctgctcaatctcataacccaattgagaacaaattacatcaaaagatgttggcttcaaacttctttgctcaagctgaagctttaatggttggtaaggatgaagaacaagttaaggctgaaggtgccactggtggtttggtcccacacaaggtcttctcaggtaacagaccaactacctctatcttggctcaaaagattactccagctactttgggtgctttgattgcctactacgaacatgttactttcactgaaggtgccatttggaatatcaactctttcgaccaatggggtgttgaattgggtaaagtcttggctaaagtcatcggcaaggaattggacaactcctccaccatttctacccacgatgcttctaccaacggtttaatcaatcaattcaaggaatggatgtga

SEQ ID NO: 54 - protein, 554 aa, Saccharomyces cerevisiae
  source 1..554: note = The sequence is a translation of SEQ ID NO. 53
MSNNSFTNFKLATELPAWSKLQKIYESQGKTLSVKQEFQKDAKRFEKLNKTFTNYDGSKILFDYSKNLVNDEIIAALIELAKEANVTGLRDAMFKGEHINSTEDRAVYHVALRNRANKPMYVDGVNVAPEVDSVLKHMKEFSEQVRSGEWKGYTGKKITDVVNIGIGGSDLGPVMVTEALKHYAGVLDVHFVSNIDGTHIAETLKVVDPETTLFLIASKTFTTAETITNANTAKNWFLSKTGNDPSHIAKHFAALSTNETEVAKFGIDTKNMFGFESWVGGRYSVWSAIGLSVALYIGYDNFEAFLKGAEAVDNHFTQTPLEDNIPLLGGLLSVWYNNFFGAQTHLVAPFDQYLHRFPAYLQQLSMESNGKSVTRGNVFTDYSTGSILFGEPATNAQHSFFQLVHQGTKLIPSDFILAAQSHNPIENKLHQKMLASNFFAQAEALMVGKDEEQVKAEGATGGLVPHKVFSGNRPTTSILAQKITPATLGALIAYYEHVTFTEGAIWNINSFDQWGVELGKVLAKVIGKELDNSSTISTHDASTNGLINQFKEWM

SEQ ID NO: 55 - DNA, 759 bp, Saccharomyces cerevisiae
  source 1..759: mol_type = genomic DNA
  CDS 1..759: note = S. cerevisiae essential gene NEP1 EMG1 YLR186W SGDID:S000004176
atggtcgaagattccagagttagagacgccctcaaaggtggtgatcagaaggcattaccggcctctttggttcctcaagcacctcctgtcttgacatcaaaggataagattactaagcggatgattgtggtattagcgatggcatccctcgagacacacaagatatcgtccaacgggcctggtggtgacaaatatgtccttttgaactgtgacgaccatcaaggtttattaaaaaaaatgggtagagacattagtgaagcaagacctgatattacccaccaatgtcttttgacgttgctagattctccaatcaacaaagccggaaagctgcaggtctatattcaaacaagtcgaggaattctgatcgaggttaaccccactgttcgtataccaagaactttcaaaagattttcaggtttaatggttcagttactacataagctttctatcagatcggtaaattctgaagaaaagttacttaaagtcattaagaacccaattaccgatcacctacctactaagtgccgtaaggtgacattatcctttgacgcaccagttatccgcgttcaagattacatcgaaaaactagacgatgatgaaagtatatgtgtctttgttggtgccatggcaagaggtaaagataactttgcggatgaatacgtcgacgaaaaagtcggcttgtccaattacccattgtctgcctcagttgcatgttctaaattttgccatggcgctgaagatgcttggaatattttatag

SEQ ID NO: 56 - protein, 252 aa, Saccharomyces cerevisiae
  source 1..252: note = The sequence is a translation of SEQ ID NO. 55
MVEDSRVRDALKGGDQKALPASLVPQAPPVLTSKDKITKRMIVVLAMASLETHKISSNGPGGDKYVLLNCDDHQGLLKKMGRDISEARPDITHQCLLTLLDSPINKAGKLQVYIQTSRGILIEVNPTVRIPRTFKRFSGLMVQLLHKLSIRSVNSEEKLLKVIKNPITDHLPTKCRKVTLSFDAPVIRVQDYIEKLDDDESICVFVGAMARGKDNFADEYVDEKVGLSNYPLSASVACSKFCHGAEDAWNIL

SEQ ID NO: 57 - DNA, 804 bp, Saccharomyces cerevisiae
  source 1..804: mol_type = genomic DNA
  CDS 1..804: note = S. cerevisea essential gene URA3
atgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaa

SEQ ID NO: 58 - protein, 267 aa, Saccharomyces cerevisiae
  source 1..267: note = The sequence is a translation of SEQ ID NO. 57
MSKATYKERAATHPSPVAAKLFNIMHEKQTNLCASLDVRTTKELLELVEALGPKICLLKTHVDILTDFSMEGTVKPLKALSAKYNFLLFEDRKFADIGNTVKLQYSAGVYRIAEWADITNAHGVVGPGIVSGLKQAAEEVTKEPRGLLMLAELSCKGSLSTGEYTKGTVDIAKSDKDFVIGFIAQRDMGGRDEGYDWLIMTPGVGLDDKGDALGQQYRTVDDVVSTGSDIIIVGRGLFAKGRDAKVEGERYRKAGWEAYLRRCGQQN

SEQ ID NO: 59 - DNA, 1095 bp, Saccharomyces cerevisiae
  source 1..1095: mol_type = genomic DNA
  CDS 1..1095: note = S. ceverisiae essential gene LEU2
atgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgtcccacttccagatgaggcgctggaagcctccaagaaggttgatgccgttttgttaggtgctgtggctggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgatggtgatggtgtcgcttgggatagtgaacaatacaccgttccagaagtgcaaagaatcacaagaatggccgctttcatggccctacaacatgagccaccattgcctatttggtccttggataaagctaatcttttggcctcttcaagattatggagaaaaactgtggaggaaaccatcaagaacgaattccctacattgaaggttcaacatcaattgattgattctgccgccatgatcctagttaagaacccaacccacctaaatggtattataatcaccagcaacatgtttggtgatatcatctccgatgaagcctccgttatcccaggttccttgggtttgttgccatctgcgtccttggcctctttgccagacaagaacaccgcatttggtttgtacgaaccatgccacggttctgctccagatttgccaaagaataaggttgaccctatcgccactatcttgtctgctgcaatgatgttgaaattgtcattgaacttgcctgaagaaggtaaggccattgaagatgcagttaaaaaggttttggatgcaggtatcagaactggtgatttaggtggttccaacagtaccaccgaagtcggtgatgctgtcgccgaagaagttaagaaaatccttgcttaa

SEQ ID NO: 60 - protein, 364 aa, Saccharomyces cerevisiae
  source 1..364: note = The sequence is a translation of SEQ ID NO. 59
MSAPKKIVVLPGDHVGQEITAEAIKVLKAISDVRSNVKFDFENHLIGGAAIDATGVPLPDEALEASKKVDAVLLGAVAGPKWGTGSVRPEQGLLKIRKELQLYANLRPCNFASDSLLDLSPIKPQFAKGTDFVVVRELVGGIYFGKRKEDDGDGVAWDSEQYTVPEVQRITRMAAFMALQHEPPLPIWSLDKANLLASSRLWRKTVEETIKNEFPTLKVQHQLIDSAAMILVKNPTHLNGIIITSNMFGDIISDEASVIPGSLGLLPSASLASLPDKNTAFGLYEPCHGSAPDLPKNKVDPIATILSAAMMLKLSLNLPEEGKAIEDAVKKVLDAGIRTGDLGGSNSTTEVGDAVAEEVKKILA

SEQ ID NO: 61 - DNA, 675 bp, Saccharomyces cerevisiae
  source 1..675: mol_type = genomic DNA
  CDS 1..675: note = S. cerevisiae essential gene TRP1
atgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaatag

SEQ ID NO: 62 - protein, 224 aa, Saccharomyces cerevisiae
  source 1..224: note = The sequence is a translation of SEQ ID NO. 61
MSVINFTGSSGPLVKVCGLQSTEAAECALDSDADLLGIICVPNRKRTIDPVIARKISSLVKAYKNSSGTPKYLVGVFRNQPKEDVLALVNDYGIDIVQLHGDESWQEYQEFLGLPVIKRLVFPKDCNILLSAASQKPHSFIPLFDSEAGGTGELLDWNSISDWVGRQESPESLHFMLAGGLTPENVGDALRLNGVIGVDVSGGVETNGVKDSNKIANFVKNAKK

SEQ ID NO: 63 - DNA, 657 bp, Saccharomyces cerevisiae
  source 1..657: mol_type = genomic DNA
  CDS 1..657: note = S. cerevisiae essential gene HIS3
atgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctactggcgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatg

SEQ ID NO: 64 - protein, 219 aa, Saccharomyces cerevisiae
  source 1..219: note = The sequence is a translation of SEQ ID NO. 63
MTEQKALVKRITNETKIQIAISLKGGPLAIEHSIFPEKEAEAVAEQATQSQVINVHTGIGFLDHMIHALAKHSGWSLIVECIGDLHIDDHHTTEDCGIALGQAFKEALLARGVKRFGSGFAPLDEALSRAVVDLSNRPYAVVELGLQREKVGDLSCEMIPHFLESFAEASRITLHVDCLRGKNDHHRSESAFKALAVAIREATSPNGTNDVPSTKGVLM

SEQ ID NO: 65 - DNA, 290 bp, Escherichia coli
  regulatory 1..290: note = promoter - burden sensing promoter p(htpG); regulatory_class = promoter
  source 1..290: mol_type = genomic DNA
gccaccaaccccacggttgaaggtgaagctaccgctaactacattgccgagctttgcgcgcaatatgacgtggaagccagccgaatcgctcatggcgtaccggttggcggcgaactggaaatggtcgatggcaccacgctgtcacactcccttgccgggcgtcataagattcgtttttaagcaaacgagagcaggatcacctgctctcgcttgaaattattctcccttgtccccatctctccctcatcctgtttttaaccttaaaatggcattattgaggtagacctaca

SEQ ID NO: 66 - DNA, 257 bp, Escherichia coli
  regulatory 1..257: note = promoter - burden sensing promoter p(ibpA); regulatory_class = promoter
  source 1..257: mol_type = genomic DNA
atcccttgctgaaaataacatcatcattacgtcgcactgtggcggctatcgcactttaacgtttcgtgctgccccctcagtctatgcaatagaccataaactgcaaaaaaaagtccgctgataaggcttgaaaagttcatttccagacccatttttacatcgtagccgatgaggacgcgcctgatgggtgttctggctacctgacctgtccattgtggaaggtcttacattctcgctgatttcaggagctattgatt

SEQ ID NO: 67 - DNA, 298 bp, Escherichia coli
  regulatory 1..298: note = promoter - burden sensing promoter p(clpB); regulatory_class = promoter
  source 1..298: mol_type = genomic DNA
gataagtatctggcggatatttatctgcttgcccggcagcgtctggcgagcgttggtgttgaacaaattttcggcggcgaccgttgtacatatacggaaaatgagactttcttctcttatcgtcgcgacaagaccaccgggcgtatggcaagtttcatttggctgatataacctaaagaatcaagacgatccggtacgcgtgattttcttttcacattaatctggtcaataaccttgaataattgagggatgacctcatttaatctccagtagcaactttgatccgttatgggaggag

SEQ ID NO: 68 - DNA, 300 bp, Escherichia coli
  regulatory 1..300: note = promoter - burden sensing promoter p(yccV); regulatory_class = promoter
  source 1..300: mol_type = genomic DNA
ggttataaagacatcaacatgctggcgattcagctgctgaatgaaggcggtattctcctgactttctcctgttccggtctgatgaccagcgatttatttcagaaaatcatcgcggatgccgcaattgatgccggtcgtgatgtacaatttatagagcagttccgtcaggcagccgatcatccggtgatcgctacctatccggaagggctatatctgaaagggtttgcctgtcgcgtcatgtaacttgaaatgtggaattttacccttatatcaaaggtaaagatttcccgggaggtgact

SEQ ID NO: 69 - DNA, 300 bp, Escherichia coli
  regulatory 1..300: note = promoter - burden sensing promoter p(grpE); regulatory_class = promoter
  source 1..300: mol_type = genomic DNA
tctccgcgagcgtgccagttttcacattcttcagttgcagttcgtgagcgatttgttgctcaacgatgacctcgtaaccttttgtgcacagccagcggtagagcatttcatgtgttgtcagtgcagtggggtgccgtgggtgtcccacaatgccaatacacttgaaatgattattcatttttccgaggtccttgttgcgaagattgatgacaatgtgagtgcttcccttgaaaccctgaaactgatccccataataagcgaagttagcgagatgaatgcgaaaaaaacgcggagaaattc

SEQ ID NO: 70 - DNA, 300 bp, Escherichia coli
  regulatory 1..300: note = promoter - burden sensing promoter p(ycjX); regulatory_class = promoter
  source 1..300: mol_type = genomic DNA
ttggctgatactgtaattcttcaatggcccgtaacactttttcgcgcgcttccggggagatattagtttgcttattcagcacgcgtgatacggtggattttgatacgcctgcaaccctggcaatatcataaatagtaggggacataggtcaggaactccgtccgaacgtgaatgctgcacatcttatggagttcgccggtcgatgacaacacgggcagttgataatcaatggcctggcccccacattcatatccttacgaatgattttttttctggtcttcagagcagcacaggacagca

SEQ ID NO: 71 - DNA, 300 bp, Escherichia coli
  regulatory 1..300: note = promoter - burden sensing promoter p(ldhA); regulatory_class = promoter
  source 1..300: mol_type = genomic DNA
gggaacccacagcccgagcgtcatcagcagcgtcaacggcacaagaataatcagtaataacagcgcgagaacggctttatatttacccagcatgggtagttaatatcctgatttagcgaaaaattaagcattcaatacgggtattgtggcatgtttaaccgttcagttgaaggttgcgcctacactaagcatagttgttgatgaatttttcaatatcgccatagctttcaattatatttgaaattttgtaaaatatttttagtagcttaaatgtgattcaacatcactggagaaagtctt

SEQ ID NO: 72 - DNA, 300 bp, Escherichia coli
  regulatory 1..300: note = promoter - burden sensing promoter p(mutM); regulatory_class = promoter
  source 1..300: mol_type = genomic DNA
ggcgaaaagtactaagtacttagaggaaataaatcatggctaaaggtattcgtgagaaaatcaagctggtttcttctgctggtactggtcacttctataccactacgaagaacaaacgtactaagccggaaaaactggaactgaaaaaattcgatccagttgttcgccagcacgtgatctacaaagaagcgaaaatcaaataattctcgctttgatgtaacaaaaaacctcgctccggcggggttttttgttatctgcttgcccccatattgactgcatctgttcattcctggagatgct

SEQ ID NO: 73 - DNA, 300 bp, Escherichia coli
  regulatory 1..300: note = promoter - burden sensing promoter p(ybbN); regulatory_class = promoter
  source 1..300: mol_type = genomic DNA
cccattcgtactcgcttcaccgacaacgtcaaccagacgcaaagtgataaaccagtcgaaaatcccggcatcgccgcccgctttacgttgggaccggaagcggtggtggacaaagtacgccatgcttttattagcgagaagccgaagatgcgctatccggtaacgctggtgacctgggcagtaatggtgcttaagcgcctgctgccggggcgcgtgatggacaaaatattgcaggggtgagttgaagcgcgcgcttaagcccccatgtcaaagaaaatatcgacaacagagagcgactcc

SEQ ID NO: 74 - DNA, 298 bp, Escherichia coli
  regulatory 1..298: note = promoter - burden sensing promoter p(prlC); regulatory_class = promoter
  source 1..298: mol_type = genomic DNA
agttcttgtttgagtccactcatgggttgatgtccgattgcgcccaaattttgggcaactgcgtagattttcgatggtagcacaatcagattcgcttatgacggtgatgaagaaattgcgatgaaatgtgaggtgaatcagggttttcacccgattttgtgctgatcagaattttttttctttttcccccttgaaggggcgaagcctcatccccatttctctggtcaccagccgggaaaccacgtaagctccggcgtcacccataacagatacggactttctcaaaggagagttatca

SEQ ID NO: 75 - DNA, 298 bp, Escherichia coli; identical to SEQ ID NO: 74
  regulatory 1..298: note = promoter - burden sensing promoter p(groES); regulatory_class = promoter
  source 1..298: mol_type = genomic DNA

SEQ ID NO: 76 - DNA, 392 bp, Escherichia coli
  regulatory 1..392: note = promoter - burden sensing promoter p(fxsA); regulatory_class = promoter
  misc_feature 328: note = N is A, T, C or G
  source 1..392: mol_type = genomic DNA
gatccccggaatattcatatgttaaacacacagaatatatgtggtttcgaatgtttttcgaccgacgattatcccctgcatcgaccgaatacccgagatcatatgctgcttgaggatttctaccgtaatctggatcactttaagtgtcggtttttaccccttaattattaatttgtgaaatagatcaccgctttgggattactaccaaaaatagttgcgcaaacatcttgaaattttgctaatgaccacaatataagctgaacgcgattcgcaacccattcaggtagccgggttttaccggctgctattacaggagaaacctacttgnaaggtatgaaactctttgcccagggtacttcactggaccttagccatcctcacgtaatggggat

SEQ ID NO: 77 - DNA, 300 bp, Escherichia coli
  regulatory 1..300: note = promoter - burden sensing promoter p(htpX); regulatory_class = promoter
  source 1..300: mol_type = genomic DNA
agattaccaggagccggatccttatctggatgagacggtgaatatcgcactcgatctggcgaagcttgaaaaagccagacccgcggaacaacccgctcccgtcaagtaatatcaatcaggcacaagaaattgtgcctgattttttaacagcggcaagatgccgtaaatcagatgctacaaaatgtaaagttgtgtctttctggtgacttacgcactatccagacttgaaaatagtcgcgtaacccatacgatgtgggtatcgcatattgcgttttgttaaactgaggtaaaaagaaaatt

SEQ ID NO: 78 - DNA, 275 bp, Escherichia coli
  regulatory 1..275: note = promoter - burden sensing promoter p(rrnB); regulatory_class = promoter
  source 1..275: mol_type = genomic DNA
gcaaacacgccgccgggtcagcggggttctcctgagaattccggcagagaaagcaaaaataaatgcttgactctgtagcgggaaagcgtattatgcacaccccgcgccgctgagaaaaagcgaagcggcactgctctttaacaatttatcagacaatctgtgtgggcactcgaagatacggattcttaacgtcgcaagacgaaaaatgaataccaagtctcaagagtgaacacgtaattcattacgaagtttaattctttgagcgtcaaactttt

SEQ ID NO: 79 - DNA, 296 bp, Escherichia coli
  regulatory 1..296: note = promoter - burden sensing promoter p(rrnE); regulatory_class = promoter
  source 1..296: mol_type = genomic DNA
aatgcgcctccgttgagacgacaacgtgaaacacttcacaggatggtcggaacaacgaagagaaaaaaatcctgaaattcagggttgactctgaaagaggaaagcgtaatatacgccacctcgcgacagtgagctgaaagccgcgtcgcaactgctctttaacaatttatcagacaatctgtgtgggcactcgaagatacggattcttaacgtcgcaagacgaaaaatgaataccaagtctcaagagtgaacacgtaattcattacgaagtttaattctttgagcatcaaactttt

SEQ ID NO: 80 - DNA, 300 bp, Escherichia coli
  regulatory 1..300: note = promoter - burden sensing promoter p(cspD); regulatory_class = promoter
  source 1..300: mol_type = genomic DNA
cggttatcggcagaacgccctgccgctgacaagagcttatttgcctgaccaatctactccacatgatagataactatcatctatcgttggcatcagcgacatctgtcacattcctgtcaatagcgttaactgcttcaaatttttgattcatacctgccccctgtcccctgtcaaatgcttgacggctcgccctaattctctaaattgtatttctagagttggcgaggttttgaacagccccctctctgaccccggtttattccatcttacttgtataagatttgcgaaggatgtcgaagc

SEQ ID NO: 81 - DNA, 299 bp, Escherichia coli
  regulatory 1..299: note = promoter - burden sensing promoter p(katE); regulatory_class = promoter
  source 1..299: mol_type = genomic DNA
taaatccatctttatcgcatgttctggcggcgcgggttccgtgcgtgggacatagctaataatctggcggttttgctggcggagcggtttcttcattactggcttcactaaacgcatattaaaaatcagaaaaactgtagtttagccgatttagcccctgtacgtcccgctttgcgtgtatttcataacaccgtttccagaatagtctccgaagcgggatctggctggtggtctatagttagagagttttttgaccaaaacagcggccctttcagtaataaattaaggagacgagttca

SEQ ID NO: 82 - DNA, 300 bp, Escherichia coli
  regulatory 1..300: note = promoter - burden sensing promoter p(xthA); regulatory_class = promoter
  source 1..300: mol_type = genomic DNA
caaaccatatgcatttaaagtgcatataaagtgaatacgtttgcgatgtgggtgaataaaaagaataaaaaacgcaatgttatgcagaagtaaaatataattctggaattgtgatcattgacgaaatttactggaaattactgcgccattctgacgcagcgcgcaccaaaagcgggcattttttgcgccatcgttgacatcattaacaatcatcgatcaaatcacttaacaacaggcggtaagcaacgcgaaattctgctaccatccacgcactctttatctgaataaatggcagcgact

SEQ ID NO: 83 - DNA, 301 bp, Escherichia coli
  regulatory 1..301: note = promoter - burden sensing promoter p(uspE); regulatory_class = promoter
  source 1..301: mol_type = genomic DNA
taatatctgggcctgacggtagaaaccatcagccgtctgctgggtcgcttccagaaaagcggtatgctggcagtcaaaggtaaatacatcaccatcgaaaataacgatgcgctggcccagcttgctggtcatacgcgtaacgttgcctgatttttccgcataactcactatccttctgtcatatcattaaatttttctgatttattgatctggcagaaggttcatcactgtttcattcaccagatatgggttaatcttttaattacaaactgcgttgacagttgttgtaaggagaccctgt

SEQ ID NO: 84 - DNA, 300 bp, Escherichia coli
  regulatory 1..300: note = promoter - burden sensing promoter p(gadB); regulatory_class = promoter
  source 1..300: mol_type = genomic DNA
taattttataaatgcgttcaaaataataatcaagtactaatagtgatattttaaggtctgatttttacgtgataattcaggaggcacagaatgcgcataaaaataacagcataaaacaccttaccaccacccaagaatttcatattgtattgtttttcaatgaaaaaatattattcgcgtaatatctcacgataaataacattaggattttgttatttaaacacgagtcctttgcacttgcttactttatcgataaatcctacttttttaatgcgatccaatcattttaaggagtttaaa

SEQ ID NO: 85 - DNA, 300 bp, Escherichia coli
  regulatory 1..300: note = promoter - burden sensing promoter p(ahpC); regulatory_class = promoter
  source 1..300: mol_type = genomic DNA
tagatcaggtgattgccctttgtttatgagggtgttgtaatccatgtcgttgttgcatttgtaagggcaacacctcagcctgcaggcaggcactgaagataccaaagggtagttcagattacacggtcacctggaaagggggccattttactttttatcgccgctggcggtgcaaagttcacaaagttgtcttacgaaggttgtaaggtaaaacttatcgatttgataatggaaacgcattagccgaatcggcaaaaattggttaccttacatctcatcgaaaacacggaggaagtatag

SEQ ID NO: 86 - DNA, 300 bp, Escherichia coli
  regulatory 1..300: note = promoter - burden sensing promoter p(katG); regulatory_class = promoter
  source 1..300: mol_type = genomic DNA
gtgatcacaaattttaaacagagcacaaaatgctgcctcgaaatgagggcgggaaaataaggttatcagccttgttttctccctcattacttgaaggatatgaagctaaaacccttttttataaagcatttgtccgaattcggacataatcaaaaaagcttaattaagatcaatttgatctacatctctttaaccaacaatatgtaagatctcaactatcgcatccgtggattaattcaattataacttctctctaacgctgtgtatcgtaacggtaacactgtagaggggagcacattg

SEQ ID NO: 87 - DNA, 300 bp, Escherichia coli
  regulatory 1..300: note = promoter - burden sensing promoter p(grxA); regulatory_class = promoter
  source 1..300: mol_type = genomic DNA
tagaatctgcacttctggccgagaaaatggctcaggcagcgacagggagtcgcttaccgacagcagcgccaccgccagtagcatcattccgataaattccagtatcaacacgcctttaggcaatttaccgatcgcgcgcatacgcttccctctgcaaagtgagccttcagtctaaaacttttcactgtattgtgtttaacagttatagcttttagcaattaatgcaacaggttaaacctactttcagcgaatacattttagcgtgatcattacaggcataaatctatgaggagagaaata

SEQ ID NO: 88 - DNA, 299 bp, Escherichia coli
  regulatory 1..299: note = promoter - burden sensing promoter p(oxyS); regulatory_class = promoter
  source 1..299: mol_type = genomic DNA
taactgatccaccagcagcattcccgcctgggtgaacaacactttacggctggtccgctccagcaacatcacgcccagctcatcttccagcttacgaatttgcccgctaagcgtcggctggctaacgtggcaggaatctgccgcacgccgaaaatggcggtgttcagccaatgccaccaggtactcaagatcacgaatattcattatccatcctccatcgccacgatagttcatggcgataggtagaatagcaatgaacgattatccctatcaagcattctgactgataattgctcaca

SEQ ID NO: 89 - DNA, 300 bp, Escherichia coli
  regulatory 1..300: note = promoter - burden sensing promoter p(poxB); regulatory_class = promoter
  source 1..300: mol_type = genomic DNA
taggttgtcgctgcctgccgtgcgggtgtttgcggctgctgtaagacaaaagtggtttccggtgaatatacggtgagcagcacaatgacgctgaccgacgccgaaatcgctgaaggttacgtactggcctgctcctgccatccgcagggggatttggttctcgcataatcgccttatgcccgatgatattcctttcatcgggctatttaaccgttagtgcctcctttctctcccatcccttccccctccgtcagatgaactaaacttgttaccgttatcacattcaggagatggagaacc

SEQ ID NO: 90 - DNA, 299 bp, Escherichia coli
  regulatory 1..299: note = promoter - burden sensing promoter p(trxC); regulatory_class = promoter
  source 1..299: mol_type = genomic DNA
tagcgggttgtgggtacgtttatcagaatcatcatcactgcggacatacatcactttgaccttgccgcttttacctttcatttcatcgttcatgcttttctccaccagcgctgcgcgaagcgcgcagattacccgaagtccgcgcggttcgccatgatttcgtaccaaagcctgcgactatcatacctattgaataaaacagattgttgtctggaacaatgtccccgataatatgtaacatattagaaacataccggcgtcgttgccgataagtctccttactcatcccgaggttagtt

SEQ ID NO: 91 - DNA, 1000 bp, Saccharomyces cerevisiae
  regulatory 1..1000: note = promoter - burden sensing promoter pKAR2; regulatory_class = promoter
  source 1..1000: mol_type = genomic DNA
gtgggagtcaatcaaatcccaatcgacgctattactggacaagtcaactttattcgcaaaacaagtcctgtaatcatcaccaaaagtctcaatgaatgcttcttcattaagatactttgaccattccataggcggaaacgtgtttttatccaacactctgtcactcttggaccgtggctttggcgccctttcattttgtcttacataaaagtatctcagcagcattattgcttctttcctcaagatccccggtatactctcgtaccctgccgcactattgttcttgggcactaatgtacacgtatcatgatttactgacaagacagtaccgttgcctccaaatctctcgttgccacaaccgaacaccacctttccaatgtctaattgcttgagagcagatgcacacattatacacggttctacagtaacatatagagtaatgtctttgaacacgtcaacaactcctcgggagcccaacatcgccttgatctgatcgatccccatgaactcagcatgtgctactccagttaatgacttgttcgtatcgttcatgccataagccatcacctggccagttggcgtatgtacaaagatgcaagctaccggtgtctcatcgtggtcaagagcgtatctagccaaacggacagctgtcctcatatgtttaatatgctgcatagtgtgagtcctctagtttttaccgcagccaccagccgcttctcgagcaaagtgtagatcccattaggactcatcattcatctaattttgctatgttagctgcaactttctattttaatagaaccttctggaaatttcacccggcgcggcacccgaggaactggacagcgtgtcgaaaaagttgcttttttatataaaggacacgaaaagggttctctggaagatataaatatggctatgtaattctaaagattaacgtgttactgttttacttttttaaagtccccaagagtagtctcaagggaaaaagcgtatcaaacatacc

SEQ ID NO: 92 - DNA, 1000 bp, Saccharomyces cerevisiae
  regulatory 1..1000: note = promoter - burden sensing promoter pPDI1; regulatory_class = promoter
  source 1..1000: mol_type = genomic DNA
caggtcgtcagatgtgacgttctcatcgtctagcaaatcatcgggaatcttggacttcatttgctccatggagaaagtatgatctccatgcaagttaacagaacatatacggaaattggtaccacccaggtcggcggctagtaaaacaccgcgctccgtcccgttgggtgacccggtgacgaacgccggaatcataggaagacctttgtccgaggccaatgtgtggccttcctttggtggagctagacctttttccatttgttcgatgaagtaagcagttaattcgtccagcttctcgggggtaacctcgaaatcgtcgcagatctggtccacggcctggatgaccgctctctcagtggctttgtgtaagtcgtcgaatgacatcttgtgtatgatagagttgtattagtggtggtgttgtggtttacggaagctgagttgggcactgatgggggggttgggggttctgttgatagcggatatttatatgggcgcgttttcattagtgcccaccgtttgagcgtggtgtgacaccacgcccaagatagggggccccctgaaaggggttgaccgtccgtcggcacaccacttataatgcggggtgcaagcgccgcgtctaaaattttttttttttccatttttgtcgttattgttatttcccgttttttgttttttttgattttttcggagcgacaaacctttcgaaacacgtgtcctgaaaattatcctgggctgcacgtgataatatgttaccctgtcgggcggcgcctctttttcccttttctctcactagtctctttttccaatttgccaccgtgtagcattttgttgtgctgttacaaccacaacaaaacgaaaaacccgtatggacatacatatatatatatatatatatatatattttgttacgcgtgcattttcttgttgcaagcagcatgtctaattggtaattttaaagctgccaagctctacataaagaaaaacatacatctatcccgtt

SEQ ID NO: 93 - DNA, 1000 bp, Saccharomyces cerevisiae
  regulatory 1..1000: note = promoter - burden sensing promoter pSSA1; regulatory_class = promoter
  source 1..1000: mol_type = genomic DNA
cactcatatcgggggtcttacttcccatcataaacacggaccaaagaattaaattcatcattcacataagcgaaaaaaagtaatcattattagttaacttccctttacatgtaaccataggaaaaaaaaaaaagaaagaaaaacaacgatctataaagtaaactaaatcggaacatacccttcaatgtatgacttgtcagccaaagaagcgtttaattgtttcaaagtttcaatcttggagaaatcggtggatgccattatgtgtgtatatattcggttggactcttcagtgctgaaaggtagaaagcatgaggtaaaaagataaagagtagaaggaaaaaaaaaagaaccaactaaatatagtgggtagataaccttaaactatttatttagaggcgacaatgtgaaaaattttttcttcaaaggctcggttgtcgacaaattgttacgttgtgctttgatttctaaagcgcttcttcacctgcaggttctgagccctaagaaaaaaaatttccttggttgaaaatggcggaaaaaaaaaattcagaaaaagaaataaagcacgtgtgcgcggtgtgtggatgatggtttcatcattgtcaacggcattttcgttcttgtggattgttgtaaactttccagaacattctagaaagaaagcacacggaacgtttagaagctgtcatttgcgttttttctccagattttagttgagaaagtaattaaattattcttctttttccagaacgttccatcggcggcaaaagggagagaaagaacccaaaaagaaggggggccatttagattagctgatcgtttcgaggacttcaaggttatataaggggtggattgatgtatcttcgagaagggattgagttgtagtttcgtttcccaattcttacttaagttgttttattttctctatttgtaagataagcacatcaaaagaaaagtaatcaagtattacaagaaacaaaaattcaagtaaataacagataat

SEQ ID NO: 94 - DNA, 1000 bp, Saccharomyces cerevisiae
  regulatory 1..1000: note = promoter - burden sensing promoter pFPR2; regulatory_class = promoter
  source 1..1000: mol_type = genomic DNA
ctgatcttttcacgcagttaggtaaggaaaatcgtggccaaataaatttcattgcattaaactctacaatgttcccacaccacgttagattcctaaatatgagagaacagttcccattatttgctatccataatatgatcaataatctgaaatatggtttaccacaactaccagaagaagagtacgcgaaattagaaaaaccacaaccactagacagagatatgatcgttcagttggtaaaagattaccgtgaaggtactgccaagccaattgttaagtcagaagagattccaaaagaacaaaagtccaatgtttataaaatagttgggaagacacatgacgacattgttcatgatgatgacaaggatgtccttgtcaaatattacgcgacatggtgtattcatagtaaaaggtttgcgcctatttacgaagaaattgcaaatgtcttagcatctgatgaatctgttcgcgataaaatcttgatcgccgaagtagattcaggggcaaatgatatcttaagttttcctgtgacaggatatccaaccattgctttgtatcctgccggaaataactctaagcctattatcttcaataaaattagaaatttggaagatgttttcgaatttatcaaggaatcaggtacacatcacattgacggccaggcaatttatgataaattgcaccaggccaaggattctgaagtgtctactgaagataccgtacatgatgaattataatcaataaataaagcatatataatgcacatttttaacatctgattactcgcatcgtttctggaagaaaatagctaatattcgttatttatggcatcacgattattctcaccagttacccgtttatgctcttgaagagatttagcattactgccagcgcatcttcaaatacaggtttatatgagacccattactataaccctaagaagagaaaaaggagtgctttcgttttcaatcacattctagtttacagtaattgagtctcg

SEQ ID NO: 95 - DNA, 1000 bp, Saccharomyces cerevisiae
  regulatory 1..1000: note = promoter - burden sensing promoter pRPL3; regulatory_class = promoter
  source 1..1000: mol_type = genomic DNA
cctttcctgcgggatagcttttgcttcaggttcttctgaggttctgaaaatcaatatatttttccgacaatttctcactatgcctctatcccccttaaaaagaaacactttctaccctcttttctaaactctgcacactagaccgagacagcaaatcggagcccgagagaaaatccggaggacggaaaaactagaagtaatcttatgcggggagcatgcaccagaacgcccagaaaacgtgaatgacactacatactttgatacggatgttggtatttttccggctttttctattttatcgatgccgtgtctgctgccattacgtacaggtaggcaaaggcaaacaagaatagcactttttcacttgctgcttttttttgtgaatgaagtttatcgtatattctgttagtgccgctcgtatcagccacttacgttatcattctaaagtaagtatacgaaatgtgcaaaccttagtagacgttcaagaattgctcgataattgcgaacaaaccagctgctaagccgggaagttaagtcctcaacacagaaatgatgcaaacaatttattaaattcagtggtaatgcaacagcaagaggaaaggtggagggattaacgcatttcagacagctttatagggggaaagaaagcactcgcaaacttgctgcctgttcgcagtcattggttgcaaaaactaaactctactcacgcacactggaatgaatggcaatattcttttttaggttaaccggccggacagtaatatagtaatcgttttgtacgtttttcaagaagcgacgcacaactgttttccattttttttttttttttttcagtgatcatcgtccatgaaaaaaatttttcatttgtctctttcgtgcttcctggatatataaaatacgatttatttagttgtctttgtcaatcctcatctttctttactcattatttcatttcggttttgtcatctctagaacaacacagttactacaacaatcaatc

SEQ ID NO: 96 - DNA, 1000 bp, Saccharomyces cerevisiae
  regulatory 1..1000: note = promoter - burden sensing promoter pRPL6A; regulatory_class = promoter
  source 1..1000: mol_type = genomic DNA
attctcgtgcaagcagttttgcgcgtactttagctcctcatggcacttacaatggttttattaccgtggttgctgcggaaaacgttgccaagcatgttcaaattaagatctctttaactcaaggtggtagactaaaacacatatacaaaacgaaaagccaaaaagccaataatgatggtgttgccgtatttgatgaagagtgctcgttcaaggcttctcccgaagccaatttggtactgggtgcaatttcccatcaaagactatcgagggacaaagatcttggtattgctcaaatcaacttgggtgaccctcaaattcaacaagatggccaaatttctgtaaaattaggagacggtcatctgattgtaaagattaattacggtaaagacaagaatggtcaggtacctcccgtgccagaagttcctcaagaatacacgcagtaataaacagtgaaaactattcttttatacgctcaataagggcacatttggtgtgttaacaggtgtcttttctgtgtttgttataattttgcgctattattatataacgttatatttactataatatattacaacatatattacaacatatttcaacgctctcctgttactgcttagggagaagaaagaaatgtgaagactgctattaaatccgtacattattaaaatgtatggatttgttatatttttcttcagttttttccttaaccagattcgaaaagcggcccgcctagaccattgcggttgcggctaaagcgttctggagagggcttctccgctgggcggacaacagaagcgagagacacgtttcccgtctgggaggactcaccggatttgtccagtttcccactggagtgtgctttgttccttttgcaaaatttcgttggtatggaggttatcgaatggaagggttacatgaatagattaaatgtactactcattaaagattgaatatcagttttacattaggcgaaacaacaaagtaatagccatccaacgaa

SEQ ID NO: 97 - DNA, 1000 bp, Saccharomyces cerevisiae
  regulatory 1..1000: note = promoter - burden sensing promoter pRPL28; regulatory_class = promoter
  source 1..1000: mol_type = genomic DNA
gggatagccctccatgggaatgtcaaattctgagcacgaaagcacctgctgaggtgcatttaattccgacaagtggtaaccaaattgtattgaaccgattgaggctactataattgcataacataacgcctttgtaattttatgcttgttttgccttggcatgctaacattggatagcaacgatgcactagatagaatcctattcatactatgctacgtacctgtttaactcttctcattttatccgttttctttcttcaccgttcctcttaagttctttatttttttcataacagaatgatcacccctttcactttgccgccaatataatattaacacacaagaaataagtatgaggtagtttgctctcgaaaaaaccaagtaatagttcaaaaaatgaaaaaaagaaaacaaataccaaattatgccaccgttaccttacgtttcatggttaatcatcgtttactgccgcctatgagcgtaagctaatgttataaagaaacaagctataatattgttaaatatagttgatcaacagcattgtaatgattacaagagacgaggtggaatgaaccttatgaaatgcgtattatatataaactgtaataagagctaagttgaattgaaatctacgatacttgatgttgacattatagcactagttcccaggaaaccctttcgaaaaacacagcaaaaacaagagtactgtaaccaatgtaacatctgtacaccagggacccacacattaccaaaatcaaaattatttttctaatgcctgttatttttcctatttttcctctggcgcgtgaatagcccgcagagacgcaaacaattttcctcgcagtttttcgcttgtttaatgcgtattttcccagataggttcaaacctttcatctgtatcccgtatatttaagatggcgtttgctttctccgttgatttttttccttcttagtgatttttttgcattaaatcccagaacaatcatccaactaatcaaga

SEQ ID NO: 98 - DNA, 580 bp, Saccharomyces cerevisiae
  regulatory 1..580: note = promoter - burden sensing promoter pOGG1; regulatory_class = promoter
  source 1..580: mol_type = genomic DNA
tgcaatcaggttcttcccaatcatccgaatcagagagcaaatctaggtcttcttttgaaagtatggaaggatgcttcaattgtgcttgaataaagttattagacctgaaaccacgactactcatagaaaacgatagtttggcgtgcgatacgtttttgagtaaaagaaaactgttgaaactacagataaatggccttcttggtataatatgattcaatgttgatcttatccactttggcatgtaaaattgattgatacaagtaagctaatcgctcaatggataatattacaaataaaaatatcgaggcaataccagtatttcaatgtctggccaaataaatagtttagtacagcaatttgattaacgttaaagttactctcctctttgaataatgtcgtggttaaaaagtaaagtatttgtttatacattaagtgaagaaaattaagacgcgcctttctttaatgtaacgccaaaaattttcaagagatgagctgaacagcatcacaaaactactatttccagcggaagaaggcatttgaagcgtcctgattcataattgcgattttatttatcaaccag

SEQ ID NO: 99 - DNA, 1000 bp, Saccharomyces cerevisiae
  regulatory 1..1000: note = promoter - burden sensing promoter pRAD51; regulatory_class = promoter
  source 1..1000: mol_type = genomic DNA
ccgcaataaagggcttcccggacttggaattaatacctgctaccaccggccccacaaagtatggaccgaatcttctttcataaagtgaacttgacactaattgggtaaacgtttcaggctcaatggccctttcttccttcaacttatataggttcgtcttgtagcggaacatctcgttcagagtagtcacgtcggtagccagaccggtaatccctaagaacacatgcccatagtggaagattttttcaaacttgttcgacacacctagtgactggcttccgagacgcaaatcacaggcaatggccacacagtctttacctgtcatcgcaacgacgataccaccgttaatagaacttggatccgacattttttttttatgctttattcactgttcaatattttcaccacaattcgcaagaaacgcactctacttcgaaactacggttcaaacttacttagcagcttcccgatttaattggcctttctactatgccataaactctttcttcctctcttttcatcgcccctgcatttgcacttttttgccaccggcagtgccatccggtcacatgactacaccacgttaatagcgatctggcttatcattgtcacagagtaaattaaaatggacggtaaatgttggaaatgcaccactaccgttcttcaaccaatctagtttagctatcctgcaacaggtggccttcttgagcattccctgagcattccaaccggttgtatcagtgttttatcaccgtctcaccatatcccacgactaggccacacttcgttaccctatgctacgcgtcatttccgctatttctgtcctggtttgtttacagtacgcgtggtgggaccataaaggggaatagtggggactggagaaaaaattttctcagttacttcttctatcttccgtagtttccatatactagtagttgagtgtagcgacaaagagcagacgtagttatttgttaaaggcctactaatttgttatcgtcat

SEQ ID NO: 100 - DNA, 1000 bp, Saccharomyces cerevisiae
  regulatory 1..1000: note = promoter - burden sensing promoter pRAD54; regulatory_class = promoter
  source 1..1000: mol_type = genomic DNA
tgcgctacggttcctgccgctcgtgggaaccccacgcaaaacatattattcgcttctctctgctgacaactccggtttacgttataccgtattaggatcactataagggttccttcgggaggaggggggaggggaagaatgtacatcgtcataaggcctttatggtgtgaagtgggttttgcgtggaaaattcgttttcaatgatatagagcccacgcatatacgtacatactagtggccaaaagcgtggggtgggcggacaaagctacactggtaaaatacaggattctatgaacaataacaacaaccagctcacgttgctgaacagccgaggtcagccgatgcaaccgaggtttccaaagtagcatttctgtgctagctatgtctgtaggtttacatttaatggtgcgtggttccagcttcatgtgcttgcatgtgatgtcctgcagatggtaagaagattctgaaagccgcgctaggagaaaaatattctgctcgaagatctgtcctcttaagtagaaagcgtgaaattgttgcgttcttgcattactactcaacgcgtacgcaaatgcgtctactgcacctgcatgataaagcttatgtatcaaaaatttaacatcttgaaaatacacaagtggtgcaaagatgtgtcacgttctggacctgagtggtgccatgtatgctatttaacatgcaaaggggaagacccttccgccttactgcaataataaaaagtattttacgcgttacccaatatagcaaagtttcgcgcaaaaaaaaaaataaaaaacaattacaaacaaaaagaaaaaaaaggaaataatagaagatctaactgaagcgaaggccaaaactcttctcacttgacgtaatagccgatacaaaatctagagcagcaacttttctctttcttcactaaagctgctacgaaagtatagaaaaatcaaacgctcagaacttagctctatttcaaggtaccatatatatttccttataactg

SEQ ID NO: 101 - DNA, 5776 bp, synthetic construct
  misc_feature 1..5776: note = plasmid pEG34
  source 1..5776: mol_type = other DNA
  CDS 463..1758: note = hGH-glycine linker-GFP
atcgatgcatgcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatatcagctgaattcattaaagaggagaaaggtaccatgggtctgggcaagaaaaagaaactgctgctgctgttactgagcagcgcagttgcagcaagctttatgagcctgaccattagcctgcctggtgttcaggcatttccgaccattccgctgagccgtctgtttgataatgcaatgctgcgtgcacatcgtctgcatcagctggcatttgatacctatcaagaatttgaagaagcgtatatcccgaaagagcagaaatatagcttcctgcagaatccgcagaccagcctgtgttttagcgaaagcattccgacaccgagcaatcgtgaagaaacccagcagaaaagcaatctggaactgctgcgtattagtctgctgctgattcagagctggctggaaccggtgcagtttctgcgtagcgtttttgcaaatagcctggtttatggtgcaagcgatagcaatgtttatgatctgctgaaagatctggaagaaggtattcagaccctgatgggtcgtctggaagatggttcaccgcgtaccggtcagatctttaaacagacctatagcaaattcgataccaacagccataatgatgatgccctgctgaaaaactatggtctgctgtattgtttccgcaaagatatggataaagtggaaacctttctgcgcattgttcagtgtcgtagcgttgaaggtagctgtggttttggtggcatgcgtaaaggcgaagagctgttcactggtttcgtcactattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccacatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcacatggttctgctggagttcgtaaccgcagcgggcatcacgcatggtatggatgaactgtacaaataagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatc

SEQ ID NO: 102 - protein, 431 aa, synthetic construct
  source 1..431: note = The sequence is a translation of CDS hGH-glycine linker-GFP nt (463)..(1758) of SEQ ID NO. 101
FPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGMRKGEELFTGFVTILVELDGDVNGHKFSVRGEGEGDATNGKLTLKFICTTGKLPVPWPTLVTTLTYGVQCFARYPDHMKQHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 103 - DNA, 660 bp, synthetic construct
  source 1..660: mol_type = other DNA
  CDS 1..660: note = Chloramphenicol_acetyltransferase
atggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaa

SEQ ID NO: 104 - protein, 219 aa, synthetic construct
  source 1..219: note = The sequence is a translation of SEQ ID NO. 103
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 105 - DNA, 4347 bp, synthetic construct
  misc_feature 1..4347: note = plasmid pEG0
  source 1..4347: mol_type = other DNA
atcgatgcatgcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatatcgaattcctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatc

SEQ ID NO: 106 - DNA, 6076 bp, synthetic construct
  misc_feature 1..6076: note = Plasmid pENDU5CAM
  source 1..6076: mol_type = other DNA
  CDS 108..911: note = ompA-lysostaphin
gatctcgatcccgcgaaattaatacgactcactataggggaattgtgagcggataacaattcccctctagaaataattttgtttaactttaagaaggagatatacatatgaaaaagacagctatcgcgattgcagtggcactggctggtttcgctaccgtagcgcaggctgcagcaacccatgaacatagcgcacagtggctgaataattacaaaaaaggttatggctatggtccgtatccgctgggtattaatggtggtatgcattatggtgtggatttctttatgaatattggcacaccggttaaagcaattagcagcggtaaaattgttgaagcaggttggagcaattatggtggtggtaatcagattggcctgattgaaaatgatggtgttcatcgtcagtggtatatgcatctgagcaaatacaatgtgaaagtgggcgattatgttaaagccggtcagattattggttggagcggtagcaccggttatagtaccgcaccgcatctgcattttcagcgtatggttaatagctttagcaatagcaccgcacaggatccgatgccgtttctgaaaagcgcaggttatggtaaagcaggcggtacagttaccccgacaccgaataccggttggaaaaccaacaaatatggcaccctgtataaaagcgaaagcgcaagctttaccccgaatacagatattatcacccgtaccaccggtccgtttcgtagcatgccgcagagcggtgttctgaaagcaggtcagaccattcattatgatgaagtgatgaaacaggatggtcatgtttgggttggttataccggtaatagcggtcagcgtatttatctgccggttcgtacctggaataaaagcaccaatacactgggtgttctgtggggcaccatcaaataatgagatccggctgctaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcattgttcatgaccaaaatcccttaacgtgagttttcgttccactgagcgtcagaccccgtagaaaagattgttcatgaccaaaatcccttaacgtgatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatatccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcga

SEQ ID NO: 107 - protein, 267 aa, synthetic construct
  source 1..267: note = The sequence is a translation of CDS ompA-lysostaphin nt (108)..(911) of SEQ ID NO. 106
MKKTAIAIAVALAGFATVAQAAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 108 - DNA, 8593 bp, synthetic construct
  misc_feature 1..8593: note = Plasmid pMevT
  source 1..8593: mol_type = other DNA
  CDS 380..1564: note = Acetyl-CoA_acetyltransferase
  CDS 1578..3053: note = 3-hydroxy-3-methylglutaryl-coenzyme_A_synthase
  CDS 3067..4575: note = N-Terminally_Truncated_3-Hydroxy-3-Methylglutaryl_C oenzyme_A_Reductase
atcgatgcatgcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttgatatcgaattcctgcagcccggggatcctctagagtcgactaggaggaatataaaatgaaaaattgtgtcatcgtcagtgcggtacgtactgctatcggtagttttaacggttcactcgcttccaccagcgccatcgacctgggggcgacagtaattaaagccgccattgaacgtgcaaaaatcgattcacaacacgttgatgaagtgattatgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcgaaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaacctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggtcagggaattgcgatggtgattgaacggttgaattaaggaggacagctaaatgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataaggaggattacactatggttttaaccaataaaacagtcatttctggatcgaaagtcaaaagtttatcatctgcgcaatcgagctcatcaggaccttcatcatctagtgaggaagatgattcccgcgatattgaaagcttggataagaaaatacgtcctttagaagaattagaagcattattaagtagtggaaatacaaaacaattgaagaacaaagaggtcgctgccttggttattcacggtaagttacctttgtacgctttggagaaaaaattaggtgatactacgagagcggttgcggtacgtaggaaggctctttcaattttggcagaagctcctgtattagcatctgatcgtttaccatataaaaattatgactacgaccgcgtatttggcgcttgttgtgaaaatgttataggttacatgcctttgcccgttggtgttataggccccttggttatcgatggtacatcttatcatataccaatggcaactacagagggttgtttggtagcttctgccatgcgtggctgtaaggcaatcaatgctggcggtggtgcaacaactgttttaactaaggatggtatgacaagaggcccagtagtccgtttcccaactttgaaaagatctggtgcctgtaagatatggttagactcagaagagggacaaaacgcaattaaaaaagcttttaactctacatcaagatttgcacgtctgcaacatattcaaacttgtctagcaggagatttactcttcatgagatttagaacaactactggtgacgcaatgggtatgaatatgatttctaaaggtgtcgaatactcattaaagcaaatggtagaagagtatggctgggaagatatggaggttgtctccgtttctggtaactactgtaccgacaaaaaaccagctgccatcaactggatcgaaggtcgtggtaagagtgtcgtcgcagaagctactattcctggtgatgttgtcagaaaagtgttaaaaagtgatgtttccgcattggttgagttgaacattgctaagaatttggttggatctgcaatggctgggtctgttggtggatttaacgcacatgcagctaatttagtgacagctgttttcttggcattaggacaagatcctgcacaaaatgttgaaagttccaactgtataacattgatgaaagaagtggacggtgatttgagaatttccgtatccatgccatccatcgaagtaggtaccatcggtggtggtactgttctagaaccacaaggtgccatgttggacttattaggtgtaagaggcccgcatgctaccgctcctggtaccaacgcacgtcaattagcaagaatagttgcctgtgccgtcttggcaggtgaattatccttatgtgctgccctagcagccggccatttggttcaaagtcatatgacccacaacaggaaacctgctgaaccaacaaaacctaacaatttggacgccactgatataaatcgtttgaaagatgggtccgtcacctgcattaaatcctaagtcgacctgcaggcatgcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcagcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaacttgaacaacactcaaccctatctcgggctattcttttgatttataagggattttgccgatttcggcctattggttaaaaaatgagctgatttaacaaaaatttaacgcgaattttaacaaaatattaacgtttacaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttgctcatgagcccgaagtggcgagcccgatcttccccatcggtgatgtcggcgatataggcgccagcaaccgcacctgtggcgccggtgatgccggccacgatgcgtccggcgtagaggatctgctcatgtttgacagcttatc

SEQ ID NO: 109 - protein, 394 aa, synthetic construct
  source 1..394: note = The sequence is a translation of CDS Acetyl-CoA_acetyltransferase nt (308)..(1564) of SEQ ID NO. 108
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKNLGFDSEKVNVNGGAIALGHPIGASGARILVTLLHAMQARDKTLGLATLCIGGGQGIAMVIERLN

SEQ ID NO: 110 - protein, 491 aa, synthetic construct
  source 1..491: note = The sequence is a translation of CDS 3-hydroxy-3-methylglutaryl-coenzyme_A_synthase nt (1578)..(3053) of SEQ ID NO. 108
MKLSTKLCWCGIKGRLRPQKQQQLHNTNLQMTELKKQKTAEQKTRPQNVGIKGIQIYIPTQCVNQSELEKFDGVSQGKYTIGLGQTNMSFVNDREDIYSMSLTVLSKLIKSYNIDTNKIGRLEVGTETLIDKSKSVKSVLMQLFGENTDVEGIDTLNACYGGTNALFNSLNWIESNAWDGRDAIVVCGDIAIYDKGAARPTGGAGTVAMWIGPDAPIVFDSVRASYMEHAYDFYKPDFTSEYPYVDGHFSLTCYVKALDQVYKSYSKKAISKGLVSDPAGSDALNVLKYFDYNVFHVPTCKLVTKSYGRLLYNDFRANPQLFPEVDAELATRDYDESLTDKNIEKTFVNVAKPFHKERVAQSLIVPTNTGNMYTASVYAAFASLLNYVGSDDLQGKRVGLFSYGSGLAASLYSCKIVGDVQHIIKELDITNKLAKRITETPKDYEAAIELRENAHLKKNFKPQGSIEHLQSGVYYLTNIDDKFRRSYDVKK

SEQ ID NO: 111 - protein, 502 aa, synthetic construct
  source 1..502: note = The sequence is a translation of CDS N-Terminally_T runcated_3-Hydroxy-3-Methylglutaryl_Coenzyme_A_Reductase nt (3067)..(4575) of SEQ ID NO. 108
MVLTNKTVISGSKVKSLSSAQSSSSGPSSSSEEDDSRDIESLDKKIRPLEELEALLSSGNTKQLKNKEVAALVIHGKLPLYALEKKLGDTTRAVAVRRKALSILAEAPVLASDRLPYKNYDYDRVFGACCENVIGYMPLPVGVIGPLVIDGTSYHIPMATTEGCLVASAMRGCKAINAGGGATTVLTKDGMTRGPVVRFPTLKRSGACKIWLDSEEGQNAIKKAFNSTSRFARLQHIQTCLAGDLLFMRFRTTTGDAMGMNMISKGVEYSLKQMVEEYGWEDMEVVSVSGNYCTDKKPAAINWIEGRGKSVVAEATIPGDVVRKVLKSDVSALVELNIAKNLVGSAMAGSVGGFNAHAANLVTAVFLALGQDPAQNVESSNCITLMKEVDGDLRISVSMPSIEVGTIGGGTVLEPQGAMLDLLGVRGPHATAPGTNARQLARIVACAVLAGELSLCAALAAGHLVQSHMTHNRKPAEPTKPNNLDATDINRLKDGSVTCIKS

SEQ ID NO: 112 - DNA, 32 bp, synthetic construct
  primer_bind 1..32: note = yccV forward primer
  source 1..32: mol_type = other DNA
  misc_feature 10: note = Uracil instead of thymine in this location
attcatatgtggttataaagacatcaacatgc

SEQ ID NO: 113 - DNA, 90 bp, synthetic construct
  primer_bind 1..90: note = yccV reverse primer
  misc_feature 65: note = N is A, T, C or G
  source 1..90: mol_type = other DNA
atccccattacgtgaggatggctaaggtccagtgaagtaccctgggcaaagagtttcataccttncaagtagtcacctcccgggaaatct

SEQ ID NO: 114 - DNA, 32 bp, synthetic construct
  primer_bind 1..32: note = ycjX forward primer
  source 1..32: mol_type = other DNA
  misc_feature 10: note = Uracil instead of thymine in this location
attcatatgtttggctgatactgtaattcttc

SEQ ID NO: 115 - DNA, 90 bp, synthetic construct
  primer_bind 1..90: note = ycjX reverse primer
  misc_feature 65: note = N is A, T, C or G
  source 1..90: mol_type = other DNA
atccccattacgtgaggatggctaaggtccagtgaagtaccctgggcaaagagtttcataccttncaagttgctgtcctgtgctgctctg

SEQ ID NO: 116 - DNA, 32 bp, synthetic construct
  primer_bind 1..32: note = mutM forward primer
  source 1..32: mol_type = other DNA
  misc_feature 10: note = Uracil instead of thymine in this location
attcatatgtggcgaaaagtactaagtactta

SEQ ID NO: 117 - DNA, 90 bp, synthetic construct
  primer_bind 1..90: note = mutM reverse primer
  misc_feature 65: note = N is A, T, C or G
  source 1..90: mol_type = other DNA
atccccattacgtgaggatggctaaggtccagtgaagtaccctgggcaaagagtttcataccttncaagtagcatctccaggaatgaaca

SEQ ID NO: 118 - DNA, 19 bp, synthetic construct
  primer_bind 1..19: note = primer P492
  source 1..19: mol_type = other DNA
tgcaactcttcttcaacgc

SEQ ID NO: 119 - DNA, 19 bp, synthetic construct
  primer_bind 1..19: note = primer P493
  source 1..19: mol_type = other DNA
aagaaccaggcgcttctaa

SEQ ID NO: 120 - DNA, 19 bp, synthetic construct
  primer_bind 1..19: note = primer P525
  source 1..19: mol_type = other DNA
attgatgacctgatggcac

SEQ ID NO: 121 - DNA, 18 bp, synthetic construct
  primer_bind 1..18: note = primer P526
  source 1..18: mol_type = other DNA
gatccagacttcgaagcg

SEQ ID NO: 122 - DNA, 90 bp, synthetic construct
  primer_bind 1..90: note = ibpA forward primer
  misc_feature 65: note = N is A, T, C or G
  source 1..90: mol_type = other DNA
atccccattacgtgaggatggctaaggtccagtgaagtaccctgggcaaagagtttcataccttncaagtaatcaatagctcctgaaatc

SEQ ID NO: 123 - DNA, 31 bp, synthetic construct
  primer_bind 1..31: note = ibpA reverse primer
  source 1..31: mol_type = other DNA
attcatatgtttggcctgatgagttatagcg

SEQ ID NO: 124 - DNA, 32 bp, synthetic construct
  primer_bind 1..32: note = grpE forward primer
  source 1..32: mol_type = other DNA
  misc_feature 10: note = Uracil instead of thymine in this location
attcatatgttctccgcgagcgtgccagtttt

SEQ ID NO: 125 - DNA, 90 bp, synthetic construct
  primer_bind 1..90: note = grpE reverse primer
  misc_feature 65: note = N is A, T, C or G
  source 1..90: mol_type = other DNA
atccccattacgtgaggatggctaaggtccagtgaagtaccctgggcaaagagtttcataccttncaagtgaatttctccgcgttttttt

SEQ ID NO: 126 - DNA, 32 bp, synthetic construct
  primer_bind 1..32: note = ldhA forward primer
  source 1..32: mol_type = other DNA
  misc_feature 10: note = Uracil instead of thymine in this location
attcatatgtgggaacccacagcccgagcgtc

SEQ ID NO: 127 - DNA, 90 bp, synthetic construct
  primer_bind 1..90: note = ldhA reverse primer
  misc_feature 65: note = N is A, T, C or G
  source 1..90: mol_type = other DNA
atccccattacgtgaggatggctaaggtccagtgaagtaccctgggcaaagagtttcataccttncaagtaagactttctccagtgatgt

SEQ ID NO: 128 - DNA, 122 bp, synthetic construct
  primer_bind 1..122: note = ybbN forward primer
  misc_feature 97: note = N is A, T, C or G
  source 1..122: mol_type = other DNA
  misc_feature 10: note = Uracil instead of thymine in this location
attcatatgtcccattcgtactcgcttcaccgatccccattacgtgaggatggctaaggtccagtgaagtaccctgggcaaagagtttcataccttncaagtggagtcgctctctgttgtcg

SEQ ID NO: 129 - DNA, 90 bp, synthetic construct
  primer_bind 1..90: note = ybbN reverse primer
  misc_feature 65: note = N is A, T, C or G
  source 1..90: mol_type = other DNA
atccccattacgtgaggatggctaaggtccagtgaagtaccctgggcaaagagtttcataccttncaagtggagtcgctctctgttgtcg

SEQ ID NO: 130 - DNA, 32 bp, synthetic construct
  primer_bind 1..32: note = fxsA forward primer
  source 1..32: mol_type = other DNA
  misc_feature 10: note = Uracil instead of thymine in this location
attcatatgttaaacacacagaatatatgtgg

SEQ ID NO: 131 - DNA, 90 bp, synthetic construct
  primer_bind 1..90: note = fxsA reverse primer
  misc_feature 65: note = N is A, T, C or G
  source 1..90: mol_type = other DNA
atccccattacgtgaggatggctaaggtccagtgaagtaccctgggcaaagagtttcataccttncaagtaggtttctcctgtaatagca

SEQ ID NO: 132 - DNA, 31 bp, synthetic construct
  primer_bind 1..31: note = rrnB forward primer
  source 1..31: mol_type = other DNA
  misc_feature 10: note = Uracil instead of thymine in this location
attcatatgtgcaaacacgccgccgggtcag

SEQ ID NO: 133 - DNA, 90 bp, synthetic construct
  primer_bind 1..90: note = rrnB reverse primer
  misc_feature 65: note = N is A, T, C or G
  source 1..90: mol_type = other DNA
atccccattacgtgaggatggctaaggtccagtgaagtaccctgggcaaagagtttcataccttncaagtaaaagtttgacgctcaaaga

SEQ ID NO: 134 - DNA, 35 bp, synthetic construct
  primer_bind 1..35: note = rrnE forward primer
  source 1..35: mol_type = other DNA
  misc_feature 10: note = Uracil instead of thymine in this location
attcatatgtaatgcgcctccgttgagacgacaac

SEQ ID NO: 135 - DNA, 90 bp, synthetic construct
  primer_bind 1..90: note = rrnE reverse primer
  misc_feature 65: note = N is A, T, C or G
  source 1..90: mol_type = other DNA
atccccattacgtgaggatggctaaggtccagtgaagtaccctgggcaaagagtttcataccttncaagtaaaagtttgatgctcaaaga

SEQ ID NO: 136 - DNA, 32 bp, synthetic construct
  primer_bind 1..32: note = poxB forwrad primer
  source 1..32: mol_type = other DNA
  misc_feature 10: note = Uracil instead of thymine in this location
attcatatgttaggttgtcgctgcctgccgtg

SEQ ID NO: 137 - DNA, 90 bp, synthetic construct
  primer_bind 1..90: note = poxB reverse primer
  misc_feature 65: note = N is A, T, C or G
  source 1..90: mol_type = other DNA
atccccattacgtgaggatggctaaggtccagtgaagtaccctgggcaaagagtttcataccttncaagtggttctccatctcctgaatg

SEQ ID NO: 138 - DNA, 32 bp, synthetic construct
  primer_bind 1..32: note = cspD forward primer
  source 1..32: mol_type = other DNA
  misc_feature 10: note = Uracil instead of thymine in this location
attcatatgtcggttatcggcagaacgccctg

SEQ ID NO: 139 - DNA, 90 bp, synthetic construct
  primer_bind 1..90: note = cspD reverse primer
  misc_feature 65: note = N is A, T, C or G
  source 1..90: mol_type = other DNA
atccccattacgtgaggatggctaaggtccagtgaagtaccctgggcaaagagtttcataccttncaagtgcttcgacatccttcgcaaa

SEQ ID NO: 140 - DNA, 1902 bp, synthetic construct
  misc_feature 1..1902: note = integration sequence s9_pmutM_folP
  regulatory 1533..1832: note = promoter - burden sensing promoter p(mutM); regulatory_class = promoter
  regulatory 1833..1838: note = RBS - rbs catalog; regulatory_class = ribosome_binding_site
  misc_feature 1838: note = N is A, T, C or G
  misc_feature 1844..1902: note = beginning of folP gene
  source 1..1902: mol_type = other DNA
  CDS 299..1093: note = kanR
aagaaccaggcgcttctaacaattctggcgacaatggtagtccaaaggctcctcgtccggttgatgaaccgcgtacgccgaacccgggtaacaccatgtcagagcagttaggcgacaagtaagttcccgcatcagatgactgtatttgtaccgaaaaccccggggcgtgctccggggttttttcttatcaattcataccagggataacatcagcgattgtgtaggctggagctgcttcgaagttcctatactttctagagaataggaacttcggaataggaacttcaagatccccttattagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgagcttcaaaagcgctctgaagttcctatactttctagagaataggaacttcgaactgcaggtcgacggatccccggaatattcatatgtggcgaaaagtactaagtacttagaggaaataaatcatggctaaaggtattcgtgagaaaatcaagctggtttcttctgctggtactggtcacttctataccactacgaagaacaaacgtactaagccggaaaaactggaactgaaaaaattcgatccagttgttcgccagcacgtgatctacaaagaagcgaaaatcaaataattctcgctttgatgtaacaaaaaacctcgctccggcggggttttttgttatctgcttgcccccatattgactgcatctgttcattcctggagatgctacttgnaaggtatgaaactctttgcccagggtacttcactggaccttagccatcctcacgtaatggggat

SEQ ID NO: 141 - protein, 161 aa, synthetic construct
LEELVKKAIEGDALRIGSGDTVKHEEAVSPFAAKLFSNITGSQRYVLIAVRHTQPATVDESRKAAIFHHDIRQAGIAMGHDEILAVGHARLEPGEQFGWREPLMLFVQIILIDKTGFHPSTCSLDAMFRLVVEWAGSRIKRMQPPHCISHDGYFLGRSKVR

SEQ ID NO: 142 - protein, 10 aa, synthetic construct
QEILPRHFAQ

SEQ ID NO: 143 - protein, 25 aa, synthetic construct
QPVPSRFSDNVEHSCARNARRGQPR

SEQ ID NO: 144 - protein, 26 aa, synthetic construct
PRCLVLQFIQGTGQVGLDKKNRAPLR

SEQ ID NO: 145 - protein, 20 aa, synthetic construct
QPEHGGIRAADCLLCPVIAE

SEQ ID NO: 146 - protein, 18 aa, synthetic construct
PLHPSGRRTCVQSILFNH

SEQ ID NO: 147 - DNA, 1347 bp, Bacillus licheniformis
  source 1..1347: mol_type = genomic DNA
  CDS 1..1347: note = Bacillus licheniformis essential gene glmM
atgggcaagtattttggtacagacggtgtaagaggcgtggcaaacagtgaacttacacctgagctggcctttaaagtcggacgctttggcggatatgtcctaacaaaagataaggagcgtcccaaggttctgatcggccgtgacacgcgcatttcagggcatatgctggaaggggcccttgtagcaggccttctttcgataggagcagaagtcatgcgtctcggcgtgatttcgacgcccggagtcgcatatttgacgaaggctatggacgcggaggcgggtgtgatgatttccgcttcccacaaccctgtccaggacaacggaattaagtttttcggcggcgacggctttaagctttccgatgaacaggagcttgaaatcgagcgtctgatggatcagccggaagatcacctgccaaggcctgtaggcgctgatctgggcatggtgaacgactactttgaaggcggacagaaatatttgcagtttcttaagcagtctgctgatgaagacttcacaggcattcatgtcgcgcttgactgtgcgcacggggcgacatcttcgctggcgacccacctgtttgccgatcttgacgcagatgtatcgacgatggggacttctccgaacggattaaacatcaatgacggagtcggatcaacccaccctgaagccctggcagaatttgtgaaggaaaagggagctgacgtcggcatggcgtttgacggggatggcgaccgcttaatcgctgtcgacgaaaaagggaatatcgtagacggcgatcaaatcatgtacatatgcgcgaagtacttgaagagcgaggggcgcctgacggacaataccgttgtctccacggtgatgagcaacctcggtttctacaaagcgcttgaagcggaaggcattaaaagcgttcagacagcggtcggcgaccgctatgtagtggaggcgatgaaaaagggcggatttaccctcggcggagaacagtcgggacacctgattttccttgattacaatacgacaggcgacggactgctttctgcgatcatgctgatgaacacgattaaaatgacaggcaagccgctgtctgagcttgcggcagagatgcagaagtttccgcagctcctcttgaatgtgaaagtaaccgataaacataaagtaacggaaaacgaaaaggtaaaagcggtcatcgaagaagttgagaaggaaatgaacggtgacggccgcatccttgtgcgcccatccggcaccgagccgcttgtccgtgtcatggctgaagcgaaaacgaaggagctttgcgagaagtacgtaggccgcattgcagatgtcgtgaaagctgaaatgggagcagagtaa

SEQ ID NO: 148 - protein, 448 aa, Bacillus licheniformis
  source 1..448: note = The sequence is a translation of SEQ ID NO. 147
MGKYFGTDGVRGVANSELTPELAFKVGRFGGYVLTKDKERPKVLIGRDTRISGHMLEGALVAGLLSIGAEVMRLGVISTPGVAYLTKAMDAEAGVMISASHNPVQDNGIKFFGGDGFKLSDEQELEIERLMDQPEDHLPRPVGADLGMVNDYFEGGQKYLQFLKQSADEDFTGIHVALDCAHGATSSLATHLFADLDADVSTMGTSPNGLNINDGVGSTHPEALAEFVKEKGADVGMAFDGDGDRLIAVDEKGNIVDGDQIMYICAKYLKSEGRLTDNTVVSTVMSNLGFYKALEAEGIKSVQTAVGDRYVVEAMKKGGFTLGGEQSGHLIFLDYNTTGDGLLSAIMLMNTIKMTGKPLSELAAEMQKFPQLLLNVKVTDKHKVTENEKVKAVIEEVEKEMNGDGRILVRPSGTEPLVRVMAEAKTKELCEKYVGRIADVVKAEMGAE

SEQ ID NO: 149 - DNA, 282 bp, Bacillus licheniformis
  source 1..282: mol_type = genomic DNA
  CDS 1..282: note = Bacillus licheniformis essential gene ylaN
ttggcgtctgagattgtagtcgaccatcgtgaaaaagcacttgctctgttaaaacgtgatgctgataaaatcttaaagctgattcaagtgcaaatggacaacttaacgatgcctcaatgtcctctttatgaagaggttttagatacacaaatgttcggactctctagagaaattgattttgctgtccgccttggattagtggatgaagaagagggtaaagaactgctttacaggctggagcgtgaattgtctgcattacatgatgcatttacaaaaaaataa

SEQ ID NO: 150 - protein, 93 aa, Bacillus licheniformis
  source 1..93: note = The sequence is a translation of SEQ ID NO. 149
LASEIVVDHREKALALLKRDADKILKLIQVQMDNLTMPQCPLYEEVLDTQMFGLSREIDFAVRLGLVDEEEGKELLYRLERELSALHDAFTKK

SEQ ID NO: 151 - DNA, 219 bp, Bacillus licheniformis
  source 1..219: mol_type = genomic DNA
  CDS 1..219: note = Bacillus licheniformis essential gene infA
atggcgaaagacgatgtaattgaagtggaaggtactgtagtcgaaacgctgccaaacgcaatgttcaaagttgaacttgagaatggccacacggttttggctcacgtatctggtaaaatccgcatgcacttcattcgcattttacctggagacaaagttacggtagaattatctccatatgacttaactcgtggtagaattacgtaccgttacaaataa

SEQ ID NO: 152 - protein, 72 aa, Bacillus licheniformis
  source 1..72: note = The sequence is a translation of SEQ ID NO. 151
MAKDDVIEVEGTVVETLPNAMFKVELENGHTVLAHVSGKIRMHFIRILPGDKVTVELSPYDLTRGRITYRYK

SEQ ID NO: 153 - DNA, 870 bp, Bacillus licheniformis
  source 1..870: mol_type = genomic DNA
  CDS 1..870: note = Bacillus licheniformis essential gene dapA
atgaacttcggaaatatcgcaaccgcgatggttacgccctttgacaaaaatgaaaatatcgattttcaaaaactgtcaaagctgatcgattatttactaaacaatgggacggattccctggtcgttgccggaacaaccggagaatcgccgacgctttccgaagaagaaaaggtagcgctcattcaatactctgtcaaggaagcggcaggccgtgccccaatcatcgccggcacgggaagcaacaacacgaaggcgtcgatcaagctgacgaaaaaagctgaagaagccggcgctgacgctgtcatgctcgtcactccgtactacaacaagccttctcaggaaggcatgtaccgccatttcagagcgattgcggaggaaacatcgctgcctgtcatgctttataatgtgccgggaagaacggctgcttcgctcgcaccagagacgacaatccgccttgcggagatcccgaatattatcgccattaaagaagcgagcggcgacctagatgcgataacaaaaatcgtagccgaaacccctgaggactttgcagtttattcaggagatgacagcctgacactgccggcgctgtccgtgggagcgagaggaatcgtttccgttgcatcacacatcatcggaccggaaatgcaggaaatgatcaaacattataccgaggggaacacggctcaggctgctttgatccatcaaaagctgcttccgctgatgaaagggctttttgccgcaccgaacccgtctccattaaagacggctcttcagctgaagggtcttgacgtcgggtccgtccggctccctttgattccgctaaatgaagatgaacgactccgtttaagcagcctgatgaacggcctgtaa

SEQ ID NO: 154 - protein, 289 aa, Bacillus licheniformis
  source 1..289: note = The sequence is a translation of SEQ ID NO. 153
MNFGNIATAMVTPFDKNENIDFQKLSKLIDYLLNNGTDSLVVAGTTGESPTLSEEEKVALIQYSVKEAAGRAPIIAGTGSNNTKASIKLTKKAEEAGADAVMLVTPYYNKPSQEGMYRHFRAIAEETSLPVMLYNVPGRTAASLAPETTIRLAEIPNIIAIKEASGDLDAITKIVAETPEDFAVYSGDDSLTLPALSVGARGIVSVASHIIGPEMQEMIKHYTEGNTAQAALIHQKLLPLMKGLFAAPNPSPLKTALQLKGLDVGSVRLPLIPLNEDERLRLSSLMNGL

SEQ ID NO: 155 - DNA, 2830 bp, synthetic construct
  regulatory 1..1000: note = promoter - pAOX1 promoter; regulatory_class = promoter
  misc_feature 1..2830: note = pAOX1-ALB construct
  source 1..2830: mol_type = other DNA
  CDS 1001..2830: note = cds encoding Human_serum_albumin (hAS) protein
atgttggtattgtgaaatagacgcagatcgggaacactgaaaaataacagttattattcgagatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacgatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttataa

SEQ ID NO: 156 - protein, 609 aa, synthetic construct [sequence omitted]

SEQ ID NO: 157 - DNA, 3505 bp, synthetic construct
  misc_feature 1..3505: note = construct KU70(up)-pPDI1-kanMX-terminator-KU70
  misc_feature 1..1002: note = upstream region
  regulatory 1002..1542: note = promoter - promoter pPDI1; regulatory_class = promoter
  regulatory 2353..2555: note = terminator - terminator; regulatory_class = terminator
  misc_feature 2556..3505: note = downstream region
  source 1..3505: mol_type = other DNA
  CDS 1543..2352: note = kanMX
gctcttcagaagcctaactttaatggggtaaaatatcagcagtaggctgaatatagagcatggcaacgtttacgtcaacagagtttaatagtgaaaactacgataggttcaggcctgtatacccagacgagttgtatcagcaattagtggattatcatgttggtgctaaaggattgtgtgtcgatgttggatgtggctctggccaggcaacgttcaccctgaaaaagtactttgacaaggtcataggatctgatatctcagaaaatcagttggcagtggctaggaaaagacagcctgctggaattgaatttaggttaggaacgggcgaggatttttcttggctaacggagacgccagatgtaatcaccgccgctgaatgcctgcactgggtagacccacagaaattcgttgcaaatgtcgccaattctctaagggatcatggcaccttgagttactggctttatacagaaccaatttttcagaacgaaagggccaatcaggtttacaacaaattcacctacgggtctgactacctgggcccatattgggatcctggccgtacacatttcagaaatcatttaaaggagttgaatcacatcttactggatagcgagctttttgacgaagtgaaaatttctaattttaaacaagaggaaggggtcaaaaacggagatatcttatacttggaaaaagagatgacaatcagtgatttcatcaattttgtatctagttggccttctgtgttttcgtggaagcagcaacgaggaaaggagggtatcctagatgatttttacaacgaactgaacgactgctttgaggggggtaacatgaaagtaatatggaactccgtcctagtatttgccaggaggaagcaaagggttgtataggctttagtacttatagaggaaacggggttacgtgcaagcgcgcatgcctgagctttgaggggggggactttcacatctcttcttctcacacttagccctaacacagagatcaactactcaaggcagcaccaaaagacgccagaaattatatggtccaggaaaaaacaactctctttactacaaaatttgaacaggtgtcctgcctgcggaaactacaaacgatcacacaccctctgcatgcattgcgtaggacaaatcaggagacattggaacgactctgttcctcaacaggaggcatttcgtgaagagtttgttaatcctttggatgagaagattctttatccaggaaagaaagaactgcccgatgaacgaactttacgtaagaaggagtggctgaagagaagaccccgaacactccctgttgaatagaacacgaacactgtaaatagaataaaagaaaacttggatagtagaacttcaatgtagtgtttctattgtcttacgcggctctttagattgcaatccccagaatggaatcgtccatctttctcaacccactcaaagataatctaccagacatacctacgccctccatcccagcaccacgtcgcgatcacccctaaaacttcaataattgaacacgtactgatttccaatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgttcatcttagagaatgtcagtgaggttatttctgagctaatcattaccttgcccggtacaggaatagggtgttaccttattaattacgacggtggtcaaaacgacgaaatttaccccatttttgagttacaagacctgaatttggaaatgatgaaacaattgtaccaagtcttggaggaccatgtaagtgggcttaatcctctcgagaagcaattcccaattgaacacagtaaaccgttatcagccactctgttctttcacttaaggtctcttttttacatggcgaagactcataagcgtactggaagacattacaacttgaaaaagattttcttgttcactaataacgataaaccttacaatggaaactctcagctgagagttcccttgaagaaaaccctggctgattacaatgacgtagacattactttgattccgtttcttctgaacaagccttcaggtgtcaagtttgacaagacggaatactcagaaattttgttctatgataaagatgcttgttcgatgtcaattgaggagatccgccaacgaatttctagacataaggagatcaagcgggtttacttcacctgtcctttgaaaatcgcaaataacttgtgcatttctgtgaaaggttattctatgttttatcatgaaactccaaggaagatcaaatttgtcgtcaatgagggttcaactttcaaagatgtggagacaaaatctcagtttgtcgatccaacatccggaaaagagttttccagtgaacagctgatcaaagcatatcctctaggtgccgatgcttacattcctttaaactcagagcaagtcaaaacaataaatcgatttaatgatatcatcaatatcccctctttggaaattctaggtttcagggatatatctaattggttgccacagtatcagtttggcaaagcatcgtttttatcccctaataa

SEQ ID NO: 158 - protein, 269 aa, synthetic construct
  source 1..269: note = The sequence is a translation of CDS kanMX nt (1543)..(2352) of SEQ ID NO. 157
MGKEKTHVSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 159 - DNA, 3533 bp, synthetic construct
  misc_feature 1..3533: note = KU70(up)-pFPR2-kanMX-terminator-KU70 construct
  misc_feature 1..1002: note = KU70 upstream region
  regulatory 1003..1570: note = promoter - promoter pFPR2; regulatory_class = promoter
  regulatory 2381..2583: note = terminator - terminator; regulatory_class = terminator
  misc_feature 2584..3533: note = KU70 downstream region
  source 1..3533: mol_type = other DNA
  CDS 1571..2380: note = kanMX
gctcttcagaagcctaactttaatggggtaaaatatcagcagtaggctgaatatagagcatggcaacgtttacgtcaacagagtttaatagtgaaaactacgataggttcaggcctgtatacccagacgagttgtatcagcaattagtggattatcatgttggtgctaaaggattgtgtgtcgatgttggatgtggctctggccaggcaacgttcaccctgaaaaagtactttgacaaggtcataggatctgatatctcagaaaatcagttggcagtggctaggaaaagacagcctgctggaattgaatttaggttaggaacgggcgaggatttttcttggctaacggagacgccagatgtaatcaccgccgctgaatgcctgcactgggtagacccacagaaattcgttgcaaatgtcgccaattctctaagggatcatggcaccttgagttactggctttatacagaaccaatttttcagaacgaaagggccaatcaggtttacaacaaattcacctacgggtctgactacctgggcccatattgggatcctggccgtacacatttcagaaatcatttaaaggagttgaatcacatcttactggatagcgagctttttgacgaagtgaaaatttctaattttaaacaagaggaaggggtcaaaaacggagatatcttatacttggaaaaagagatgacaatcagtgatttcatcaattttgtatctagttggccttctgtgttttcgtggaagcagcaacgaggaaaggagggtatcctagatgatttttacaacgaactgaacgactgctttgaggggggtaacatgaaagtaatatggaactccgtcctagtatttgccaggaggaagcaaagggttgtataggctttagtacttatagaggaaacggggttacgtgcaagcgcgcatgcctgagctttgaggggggggactttcacatctcttcttctcacacttagccctaacacagagatcaactacaagcggcaattcctacagaaagtgtttgcaaagttcctaatctcggtttgtcaccaagtcacttttctacgaaccctgagtttcctcacgaggaatcaagttttgaaatgcccgatttgtcgcagtttgatagttttgatgaattttttaatggaatctttccagaatttaaaagagtcaagaatatttaacttgtgagcgaacgaacatgaaaaccgaaagaatagttgatgatgatgttgaagcaatgcaagtaccatataaattaggagaagtcctttgtgtattggcgattccttttgatgcaagtgagcgtaaatggttaacaggctgatacatttactcaagctagtagtgaaagccattgcgaaatcagtttatcatagtagtggacgtcacaatggaactcatctctgcgaatcgttgtagaataactgtaggtaaatatgcaattctaacttctggtctaaaatgttcagtgacaatgcttatttctgtagtcgcgaagaattgcttctgaggagacggagcgagccatgtattctcgccaacttgacaagaacgccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgttcatcttagagaatgtcagtgaggttatttctgagctaatcattaccttgcccggtacaggaatagggtgttaccttattaattacgacggtggtcaaaacgacgaaatttaccccatttttgagttacaagacctgaatttggaaatgatgaaacaattgtaccaagtcttggaggaccatgtaagtgggcttaatcctctcgagaagcaattcccaattgaacacagtaaaccgttatcagccactctgttctttcacttaaggtctcttttttacatggcgaagactcataagcgtactggaagacattacaacttgaaaaagattttcttgttcactaataacgataaaccttacaatggaaactctcagctgagagttcccttgaagaaaaccctggctgattacaatgacgtagacattactttgattccgtttcttctgaacaagccttcaggtgtcaagtttgacaagacggaatactcagaaattttgttctatgataaagatgcttgttcgatgtcaattgaggagatccgccaacgaatttctagacataaggagatcaagcgggtttacttcacctgtcctttgaaaatcgcaaataacttgtgcatttctgtgaaaggttattctatgttttatcatgaaactccaaggaagatcaaatttgtcgtcaatgagggttcaactttcaaagatgtggagacaaaatctcagtttgtcgatccaacatccggaaaagagttttccagtgaacagctgatcaaagcatatcctctaggtgccgatgcttacattcctttaaactcagagcaagtcaaaacaataaatcgatttaatgatatcatcaatatcccctctttggaaattctaggtttcagggatatatctaattggttgccacagtatcagtttggcaaagcatcgtttttatcccctaataa

SEQ ID NO: 160 - protein, 269 aa, synthetic construct; identical to SEQ ID NO: 158
  source 1..269: note = The sequence is a translation of CDS kanMX nt (1571)..(2380) of SEQ ID NO. 159

SEQ ID NO: 161 - DNA, 3632 bp, synthetic construct
  misc_feature 1..3632: note = construct KU70(up)-pRPL3-kanMX-terminator-KU70(dw)
  misc_feature 1..1002: note = KU70 upstream region
  regulatory 1003..1669: note = promoter - pRPL3 promoter; regulatory_class = promoter
  regulatory 2480..2682: note = terminator - terminator; regulatory_class = terminator
  misc_feature 2480..2682: note = KU70 downstream region
  source 1..3632: mol_type = other DNA
  CDS 1670..2479: note = kanMX
gctcttcagaagcctaactttaatggggtaaaatatcagcagtaggctgaatatagagcatggcaacgtttacgtcaacagagtttaatagtgaaaactacgataggttcaggcctgtatacccagacgagttgtatcagcaattagtggattatcatgttggtgctaaaggattgtgtgtcgatgttggatgtggctctggccaggcaacgttcaccctgaaaaagtactttgacaaggtcataggatctgatatctcagaaaatcagttggcagtggctaggaaaagacagcctgctggaattgaatttaggttaggaacgggcgaggatttttcttggctaacggagacgccagatgtaatcaccgccgctgaatgcctgcactgggtagacccacagaaattcgttgcaaatgtcgccaattctctaagggatcatggcaccttgagttactggctttatacagaaccaatttttcagaacgaaagggccaatcaggtttacaacaaattcacctacgggtctgactacctgggcccatattgggatcctggccgtacacatttcagaaatcatttaaaggagttgaatcacatcttactggatagcgagctttttgacgaagtgaaaatttctaattttaaacaagaggaaggggtcaaaaacggagatatcttatacttggaaaaagagatgacaatcagtgatttcatcaattttgtatctagttggccttctgtgttttcgtggaagcagcaacgaggaaaggagggtatcctagatgatttttacaacgaactgaacgactgctttgaggggggtaacatgaaagtaatatggaactccgtcctagtatttgccaggaggaagcaaagggttgtataggctttagtacttatagaggaaacggggttacgtgcaagcgcgcatgcctgagctttgaggggggggactttcacatctcttcttctcacacttagccctaacacagagatcaactccaacgacttgaggaaatgacgattatttcttaagatattcatggtgaggttttccaaatgtgatctgaaatgtaatcttggcaagtttggttcataaaacgtcaaaagaccacctgaagataaaaaataaagtggaaggagaagaaaaagaggtttttttccagatgagtctatgactaagatttctcttcaaagaattttttcaaggtttgagagggatgaaagcgaggctgctgcacgattatcgatgtcattcaatagggaggtagaagagaactgagggagaaaaggctacaactgaacctaaaactaactgaatttggaatgaagcctattacacgagaggtgtggcgcctttccaaagtcatcaggttcaagttattgctttctggagcaataaatgatgtcaatcccatacggggtctccacccttatagaattacttatctaccggctaggtgttgcatcgcggaccagcccaatcgggtaattttagttgaaaaggtttttagcgcgagtctagttattacccggatagtctaatgctgaaaatttctcggacatttttgatccgtacgtatatttcagtacggagaatcatcaacaagaatctttcaatgccaccaaaaaaaaagacatttttcaccatcgcgcattcacagtagcatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgttcatcttagagaatgtcagtgaggttatttctgagctaatcattaccttgcccggtacaggaatagggtgttaccttattaattacgacggtggtcaaaacgacgaaatttaccccatttttgagttacaagacctgaatttggaaatgatgaaacaattgtaccaagtcttggaggaccatgtaagtgggcttaatcctctcgagaagcaattcccaattgaacacagtaaaccgttatcagccactctgttctttcacttaaggtctcttttttacatggcgaagactcataagcgtactggaagacattacaacttgaaaaagattttcttgttcactaataacgataaaccttacaatggaaactctcagctgagagttcccttgaagaaaaccctggctgattacaatgacgtagacattactttgattccgtttcttctgaacaagccttcaggtgtcaagtttgacaagacggaatactcagaaattttgttctatgataaagatgcttgttcgatgtcaattgaggagatccgccaacgaatttctagacataaggagatcaagcgggtttacttcacctgtcctttgaaaatcgcaaataacttgtgcatttctgtgaaaggttattctatgttttatcatgaaactccaaggaagatcaaatttgtcgtcaatgagggttcaactttcaaagatgtggagacaaaatctcagtttgtcgatccaacatccggaaaagagttttccagtgaacagctgatcaaagcatatcctctaggtgccgatgcttacattcctttaaactcagagcaagtcaaaacaataaatcgatttaatgatatcatcaatatcccctctttggaaattctaggtttcagggatatatctaattggttgccacagtatcagtttggcaaagcatcgtttttatcccctaataa

SEQ ID NO: 162 - protein, 269 aa, synthetic construct; identical to SEQ ID NO: 158
  source 1..269: note = The sequence is a translation of CDS kanMX nt (1670)..(2479) of SEQ ID NO. 161

SEQ ID NO: 163 - DNA, 3573 bp, synthetic construct
  misc_feature 1..3573: note = construct KU70(up)-pRPL6A-kanMX-terminator-KU70(dw)
  misc_feature 1..1002: note = KU70 upstream region
  regulatory 1003..1610: note = promoter - promoter pRPL6A; regulatory_class = promoter
  regulatory 2421..2623: note = terminator - terminator; regulatory_class = terminator
  misc_feature 2624..3573: note = KU70 downstream region
  source 1..3573: mol_type = other DNA
  CDS 1611..2420: note = kanMX
gctcttcagaagcctaactttaatggggtaaaatatcagcagtaggctgaatatagagcatggcaacgtttacgtcaacagagtttaatagtgaaaactacgataggttcaggcctgtatacccagacgagttgtatcagcaattagtggattatcatgttggtgctaaaggattgtgtgtcgatgttggatgtggctctggccaggcaacgttcaccctgaaaaagtactttgacaaggtcataggatctgatatctcagaaaatcagttggcagtggctaggaaaagacagcctgctggaattgaatttaggttaggaacgggcgaggatttttcttggctaacggagacgccagatgtaatcaccgccgctgaatgcctgcactgggtagacccacagaaattcgttgcaaatgtcgccaattctctaagggatcatggcaccttgagttactggctttatacagaaccaatttttcagaacgaaagggccaatcaggtttacaacaaattcacctacgggtctgactacctgggcccatattgggatcctggccgtacacatttcagaaatcatttaaaggagttgaatcacatcttactggatagcgagctttttgacgaagtgaaaatttctaattttaaacaagaggaaggggtcaaaaacggagatatcttatacttggaaaaagagatgacaatcagtgatttcatcaattttgtatctagttggccttctgtgttttcgtggaagcagcaacgaggaaaggagggtatcctagatgatttttacaacgaactgaacgactgctttgaggggggtaacatgaaagtaatatggaactccgtcctagtatttgccaggaggaagcaaagggttgtataggctttagtacttatagaggaaacggggttacgtgcaagcgcgcatgcctgagctttgaggggggggactttcacatctcttcttctcacacttagccctaacacagagatcaactcgcttcagcatttcgacagtgtaaagaagctcatctcacttgaccagaatgcttttgctatctgttatttcaaagacctagagtacctcccaattgccatagaagcttggaaactttatgaaccttcttctaaagtaccggtggtattcgctcaagtgacacaactccccaaatcagctccaatcgaatggagcgggtcagtgtatcaagagacaccaaactacgatgattactacggtgattcgtcagaaaatgaagatgagtccacacagattgccaacaacttgtcctcgctagaattacagggacaatcggtcacatttcaagacggctacgttagcttgacattcgccccaggcctatcagccacagcacacaacacggtaattttaaacaaggggcaggcaagcaagctggaaaccattccacaggaatattccgtcgacatcatccccgtcaacagactgttcgatttcacagggaaagatcaggacctagccgtactccacaggctgaacatctgagatcaggcatgagaatccttacaccatctatgttgctctagggctgtcaaccctattatgcacgtgacggtgcgactctttcaaatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgttcatcttagagaatgtcagtgaggttatttctgagctaatcattaccttgcccggtacaggaatagggtgttaccttattaattacgacggtggtcaaaacgacgaaatttaccccatttttgagttacaagacctgaatttggaaatgatgaaacaattgtaccaagtcttggaggaccatgtaagtgggcttaatcctctcgagaagcaattcccaattgaacacagtaaaccgttatcagccactctgttctttcacttaaggtctcttttttacatggcgaagactcataagcgtactggaagacattacaacttgaaaaagattttcttgttcactaataacgataaaccttacaatggaaactctcagctgagagttcccttgaagaaaaccctggctgattacaatgacgtagacattactttgattccgtttcttctgaacaagccttcaggtgtcaagtttgacaagacggaatactcagaaattttgttctatgataaagatgcttgttcgatgtcaattgaggagatccgccaacgaatttctagacataaggagatcaagcgggtttacttcacctgtcctttgaaaatcgcaaataacttgtgcatttctgtgaaaggttattctatgttttatcatgaaactccaaggaagatcaaatttgtcgtcaatgagggttcaactttcaaagatgtggagacaaaatctcagtttgtcgatccaacatccggaaaagagttttccagtgaacagctgatcaaagcatatcctctaggtgccgatgcttacattcctttaaactcagagcaagtcaaaacaataaatcgatttaatgatatcatcaatatcccctctttggaaattctaggtttcagggatatatctaattggttgccacagtatcagtttggcaaagcatcgtttttatcccctaataa

SEQ ID NO: 164 - protein, 269 aa, synthetic construct; identical to SEQ ID NO: 158
  source 1..269: note = The sequence is a translation of CDS kanMX nt (1611)..(2420) of SEQ ID NO. 163

SEQ ID NO: 165 - DNA, 786 bp, Bacillus sp.
  source 1..786: mol_type = genomic DNA
  CDS 1..786: note = Essential gene sufC in the iscU operon
atggctgcttcaacattaacgatcaaagatcttcacgttgaaatcgaagggaaagagatcttaaagggtgtaaaccttgaaataaaaggtggagaattccacgcagtaatgggcccgaacggaactggtaaatccactttatcagctgctattatggggcatcctaaatatgaagtaacaaaaggcagcatcacgcttgacggcaaagatgtactggagatggaagtggacgagcgtgcgcaggcaggcttattccttgcaatgcagtacccaagtgaaatcagcggtgtgacaaatgccgacttccttcgctcagcaatcaacgcgcgcagagaagaaggcgatgaaatttctctcatgaaattcatccgcaaaatggacgaaaacatggagttccttgaaatggaccctgaaatggctcagcgctatcttaacgaaggcttctcaggcggggagaaaaaacgcaacgaaatccttcaattaatgatgattgaaccgaaaatcgccatccttgatgaaattgactcaggccttgatattgacgctttgaaagttgtatcaaaagggatcaacaaaatgcgcagcgaaaacttcggctgcctgatgatcactcactatcagcgcctgttaaactacatcactccggatgttgttcacgttatgatgcaaggccgcgttgtaaaatccggcggtgcagagcttgcacagcgtcttgaagcagaaggttatgactggatcaaacaagaacttggcattgaagacgaaactgttggccaagaagcgtaa

SEQ ID NO: 166 - protein, 261 aa, Bacillus sp.
  source 1..261: note = The sequence is a translation of SEQ ID NO. 165
MAASTLTIKDLHVEIEGKEILKGVNLEIKGGEFHAVMGPNGTGKSTLSAAIMGHPKYEVTKGSITLDGKDVLEMEVDERAQAGLFLAMQYPSEISGVTNADFLRSAINARREEGDEISLMKFIRKMDENMEFLEMDPEMAQRYLNEGFSGGEKKRNEILQLMMIEPKIAILDEIDSGLDIDALKVVSKGINKMRSENFGCLMITHYQRLLNYITPDVVHVMMQGRVVKSGGAELAQRLEAEGYDWIKQELGIEDETVGQEA

SEQ ID NO: 167 - DNA, 1314 bp, Bacillus sp.
  source 1..1314: mol_type = genomic DNA
  CDS 1..1314: note = Essential gene sufD in the iscU operon
atgacactaggtacaaaactatccgtagatcaggagtatctcaaaagcttttccgaaaagcaccaagaacctgcctggctgaagaacctgcgcttacaggctcttgaacaagctgaggatctgccgatgccgaaacctgacaaaacaaaaatcacaaactggaacttcacgaatttcgcgaagcatacagtggataacgaaccgctttcttcattagaagatttgactgatgaagtaaaagcgctgatcgacattgaaaacgaagataaaacattatacgtacagcgtgaccagacgccggcacatctttctctttctcaagaattgaaagacaaaggcgtcatcttcactgatattctgactgctgcacgcgagcacagcgacttggtggagaaatactttatgaaggacggcgtgaaggttgacgaacataagttaactgcacttcacgcggctcttgttaacggaggagcattcctttatgttccgaaaaatgttcaggtggagacgccggttcaggctgtttatgtccacgaaagcaatgatacagctctattcaaccacgtgctgattgtggctgaagaccacagctctgtaacatatgtagaaaactacatcagcactgtaaacccgaaagacgctgtcttcaatattatcagtgaggtcatcacaggtgataatgccagcgtgacatacggtgcggtggataatctgtcaagcggtgtgacaacttatgtgaaccgccgcggtgctgcgcgcggacgcgacagcaaaatcgagtgggctctcggcctgatgaacgatggcgacacgatttccgaaaacacaacaaacctttacggtgacggcacatacggcgatacgaaaacggttgttgtcggaagaggagagcaaacagaaaacttcacaacacaaatcatccatttcggtaaagcttctgaaggctatatcttgaagcacggtgtgatgaaggattccgcttcttctatctttaacggaatcggcaaaatcgaacatggcgcttctaaagcgaatgcggagcaagaatcacgcgtgctgatgcttagcgaaaaagcacgcggagacgcaaaccctattcttttaattgacgaagacgatgtaactgcaggacatgcggcatctgtcggccgtgtggaccctattcagctttactacctgatgagccgcggaattccgaaagaagaagcagaacgcttagtcatttacggattccttgcaccggtagtaaatgaacttccgattgaaggcgttaagaaacaattagtttctgttatcgaaaggaaagtgaagtaa

SEQ ID NO: 168 - protein, 437 aa, Bacillus sp.
  source 1..437: note = The sequence is a translation of SEQ ID NO. 167
MTLGTKLSVDQEYLKSFSEKHQEPAWLKNLRLQALEQAEDLPMPKPDKTKITNWNFTNFAKHTVDNEPLSSLEDLTDEVKALIDIENEDKTLYVQRDQTPAHLSLSQELKDKGVIFTDILTAAREHSDLVEKYFMKDGVKVDEHKLTALHAALVNGGAFLYVPKNVQVETPVQAVYVHESNDTALFNHVLIVAEDHSSVTYVENYISTVNPKDAVFNIISEVITGDNASVTYGAVDNLSSGVTTYVNRRGAARGRDSKIEWALGLMNDGDTISENTTNLYGDGTYGDTKTVVVGRGEQTENFTTQIIHFGKASEGYILKHGVMKDSASSIFNGIGKIEHGASKANAEQESRVLMLSEKARGDANPILLIDEDDVTAGHAASVGRVDPIQLYYLMSRGIPKEEAERLVIYGFLAPVVNELPIEGVKKQLVSVIERKVK

SEQ ID NO: 169 - DNA, 1221 bp, Bacillus sp.
  source 1..1221: mol_type = genomic DNA
  CDS 1..1221: note = Essential gene sufS in the iscU operon
atgaatatcacagatattcgtgaacagttcccgatccttcatcagcaagtgaacggacatgatctcgtttatctcgacagcgcggcgacttcccagaagccgcgtgctgtcattgaaacactggataagtattataaccaatacaattccaatgttcaccggggcgtccatacacttggaaccagagcgacagatggttatgaaggagcgcgtgaaaaagtccgcaagtttattaacgcgaagtcaatggctgagattattttcacaaaaggcacgaccacatcactgaatatggtggcgttaagctatgcgcgcgccaacctgaaacctggtgatgaagtggtcatcacctacatggagcatcatgcgaatatcattccgtggcagcaggctgtaaaagcaactggcgccacattaaaatatattccgttgcaggaagacggaacgatttctttagaagacgtcagagaaacggtcacaagcaacacaaaaattgttgcggtgtctcatgtatctaatgttctcggcacggtcaacccgattaaagaaatggcgaaaatcgctcatgacaatggggctgtcattgtggtggacggtgcacaaagcacgcctcacatgaaaattgacgtgcaggatctcgattgcgacttctttgcgctttcttcccataaaatgtgcggacctaccggtgttggtgtgctgtacggaaagaaagccctgctggaaaacatggagcctgccgaattcggcggtgaaatgatcgactttgtcgggctttatgaatcaacttggaaagagcttccgtggaaattcgaagcgggtacgccgattattgcaggagcaatcggcctcggtgccgccattgattttctcgaagaaatcggtcttgatgagatttcccgccatgagcacaagcttgcagcttatgcgcttgaacgcttccgccagcttgacggcgtaacggtttacgggccggaagagcgtgccgggcttgtcacatttaaccttgatgatgttcatccgcacgatgtggcgactgtgcttgatgcggagggaatcgcagtcagagccggccaccattgtgcgcagccgctgatgaaatggctggatgtaactgctactgcaagagcaagcttttatctgtataatacagaggaagagattgataagcttgtggaagctcttcaaaagacaaaggagtattttacaaatgtcttttaa

SEQ ID NO: 170 - protein, 406 aa, Bacillus sp.
  source 1..406: note = The sequence is a translation of SEQ ID NO. 169
MNITDIREQFPILHQQVNGHDLVYLDSAATSQKPRAVIETLDKYYNQYNSNVHRGVHTLGTRATDGYEGAREKVRKFINAKSMAEIIFTKGTTTSLNMVALSYARANLKPGDEVVITYMEHHANIIPWQQAVKATGATLKYIPLQEDGTISLEDVRETVTSNTKIVAVSHVSNVLGTVNPIKEMAKIAHDNGAVIVVDGAQSTPHMKIDVQDLDCDFFALSSHKMCGPTGVGVLYGKKALLENMEPAEFGGEMIDFVGLYESTWKELPWKFEAGTPIIAGAIGLGAAIDFLEEIGLDEISRHEHKLAAYALERFRQLDGVTVYGPEERAGLVTFNLDDVHPHDVATVLDAEGIAVRAGHHCAQPLMKWLDVTATARASFYLYNTEEEIDKLVEALQKTKEYFTNVF

SEQ ID NO: 171 - DNA, 444 bp, Bacillus sp.
  source 1..444: mol_type = genomic DNA
  CDS 1..444: note = Essential gene sufU (synonym iscU) in the iscU operon
atgtcttttaatgcaaacttagatacattgtacagacaggtgattatggatcattacaaaaacccgcgaaataaaggggttttaaatgacagcattgtcgtggatatgaacaatccgacatgcggcgaccgcatcagactgacaatgaagcttgacggagacatcgtggaagatgcgaagtttgaaggggaaggctgttccatttcaatggcatccgcttcgatgatgactcaggcgattaaagggaaagatattgaaactgccctttccatgtctaagattttctcggatatgatgcaagggaaagagtatgatgattctatagatctcggggatattgaagcccttcaaggcgtttcaaaattccctgcccgtatcaaatgtgcaaccctgtcatggaaagcacttgaaaaaggagtcgcgaaagaagaaggcggcaattaa

SEQ ID NO: 172 - protein, 147 aa, Bacillus sp.
  source 1..147: note = The sequence is a translation of SEQ ID NO. 171
MSFNANLDTLYRQVIMDHYKNPRNKGVLNDSIVVDMNNPTCGDRIRLTMKLDGDIVEDAKFEGEGCSISMASASMMTQAIKGKDIETALSMSKIFSDMMQGKEYDDSIDLGDIEALQGVSKFPARIKCATLSWKALEKGVAKEEGGN

SEQ ID NO: 173 - DNA, 1398 bp, Bacillus sp.
  source 1..1398: mol_type = genomic DNA
  CDS 1..1398: note = Essential gene sufB in the iscU operon
atggctaaaaaaatgcctgatattggtgaatacaagtatggttttcacgacaaggacgtttccattttccgttcagagcgcggattgacaaaagaaatcgtagaagaaatttctcgcatgaaagaagagcctcaatggatgcttgacttccgcttgaaatcacttgagcatttctacaatatgccgatgcctcaatggggcggagatttaaactcactgaactttgacgaaattacgtactacgtaaaaccgtctgagcgttcagagcgttcttgggatgaagttcctgaagaaattaaacaaacattcgacaagctcggtattcctgaagctgaacaaaaataccttgcgggtgtttctgctcagtacgaatctgaggttgtataccacaacatgaaagaagatcttgaagcacaaggcatcgtctttaaagacactgacagcgcgctgaaagagaatgaagatattttccgtgagcactgggcaaaagtaatcccgccgactgataacaagtttgcggcgcttaactcagctgtttggtccggcggttctttcatctacgtgcctaaaggcgtgaaggttgaaacaccgctacaagcttacttccgtatcaactctgagaacatgggtcagttcgaacgtacactgatcattgttgacgaagaagcaagcgttcattatgttgaaggctgtactgcgcctgtttacacaacaaactcacttcacagtgcggtcgttgagatcatcgtgaaaaaaggcggctactgccgttatacaacgatccaaaactgggcgaacaacgtttacaaccttgtaacaaagcgtacagtctgtgaagaaaacgcaacaatggaatggatcgacggcaacatcggttctaagctgacaatgaaatatccggcttgtatcctaaaaggcgaaggcgcgcgcggtatgacgctttctatcgctcttgcgggtaaaggccagcatcaggatgcaggtgcgaaaatgattcaccttgcaccaaacacatcatctacaatcgtatcaaaatcgatttcaaaacagggcggaaaagtaacgtaccgcggaatcgtccacttcggacgcaaagcggaaggcgcccgctctaacatcgagtgtgatacgctcattatggataacaaatcaacttctgacacaattccttacaatgaaatcttaaatgataacatttcattggagcacgaagcgaaagtgtctaaagtatctgaagagcagctcttctacttgatgagccgcggtatttctgaagaagaagcaactgaaatgatcgtaatgggctttatcgagccattcacaaaagagcttccgatggaatacgcagttgaaatgaaccgtttgattaagttcgaaatggaaggttctatcggttaa

SEQ ID NO: 174 - protein, 465 aa, Bacillus sp.
  source 1..465: note = The sequence is a translation of SEQ ID NO. 173
MAKKMPDIGEYKYGFHDKDVSIFRSERGLTKEIVEEISRMKEEPQWMLDFRLKSLEHFYNMPMPQWGGDLNSLNFDEITYYVKPSERSERSWDEVPEEIKQTFDKLGIPEAEQKYLAGVSAQYESEVVYHNMKEDLEAQGIVFKDTDSALKENEDIFREHWAKVIPPTDNKFAALNSAVWSGGSFIYVPKGVKVETPLQAYFRINSENMGQFERTLIIVDEEASVHYVEGCTAPVYTTNSLHSAVVEIIVKKGGYCRYTTIQNWANNVYNLVTKRTVCEENATMEWIDGNIGSKLTMKYPACILKGEGARGMTLSIALAGKGQHQDAGAKMIHLAPNTSSTIVSKSISKQGGKVTYRGIVHFGRKAEGARSNIECDTLIMDNKSTSDTIPYNEILNDNISLEHEAKVSKVSEEQLFYLMSRGISEEEATEMIVMGFIEPFTKELPMEYAVEMNRLIKFEMEGSIG

SEQ ID NO: 175 - DNA, 480 bp, Bacillus sp.
  source 1..480: mol_type = genomic DNA
  CDS 1..480: note = Essential gene accB (in accC operon)
atgttaaatatcaaagaaatccacgagctgattaaagcaattgacgagtctacaattgacgaattcgtatatgaaaatgaaggtgtatccttaaaactgaaaaaacacgaagcaggcacggttcaagtcatgcagcaggcaccggcagcacctgtacaagcacaggctccgcaggcagttcagccgcaagcgcagcaagcagcggcacctgcccaagaagcaccaaagcaagatgagaatctgcataaaatcacttcaccaatggtaggaacattttatgcttcttcatcaccggaagctggcccgtatgtaacagccggttcaaaagtaaatgaaaacacagttgtctgcattgtagaagcgatgaagcttttcaacgaaatcgaagcagaagtgaaaggcgaaatcgttgaagtattagtagaaaacggccagctggtcgaatacggacaacctctatttcttgtaaaagcggagtaa

SEQ ID NO: 176 - protein, 159 aa, Bacillus sp.
  source 1..159: note = The sequence is a translation of SEQ ID NO. 175
MLNIKEIHELIKAIDESTIDEFVYENEGVSLKLKKHEAGTVQVMQQAPAAPVQAQAPQAVQPQAQQAAAPAQEAPKQDENLHKITSPMVGTFYASSSPEAGPYVTAGSKVNENTVVCIVEAMKLFNEIEAEVKGEIVEVLVENGQLVEYGQPLFLVKAE

SEQ ID NO: 177 - DNA, 1353 bp, Bacillus sp.
  source 1..1353: mol_type = genomic DNA
  CDS 1..1353: note = Essential gene accC (in accC operon)
atgattaaaaagctattgatcgccaacagaggagaaattgctgtcagaatcatcagagcctgcagagagctcggaattgagactgtcgctgtttattcagaagctgataaagatgcccttcatgttcaaatggccgatgaagctttttgtatcggaccgaaagcatcaaaagacagctatttaaacgttacaaatattgtgagtgttgcaaagctgactggcacggacgccattcatccgggatacggatttttagctgaaaatgctgatttcgctgaattatgtgaagaagtgaatgtcacgtttgtcggcccgagcgctgacgccatttcaaaaatgggaacaaaagacgttgcgcgggaaacgatgaaacaggccggcgtgccaatcgtaccgggttcacagggaattatagaaaatgtggaagaagcggtttcgcttgctaatgaaattgggtatcctgtaattataaaagccaccgcaggcggaggcggaaaaggaattagggttgcccgcactgaagaggaactgattaatggcattaagattacacagcaggaagcggcaactgcatttgggaatccaggtgtatacatcgaaaaatacatagaagattttcgccacgttgagatccaagtgcttgctgataactacggaaatacgatccatttgggcgaacgcgactgctcgatccaaagacgcctgcaaaagcttttggaagaatcaccatctcctgcccttgattcagaaatcagggagcaaatgggagatgcagcggtaaaggctgccaaagcggttggctatacaggtgctggaacagttgaatttatctatgactacaatgaacagcgctattacttcatggaaatgaacacgagaatccaggtggagcacccagtcacagaaatggtgacgggaactgacctgatcaaggaacaaatcaaagtggcttcaggaatggaactgagcctcaagcaagaagatgttgaatttgaaggctgggccatcgaatgccgaatcaacgcagaaaacccaagcaaaaatttcatgccgtcacctggtgaaattaaaatgtacctgcctcctggcggtcttggtgtccgtgtcgattcagctgcgtaccctggctattccatcccaccgtactatgacagcatgattgcaaaggtaatcacatacggaaaaacgagagatgaagcgattgcccgcatgaagcgcgcattgagcgaattcgtcatcgaaggcattgagacaacaatccctttccatttaaaactgcttgaacacgaaacatttgtgagcggagagtttaatacgaaatttttagaaacatatgatgtaatgggctcataa

SEQ ID NO: 178 - protein, 450 aa, Bacillus sp.
  source 1..450: note = The sequence is a translation of SEQ ID NO. 177
MIKKLLIANRGEIAVRIIRACRELGIETVAVYSEADKDALHVQMADEAFCIGPKASKDSYLNVTNIVSVAKLTGTDAIHPGYGFLAENADFAELCEEVNVTFVGPSADAISKMGTKDVARETMKQAGVPIVPGSQGIIENVEEAVSLANEIGYPVIIKATAGGGGKGIRVARTEEELINGIKITQQEAATAFGNPGVYIEKYIEDFRHVEIQVLADNYGNTIHLGERDCSIQRRLQKLLEESPSPALDSEIREQMGDAAVKAAKAVGYTGAGTVEFIYDYNEQRYYFMEMNTRIQVEHPVTEMVTGTDLIKEQIKVASGMELSLKQEDVEFEGWAIECRINAENPSKNFMPSPGEIKMYLPPGGLGVRVDSAAYPGYSIPPYYDSMIAKVITYGKTRDEAIARMKRALSEFVIEGIETTIPFHLKLLEHETFVSGEFNTKFLETYDVMGS

SEQ ID NO: 179 - DNA, 408 bp, Bacillus sp.
  source 1..408: mol_type = genomic DNA
  CDS 1..408: note = Essential gene yghY (in accC operon)
atgaaagacaacagcttgcttaaaatggatcacgaagatacgcatttgggtaaggttgagattgcaccggaggtcattgaggtcattgccggcattgcagcatcagaagttgatggagttgccgaaatgcgcggaaactttgcgacaggcgtcgttgagcgctttggcaaagtgaatcatggcaaaggtgtcaaagtcgacctggcggatgacggaattacgatcgatgtatactgtgtcgttacatttggcgtttcgattccgaaagttgcagcatccgttcaggaaaacattcgtcaaaccttattaaatatgacgtctctttcgattaacgagatcaatattcacatcgtcggcattcaatttgacacaaaagcccaagaagtccaaatcgacgaagaaatgtaa

SEQ ID NO: 180 - protein, 135 aa, Bacillus sp.
  source 1..135: note = The sequence is a translation of SEQ ID NO. 179
MKDNSLLKMDHEDTHLGKVEIAPEVIEVIAGIAASEVDGVAEMRGNFATGVVERFGKVNHGKGVKVDLADDGITIDVYCVVTFGVSIPKVAASVQENIRQTLLNMTSLSINEINIHIVGIQFDTKAQEVQIDEEM

SEQ ID NO: 181 - DNA, 1344 bp, Corynebacterium sp.
  source 1..1344: mol_type = genomic DNA
  CDS 1..1344: note = Corynebacterium essential gene glmM
atgactcgactttttggaactgatggcgtccgcggactagccaatgaagtactcaccgcacctttggccctgaaactgggtgccgctgcagctcacgtacttaccgcagagaaacgcgtagatggacgccgcccggttgcgatcgttggtagggatcctcgagtctctggagagatgctcgcggcagcactttcagcaggcatggccagccagggtgttgatgtcattcgtgttggtgtcatcccaaccccagctgttgcattcctcaccgatgattatggcgctgatatgggcgtgatgatttctgcatcccacaacccaatgccggacaacggaatcaagttcttctccgcaggtggacacaagcttccagaccatgtggaagacgagattgagcgtgtcatggacagcttgcctgccgaaggcccaaccgggcatggagttggccgtgtcattgaagaagcaaccgatgcacaagaccgctacctggagcacctgaaggaagctgttcctacgtcacttgaaggcatcaagattgttgtggatgcagccaatggtgcagcaagcgttgtagctccaaaggcttatgaagcagcaggtgcaactgtaattgctattcataacaagccagactcctacaacatcaacatggattgcggttccacccacattgaccaggtgcaggcggcagttctgaagcacggtgctgaccttggactcgcacacgacggcgatgctgaccgttgtttggctgtggacaaagatggaaacctcattgacggtgatcagatcatggcaatgttagccattgcgatgaaagaaaacggcgagctgcgcaagaacaccctcgtgggcaccgtcatgagcaacttgggactgaagattgccatggatgaagccggaattacgctgcgaaccaccaaggtgggcgaccgctacgtgctggaagacctcaatgcgggtggattcagccttggtggcgagcagtctggacacattgttcttccagatcacggcaccactggcgatggaactttgactggtctttccatcatggcgcgcatggctgaaaccggaaaatccttgggcgagttggcacaagcgatgacggttctgccacaggttctgatcaatgtgccagtctctgataaatccaccatcgtttctcacccaagtgtcgttgcagcaatcgctgaagctgaagcggagttgggttctaccggtcgcgtgcttcttcgcgcttctggcaccgaggagcttttccgtgtgatggtcgaagctggcgataaggaacaagcacgccgcattgcgggacgtctttctgccgtggtcgcggaagtctaa

SEQ ID NO: 182 - protein, 447 aa, Corynebacterium sp.
  source 1..447: note = The sequence is a translation of SEQ ID NO. 181
MTRLFGTDGVRGLANEVLTAPLALKLGAAAAHVLTAEKRVDGRRPVAIVGRDPRVSGEMLAAALSAGMASQGVDVIRVGVIPTPAVAFLTDDYGADMGVMISASHNPMPDNGIKFFSAGGHKLPDHVEDEIERVMDSLPAEGPTGHGVGRVIEEATDAQDRYLEHLKEAVPTSLEGIKIVVDAANGAASVVAPKAYEAAGATVIAIHNKPDSYNINMDCGSTHIDQVQAAVLKHGADLGLAHDGDADRCLAVDKDGNLIDGDQIMAMLAIAMKENGELRKNTLVGTVMSNLGLKIAMDEAGITLRTTKVGDRYVLEDLNAGGFSLGGEQSGHIVLPDHGTTGDGTLTGLSIMARMAETGKSLGELAQAMTVLPQVLINVPVSDKSTIVSHPSVVAAIAEAEAELGSTGRVLLRASGTEELFRVMVEAGDKEQARRIAGRLSAVVAEV

SEQ ID NO: 183 - DNA, 1086 bp, Corynebacterium sp.
  source 1..1086: mol_type = genomic DNA
  CDS 1..1086: note = Corynebacterium essential gene alr
atgaacttgctgaccaccaaaattgacctggatgccatcgcccataacacgagggtgcttaaacaaatggcgggtccggcgaagctgatggcggtggtgaaggcgaatgcatataaccatggcgtggagaaggtcgctccggttattgctgcgcatggtgcggatgcgtttggtgtggcaactcttgcggaggctatgcagttgcgtgatatcggcatcagccaagaggttttgtgttggatttggacaccggagcaggatttccgcgccgccattgatcgcaatattgatttggctgttatttctcccgcgcatgccaaagccttgatcgacactgatgcggagcatattcgggtgtccatcaagattgattctgggttgcatcgttcgggtgtggatgagcaggagtgggagggcgtgttcagcgcgttggctgctgccccgcacattgaggtcacgggcatgttcacgcacttggcgtgcgcggatgagccagagaatccggaaactgatcgccaaattattgcttttcgacgcgcccttgcgctcgcccgcaagcacgggcttgagtgcccggtcaaccacgtatgcaactcacctgcatttttgactcgatctgatttacacatggagatggtccgaccgggtttggccttttatgggttggaacccgtggcgggacgagagcatggtttgaagccggcgatgacgtgggaggcgaaggtgagcgtcgtaaagcaaattgaagctggacaaggcacctcttatggcctgacctggcgcgctgaggatcgcggctttgtggctgtggtgcctgcgggctatgccgatggcatgccgcggcatgcccaggggaaattctccgtcacgattgatggcgtggactatccgcaggttggacgcgtgtgcatggatcagttcgttatttctttgggcgacaatccgcacggtgtggaagctggcgcgaaggccgtgatattcggtgagaatgggcatgacgcaactgattttgcggagcgtttagacaccattaactatgaggtagtgtgccgaccaaccggccgaactgtccgcgcatatgtttaa

SEQ ID NO: 184 - protein, 361 aa, Corynebacterium sp.
  source 1..361: note = The sequence is a translation of SEQ ID NO. 183
MNLLTTKIDLDAIAHNTRVLKQMAGPAKLMAVVKANAYNHGVEKVAPVIAAHGADAFGVATLAEAMQLRDIGISQEVLCWIWTPEQDFRAAIDRNIDLAVISPAHAKALIDTDAEHIRVSIKIDSGLHRSGVDEQEWEGVFSALAAAPHIEVTGMFTHLACADEPENPETDRQIIAFRRALALARKHGLECPVNHVCNSPAFLTRSDLHMEMVRPGLAFYGLEPVAGREHGLKPAMTWEAKVSVVKQIEAGQGTSYGLTWRAEDRGFVAVVPAGYADGMPRHAQGKFSVTIDGVDYPQVGRVCMDQFVISLGDNPHGVEAGAKAVIFGENGHDATDFAERLDTINYEVVCRPTGRTVRAYV

SEQ ID NO: 185 - DNA, 1119 bp, Aspergillus oryzae
  source 1..1119: mol_type = genomic DNA
  CDS 1..1119: note = Aspergillus essential gene ARG3
atgacttgcggtctgaagctggccgccgctcgttacggcaaccataccttgcgccagaagattccactcaacgctgtgcgccgatacacttctcacactgctacttctacgactccccccacctcgccattcgctcctcgccattttctttccatcgctgatcttacctcaacggagttcgcaaccctcgtccgtaatgcatcttcacacaaacgaaccatcaagtcgggatcaatccctcagaacctgcttggttctatgaccggacagactgtagctatgctgttcagcaaacgcagtaccagaaccaggatatctactgaaggggctgtggtgcgcttgggaggacatccaatgtttctgggcaaggatgatattcaactcggtgtcaatgagtccttgtatgactctgcggttgtaatctcctccatggtctcttgtattgtagcccgagtcggcaagcacgctgaggttgcagaccttgccaagcattcaaccgtgcctgttatcaacgctctctgtgactccttccaccctctccaggccattgccgattttcagaccatctacgaaacatttacacctaaggcccatcgctctgacagtttgggtctggagggcctcaagattgcctgggtcggtgatgcaaacaacgttctgttcgacatggcaattgctgctaccaagatgggtattgatatcgccgttgcgactccgaagggctatgagattcctgccccgatgttggagctcatcaagcaggccagcaacggtgtctcaaagccaggaaagattattgagaccaacgttcctgaggaggcggttaagggtgcggatatcctggtaactgacacctgggtttccatgggacaggaggcagagtcgatcaagcgggtgaaggacttcgagggcttccagatcacttcggaacttgccaagcgcggtggtgcgaacgaaggctggaagttcatgcactgcctgcctcgtcaccctgaggaagtcagcgatgaagttttctacagcccacggtccttggtcttccccgaagcggagaatcggttgtgggctgcgatctcggccatggagggctttgttgtcaataagggaaggattgagtaa

SEQ ID NO: 186 - protein, 372 aa, Aspergillus oryzae
  source 1..372: note = The sequence is a translation of SEQ ID NO. 185
MTCGLKLAAARYGNHTLRQKIPLNAVRRYTSHTATSTTPPTSPFAPRHFLSIADLTSTEFATLVRNASSHKRTIKSGSIPQNLLGSMTGQTVAMLFSKRSTRTRISTEGAVVRLGGHPMFLGKDDIQLGVNESLYDSAVVISSMVSCIVARVGKHAEVADLAKHSTVPVINALCDSFHPLQAIADFQTIYETFTPKAHRSDSLGLEGLKIAWVGDANNVLFDMAIAATKMGIDIAVATPKGYEIPAPMLELIKQASNGVSKPGKIIETNVPEEAVKGADILVTDTWVSMGQEAESIKRVKDFEGFQITSELAKRGGANEGWKFMHCLPRHPEEVSDEVFYSPRSLVFPEAENRLWAAISAMEGFVVNKGRIE

SEQ ID NO: 187 - DNA, 2184 bp, Aspergillus oryzae
  source 1..2184: mol_type = genomic DNA
  CDS 1..2184: note = Aspergillus essential gene adeA
atgggtgctagactcacatatataaatagtatcattctcctatccgattgtaccatcgcacataatcttgtctcattccacgatttgaatgcggggttcagaaccaacattcatttacgtctccccaaacaaacatccgacttccacaccatgtcttcctctttgagcacgggttccttccaaaccctcacttttcaccctgacaacacggtgattatacaggataaaatctacggggagcacaccatctcagagccggttctagccgagctgcttcggtgtccagctctacttcgactcgcgggcatcggtcttcacggtcaaaccgatcttctgggtataacccatacagtgactcgactagagcactccataggcgcgtcgctattagtgcgcaaagtcggcggtagcatcggagagcaagtcgcaggtcttctgcacgacatcagccacacggtactcagccatgacgtcgatggggccttgtcgaagccaggggagagctatcatgaggtccaaaagtcacggtatatcatgaccactgagttgccacgaatactcaccaaacatggctttgttgatctaaagccgtttgatgaagagctttatcctctggtggagagacccgctcctcatctctgcgctgatcgtctcgattactctctacgagatgctgtcgcgttcggtaaacttgcgatagaggatgcgcgccgcgtgtatgactcattgacagcattcccggacgcgtcttcaccacatcgcttgctggttcttcgagacattgatctagccttagcttatgctagggcctatggtgaatgcgatagggatgtgtggtgcaatccggctcatgccgtcatgtccaggaagataggtcagttgattggggatctagtgcaacaaggatcgttgaaggaagaggtcctatggaatctatcggaccgcgaattttgggagctcctgaaaagtaaagttgactctaaaggattggagacgattaaacacatcgaagcagggccgcatgcggaagattatcatcgtctacctcgtggtaccaagattcgcacaatcgatcctgatctgctcctcccgggtgctgggcaaccctccccactgtcttttgtgaaacctgaatgggctaaggagagacaagacttcattcaggctcgccaggccttactagcatcttttactgtctacatcaacggcggcgtgagctgggaacaagatagtattcagccacaattcgagaacaataatcccatcatgtctgaagctctgacaaacaccgaccttcagggtgctctccctctcatcgctcgaggcaaggttcgcgacctgtacgatgtcgacgagaagaccctcctcttcgtcgcaacggaccgcatctccgcgtacgatgtgatcatggagaatggaatccccgagaaaggcatcctcctaactctatgcacgaaaacctggttcaagatcctctccgacaaaatcccctctctccgcacccacttcctcaccctcgacctcccgcctcagatccccgagtcgttgcgtcccgtcctccagaacagaagcatgcaggtgcgcaagctgaagatcctccccattgaggccatcgtccgcggttacatcaccggttccgcctggaacgagtacaagaaatccggcaccgtgcacgggatcaaggtcgccgagggtctgagggagagcgaggccttccctgatggtcctatttacactcccagtaccaaggcggagcagggcgagcatgatgagaacattcatccggatcaggctgtcgctattgttggtgaacgttatgcctctaagatcgcctctcttgcggttcagctttacaaggtcgctcacgagtatgcccttactcgtggtgtgatcattgccgacaccaagttcgagttcggtttggatcctgagactgacgaggttgtgctggccgacgaggtcctcacccccgattcatcgcgtttctggcctaaggactcgtacgagatcggtcgtggacagcagagcttcgataagcagttcttgcgtgactggttgacgagcgagggactgaagggcaagcctggtgtgcggatgaccgatgagattgcgcagaagaccagcgctaagtaccgggaggcatatgagcggatcactggtgagactaaggttcctgcggtctag

SEQ ID NO: 188 - protein, 727 aa, Aspergillus oryzae
  source 1..727: note = The sequence is a translation of SEQ ID NO. 187
MGARLTYINSIILLSDCTIAHNLVSFHDLNAGFRTNIHLRLPKQTSDFHTMSSSLSTGSFQTLTFHPDNTVIIQDKIYGEHTISEPVLAELLRCPALLRLAGIGLHGQTDLLGITHTVTRLEHSIGASLLVRKVGGSIGEQVAGLLHDISHTVLSHDVDGALSKPGESYHEVQKSRYIMTTELPRILTKHGFVDLKPFDEELYPLVERPAPHLCADRLDYSLRDAVAFGKLAIEDARRVYDSLTAFPDASSPHRLLVLRDIDLALAYARAYGECDRDVWCNPAHAVMSRKIGQLIGDLVQQGSLKEEVLWNLSDREFWELLKSKVDSKGLETIKHIEAGPHAEDYHRLPRGTKIRTIDPDLLLPGAGQPSPLSFVKPEWAKERQDFIQARQALLASFTVYINGGVSWEQDSIQPQFENNNPIMSEALTNTDLQGALPLIARGKVRDLYDVDEKTLLFVATDRISAYDVIMENGIPEKGILLTLCTKTWFKILSDKIPSLRTHFLTLDLPPQIPESLRPVLQNRSMQVRKLKILPIEAIVRGYITGSAWNEYKKSGTVHGIKVAEGLRESEAFPDGPIYTPSTKAEQGEHDENIHPDQAVAIVGERYASKIASLAVQLYKVAHEYALTRGVIIADTKFEFGLDPETDEVVLADEVLTPDSSRFWPKDSYEIGRGQQSFDKQFLRDWLTSEGLKGKPGVRMTDEIAQKTSAKYREAYERITGETKVPAV

SEQ ID NO: 189 - DNA, 750 bp, Aspergillus oryzae
  regulatory 1..750: note = promoter - Aspergillus oryzae burden sensing promoter p(bibA); regulatory_class = promoter
  source 1..750: mol_type = genomic DNA
ctatgtagaatagatagtagtaggggtgtcatcctccattatctgacaagcagataccagcatgtggccacgtgtagtgatgttgtgcaagtcattaagctgcagaggtcatgacttcacagtgactagagttgagtaacttgagatatggaaagtacacggtgtactcaaggtacagtacaattggaggttgacgacgtcctttgtccacgtatcctaaaggagcatatggccggggggctgaaaaatagggcaacttcaagaaagattacctaacaggggcttctagccaatgaaacacatttctagtatgttcggtggaccgcttgcagaagatccatgaacactttggggcaatttctacggcagaatacagggcggtggccaaggcggatgggtcattctggtccctgaaaggcgatgcaccaatcacaacgcgaggagtaacctgcagagcgatggccgtaccagccatgctgtccttttctctggcctcaagcattttaaaaaagctgatctcttcctctcacgtccttcgttcagcaacttctctcttttgcctcaactttcccttccttcccccttccattccgtcgcctgtggttgggttctctttttcttccttttgcttctctttcttgaatacaggaaactgtattgaagcacaagggatttactaccaacacgtctcccgataatcacacgcgtgccccttggcgaagaccaccactcgatatacacataggcaca

SEQ ID NO: 190 - DNA, 750 bp, Aspergillus oryzae
  regulatory 1..750: note = promoter - Aspergillus oryzae burden sensing promoter p(clxA); regulatory_class = promoter
  source 1..750: mol_type = genomic DNA
agtgtggtctgaagttttgcaaagtgagtgaggaggggaaaagaacgtcggcggggagaggggcaacgtgaagaactccattttttttttttctggatatggataattaactagtagcgggaagtagatcgcgcaatcggtatttgcaaggatagtatgcaattacttcgaagattaagaaaggaacaagaggaaaaatagaaaagaatttatttatattcatttgaaatttacattgactggattgaattgtcttatttgaattgggtgactagcatgtttgggagaagtgtactaatatgaatgaatggtatggagtactacaaataacatacagggacgctaagaccgtgacaaccaaagagagaaaacaaaaaaagcaataaaacggttctttagaatcaatggacactggacagaacattggagaatctgccactaatcctgtttgggtcggtggaactttaccccaaggggcaagcggcagcggaagaggccagattgaacttaagctaccttcccagcttcatataatcaacctccccaacactcgtcttctctttctcctctcctctctcttcttccttctgtcgtgcggttagcttgtggtacgctctcaccttgacactcttttccccctcgaacctctacggaggaccaattgcttgatcgtttgcttatttgtttctttctttcctaaagctactaccgagtcgggcttcttagtttgagagcccgttatcattcatc

SEQ ID NO: 191 - DNA, 300 bp, Corynebacterium glutamicum
  regulatory 1..300: note = promoter - C. glutamicum burden sensing promoter p(groES); regulatory_class = promoter
  source 1..300: mol_type = genomic DNA
gcgaagacatcacgttggatgaaattgagcgaattaaggacggcattcgcgccgctcgtaactaccgcgatgattatccagaggaattcaacctgtggcgcaacgctgtatataacctgcgtacgccctaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcacc

SEQ ID NO: 192 - DNA, 300 bp, Corynebacterium glutamicum
  regulatory 1..300: note = promoter - C. glutamicum burden sensing promoter p(katA); regulatory_class = promoter
  source 1..300: mol_type = genomic DNA
tttcccggatgaaatccgtgagagctgcgcgatcgccacggccggcggctagggccagctcggtgacgtgggcgtcgttacgctcttttgacttcacaattgctcagaataccagttcagaccggggtcaccataaaggtgtgtagggggtgtcaaagtgggcaaaaagtatcatacttttgtctttttgctggcagtttacgtaagggctgattatttggtggcgcgaagcgacattctgatgtagactatcaatcagacttaatccataggtaaccctcataaaaggaaggaatgcta

SEQ ID NO: 193 - DNA, 300 bp, Corynebacterium glutamicum
  regulatory 1..300: note = promoter - C. glutamicum burden sensing promoter p(cplX); regulatory_class = promoter
  source 1..300: mol_type = genomic DNA
ggaggtggcggtggtgcgacacacaacgcgcacgcccgatggttggcgttccatcgcgctaaactggggatcacagtagcgttctactatttctttgctgagtcgatgcgggcgaaggcccctgtttgtggtttcaatttcggttgcgccgtgaataaattcgggattcattagcttaagcgtaccgctctgcagagttcacaggtaaagcgctaagatggaacaacccattgccaatattgttggttagagttgtacgcagtaaatcttttcaatcgtggaagcgggtctcacagtcta

SEQ ID NO: 194 - DNA, 300 bp, Corynebacterium glutamicum
  regulatory 1..300: note = promoter - C. glutamicum burden sensing promoter p(mutM); regulatory_class = promoter
  source 1..300: mol_type = genomic DNA
tggataccaagttggcagagtttgaaacctcgctgtccaccacgttgcgttctgtcactgctgatcgttccgcactacgccgaggagctggagctactggccgcgagctgcgcgatgagcaaccggcagcgcgtggtgaatatgagcgggactacgagcgtgattatgaacgaggctacgaacgcgacgatcgagactactagtttccaagaaaccatttaggctttttcacctttccgtctagcgatcctcatctgatgtatgtagtcacgatgaggtacgagtaggatcttcctggtc

SEQ ID NO: 195 - DNA, 4694 bp, synthetic construct
  misc_feature 1..4694: note = plasmid pEG151
  misc_feature 88..651: note = homology region; iscU upstream
  regulatory 1833..2128: note = promoter - burden addiction promoter p(groES); regulatory_class = promoter
  regulatory 2129..2134: regulatory_class = ribosome_binding_site
  misc_feature 2134: note = N is A, T, C or G
  misc_feature 2144..2621: note = homology region; iscU downstream
  source 1..4694: mol_type = other DNA
aacaaaattctccagtcttcacatcggtttgaaaggaggaagcggaagaatgaagtaagagggatttttgactccgaagtaagtcttcattgaggaattcggaaacaccacgcattaagagaaggaatattccgaaaaaccattctcatttgttctgaaaggatgctttcgtctcatctgccgctatttcacgccaaatcgtttttgcggcatccaacgcagcactggtacaataaaaagcaaatatatgaagggattgattcattatttgcagcagccaggacagactgaacctcgattatacgtcagaaatggaaaaagcgatgcagctgtcacacggaattggatacgaggaatatggacgccggctggaaacaagaatgaaagtagagcggcagagagagcttgattatgaaaaaagcaagcggatttctgcgggagcttattgagaaaatcaatcttttaaaaaagggtgggaatccactctttttctttttcaatcgctttaataaagtgctgatctttgttaaacttgaggaatgtgcaaggcacgataaaaagttgtatttacttttcatttgttataaaattagaatgagaataaaatgagaataatgattatatatttttggaggtatagatatggctgctagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattaatgcggaaaactgcatgatgatatatgtcaaaataataaaccaatactgttttctcaaatggtatgtactcctttgttaagtgggtttcgttcatctacagctattgtaacataatcggtacgggggtgaaaaagctaacggaaaagggagcggaaaagaatgatgtaagcgtgaaaaattttttatcttatcacttgaaattggaagggagattctttattataagaattgtgttagcactctttagtgctgagtgctaaaattacatattcatactattgaggaggttatttcacgagntgaaatcatatggctgcttcaacattaacgatcaaagatcttcacgttgaaatcgaagggaaagagatcttaaagggtgtaaaccttgaaataaaaggtggagaattccacgcagtaatgggcccgaacggaactggtaaatccactttatcagctgctattatggggcatcctaaatatgaagtaacaaaaggcagcatcacgcttgacggcaaagatgtactggagatggaagtggacgagcgtgcgcaggcaggcttattccttgcaatgcagtacccaagtgaaatcagcggtgtgacaaatgccgacttccttcgctcagcaatcaacgcgcgcagagaagaaggcgatgaaatttctctcatgaaattcatccgcaaaatggacgaaaacatggagttccttgaaatggaccctgaaatggctcagcgctatcttaacgaaggcttctcaggcggggagaaaaaacgcaacgaaatccttcaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 196 - DNA, 4697 bp, synthetic construct
  misc_feature 1..4697: note = Plasmid pEG152
  misc_feature 88..651: note = Homology region iscU upstream
  regulatory 1833..2131: note = promoter - burden addiction promoter p(ctsR); regulatory_class = promoter
  misc_feature 2132..2137: note = rbs - RBS
  misc_feature 2137: note = N is A, T, C or G
  misc_feature 2147..2624: note = Homology region iscU downstream
  source 1..4697: mol_type = other DNA
aacaaaattctccagtcttcacatcggtttgaaaggaggaagcggaagaatgaagtaagagggatttttgactccgaagtaagtcttcattgaggaattcggaaacaccacgcattaagagaaggaatattccgaaaaaccattctcatttgttctgaaaggatgctttcgtctcatctgccgctatttcacgccaaatcgtttttgcggcatccaacgcagcactggtacaataaaaagcaaatatatgaagggattgattcattatttgcagcagccaggacagactgaacctcgattatacgtcagaaatggaaaaagcgatgcagctgtcacacggaattggatacgaggaatatggacgccggctggaaacaagaatgaaagtagagcggcagagagagcttgattatgaaaaaagcaagcggatttctgcgggagcttattgagaaaatcaatcttttaaaaaagggtgggaatccactctttttctttttcaatcgctttaataaagtgctgatctttgttaaacttgaggaatgtgcaaggcacgataaaaagttgtatttacttttcatttgttataaaattagaatgagaataaaatgagaataatgattatatatttttggaggtatagatatggctgctagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattgctcttcagcgccgatggtagtcgggggtttccccctgtgagagtaggacgccgccaagcaagcttaaacccagctcaatgagctgggttttttgtattttggtttattggtatcataaaattccacttaactgtataatataataactttataccgaattttaaatcagcaatcaggttttgtggaccgggaaaatggaaataatgaaggatagagcgagaaagttgaaaattctcgagaaacggcttatagtaagattaaagtcaaatatagtcaaagtcagtaaaggagggggttgacgagntgaaatcatatggctgcttcaacattaacgatcaaagatcttcacgttgaaatcgaagggaaagagatcttaaagggtgtaaaccttgaaataaaaggtggagaattccacgcagtaatgggcccgaacggaactggtaaatccactttatcagctgctattatggggcatcctaaatatgaagtaacaaaaggcagcatcacgcttgacggcaaagatgtactggagatggaagtggacgagcgtgcgcaggcaggcttattccttgcaatgcagtacccaagtgaaatcagcggtgtgacaaatgccgacttccttcgctcagcaatcaacgcgcgcagagaagaaggcgatgaaatttctctcatgaaattcatccgcaaaatggacgaaaacatggagttccttgaaatggaccctgaaatggctcagcgctatcttaacgaaggcttctcaggcggggagaaaaaacgcaacgaaatccttcaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 197 - DNA, 4697 bp, synthetic construct
  misc_feature 1..4697: note = Plasmid pEG153
  misc_feature 88..651: note = Homology region iscU upstream
  regulatory 1833..2131: note = promoter - burden addiction promoter p(dnaK); regulatory_class = promoter
  misc_feature 2132..2137: note = rbs - rbs
  misc_feature 2132..2137: note = N is A, T, C or G
  misc_feature 2147..2624: note = Homology region iscU downstream
  source 1..4697: mol_type = other DNA
aacaaaattctccagtcttcacatcggtttgaaaggaggaagcggaagaatgaagtaagagggatttttgactccgaagtaagtcttcattgaggaattcggaaacaccacgcattaagagaaggaatattccgaaaaaccattctcatttgttctgaaaggatgctttcgtctcatctgccgctatttcacgccaaatcgtttttgcggcatccaacgcagcactggtacaataaaaagcaaatatatgaagggattgattcattatttgcagcagccaggacagactgaacctcgattatacgtcagaaatggaaaaagcgatgcagctgtcacacggaattggatacgaggaatatggacgccggctggaaacaagaatgaaagtagagcggcagagagagcttgattatgaaaaaagcaagcggatttctgcgggagcttattgagaaaatcaatcttttaaaaaagggtgggaatccactctttttctttttcaatcgctttaataaagtgctgatctttgttaaacttgaggaatgtgcaaggcacgataaaaagttgtatttacttttcatttgttataaaattagaatgagaataaaatgagaataatgattatatatttttggaggtatagatatggctgctagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattggttcaatccccgcacacgggaatttcgattaaaatcggaaaagaaaacgactatgaagagatggaaaattgcagtctgattacggcttcttattccgtagaccagaagcagatcggctcaattgcgattatcggcccgacccgcatgaattattccagggttgtcagcctgcttcagcatgtgacttcggacttgtcaaaagcattaacaagtctgtatgatgaataagggaattttggcaaattttatcgaagggcagcacctgtccttctccttacactttgagggaggtgaacacacgagntgaaatcatatggctgcttcaacattaacgatcaaagatcttcacgttgaaatcgaagggaaagagatcttaaagggtgtaaaccttgaaataaaaggtggagaattccacgcagtaatgggcccgaacggaactggtaaatccactttatcagctgctattatggggcatcctaaatatgaagtaacaaaaggcagcatcacgcttgacggcaaagatgtactggagatggaagtggacgagcgtgcgcaggcaggcttattccttgcaatgcagtacccaagtgaaatcagcggtgtgacaaatgccgacttccttcgctcagcaatcaacgcgcgcagagaagaaggcgatgaaatttctctcatgaaattcatccgcaaaatggacgaaaacatggagttccttgaaatggaccctgaaatggctcagcgctatcttaacgaaggcttctcaggcggggagaaaaaacgcaacgaaatccttcaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 198 - DNA, 4697 bp, synthetic construct
  misc_feature 1..4697: note = plasmid pEG154
  misc_feature 88..651: note = homology region iscU upstream
  regulatory 1833..2131: note = promoter - burden addiction promoter p(perR); regulatory_class = promoter
  misc_feature 2132..2137: note = rbs - rbs
  misc_feature 2137: note = N is A, T, C or G
  misc_feature 2147..2624: note = homology region iscU downstream
  source 1..4697: mol_type = other DNA
aacaaaattctccagtcttcacatcggtttgaaaggaggaagcggaagaatgaagtaagagggatttttgactccgaagtaagtcttcattgaggaattcggaaacaccacgcattaagagaaggaatattccgaaaaaccattctcatttgttctgaaaggatgctttcgtctcatctgccgctatttcacgccaaatcgtttttgcggcatccaacgcagcactggtacaataaaaagcaaatatatgaagggattgattcattatttgcagcagccaggacagactgaacctcgattatacgtcagaaatggaaaaagcgatgcagctgtcacacggaattggatacgaggaatatggacgccggctggaaacaagaatgaaagtagagcggcagagagagcttgattatgaaaaaagcaagcggatttctgcgggagcttattgagaaaatcaatcttttaaaaaagggtgggaatccactctttttctttttcaatcgctttaataaagtgctgatctttgttaaacttgaggaatgtgcaaggcacgataaaaagttgtatttacttttcatttgttataaaattagaatgagaataaaatgagaataatgattatatatttttggaggtatagatatggctgctagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattggaagcatttgatgtgtggaagctgaagaaaaacttcggaaaagagtatatgggaatcgagcgttccacttttttaattgataaagaaggccggctcattaaagaatggagaaaagtaaaagtaaaggaccatgtagccgaagcgcttcaaaccctcaaagatatgtctgaaaagtaaatctctatgagcctatgcttacttttaccctgatatcaattgacaaattatcgtagaaagagttacactaattataaacattacaatgtaagaattttttttagaagagaggtgcatgacggcgagntgaaatcatatggctgcttcaacattaacgatcaaagatcttcacgttgaaatcgaagggaaagagatcttaaagggtgtaaaccttgaaataaaaggtggagaattccacgcagtaatgggcccgaacggaactggtaaatccactttatcagctgctattatggggcatcctaaatatgaagtaacaaaaggcagcatcacgcttgacggcaaagatgtactggagatggaagtggacgagcgtgcgcaggcaggcttattccttgcaatgcagtacccaagtgaaatcagcggtgtgacaaatgccgacttccttcgctcagcaatcaacgcgcgcagagaagaaggcgatgaaatttctctcatgaaattcatccgcaaaatggacgaaaacatggagttccttgaaatggaccctgaaatggctcagcgctatcttaacgaaggcttctcaggcggggagaaaaaacgcaacgaaatccttcaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 199 - DNA, 4697 bp, synthetic construct
  misc_feature 1..4697: note = plasmid pEG155
  misc_feature 88..651: note = homology region iscU upstream
  regulatory 1823..2131: note = promoter - burden addiction promoter p(hrcA); regulatory_class = promoter
  regulatory 1823..2131: note = promoter - burden addiction promoter p(hrcA); regulatory_class = promoter
  misc_feature 2132..2137: note = rbs - rbs
  misc_feature 2132..2137: note = N is A, T, C or G
  misc_feature 2147..2624: note = homology region iscU downstream
  source 1..4697: mol_type = other DNA
aacaaaattctccagtcttcacatcggtttgaaaggaggaagcggaagaatgaagtaagagggatttttgactccgaagtaagtcttcattgaggaattcggaaacaccacgcattaagagaaggaatattccgaaaaaccattctcatttgttctgaaaggatgctttcgtctcatctgccgctatttcacgccaaatcgtttttgcggcatccaacgcagcactggtacaataaaaagcaaatatatgaagggattgattcattatttgcagcagccaggacagactgaacctcgattatacgtcagaaatggaaaaagcgatgcagctgtcacacggaattggatacgaggaatatggacgccggctggaaacaagaatgaaagtagagcggcagagagagcttgattatgaaaaaagcaagcggatttctgcgggagcttattgagaaaatcaatcttttaaaaaagggtgggaatccactctttttctttttcaatcgctttaataaagtgctgatctttgttaaacttgaggaatgtgcaaggcacgataaaaagttgtatttacttttcatttgttataaaattagaatgagaataaaatgagaataatgattatatatttttggaggtatagatatggctgctagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgatttgaggaagaaatgtttttaggtttgagaaaaacagccggtgtcagcaaaaaacgattcgcagaaaaatacggccgttccctggatggacttttcccaagcgtgttaaaagacctcgctgaaaaagggctgattcacaattcggagtctgccgtttatttgactcatcaagggaaattattaggcaatgaagtttttggcgcttttttgggtgagttataattgacatttttcttgtggtttgatacttttgttatagaattagcactcgcttattgagagtgctaacagaggtgatgatgcgagntgaaatcatatggctgcttcaacattaacgatcaaagatcttcacgttgaaatcgaagggaaagagatcttaaagggtgtaaaccttgaaataaaaggtggagaattccacgcagtaatgggcccgaacggaactggtaaatccactttatcagctgctattatggggcatcctaaatatgaagtaacaaaaggcagcatcacgcttgacggcaaagatgtactggagatggaagtggacgagcgtgcgcaggcaggcttattccttgcaatgcagtacccaagtgaaatcagcggtgtgacaaatgccgacttccttcgctcagcaatcaacgcgcgcagagaagaaggcgatgaaatttctctcatgaaattcatccgcaaaatggacgaaaacatggagttccttgaaatggaccctgaaatggctcagcgctatcttaacgaaggcttctcaggcggggagaaaaaacgcaacgaaatccttcaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 200 - DNA, 4697 bp, synthetic construct
  misc_feature 1..4697: note = plasmid pEG156
  misc_feature 88..651: note = homology region iscU upstream
  regulatory 1833..2131: note = promoter - burden addiction promoter p(spxA); regulatory_class = promoter
  misc_feature 2132..2137: note = rbs - rbs
  misc_feature 2137: note = N is A, T, C or G
  misc_feature 2147..2624: note = homology region iscU downstream
  source 1..4697: mol_type = other DNA
aacaaaattctccagtcttcacatcggtttgaaaggaggaagcggaagaatgaagtaagagggatttttgactccgaagtaagtcttcattgaggaattcggaaacaccacgcattaagagaaggaatattccgaaaaaccattctcatttgttctgaaaggatgctttcgtctcatctgccgctatttcacgccaaatcgtttttgcggcatccaacgcagcactggtacaataaaaagcaaatatatgaagggattgattcattatttgcagcagccaggacagactgaacctcgattatacgtcagaaatggaaaaagcgatgcagctgtcacacggaattggatacgaggaatatggacgccggctggaaacaagaatgaaagtagagcggcagagagagcttgattatgaaaaaagcaagcggatttctgcgggagcttattgagaaaatcaatcttttaaaaaagggtgggaatccactctttttctttttcaatcgctttaataaagtgctgatctttgttaaacttgaggaatgtgcaaggcacgataaaaagttgtatttacttttcatttgttataaaattagaatgagaataaaatgagaataatgattatatatttttggaggtatagatatggctgctagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattgaaaacattcatacttataaagatgagaaatggtacggagaatcgtacgaaaaaacggatgaggtcttggaaatcatcgatgaagaaaaacaaaaaaatattttcgatcagttaagctaattgagaataagaacatatcatgaacattaaaaatttgctggattgacacatttttttcttaggatactgttcacacttacttttttatagtataatacctgtaaagattccttttttagagtaatttcaatttaagaataaaatagatgttcatcctactaattagaggagtgaagatgacgagntgaaatcatatggctgcttcaacattaacgatcaaagatcttcacgttgaaatcgaagggaaagagatcttaaagggtgtaaaccttgaaataaaaggtggagaattccacgcagtaatgggcccgaacggaactggtaaatccactttatcagctgctattatggggcatcctaaatatgaagtaacaaaaggcagcatcacgcttgacggcaaagatgtactggagatggaagtggacgagcgtgcgcaggcaggcttattccttgcaatgcagtacccaagtgaaatcagcggtgtgacaaatgccgacttccttcgctcagcaatcaacgcgcgcagagaagaaggcgatgaaatttctctcatgaaattcatccgcaaaatggacgaaaacatggagttccttgaaatggaccctgaaatggctcagcgctatcttaacgaaggcttctcaggcggggagaaaaaacgcaacgaaatccttcaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 201 - DNA, 4697 bp, synthetic construct
  misc_feature 1..4697: note = plasmid pEG157
  misc_feature 88..651: note = homology region iscU upstream
  regulatory 1833..2131: note = promoter - burden addiction promoter p(sigB); regulatory_class = promoter
  misc_feature 2132..2137: note = rbs - rbs
  misc_feature 2132..2137: note = N is A, T, C or G
  misc_feature 2147..2624: note = homology region iscU downstream
  source 1..4697: mol_type = other DNA
aacaaaattctccagtcttcacatcggtttgaaaggaggaagcggaagaatgaagtaagagggatttttgactccgaagtaagtcttcattgaggaattcggaaacaccacgcattaagagaaggaatattccgaaaaaccattctcatttgttctgaaaggatgctttcgtctcatctgccgctatttcacgccaaatcgtttttgcggcatccaacgcagcactggtacaataaaaagcaaatatatgaagggattgattcattatttgcagcagccaggacagactgaacctcgattatacgtcagaaatggaaaaagcgatgcagctgtcacacggaattggatacgaggaatatggacgccggctggaaacaagaatgaaagtagagcggcagagagagcttgattatgaaaaaagcaagcggatttctgcgggagcttattgagaaaatcaatcttttaaaaaagggtgggaatccactctttttctttttcaatcgctttaataaagtgctgatctttgttaaacttgaggaatgtgcaaggcacgataaaaagttgtatttacttttcatttgttataaaattagaatgagaataaaatgagaataatgattatatatttttggaggtatagatatggctgctagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattaacaattcgatcagcatctggaaaagggcgatatgattgttttattttctgacggcgtcacagaatgcagaacggaaaacggtttcttggagcgtcctgatctgcagaagctcattgaggaacatatgtgttcctctgcgcaggaaatggtcaaaaacatttatgacagcctcctcaaattgcaggattttcagcttcacgatgattttacgttaattgttttgcggagaaaggtttaacgtctgtcagacgagggtataaagcaactagtgatttgaaggaaaatttgaggtgatacgacgagntgaaatcatatggctgcttcaacattaacgatcaaagatcttcacgttgaaatcgaagggaaagagatcttaaagggtgtaaaccttgaaataaaaggtggagaattccacgcagtaatgggcccgaacggaactggtaaatccactttatcagctgctattatggggcatcctaaatatgaagtaacaaaaggcagcatcacgcttgacggcaaagatgtactggagatggaagtggacgagcgtgcgcaggcaggcttattccttgcaatgcagtacccaagtgaaatcagcggtgtgacaaatgccgacttccttcgctcagcaatcaacgcgcgcagagaagaaggcgatgaaatttctctcatgaaattcatccgcaaaatggacgaaaacatggagttccttgaaatggaccctgaaatggctcagcgctatcttaacgaaggcttctcaggcggggagaaaaaacgcaacgaaatccttcaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 202 - DNA, 4697 bp, synthetic construct
  misc_feature 1..4697: note = plasmid pEG158
  misc_feature 88..651: note = homology region iscU upstream
  regulatory 1833..2131: note = promoter - burden adiction promoter p(yflT); regulatory_class = promoter
  misc_feature 2132..2137: note = rbs - rbs
  misc_feature 2137: note = N is A, T, C or G
  misc_feature 2147..2624: note = homology region iscU downstream
  source 1..4697: mol_type = other DNA
aacaaaattctccagtcttcacatcggtttgaaaggaggaagcggaagaatgaagtaagagggatttttgactccgaagtaagtcttcattgaggaattcggaaacaccacgcattaagagaaggaatattccgaaaaaccattctcatttgttctgaaaggatgctttcgtctcatctgccgctatttcacgccaaatcgtttttgcggcatccaacgcagcactggtacaataaaaagcaaatatatgaagggattgattcattatttgcagcagccaggacagactgaacctcgattatacgtcagaaatggaaaaagcgatgcagctgtcacacggaattggatacgaggaatatggacgccggctggaaacaagaatgaaagtagagcggcagagagagcttgattatgaaaaaagcaagcggatttctgcgggagcttattgagaaaatcaatcttttaaaaaagggtgggaatccactctttttctttttcaatcgctttaataaagtgctgatctttgttaaacttgaggaatgtgcaaggcacgataaaaagttgtatttacttttcatttgttataaaattagaatgagaataaaatgagaataatgattatatatttttggaggtatagatatggctgctagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattgactaaatcactcacaatccaagcattgtattgctgttctggtgaaaagtattgcattagacatacctcctgctcgtacggataaaggcagcgtttcatggtcgtgtgctccgtgcagcggcttctccttaattttgatttttctgaaaataggtcccgttcctatcactttaccatggacggaaaacaaatagctactaccattcctcctgtttttctcttcaatgttctggaatctgtttcaggtacagacgatcgggtatgaaagaaatatagaaaacatgaaggaggaatatcgaccgagntgaaatcatatggctgcttcaacattaacgatcaaagatcttcacgttgaaatcgaagggaaagagatcttaaagggtgtaaaccttgaaataaaaggtggagaattccacgcagtaatgggcccgaacggaactggtaaatccactttatcagctgctattatggggcatcctaaatatgaagtaacaaaaggcagcatcacgcttgacggcaaagatgtactggagatggaagtggacgagcgtgcgcaggcaggcttattccttgcaatgcagtacccaagtgaaatcagcggtgtgacaaatgccgacttccttcgctcagcaatcaacgcgcgcagagaagaaggcgatgaaatttctctcatgaaattcatccgcaaaatggacgaaaacatggagttccttgaaatggaccctgaaatggctcagcgctatcttaacgaaggcttctcaggcggggagaaaaaacgcaacgaaatccttcaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 203 - DNA, 4696 bp, synthetic construct
  misc_feature 1..4696: note = plasmid pEG159
  misc_feature 88..676: note = homology region aaC upstream
  regulatory 1858..2153: note = promoter - burden adiction promoter p(groES); regulatory_class = promoter
  misc_feature 2154..2159: note = rbs - rbs
  misc_feature 2154..2159: note = N is A, T, C or G
  misc_feature 2169..2623: note = homology region aaC downstream
  source 1..4696: mol_type = other DNA
aacaaaattctccagtcttcacatcggtttgaaaggaggaagcggaagaatgaagtaagagggatttttgactccgaagtaagtcttagggaacgaaagattcatcagcagacaaggaaacatccgctgaagccagtgaaaaaggaactgttgttacggaaacagccgatgatgatttgtttacaacgtaccgtcttgatttagaagatgccagaagcaaagaaagagaagagctgaatgccattgtgtcaagcgatgatgcaacagccaaggagaaaagcgaagcatacgataaaatgacggctctcagtgaagtggaaggaacagaaaaacagctggaaacgctgattaaaacacaaggttacgaggatgcgcttgttaatgctgaaggagataaaatcaatattacagtcaaatcagacaaacactctaaatcgaaggcgacagccattatagaccttgtggcaaaagaaatcaaaacaatgaaagatgtcgctgtcacatttgaaccctctaaataagaatgagggaaaaaagcccgctaaacaagcgggctttttgcgttgctgtcatattagagttgaattcaaaagtccgctcctgtaagatgaacatagtatgtacttttagtagtaacctataaaaaaagaaatttcatacataggagtgcgattgaagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattaatgcggaaaactgcatgatgatatatgtcaaaataataaaccaatactgttttctcaaatggtatgtactcctttgttaagtgggtttcgttcatctacagctattgtaacataatcggtacgggggtgaaaaagctaacggaaaagggagcggaaaagaatgatgtaagcgtgaaaaattttttatcttatcacttgaaattggaagggagattctttattataagaattgtgttagcactctttagtgctgagtgctaaaattacatattcatactattgaggaggttatttcacgagntgaaatcatatgttaaatatcaaagaaatccacgagctgattaaagcaattgacgagtctacaattgacgaattcgtatatgaaaatgaaggtgtatccttaaaactgaaaaaacacgaagcaggcacggttcaagtcatgcagcaggcaccggcagcacctgtacaagcacaggctccgcaggcagttcagccgcaagcgcagcaagcagcggcacctgcccaagaagcaccaaagcaagatgagaatctgcataaaatcacttcaccaatggtaggaacattttatgcttcttcatcaccggaagctggcccgtatgtaacagccggttcaaaagtaaatgaaaacacagttgtctgcattgtagaagcgatgaagcttttcaacgaaatcgaagcagaagtgaaaggcgaaatcgttgaagtattagtagaaaacggccagctggtcgaatacggacaaccaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 204 - DNA, 4699 bp, synthetic construct
  misc_feature 1..4699: note = plasmid pEG160
  misc_feature 88..676: note = homology region accC upstream
  regulatory 1858..2156: note = promoter - burden adiction promoter p(ctsR); regulatory_class = promoter
  misc_feature 2157..2162: note = rbs - rbs
  misc_feature 2162: note = N is A, T, C or G
  misc_feature 2172..2626: note = homology region accC downstream
  source 1..4699: mol_type = other DNA
aacaaaattctccagtcttcacatcggtttgaaaggaggaagcggaagaatgaagtaagagggatttttgactccgaagtaagtcttagggaacgaaagattcatcagcagacaaggaaacatccgctgaagccagtgaaaaaggaactgttgttacggaaacagccgatgatgatttgtttacaacgtaccgtcttgatttagaagatgccagaagcaaagaaagagaagagctgaatgccattgtgtcaagcgatgatgcaacagccaaggagaaaagcgaagcatacgataaaatgacggctctcagtgaagtggaaggaacagaaaaacagctggaaacgctgattaaaacacaaggttacgaggatgcgcttgttaatgctgaaggagataaaatcaatattacagtcaaatcagacaaacactctaaatcgaaggcgacagccattatagaccttgtggcaaaagaaatcaaaacaatgaaagatgtcgctgtcacatttgaaccctctaaataagaatgagggaaaaaagcccgctaaacaagcgggctttttgcgttgctgtcatattagagttgaattcaaaagtccgctcctgtaagatgaacatagtatgtacttttagtagtaacctataaaaaaagaaatttcatacataggagtgcgattgaagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattgctcttcagcgccgatggtagtcgggggtttccccctgtgagagtaggacgccgccaagcaagcttaaacccagctcaatgagctgggttttttgtattttggtttattggtatcataaaattccacttaactgtataatataataactttataccgaattttaaatcagcaatcaggttttgtggaccgggaaaatggaaataatgaaggatagagcgagaaagttgaaaattctcgagaaacggcttatagtaagattaaagtcaaatatagtcaaagtcagtaaaggagggggttgacgagntgaaatcatatgttaaatatcaaagaaatccacgagctgattaaagcaattgacgagtctacaattgacgaattcgtatatgaaaatgaaggtgtatccttaaaactgaaaaaacacgaagcaggcacggttcaagtcatgcagcaggcaccggcagcacctgtacaagcacaggctccgcaggcagttcagccgcaagcgcagcaagcagcggcacctgcccaagaagcaccaaagcaagatgagaatctgcataaaatcacttcaccaatggtaggaacattttatgcttcttcatcaccggaagctggcccgtatgtaacagccggttcaaaagtaaatgaaaacacagttgtctgcattgtagaagcgatgaagcttttcaacgaaatcgaagcagaagtgaaaggcgaaatcgttgaagtattagtagaaaacggccagctggtcgaatacggacaaccaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 205 - DNA, 4699 bp, synthetic construct
  misc_feature 1..4699: note = plasmid pEG161
  misc_feature 88..676: note = homology region accC upstream
  regulatory 1858..2156: note = promoter - burden adiction promoter p(dnaK); regulatory_class = promoter
  misc_feature 2157..2162: note = rbs - rbs
  misc_feature 2162: note = N is A, T, C or G
  misc_feature 2172..2626: note = homology region accC downstream
  source 1..4699: mol_type = other DNA
aacaaaattctccagtcttcacatcggtttgaaaggaggaagcggaagaatgaagtaagagggatttttgactccgaagtaagtcttagggaacgaaagattcatcagcagacaaggaaacatccgctgaagccagtgaaaaaggaactgttgttacggaaacagccgatgatgatttgtttacaacgtaccgtcttgatttagaagatgccagaagcaaagaaagagaagagctgaatgccattgtgtcaagcgatgatgcaacagccaaggagaaaagcgaagcatacgataaaatgacggctctcagtgaagtggaaggaacagaaaaacagctggaaacgctgattaaaacacaaggttacgaggatgcgcttgttaatgctgaaggagataaaatcaatattacagtcaaatcagacaaacactctaaatcgaaggcgacagccattatagaccttgtggcaaaagaaatcaaaacaatgaaagatgtcgctgtcacatttgaaccctctaaataagaatgagggaaaaaagcccgctaaacaagcgggctttttgcgttgctgtcatattagagttgaattcaaaagtccgctcctgtaagatgaacatagtatgtacttttagtagtaacctataaaaaaagaaatttcatacataggagtgcgattgaagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattggttcaatccccgcacacgggaatttcgattaaaatcggaaaagaaaacgactatgaagagatggaaaattgcagtctgattacggcttcttattccgtagaccagaagcagatcggctcaattgcgattatcggcccgacccgcatgaattattccagggttgtcagcctgcttcagcatgtgacttcggacttgtcaaaagcattaacaagtctgtatgatgaataagggaattttggcaaattttatcgaagggcagcacctgtccttctccttacactttgagggaggtgaacacacgagntgaaatcatatgttaaatatcaaagaaatccacgagctgattaaagcaattgacgagtctacaattgacgaattcgtatatgaaaatgaaggtgtatccttaaaactgaaaaaacacgaagcaggcacggttcaagtcatgcagcaggcaccggcagcacctgtacaagcacaggctccgcaggcagttcagccgcaagcgcagcaagcagcggcacctgcccaagaagcaccaaagcaagatgagaatctgcataaaatcacttcaccaatggtaggaacattttatgcttcttcatcaccggaagctggcccgtatgtaacagccggttcaaaagtaaatgaaaacacagttgtctgcattgtagaagcgatgaagcttttcaacgaaatcgaagcagaagtgaaaggcgaaatcgttgaagtattagtagaaaacggccagctggtcgaatacggacaaccaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 206 - DNA, 4699 bp, synthetic construct
  misc_feature 1..4699: note = plasmid pEG162
  misc_feature 88..676: note = homology region accC upstream
  regulatory 1858..2156: note = promoter - burden adictionpromoter p(perR); regulatory_class = promoter
  misc_feature 2157..2162: note = rbs - rbs
  misc_feature 2162: note = N is A, T, C or G
  misc_feature 2172..2626: note = homology region accC downstream
  source 1..4699: mol_type = other DNA
aacaaaattctccagtcttcacatcggtttgaaaggaggaagcggaagaatgaagtaagagggatttttgactccgaagtaagtcttagggaacgaaagattcatcagcagacaaggaaacatccgctgaagccagtgaaaaaggaactgttgttacggaaacagccgatgatgatttgtttacaacgtaccgtcttgatttagaagatgccagaagcaaagaaagagaagagctgaatgccattgtgtcaagcgatgatgcaacagccaaggagaaaagcgaagcatacgataaaatgacggctctcagtgaagtggaaggaacagaaaaacagctggaaacgctgattaaaacacaaggttacgaggatgcgcttgttaatgctgaaggagataaaatcaatattacagtcaaatcagacaaacactctaaatcgaaggcgacagccattatagaccttgtggcaaaagaaatcaaaacaatgaaagatgtcgctgtcacatttgaaccctctaaataagaatgagggaaaaaagcccgctaaacaagcgggctttttgcgttgctgtcatattagagttgaattcaaaagtccgctcctgtaagatgaacatagtatgtacttttagtagtaacctataaaaaaagaaatttcatacataggagtgcgattgaagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattggaagcatttgatgtgtggaagctgaagaaaaacttcggaaaagagtatatgggaatcgagcgttccacttttttaattgataaagaaggccggctcattaaagaatggagaaaagtaaaagtaaaggaccatgtagccgaagcgcttcaaaccctcaaagatatgtctgaaaagtaaatctctatgagcctatgcttacttttaccctgatatcaattgacaaattatcgtagaaagagttacactaattataaacattacaatgtaagaattttttttagaagagaggtgcatgacggcgagntgaaatcatatgttaaatatcaaagaaatccacgagctgattaaagcaattgacgagtctacaattgacgaattcgtatatgaaaatgaaggtgtatccttaaaactgaaaaaacacgaagcaggcacggttcaagtcatgcagcaggcaccggcagcacctgtacaagcacaggctccgcaggcagttcagccgcaagcgcagcaagcagcggcacctgcccaagaagcaccaaagcaagatgagaatctgcataaaatcacttcaccaatggtaggaacattttatgcttcttcatcaccggaagctggcccgtatgtaacagccggttcaaaagtaaatgaaaacacagttgtctgcattgtagaagcgatgaagcttttcaacgaaatcgaagcagaagtgaaaggcgaaatcgttgaagtattagtagaaaacggccagctggtcgaatacggacaaccaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 207 - DNA, 4699 bp, synthetic construct
  misc_feature 1..4699: note = plasmid pEG163
  misc_feature 88..676: note = homology region accC upstream
  regulatory 1858..2156: note = promoter - burden adiction promoter p(hrcA); regulatory_class = promoter
  misc_feature 2157..2162: note = rbs - rbs
  misc_feature 2162: note = N is A, T , C or G
  misc_feature 2172..2626: note = homology region accC downstream
  source 1..4699: mol_type = other DNA
aacaaaattctccagtcttcacatcggtttgaaaggaggaagcggaagaatgaagtaagagggatttttgactccgaagtaagtcttagggaacgaaagattcatcagcagacaaggaaacatccgctgaagccagtgaaaaaggaactgttgttacggaaacagccgatgatgatttgtttacaacgtaccgtcttgatttagaagatgccagaagcaaagaaagagaagagctgaatgccattgtgtcaagcgatgatgcaacagccaaggagaaaagcgaagcatacgataaaatgacggctctcagtgaagtggaaggaacagaaaaacagctggaaacgctgattaaaacacaaggttacgaggatgcgcttgttaatgctgaaggagataaaatcaatattacagtcaaatcagacaaacactctaaatcgaaggcgacagccattatagaccttgtggcaaaagaaatcaaaacaatgaaagatgtcgctgtcacatttgaaccctctaaataagaatgagggaaaaaagcccgctaaacaagcgggctttttgcgttgctgtcatattagagttgaattcaaaagtccgctcctgtaagatgaacatagtatgtacttttagtagtaacctataaaaaaagaaatttcatacataggagtgcgattgaagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgatttgaggaagaaatgtttttaggtttgagaaaaacagccggtgtcagcaaaaaacgattcgcagaaaaatacggccgttccctggatggacttttcccaagcgtgttaaaagacctcgctgaaaaagggctgattcacaattcggagtctgccgtttatttgactcatcaagggaaattattaggcaatgaagtttttggcgcttttttgggtgagttataattgacatttttcttgtggtttgatacttttgttatagaattagcactcgcttattgagagtgctaacagaggtgatgatgcgagntgaaatcatatgttaaatatcaaagaaatccacgagctgattaaagcaattgacgagtctacaattgacgaattcgtatatgaaaatgaaggtgtatccttaaaactgaaaaaacacgaagcaggcacggttcaagtcatgcagcaggcaccggcagcacctgtacaagcacaggctccgcaggcagttcagccgcaagcgcagcaagcagcggcacctgcccaagaagcaccaaagcaagatgagaatctgcataaaatcacttcaccaatggtaggaacattttatgcttcttcatcaccggaagctggcccgtatgtaacagccggttcaaaagtaaatgaaaacacagttgtctgcattgtagaagcgatgaagcttttcaacgaaatcgaagcagaagtgaaaggcgaaatcgttgaagtattagtagaaaacggccagctggtcgaatacggacaaccaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 208 - DNA, 4699 bp, synthetic construct
  misc_feature 1..4699: note = plasmid pEG164
  misc_feature 88..676: note = homology region accC upstream
  regulatory 1858..2156: note = promoter - burden adiction promoter p(spxA); regulatory_class = promoter
  misc_feature 2157..2162: note = rbs - rbs
  misc_feature 2162: note = N is A, T, C or G
  misc_feature 2172..2626: note = homology region accC downstream
  source 1..4699: mol_type = other DNA
aacaaaattctccagtcttcacatcggtttgaaaggaggaagcggaagaatgaagtaagagggatttttgactccgaagtaagtcttagggaacgaaagattcatcagcagacaaggaaacatccgctgaagccagtgaaaaaggaactgttgttacggaaacagccgatgatgatttgtttacaacgtaccgtcttgatttagaagatgccagaagcaaagaaagagaagagctgaatgccattgtgtcaagcgatgatgcaacagccaaggagaaaagcgaagcatacgataaaatgacggctctcagtgaagtggaaggaacagaaaaacagctggaaacgctgattaaaacacaaggttacgaggatgcgcttgttaatgctgaaggagataaaatcaatattacagtcaaatcagacaaacactctaaatcgaaggcgacagccattatagaccttgtggcaaaagaaatcaaaacaatgaaagatgtcgctgtcacatttgaaccctctaaataagaatgagggaaaaaagcccgctaaacaagcgggctttttgcgttgctgtcatattagagttgaattcaaaagtccgctcctgtaagatgaacatagtatgtacttttagtagtaacctataaaaaaagaaatttcatacataggagtgcgattgaagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattgaaaacattcatacttataaagatgagaaatggtacggagaatcgtacgaaaaaacggatgaggtcttggaaatcatcgatgaagaaaaacaaaaaaatattttcgatcagttaagctaattgagaataagaacatatcatgaacattaaaaatttgctggattgacacatttttttcttaggatactgttcacacttacttttttatagtataatacctgtaaagattccttttttagagtaatttcaatttaagaataaaatagatgttcatcctactaattagaggagtgaagatgacgagntgaaatcatatgttaaatatcaaagaaatccacgagctgattaaagcaattgacgagtctacaattgacgaattcgtatatgaaaatgaaggtgtatccttaaaactgaaaaaacacgaagcaggcacggttcaagtcatgcagcaggcaccggcagcacctgtacaagcacaggctccgcaggcagttcagccgcaagcgcagcaagcagcggcacctgcccaagaagcaccaaagcaagatgagaatctgcataaaatcacttcaccaatggtaggaacattttatgcttcttcatcaccggaagctggcccgtatgtaacagccggttcaaaagtaaatgaaaacacagttgtctgcattgtagaagcgatgaagcttttcaacgaaatcgaagcagaagtgaaaggcgaaatcgttgaagtattagtagaaaacggccagctggtcgaatacggacaaccaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 209 - DNA, 4699 bp, synthetic construct
  misc_feature 1..4699: note = plasmid pEG165
  misc_feature 88..676: note = homology region accC upstream
  regulatory 1858..2156: note = promoter - burden adiction promoter p(sigB); regulatory_class = promoter
  misc_feature 2157..2162: note = rbs - rbs
  misc_feature 2162: note = N is A, T, C or G
  misc_feature 2172..2626: note = homology region accC downstream
  source 1..4699: mol_type = other DNA
aacaaaattctccagtcttcacatcggtttgaaaggaggaagcggaagaatgaagtaagagggatttttgactccgaagtaagtcttagggaacgaaagattcatcagcagacaaggaaacatccgctgaagccagtgaaaaaggaactgttgttacggaaacagccgatgatgatttgtttacaacgtaccgtcttgatttagaagatgccagaagcaaagaaagagaagagctgaatgccattgtgtcaagcgatgatgcaacagccaaggagaaaagcgaagcatacgataaaatgacggctctcagtgaagtggaaggaacagaaaaacagctggaaacgctgattaaaacacaaggttacgaggatgcgcttgttaatgctgaaggagataaaatcaatattacagtcaaatcagacaaacactctaaatcgaaggcgacagccattatagaccttgtggcaaaagaaatcaaaacaatgaaagatgtcgctgtcacatttgaaccctctaaataagaatgagggaaaaaagcccgctaaacaagcgggctttttgcgttgctgtcatattagagttgaattcaaaagtccgctcctgtaagatgaacatagtatgtacttttagtagtaacctataaaaaaagaaatttcatacataggagtgcgattgaagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattaacaattcgatcagcatctggaaaagggcgatatgattgttttattttctgacggcgtcacagaatgcagaacggaaaacggtttcttggagcgtcctgatctgcagaagctcattgaggaacatatgtgttcctctgcgcaggaaatggtcaaaaacatttatgacagcctcctcaaattgcaggattttcagcttcacgatgattttacgttaattgttttgcggagaaaggtttaacgtctgtcagacgagggtataaagcaactagtgatttgaaggaaaatttgaggtgatacgacgagntgaaatcatatgttaaatatcaaagaaatccacgagctgattaaagcaattgacgagtctacaattgacgaattcgtatatgaaaatgaaggtgtatccttaaaactgaaaaaacacgaagcaggcacggttcaagtcatgcagcaggcaccggcagcacctgtacaagcacaggctccgcaggcagttcagccgcaagcgcagcaagcagcggcacctgcccaagaagcaccaaagcaagatgagaatctgcataaaatcacttcaccaatggtaggaacattttatgcttcttcatcaccggaagctggcccgtatgtaacagccggttcaaaagtaaatgaaaacacagttgtctgcattgtagaagcgatgaagcttttcaacgaaatcgaagcagaagtgaaaggcgaaatcgttgaagtattagtagaaaacggccagctggtcgaatacggacaaccaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 210 - DNA, 4699 bp, synthetic construct
  misc_feature 1..4699: note = plasmid pEG166
  misc_feature 88..676: note = homology region accC upstream
  regulatory 1858..2156: note = promoter - burden adiction promoter p(yflT); regulatory_class = promoter
  misc_feature 2157..2162: note = rbs - rbs
  misc_feature 2162: note = N is A, T, C or G
  misc_feature 2172..2626: note = homology region accC downstream
  source 1..4699: mol_type = other DNA
aacaaaattctccagtcttcacatcggtttgaaaggaggaagcggaagaatgaagtaagagggatttttgactccgaagtaagtcttagggaacgaaagattcatcagcagacaaggaaacatccgctgaagccagtgaaaaaggaactgttgttacggaaacagccgatgatgatttgtttacaacgtaccgtcttgatttagaagatgccagaagcaaagaaagagaagagctgaatgccattgtgtcaagcgatgatgcaacagccaaggagaaaagcgaagcatacgataaaatgacggctctcagtgaagtggaaggaacagaaaaacagctggaaacgctgattaaaacacaaggttacgaggatgcgcttgttaatgctgaaggagataaaatcaatattacagtcaaatcagacaaacactctaaatcgaaggcgacagccattatagaccttgtggcaaaagaaatcaaaacaatgaaagatgtcgctgtcacatttgaaccctctaaataagaatgagggaaaaaagcccgctaaacaagcgggctttttgcgttgctgtcatattagagttgaattcaaaagtccgctcctgtaagatgaacatagtatgtacttttagtagtaacctataaaaaaagaaatttcatacataggagtgcgattgaagttcggattagatccccctatgcaagggtttattgttttctaaaatctgattaccaattagaatgaatatttcccaaatattaaataataaaacaaaaaaattgaaaaaagtgtttccaccattttttcaatttttttataatttttttaatctgttatttaaatagtttatagttaaatttacattttcattagtccattcaatattctctccaagataactacgaactgctaacaaaattctctccctatgttctaatggagaagattcagccactgcatttcccgcaatatcttttggtatgattttacccgtgtccatagttaaaatcatacggcataaagttaatatagagttggtttcatcatcctgataattatctattaattcctctgacgaatccataatggctcttctcacatcagaaaatggaatatcaggtagtaattcctctaagtcataatttccgtatattcttttattttttcgttttgcttggtaaagcattatggttaaatctgaatttaattccttctgaggaatgtatccttgttcataaagctcttgtaaccattctccataaataaattcttgtttgggaggatgattccacggtaccatttcttgctgaataataattgttaattcaatatatcgtaagttgcttttatctcctattttttttgaaataggtctaattttttgtataagtatttctttactttgatctgtcaatggttcagatacgacgactaaaaagtcaagatcactatttggttttagtccactctcaactcctgatccaaacatgtaagtaccaataaggttattttttaaatgtttccgaagtatttttttcactttattaatttgttcgtatgtattcaaatatatcctcctcactattttgattagtacctattttatatccatagttgttaattaaataaacttaatttagtttatttatagatttcattggcttctaaattttttatctagataataattattttagttaattttattctagattatatatgatatgatctttcatttccataaaactaaagtaagtgtaaacctattcattgttttaaaaatatctcttgccagtcacgttacgttattagttatagttattataacatgtattcacgaacgaaaatcgcatcgattgactaaatcactcacaatccaagcattgtattgctgttctggtgaaaagtattgcattagacatacctcctgctcgtacggataaaggcagcgtttcatggtcgtgtgctccgtgcagcggcttctccttaattttgatttttctgaaaataggtcccgttcctatcactttaccatggacggaaaacaaatagctactaccattcctcctgtttttctcttcaatgttctggaatctgtttcaggtacagacgatcgggtatgaaagaaatatagaaaacatgaaggaggaatatcgaccgagntgaaatcatatgttaaatatcaaagaaatccacgagctgattaaagcaattgacgagtctacaattgacgaattcgtatatgaaaatgaaggtgtatccttaaaactgaaaaaacacgaagcaggcacggttcaagtcatgcagcaggcaccggcagcacctgtacaagcacaggctccgcaggcagttcagccgcaagcgcagcaagcagcggcacctgcccaagaagcaccaaagcaagatgagaatctgcataaaatcacttcaccaatggtaggaacattttatgcttcttcatcaccggaagctggcccgtatgtaacagccggttcaaaagtaaatgaaaacacagttgtctgcattgtagaagcgatgaagcttttcaacgaaatcgaagcagaagtgaaaggcgaaatcgttgaagtattagtagaaaacggccagctggtcgaatacggacaaccaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaatt

SEQ ID NO: 211 - DNA, 783 bp, synthetic construct
  misc_feature 1..783: note = Gene encoding SpcR (resistance marker)
  source 1..783: mol_type = other DNA
gtactaatcaaaatagtgaggaggatatatttgaatacatacgaacaaattaataaagtgaaaaaaatacttcggaaacatttaaaaaataaccttattggtacttacatgtttggatcaggagttgagagtggactaaaaccaaatagtgatcttgactttttagtcgtcgtatctgaaccattgacagatcaaagtaaagaaatacttatacaaaaaattagacctatttcaaaaaaaataggagataaaagcaacttacgatatattgaattaacaattattattcagcaagaaatggtaccgtggaatcatcctcccaaacaagaatttatttatggagaatggttacaagagctttatgaacaaggatacattcctcagaaggaattaaattcagatttaaccataatgctttaccaagcaaaacgaaaaaataaaagaatatacggaaattatgacttagaggaattactacctgatattccattttctgatgtgagaagagccattatggattcgtcagaggaattaatagataattatcaggatgatgaaaccaactctatattaactttatgccgtatgattttaactatggacacgggtaaaatcataccaaaagatattgcgggaaatgcagtggctgaatcttctccattagaacatagggagagaattttgttagcagttcgtagttatcttggagagaatattgaatggactaatgaaaatgtaaatttaactataaactatttaaataacagattaaaaaaattataa

SEQ ID NO: 212 - DNA, 296 bp, Bacillus sp.
  regulatory 1..296: note = promoter - Bacillus bruden sensing promoter p(groES); regulatory_class = promoter
  source 1..296: mol_type = genomic DNA
aatgcggaaaactgcatgatgatatatgtcaaaataataaaccaatactgttttctcaaatggtatgtactcctttgttaagtgggtttcgttcatctacagctattgtaacataatcggtacgggggtgaaaaagctaacggaaaagggagcggaaaagaatgatgtaagcgtgaaaaattttttatcttatcacttgaaattggaagggagattctttattataagaattgtgttagcactctttagtgctgagtgctaaaattacatattcatactattgaggaggttatttc

SEQ ID NO: 213 - DNA, 299 bp, Bacillus sp.
  regulatory 1..299: note = promoter - Bacillus burden sensing promoter p(ctsR); regulatory_class = promoter
  source 1..299: mol_type = genomic DNA
gctcttcagcgccgatggtagtcgggggtttccccctgtgagagtaggacgccgccaagcaagcttaaacccagctcaatgagctgggttttttgtattttggtttattggtatcataaaattccacttaactgtataatataataactttataccgaattttaaatcagcaatcaggttttgtggaccgggaaaatggaaataatgaaggatagagcgagaaagttgaaaattctcgagaaacggcttatagtaagattaaagtcaaatatagtcaaagtcagtaaaggagggggttg

SEQ ID NO: 214 - DNA, 299 bp, Bacillus sp.
  regulatory 1..299: note = promoter - Bacillus burden sensing promoter p(dnaK); regulatory_class = promoter
  source 1..299: mol_type = genomic DNA
ggttcaatccccgcacacgggaatttcgattaaaatcggaaaagaaaacgactatgaagagatggaaaattgcagtctgattacggcttcttattccgtagaccagaagcagatcggctcaattgcgattatcggcccgacccgcatgaattattccagggttgtcagcctgcttcagcatgtgacttcggacttgtcaaaagcattaacaagtctgtatgatgaataagggaattttggcaaattttatcgaagggcagcacctgtccttctccttacactttgagggaggtgaacac

SEQ ID NO: 215 - DNA, 299 bp, Bacillus sp.
  regulatory 1..299: note = promoter - Bacillus burden addiction sensing p(perR); regulatory_class = promoter
  source 1..299: mol_type = genomic DNA
ggaagcatttgatgtgtggaagctgaagaaaaacttcggaaaagagtatatgggaatcgagcgttccacttttttaattgataaagaaggccggctcattaaagaatggagaaaagtaaaagtaaaggaccatgtagccgaagcgcttcaaaccctcaaagatatgtctgaaaagtaaatctctatgagcctatgcttacttttaccctgatatcaattgacaaattatcgtagaaagagttacactaattataaacattacaatgtaagaattttttttagaagagaggtgcatgacg

SEQ ID NO: 216 - DNA, 299 bp, Bacillus sp.
  regulatory 1..299: note = promoter - Bacillus burden sensing promoter p(hrcA); regulatory_class = promoter
  source 1..299: mol_type = genomic DNA
tgaggaagaaatgtttttaggtttgagaaaaacagccggtgtcagcaaaaaacgattcgcagaaaaatacggccgttccctggatggacttttcccaagcgtgttaaaagacctcgctgaaaaagggctgattcacaattcggagtctgccgtttatttgactcatcaagggaaattattaggcaatgaagtttttggcgcttttttgggtgagttataattgacatttttcttgtggtttgatacttttgttatagaattagcactcgcttattgagagtgctaacagaggtgatgat

SEQ ID NO: 217 - DNA, 299 bp, Bacillus sp.
  regulatory 1..299: note = promoter - Bacillus burden sensing promoter p(spxA); regulatory_class = promoter
  source 1..299: mol_type = genomic DNA
gaaaacattcatacttataaagatgagaaatggtacggagaatcgtacgaaaaaacggatgaggtcttggaaatcatcgatgaagaaaaacaaaaaaatattttcgatcagttaagctaattgagaataagaacatatcatgaacattaaaaatttgctggattgacacatttttttcttaggatactgttcacacttacttttttatagtataatacctgtaaagattccttttttagagtaatttcaatttaagaataaaatagatgttcatcctactaattagaggagtgaagatg

SEQ ID NO: 218 - DNA, 299 bp, Bacillus sp.
  regulatory 1..299: note = promoter - Bacillus burden sensing promoter p(sigB); regulatory_class = promoter
  source 1..299: mol_type = genomic DNA
aacaattcgatcagcatctggaaaagggcgatatgattgttttattttctgacggcgtcacagaatgcagaacggaaaacggtttcttggagcgtcctgatctgcagaagctcattgaggaacatatgtgttcctctgcgcaggaaatggtcaaaaacatttatgacagcctcctcaaattgcaggattttcagcttcacgatgattttacgttaattgttttgcggagaaaggtttaacgtctgtcagacgagggtataaagcaactagtgatttgaaggaaaatttgaggtgatacg

SEQ ID NO: 219 - DNA, 299 bp, Bacillus sp.
  regulatory 1..299: note = promoter - Bacillus burden sensing promoter p(yflT); regulatory_class = promoter
  source 1..299: mol_type = genomic DNA
gactaaatcactcacaatccaagcattgtattgctgttctggtgaaaagtattgcattagacatacctcctgctcgtacggataaaggcagcgtttcatggtcgtgtgctccgtgcagcggcttctccttaattttgatttttctgaaaataggtcccgttcctatcactttaccatggacggaaaacaaatagctactaccattcctcctgtttttctcttcaatgttctggaatctgtttcaggtacagacgatcgggtatgaaagaaatatagaaaacatgaaggaggaatatcga

SEQ ID NO: 220 - DNA, 651 bp, synthetic construct
  source 1..651: mol_type = other DNA
  CDS 1..651: note = chlR gene encoding camR resistance marker
atgaactttaataaaattgatttagacaattggaagagaaaagagatatttaatcattatttgaaccaacaaacgacttttagtataaccacagaaattgatattagtgttttataccgaaacataaaacaagaaggatataaattttaccctgcatttattttcttagtgacaagggtgataaactcaaatacagcttttagaactggttacaatagcgacggagagttaggttattgggataagttagagccactttatacaatttttgatggtgtatctaaaacattctctggtatttggactcctgtaaagaatgacttcaaagagttttatgatttatacctttctgatgtagagaaatataatggttcggggaaattgtttcccaaaacacctatacctgaaaatgctttttctctttctattattccatggacttcatttactgggtttaacttaaatatcaataataatagtaattaccttctacccattattacagcaggaaaattcattaataaaggtaattcaatatatttaccgctatctttacaggtacatcattctgtttgtgatggttatcatgcaggattgtttatgaactctattcaggaattgtcagataggcctaatgactggcttttataa

SEQ ID NO: 221 - protein, 216 aa, synthetic construct
  source 1..216: note = The sequence is a translation of SEQ ID NO. 220
MNFNKIDLDNWKRKEIFNHYLNQQTTFSITTEIDISVLYRNIKQEGYKFYPAFIFLVTRVINSNTAFRTGYNSDGELGYWDKLEPLYTIFDGVSKTFSGIWTPVKNDFKEFYDLYLSDVEKYNGSGKLFPKTPIPENAFSLSIIPWTSFTGFNLNINNNSNYLLPIITAGKFINKGNSIYLPLSLQVHHSVCDGYHAGLFMNSIQELSDRPNDWLL

SEQ ID NO: 222 - DNA, 10063 bp, synthetic construct
  misc_feature 1..10063: note = plasmid pEG062
  misc_feature 1..637: note = homology region amyE upstream
  misc_feature 789..1583: note = resistance marker KanR (on complementary strand)
  regulatory 1744..1796: note = terminator - terminator T7; regulatory_class = terminator
  regulatory 1866..2655: note = promoter - promoter P3P; regulatory_class = promoter
  misc_feature 2680..3861: note = gene encoding IgA-V3
  misc_feature 3862..3867: note = glycine linker
  misc_feature 3868..4584: note = gene encoding sfGFP
  misc_feature 4585..4602: note = purification tag 6-his
  regulatory 4614..4682: note = terminator - terminator; regulatory_class = terminator
  misc_feature 5070..6262: note = homology region amyE downstream
  misc_feature 7207..7989: note = spc gene encoding resistance marker spectinomycunadenyltransferase
  misc_feature 8995..9855: note = bla gene encoding beta-lactamase for ampicillin resistance (oncomplementary strand)
  source 1..10063: mol_type = other DNA
ttaacaaaattctccagtcttcacatcggtttgaaaggaggaagcggaagaatgaagtaagagggatttttgactccgaagtaagtcttcaaaaaatcaaataaggagtgtcaagaatgtttgcaaaacgattcaaaacctctttactgccgttattcgctggatttttattgctgtttcatttggttctggcaggaccggcggctgcgagtgctgaaacggcgaacaaatcgaatgagcttacagcaccgtcgatcaaaagcggaaccattcttcatgcatggaattggtcgttcaatacgttaaaacacaatatgaaggatattcatgatgcaggatatacagccattcagacatctccgattaaccaagtaaaggaagggaatcaaggagataaaagcatgtcgaactggtactggctgtatcagccgacatcgtatcaaattggcaaccgttacttaggtactgaacaagaatttaaagaaatgtgtgcagccgctgaagaatatggcataaaggtcattgttgacgcggtcatcaatcataccaccagtgattatgccgcgatttccaatgaggttaagagtattccaaactggacacatggaaacacacaaattaaaaactggtctgatcggatcctagaagcttatcgaattctcatgtttgacagcttatcatcggcaatagttcggataatgagtttataaaataaaaaaagcacctgaaaaggtgtctttttttgatggttttgaacttgaagctttttagacatctaaatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagacccaggtctttttcaaagttgttttgaaagttggcccgcatcccccctagcataaccccttggggcctctaaacgggtcttgaggggttttttggagctcaaacaattaatgtgagttagctcactcattaggcaccccaggctttacactttatgcgtctattcttttggaagaaaatatagggaaaatggtatttgttaaaaattctgaatatttatacaatatcatatgtttcacattgaaatatgctgtccagactgtccgctgtgtaaaaaataggaataaaggggggttgttattattttactgatatgtaaaatataatttgtataagaaaattcgaaacgtaagatgaaaccttagataaaagtgctttttttgttgcaattgaagaattattaatgttaagcttaattaaagataatatctttgaattgtaacgcccctcaaaagtaagaactacaaaaaaagaatacgttatatagaaatatgtttgaaccttcttcagattacaaatatattcggacggactctacctcaaatgcttatctaactatagaatgacatacaagcacaaccttgaaaatttgaaaatataactaccaatgaacttgttcatgtgaattatcgctgtatttaattttctcaattcaatatataatatgccaatacattgttacaagtagaaattaagacacccttgatagccttactatacctaacatgatgtagtattaaatgaatatgtaaatatatttatgataagaagcgacttatttataatcattacatatttttctattggaatgattaagattccaatagaatagtgtataaattatttatcttgaaaggagggatgcctaaaaacgaagaacattaaaaacatatatttgcaccgtctaatggatttatgaaaaatcattttatcagtttgaaaattatgtattatgattaactaataaggaggacaaacatgatccaaaaacgcaaacgcacagttagctttagactggttctgatgtgcacactgctgtttgtttcactgccgattacaaaaacaagcgcacaagttcaactgcaagaatcaggcggaggcctggttcaagcaggcggatcactgagactgtcatgcgcagcatcaggccttacatttgatacatatgcaatgggctggtttagacaagcaccgggaaaaaaacgcgaatatgttgcagcaatttcatggacaggcatcagcacatattatgcggatattgcaaaaggacgctttacaattagccgtgataatgcgaaaaacacgctgtatctgcaaatggattcactgaaaccggaagatacagcagtctattattgcgcagcacaaaaatcactgaatgttccggcaccgtgggattattggggacaaggcacacaagttacagtttcatcagatccgtgtccgcaatgctgcaaaccgtcactgtcactgcaaccgcctgcactggcggatctgctgctgggctcaaatgcatcactgacatgcacactgtcaggactgaaaaaatctgaaggcgtttcatttacatggcaaccgtcaggcggaaaagatgcagttcaagcatcaccgacaagagattcatgcggctgctattcagtttcaagcattcttccgggatgcgctgatccgtggaataaaggcgaaacattttcatgcacagcagcacattcagaactgaaatcagcactgacagcgacaattacaaaaccgaaagtcaatacatttcgtccgcaagttcatctgcttccgcctccgagcgaagaactggcactgaatgaactggttacacttacatgcctggttagaggcttttcaccgaaagatgttcttgttagatggctgcaaggcggacaagaactgccgagagataaatatctggtttgggaatcactgccggaaccgggacaagcaattccgacatatgcagttacatcagttcttagagttgatgcggaagattggaaacagggcgatacatttagctgcatggttggccatgaagcactgccgctggcatttacacaaaaaacaattgatagactggcaggcaaaccgacacatgttaatgtttcagttgttatggcagaagcagaaggcatctgctataaagatgaactgggtggcatgcgtaaaggcgaagagctgttcactggtttcgtcactattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccacatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcacatggttctgctggagttcgtaaccgcagcgggcatcacgcatggtatggatgaactgtacaaataacaccaccatcatcaccattaaaagagcatgagaggacggatttcctgaaggaaatccgtttttttattttgcccgtcttataaatttctttgattacaaagctgttcttttataatatgagataatgccgactgtactttttacagtcggttttctaatgtcactaacctgccccgttagttgaagaaggtttttatattacagctccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacatggatgagcgatgatgatatccgtttaggctgggcggtgatagcttctcgttcaggcagtacgcctcttttcttttccagacctgagggaggcggaaatggtgtgaggttcccggggaaaagccaaataggcgatcgcgggagtgctttatttgaagatcaggctatcactgcggtcaatagatttcacaatgtgatggctggacagcctgaggaactctcgaacccgaatggaaacaaccagatatttatgaatcagcgcggctcacatggcgttgtgctggcaaatgcaggttcatcctctgtctctatcaatacggcaacaaaattgcctgatggcaggtatgacaataaagctggagcgggttcatttcaagtgaacgatggtaaactgacaggcacgatcaatgccaggtctgtagctgtgctttatcctgatgatattgcaaaagcgcctcatgttttccttgagaattacaaaacaggtgtaacacattctttcaatgatcaactgacgattaccttgcgtgcagatgcgaatacaacaaaagccgtttatcaaatcaataatggaccagacgacaggcgtttaaggatggagatcaattcacaatcggaaaaggagatccaatttggcaaaacatacaccatcatgttaaaaggaacgaacagtgatggtgtaacgaggaccgagaaatacagttttgttaaaagagatccagcgtcggccaaaaccatcggctatcaaaatccgaatcattggagccaggtaaatgcttatatctataaacatgatgggagccgagtaattgaattgaccggatcttggcctggaaaaccaatgactaaaaatgcagacggaatttacacgctgacgctgcctgcggacacggatacaaccaacgcaaaagtgatttttaataatggcagcgcccaagtgcccggtcagaatcagcctggctttgattacgtgctaaatggtttatataatgactcgggcttaagcggttctcttccccattgagggcaaggctagacgggacttaccgaaagaaaccatcaatgatggtttcttttttgttcataaatcagacaaaacttttctcttgcaaaagtttgtgaagtgttgcacaatataaatgtgaaatacttcacaaacaaaaagacatcaaagagaaacataccctgcaaggatgctgatattgtctgcatttgcgccggagcaaaccaaaaacctggtgagacacgccttgaattagtagaaaagaacttgaagattttcaaaggcatcgttagtgaagtcatggcgagcggatttgacggcattttcttagtcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctactgaagtggatttctttaagagctcctttaacttcctcaccagtagttgtatcggtaccataagtagaagcagcaacccaagtagctttaccagcatccggttcaaccagcatagtaagaatcttactggacatcggcagttcttcgaacagtgcgccaactaccagctctttctgcagttcattcagggcaccggagaacctgcgtgcaatccatcttgttcaatcatgcgaaacgatcctcatcctgtctcttgatccatggattacgcgttaacccgggcccgcggatgcatatgatcagatcttaaggcctaggtctagaggatcgatctgtataataaagaataattattaatctgtagacaaattgtgaaaggatgtacttaaacgctaacggtcagctttattgaacagtaatttaagtatatgtccaatctagggtaagtaaattgagtatcaatataaactttatatgaacataatcaacgaggtgaaatcatgagcaatttgattaacggaaaaataccaaatcaagcgattcaaacattaaaaatcgtaaaagatttatttggaagttcaatagttggagtatatctatttggttcagcagtaaatggtggtttacgcattaacagcgatgtagatgttctagtcgtcgtgaatcatagtttacctcaattaactcgaaaaaaactaacagaaagactaatgactatatcaggaaagattggaaatacggattctgttagaccacttgaagttacggttataaataggagtgaagttgtcccttggcaatatcctccaaaaagagaatttatatacggtgagtggctcaggggtgaatttgagaatggacaaattcaggaaccaagctatgatcctgatttggctattgttttagcacaagcaagaaagaatagtatttctctatttggtcctgattcttcaagtatacttgtctccgtacctttgacagatattcgaagagcaattaaggattctttgccagaactaattgaggggataaaaggtgatgagcgtaatgtaattttaaccctagctcgaatgtggcaaacagtgactactggtgaaattacctcgaaagatgtcgctgcagaatgggctatacctcttttacctaaagagcatgtaactttactggatatagctagaaaaggctatcggggagagtgtgatgataagtgggaaggactatattcaaaggtgaaagcactcgttaagtatatgaaaaattctatagaaacttctctcaattaggctaattttattgcaataacaggtgcttacttttctggagttctttagcaaatttttttattagctgaacttagtattagtggccatactcctccaatccaaagctatttagaaagattactatatcctcaaacaggcggtaaccggcctcttcatcgggaatgcgcgcgaccttcagcatcgccggcatgtccccctggcggacgggaagtatccagctcgaggtcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttgatagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaa

SEQ ID NO: 223 - DNA, 20 bp, synthetic construct
  primer_bind 1..20: note = primer E257
  source 1..20: mol_type = other DNA
ctgagtcaatttcatcaagg

SEQ ID NO: 224 - DNA, 22 bp, synthetic construct
  primer_bind 1..22: note = priomer E258
  source 1..22: mol_type = other DNA
cagaagaaaacattctcgaagc

SEQ ID NO: 225 - DNA, 20 bp, synthetic construct
  primer_bind 1..20: note = primer E261
  source 1..20: mol_type = other DNA
gaggtcgcaactgaaaaagc

SEQ ID NO: 226 - DNA, 20 bp, synthetic construct
  primer_bind 1..20: note = primer E262
  source 1..20: mol_type = other DNA
atgttcagtctccttactcc

SEQ ID NO: 227 - DNA, 30 bp, synthetic construct
  primer_bind 1..30: note = primer E372
  source 1..30: mol_type = other DNA
  misc_feature 10: note = Uracil instead of thymine in this location
attataacatgtattcacgaacgaaaatcg

SEQ ID NO: 228 - DNA, 35 bp, synthetic construct
  primer_bind 1..35: note = primer E373
  source 1..35: mol_type = other DNA
  misc_feature 10: note = Uracil instead of thymine in this location
atcagattttagaaaacaataaacccttgcatagg

SEQ ID NO: 229 - DNA, 20 bp, synthetic construct
  primer_bind 1..20: note = primer E374
  source 1..20: mol_type = other DNA
  misc_feature 10: note = Uracil instead of thymine in this location
aaaatctgatacggggcagg

SEQ ID NO: 230 - DNA, 37 bp, synthetic construct
  primer_bind 1..37: note = primer E375
  source 1..37: mol_type = other DNA
  misc_feature 10: note = Uracil instead of thymine in this location
atgttataatacccattagttcaacaaacgaaaattg

SEQ ID NO: 231 - protein, 264 aa, synthetic construct [sequence omitted]

SEQ ID NO: 232 - DNA, 2734 bp, synthetic construct
  misc_feature 1..2734: note = Integration fragment INT1 comprising P(gsh2) promoter, cia1essential gene, and TIS1 translation initiation sequence.
  regulatory 1724..2234: note = promoter - P13_pGSH2; regulatory_class = promoter
  regulatory 2229..2234: note = promoter - TIS1; regulatory_class = promoter
  gene 2235..2734: note = first part of CIA1 gene, for integration by homologousrecombination
  source 1..2734: mol_type = other DNA
  CDS 1024..1398: note = blaShe (resistance gene used to select for chromosomalintegration)
tggtcctcgtctatttgttgtttaaaagtgatagtatacttcttgaactcgctggggttaagaaattccaagtcttcatcttctacaaagaagtagttgtacgagaacaatagccatacagtaccacctatgacgaccagactaggaataaatctcaaccatgggtttctccgctttctaatgaccttttctgctttcaactccgatggtgaaggtggaggctggtgttcataagtgaccctttggttgggtcttggaatgtattgttttttcagttgaatcccttgcaacggttgaaggccgccagtcaacttgggggcgtttttcttagcagggactgcataatttctaacgagccgtagtattctctggttcatggatcatagaactgaggggaaggggaagtgtagaaatgagcagaaatggtaggggatttggagttctctctaagcagcgaacagaaaattcaagcaccctagaagcagatcccattaatgcaatccgccccatcaaccatcaactacaagatctatcacaaacacacccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgctggagcggtcgagttctggaccgaccggctcgggttctcccgggacttcgtggaggacgacttcgctggtgtggtccgggacgacgtgaccctgttcatcagcgcggtccaggaccaggtggtgccggacaacaccttggcttgggtgtgggtgcgcggtttggacgagctgtacgccgagtggtcggaggtcgtgtccacgaacttccgggacgcctccggtccagccatgaccgagatcggcgagcagccgtgggggagggagttcgccctgcgcgatccggctggcaactgcgttcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctatcaactctggcccgaggaagtcagcgaaagaggatactttacccccagaggagaatataaaattgatgacaatgcagctcaagccatgaaagactcaatgctgtacaagatgtcattttacagattcggtgaacttttcccctctggagatgccatagaccgtgtcagaggtcaaagattgagtcgctcttacgctgagtctatagatcttaacattgttgaagaagtattcacaagtgagaactggctggttcgtctttacaagctcaaagagccagacaacttgggaagaagcctactgaccctgaaggataacgagaaaaagttggccactaaaaaaggtcgcagattgagggtcaacaagaaaccctcattggatctacgggtgtagtgtagttgtgtattttagatagacagagctctggggttagaatgataacatattgcgtgcttatgtaatgatgttcacctttcgtaggtctcaaaagttagcctagtcttagcccgtaactgatatatgtctctatctttggttcacacattcacaggtcacacagataaatgctgggctacttccattcactctaaacttcctttattggcaacagtctccggggacagaacttgtcgggtctataacttggaaacgaagagactgattgtcgtactcgacgatgattctcattcaaagactctttccagcgtcgaatggaagccaactggggaattcccttcgttggcaatcggctcgtttgattctacaatctctatttgggggaatgaagaagctgtcctggaagacgaggataactggacactgatggccattatcgaaggacatgagaacgagattaaaggggtgagttggagccatgatggagtgtatctggcatcatgctcacgagacaagtccatttggatatgggaggctgatgataacaacgaagagtttgagtgcgtctttgttgtacaggaacacagccaagatgtcaagcatgtaacatggcatcagcatga

SEQ ID NO: 233 - protein, 124 aa, synthetic construct
  source 1..124: note = The sequence is a translation of CDS kanMX nt (1024)..(1398) of SEQ ID NO. 232
MAKLTSAVPVLTARDVAGAVEFWTDRLGFSRDFVEDDFAGVVRDDVTLFISAVQDQVVPDNTLAWVWVRGLDELYAEWSEVVSTNFRDASGPAMTEIGEQPWGREFALRDPAGNCVHFVAEEQD

SEQ ID NO: 234 - DNA, 2734 bp, synthetic construct
  misc_feature 1..2734: note = Integration fragment INT3 comprising P(gsh2) promoter, cia1essential gene, and TIS3 translation initiation sequence.
  regulatory 1724..2234: note = promoter - P13_pGSH2; regulatory_class = promoter
  regulatory 2229..2234: note = promoter - TIS3; regulatory_class = promoter
  gene 2235..2734: note = first part of CIA1 gene, for integration by homologousrecombination
  source 1..2734: mol_type = other DNA
tggtcctcgtctatttgttgtttaaaagtgatagtatacttcttgaactcgctggggttaagaaattccaagtcttcatcttctacaaagaagtagttgtacgagaacaatagccatacagtaccacctatgacgaccagactaggaataaatctcaaccatgggtttctccgctttctaatgaccttttctgctttcaactccgatggtgaaggtggaggctggtgttcataagtgaccctttggttgggtcttggaatgtattgttttttcagttgaatcccttgcaacggttgaaggccgccagtcaacttgggggcgtttttcttagcagggactgcataatttctaacgagccgtagtattctctggttcatggatcatagaactgaggggaaggggaagtgtagaaatgagcagaaatggtaggggatttggagttctctctaagcagcgaacagaaaattcaagcaccctagaagcagatcccattaatgcaatccgccccatcaaccatcaactacaagatctatcacaaacacacccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgctggagcggtcgagttctggaccgaccggctcgggttctcccgggacttcgtggaggacgacttcgctggtgtggtccgggacgacgtgaccctgttcatcagcgcggtccaggaccaggtggtgccggacaacaccttggcttgggtgtgggtgcgcggtttggacgagctgtacgccgagtggtcggaggtcgtgtccacgaacttccgggacgcctccggtccagccatgaccgagatcggcgagcagccgtgggggagggagttcgccctgcgcgatccggctggcaactgcgttcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctatcaactctggcccgaggaagtcagcgaaagaggatactttacccccagaggagaatataaaattgatgacaatgcagctcaagccatgaaagactcaatgctgtacaagatgtcattttacagattcggtgaacttttcccctctggagatgccatagaccgtgtcagaggtcaaagattgagtcgctcttacgctgagtctatagatcttaacattgttgaagaagtattcacaagtgagaactggctggttcgtctttacaagctcaaagagccagacaacttgggaagaagcctactgaccctgaaggataacgagaaaaagttggccactaaaaaaggtcgcagattgagggtcaacaagaaaccctcattggatctacgggtgtagtgtagttgtgtattttagatagacagagctctggggttagaatgataacatattgcgtgcttatgtaatgatgttcacctttcgtaggtctcaaaagttagcctagtcttagcccgtaacacgttcatgtctctatctttggttcacacattcacaggtcacacagataaatgctgggctacttccattcactctaaacttcctttattggcaacagtctccggggacagaacttgtcgggtctataacttggaaacgaagagactgattgtcgtactcgacgatgattctcattcaaagactctttccagcgtcgaatggaagccaactggggaattcccttcgttggcaatcggctcgtttgattctacaatctctatttgggggaatgaagaagctgtcctggaagacgaggataactggacactgatggccattatcgaaggacatgagaacgagattaaaggggtgagttggagccatgatggagtgtatctggcatcatgctcacgagacaagtccatttggatatgggaggctgatgataacaacgaagagtttgagtgcgtctttgttgtacaggaacacagccaagatgtcaagcatgtaacatggcatcagcatga

SEQ ID NO: 235 - DNA, 2734 bp, synthetic construct
  misc_feature 1..2734: note = Integration fragment INT5 comprising P(gsh2) promoter, cia1essential gene, and TIS5 translation initiation sequence.
  regulatory 1724..2234: note = promoter - P13_pGSH2; regulatory_class = promoter
  regulatory 2229..2234: note = promoter - TIS5; regulatory_class = promoter
  gene 2235..2734: note = first part of CIA1 gene, for integration by homologousrecombination
  source 1..2734: mol_type = other DNA
tggtcctcgtctatttgttgtttaaaagtgatagtatacttcttgaactcgctggggttaagaaattccaagtcttcatcttctacaaagaagtagttgtacgagaacaatagccatacagtaccacctatgacgaccagactaggaataaatctcaaccatgggtttctccgctttctaatgaccttttctgctttcaactccgatggtgaaggtggaggctggtgttcataagtgaccctttggttgggtcttggaatgtattgttttttcagttgaatcccttgcaacggttgaaggccgccagtcaacttgggggcgtttttcttagcagggactgcataatttctaacgagccgtagtattctctggttcatggatcatagaactgaggggaaggggaagtgtagaaatgagcagaaatggtaggggatttggagttctctctaagcagcgaacagaaaattcaagcaccctagaagcagatcccattaatgcaatccgccccatcaaccatcaactacaagatctatcacaaacacacccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgctggagcggtcgagttctggaccgaccggctcgggttctcccgggacttcgtggaggacgacttcgctggtgtggtccgggacgacgtgaccctgttcatcagcgcggtccaggaccaggtggtgccggacaacaccttggcttgggtgtgggtgcgcggtttggacgagctgtacgccgagtggtcggaggtcgtgtccacgaacttccgggacgcctccggtccagccatgaccgagatcggcgagcagccgtgggggagggagttcgccctgcgcgatccggctggcaactgcgttcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctatcaactctggcccgaggaagtcagcgaaagaggatactttacccccagaggagaatataaaattgatgacaatgcagctcaagccatgaaagactcaatgctgtacaagatgtcattttacagattcggtgaacttttcccctctggagatgccatagaccgtgtcagaggtcaaagattgagtcgctcttacgctgagtctatagatcttaacattgttgaagaagtattcacaagtgagaactggctggttcgtctttacaagctcaaagagccagacaacttgggaagaagcctactgaccctgaaggataacgagaaaaagttggccactaaaaaaggtcgcagattgagggtcaacaagaaaccctcattggatctacgggtgtagtgtagttgtgtattttagatagacagagctctggggttagaatgataacatattgcgtgcttatgtaatgatgttcacctttcgtaggtctcaaaagttagcctagtcttagcccgtaactaggttatgtctctatctttggttcacacattcacaggtcacacagataaatgctgggctacttccattcactctaaacttcctttattggcaacagtctccggggacagaacttgtcgggtctataacttggaaacgaagagactgattgtcgtactcgacgatgattctcattcaaagactctttccagcgtcgaatggaagccaactggggaattcccttcgttggcaatcggctcgtttgattctacaatctctatttgggggaatgaagaagctgtcctggaagacgaggataactggacactgatggccattatcgaaggacatgagaacgagattaaaggggtgagttggagccatgatggagtgtatctggcatcatgctcacgagacaagtccatttggatatgggaggctgatgataacaacgaagagtttgagtgcgtctttgttgtacaggaacacagccaagatgtcaagcatgtaacatggcatcagcatga

SEQ ID NO: 236 - DNA, 2734 bp, synthetic construct
  misc_feature 1..2734: note = Integration fragment INT9 comprising P(gsh2) promoter, cia1essential gene, and TIS9 translation initiation sequence.
  regulatory 1724..2234: note = promoter - P13_pGSH; regulatory_class = promoter
  regulatory 2229..2234: note = promoter - TIS9; regulatory_class = promoter
  gene 2235..2734: note = first part of CIA1 gene, for integration by homologousrecombination
  source 1..2734: mol_type = other DNA
tggtcctcgtctatttgttgtttaaaagtgatagtatacttcttgaactcgctggggttaagaaattccaagtcttcatcttctacaaagaagtagttgtacgagaacaatagccatacagtaccacctatgacgaccagactaggaataaatctcaaccatgggtttctccgctttctaatgaccttttctgctttcaactccgatggtgaaggtggaggctggtgttcataagtgaccctttggttgggtcttggaatgtattgttttttcagttgaatcccttgcaacggttgaaggccgccagtcaacttgggggcgtttttcttagcagggactgcataatttctaacgagccgtagtattctctggttcatggatcatagaactgaggggaaggggaagtgtagaaatgagcagaaatggtaggggatttggagttctctctaagcagcgaacagaaaattcaagcaccctagaagcagatcccattaatgcaatccgccccatcaaccatcaactacaagatctatcacaaacacacccacacaccatagcttcaaaatgtttctactccttttttactcttccagattttctcggactccgcgcatcgccgtaccacttcaaaacacccaagcacagcatactaaattttccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaatttttttttttagtttttttctctttcagtgacctccattgatatttaagttaataaacggtcttcaatttctcaagtttcagtttcatttttcttgttctattacaactttttttacttcttgttcattagaaagaaagcatagcaatctaatctaaggggcggtgttgacaattaatcatcggcatagtatatcggcatagtataatacgacaaggtgaggaactaaaccatggccaagttgaccagtgccgttccggtgctcaccgcgcgcgacgtcgctggagcggtcgagttctggaccgaccggctcgggttctcccgggacttcgtggaggacgacttcgctggtgtggtccgggacgacgtgaccctgttcatcagcgcggtccaggaccaggtggtgccggacaacaccttggcttgggtgtgggtgcgcggtttggacgagctgtacgccgagtggtcggaggtcgtgtccacgaacttccgggacgcctccggtccagccatgaccgagatcggcgagcagccgtgggggagggagttcgccctgcgcgatccggctggcaactgcgttcacttcgtggccgaggagcaggactgacacgtccgacggcggcccacgggtcccaggcctcggagatccgtcccccttttcctttgtcgatatcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcaagctatcaactctggcccgaggaagtcagcgaaagaggatactttacccccagaggagaatataaaattgatgacaatgcagctcaagccatgaaagactcaatgctgtacaagatgtcattttacagattcggtgaacttttcccctctggagatgccatagaccgtgtcagaggtcaaagattgagtcgctcttacgctgagtctatagatcttaacattgttgaagaagtattcacaagtgagaactggctggttcgtctttacaagctcaaagagccagacaacttgggaagaagcctactgaccctgaaggataacgagaaaaagttggccactaaaaaaggtcgcagattgagggtcaacaagaaaccctcattggatctacgggtgtagtgtagttgtgtattttagatagacagagctctggggttagaatgataacatattgcgtgcttatgtaatgatgttcacctttcgtaggtctcaaaagttagcctagtcttagcccgtaacaaaacaatgtctctatctttggttcacacattcacaggtcacacagataaatgctgggctacttccattcactctaaacttcctttattggcaacagtctccggggacagaacttgtcgggtctataacttggaaacgaagagactgattgtcgtactcgacgatgattctcattcaaagactctttccagcgtcgaatggaagccaactggggaattcccttcgttggcaatcggctcgtttgattctacaatctctatttgggggaatgaagaagctgtcctggaagacgaggataactggacactgatggccattatcgaaggacatgagaacgagattaaaggggtgagttggagccatgatggagtgtatctggcatcatgctcacgagacaagtccatttggatatgggaggctgatgataacaacgaagagtttgagtgcgtctttgttgtacaggaacacagccaagatgtcaagcatgtaacatggcatcagcatga

SEQ ID NO: 237 - DNA, 19 bp, synthetic construct
  primer_bind 1..19: note = primer E251
  source 1..19: mol_type = other DNA
acgcgtgtacgcatgtaac

SEQ ID NO: 238 - DNA, 20 bp, synthetic construct
  primer_bind 1..20: note = primer E627
  source 1..20: mol_type = other DNA
cataactggagcttgctagg

SEQ ID NO: 239 - DNA, 750 bp, Aspergillus oryzae
  regulatory 1..750: note = promoter - Aspergillus oryzae burden sensing promoter p(agsA); regulatory_class = promoter
  source 1..750: mol_type = genomic DNA
gttgtttcttaatataggcacacggatcccgccatcgatcggcgccctgttcgagtatggccatcctcacacattgtcgggctccgctggcgctgcctcaacatatttttaaatggggaaaccataagcaaagagactccatcgggcgcactacacatctcgcctcttctggaccctgtggatcgggactggggtggatggcaaaccgtcaagttgccgccgcttcttcgcgctgttgcggcgtcacgccatataggcgactctcccaatcatggaaagcctgcgttccagcgatattcaacgacgtggtggcttggattgcagcttcgaccagatcacagcaatagaatagcgatgattccagccgtgggcctttagggtagaaaccatactgggctaggaaacatgccgtatcaagtgagaaacaccgctaccccaccacggctgaaaagtagatagtggatggttccatgtcatctatcagcaggcgaggagtgggtctatagtagaggttgtaatggtcgtggtagaggtccctggcccatttttatccccatccttcccggcctgatggttccaagccttctttacgaaaagttttgcagctgctttttcgagtcgacaggtcgctcgttccttcgtttgatttccctcgcttttgaccacgcccagggcatgtaggatttattccgtttgagattatcgagagcctaagtcgcgcgcagttgtcggtagcaggccactgccggg

SEQ ID NO: 240 - DNA, 1200 bp, Aspergillus oryzae
  source 1..1200: mol_type = genomic DNA
  CDS 1..1200: note = ERG10 Aspergillus essential gene (mRNA)
atgtcttctcttccgccggtctatattgtttcctctgcccgcaccccagtcggctctttcttggggtcgctctcaagtctcactgccccgcagttaggctctcatgctattaaagctgcgctcagcaaagcggatggaatcaagccgtctgatatccaggaggtcttctttggcaatgtcatctccgcaaacgttggacaaaatcctgctagacagtgtgctctcggcgctggtctcaatgaatcaactgtctgtactacggttaataaggtgtgcgcgtctggcttgaaagcggttattctcggtgcacagaccatcatgactggcaatgcggatattgtcgtagcaggcggtgctgaatccatgtctaacgcccctcattaccttccaaaccttcgcgtcggtgcgaaatacggcaaccagagtctggtggacggtattatgaaggatggcttgacagacgcaggaaagcaggaactcatgggcttgcaagccgaggagtgtgctcaggatcatggctttagcagggaacaacaggatgattatgccattcgcacttacgaaaaagcacaggcggctcaaaaggctggcctttttgacgaagaaattgcgcctattgaacttcctggctttaggggcaagccaggtgtgactgtgtcacaagacgaagaaccaaagaatcttaacccggataagcttcgagctatcaagcctgcatttatccccggatccggcacggtcacagccccgaattcctcacctcttaacgacggtgctgctgctgttatcctcgtctcagaagctaaactgaaagagcttaacctaaagcctgttgcaaagattcttggctggggagatgccgcccagcagccaagcaaattcacaactgccccagctctagcaattcccaaggccctcagccatgcaggtgtggctcaggatgctgttgatgcgttcgagattaacgaagcgttcagcgtagttgctctggccaatatgaaactcctggggttggctgaagataaagtcaacatccatggtggtgcagtggctatcggtcatcctatcggcgccagcggtgctcgtatcttgactacattgctcggtgtattgaaagcgagaaagggtaagattggttgtgccgggatttgtaatggaggaggtggtgctagcgctattgttgtcgaatctctcgtctga

SEQ ID NO: 241 - protein, 399 aa, Aspergillus oryzae
  source 1..399: note = The sequence is a translation of SEQ ID NO. 240
MSSLPPVYIVSSARTPVGSFLGSLSSLTAPQLGSHAIKAALSKADGIKPSDIQEVFFGNVISANVGQNPARQCALGAGLNESTVCTTVNKVCASGLKAVILGAQTIMTGNADIVVAGGAESMSNAPHYLPNLRVGAKYGNQSLVDGIMKDGLTDAGKQELMGLQAEECAQDHGFSREQQDDYAIRTYEKAQAAQKAGLFDEEIAPIELPGFRGKPGVTVSQDEEPKNLNPDKLRAIKPAFIPGSGTVTAPNSSPLNDGAAAVILVSEAKLKELNLKPVAKILGWGDAAQQPSKFTTAPALAIPKALSHAGVAQDAVDAFEINEAFSVVALANMKLLGLAEDKVNIHGGAVAIGHPIGASGARILTTLLGVLKARKGKIGCAGICNGGGGASAIVVESLV

SEQ ID NO: 242 - DNA, 2082 bp, Aspergillus oryzae
  source 1..2082: mol_type = genomic DNA
  CDS 1..2082: note = PFS2 Aspergillus essential gene (mRNA)
atgtgcggcatcttcggctacattaactacctcgtcgagagggaccgcaagtttattcttgacactcttctcaatggactctcccgtcttgaataccgaggctacgactctgcgggtcttgccatagatggcgataagaagaatgaagtttgtgctttcaaggaagtgggcaaggttgctaagcttcgagagcttattgatgagtgcaagcctgacttgaccaaatcctttgaatctcatgctggtatttctcacactcgttgggccactcatgggacgccttcccgcttgaactgccacccccacaggtccgatctgaactgggaattttcagttgtccacaatggtatcattaccaactacaaagagctcaaggctttgttggagagcaagggattccgctttgagactgacacagacaccgaatgtattgcaaagcttaccaaatatctctatgaccaacagcccgatatcgaattcacagttttggctaaggctgttgtgaaggagcttgagggcgcgtttgggcttctcatcaaatccgtacattatccccatgaggtcattgcggctcgcaaggggtcgccgcttgttattggcgtgagaacgtcgaagaagatgaaggtggatttcgtggacgtcgaatactcagaagatggagcccttcctgcagagcaggcctcccagaacgtggccatcaagaagtctgcgaccggcctccttgccccacctgacaaatcccttttgcataggtcacagtctcgcgctttcctttccgatgatggcatcccccagccagctgagtttttcttgtcttctgacccatctgcaattgtcgagcatacgaagaaggtcctctatctcgaagatgatgatatcgctcatgtgcatgagggacagttaaacattcatcgcctcacaaaagatgatggtacttctaatgttcgcgctattcagacaattgaactcgaattgcaggagattatgaagggcaagtttgaccacttcatgcaaaaggaaattttcgagcaacccgagtctgtgatcaataccatgagaggacgactggatgttgcaaacaagcaagtcacactcggtggcctgcggcagtacatttctactatccgccgctgcagaagaatcatatttgttgcctgtggaactagttaccattcatgcatggctgtgcgtggagtctttgaagagcttactgagattcctatttctgttgaacttgcttccgatttcctggatagacaggctccagtattccgtgatgacacgtgtgttttcgtttctcagtctggcgagaccgctgactcccttatggccttgcgctactgtctcgaacgcggagcattgaccgttggtattgttaatgttgtagggtcctctatctcacttctcactcactgcggtgtgcacatcaacgctggacccgaaattggggtagcttctaccaaggcctacacgtcccagttcgttgctatggtcatgttcgctttgtctcttagtgaggatagggcgtctaagcagaagaggcgtgaggagattatggagggcctttctaaggtctccgagcaattcaaggagatcttgaaacttaacgaacccatcaaacaattgtgtgcaaacttcaagaatcaaaagagtttgcttctgctgggcaggggtggtcaattccctactgcccttgaaggtgcacttaaaatcaaagagatctcttatctccattgcgaggctgtcatgtccggcgagttgaagcatggtgttcttgcccttgtcgatgaaaacttgcccatcatcatgattcttacgagggacaacctgttcacgaagtcgttgaatgcttatcaacaagtcattgccagaggtggccgtcctattgtaatttgtaattccgatgacccggaattttcttcggctcaaaccgtgaagattgaagttccaaagactgtcgattgcctgcaaggtctcctgaatgtcatccccttgcagttaatcgcttactggttggctgtgtctgagggtctcaatgttgatttccctcgcaatcttgcgaagtcagtcacagttgagtaa

SEQ ID NO: 243 - protein, 693 aa, Aspergillus oryzae
  source 1..693: note = The sequence is a translation of SEQ ID NO. 242
MCGIFGYINYLVERDRKFILDTLLNGLSRLEYRGYDSAGLAIDGDKKNEVCAFKEVGKVAKLRELIDECKPDLTKSFESHAGISHTRWATHGTPSRLNCHPHRSDLNWEFSVVHNGIITNYKELKALLESKGFRFETDTDTECIAKLTKYLYDQQPDIEFTVLAKAVVKELEGAFGLLIKSVHYPHEVIAARKGSPLVIGVRTSKKMKVDFVDVEYSEDGALPAEQASQNVAIKKSATGLLAPPDKSLLHRSQSRAFLSDDGIPQPAEFFLSSDPSAIVEHTKKVLYLEDDDIAHVHEGQLNIHRLTKDDGTSNVRAIQTIELELQEIMKGKFDHFMQKEIFEQPESVINTMRGRLDVANKQVTLGGLRQYISTIRRCRRIIFVACGTSYHSCMAVRGVFEELTEIPISVELASDFLDRQAPVFRDDTCVFVSQSGETADSLMALRYCLERGALTVGIVNVVGSSISLLTHCGVHINAGPEIGVASTKAYTSQFVAMVMFALSLSEDRASKQKRREEIMEGLSKVSEQFKEILKLNEPIKQLCANFKNQKSLLLLGRGGQFPTALEGALKIKEISYLHCEAVMSGELKHGVLALVDENLPIIMILTRDNLFTKSLNAYQQVIARGGRPIVICNSDDPEFSSAQTVKIEVPKTVDCLQGLLNVIPLQLIAYWLAVSEGLNVDFPRNLAKSVTVE

SEQ ID NO: 244 - DNA, 1347 bp, Aspergillus oryzae
  source 1..1347: mol_type = genomic DNA
  CDS 1..1347: note = TUB1 Aspergillus essential gene (mRNA)
atgagagaagttattagtttgaacgttggtcaggctggttgccagatcgccaattcttgctgggagctctactgtcttgagcacggcatccagcccgatggttacttgaccgaggaacgcaagaaggaagaccctgaccatggtttcagcaccttcttctccgaaactggccagggcaagtatgttcctcgtaccatctacgccgatctggagcccaatgttgtcgatgaggtccgcactggcacctaccgtacccttttccaccccgagaacatgatcaccggcaaggaggatgcctcgaacaactatgcccgtggtcactacaccgttggcaaggagatgatcgaccaggtcctcgacaaggttcgccgtgtggccgacaactgcgctggtctccagggcttcctcgtcttccactctttcggtggtggtactggttccggtttcggtgctctcctgatggagcgtctgtctgtggactacggcaagaagtccaagctggagttctgcgtctaccctgccccccagaatgccacctccgtcgttgagccctacaactccatcctgactacccacaccacccttgagcactccgactgcagtttcatggttgacaacgaagccatctacgacatctgccgccgcaaccttggcatcgagcgtcccagctatgagaacctgaaccgcctgattgctcaggttgtctcctccatcaccgcctccctgcgtttcgatggttccctgaacgtggatctcaacgagttccagaccaacctggttccctacccccgtattcacttccctctcgttgcctatgctcccgtcatctccgcggccaaggcctcccacgaggccaactccgtcaacgagatcaccatgtcttgcttcgagcccaacaaccagatggtcaagtgtgacccccgcaatggcaagtacatggctacttgcttgctgtaccgtggtgatgttgtgcccaaggagacccacgccgccgttgctaccctcaagaccaagcgtaccatccagttcgtcgactggtgccctactggtttcaagatcggtatctgctaccagccccctaagcaggttcccaacggtgaccttgccaacctcagccgtgctgtctgcatgctgtctaacaccaccgccatctccgaggcctggtccgctctcgaccacaagttcgatctcatgtactccaagcgtgctttcgttcactggtatgttggagagggtatggaggagggtgaattctccgaggcccgtgaggacctggctgccctcgagcgcgactacgaggaggtcgccagcgactcgctggaggaggaggttgaggctgagtactaa

SEQ ID NO: 245 - protein, 448 aa, Aspergillus oryzae
  source 1..448: note = The sequence is a translation of SEQ ID NO. 244
MREVISLNVGQAGCQIANSCWELYCLEHGIQPDGYLTEERKKEDPDHGFSTFFSETGQGKYVPRTIYADLEPNVVDEVRTGTYRTLFHPENMITGKEDASNNYARGHYTVGKEMIDQVLDKVRRVADNCAGLQGFLVFHSFGGGTGSGFGALLMERLSVDYGKKSKLEFCVYPAPQNATSVVEPYNSILTTHTTLEHSDCSFMVDNEAIYDICRRNLGIERPSYENLNRLIAQVVSSITASLRFDGSLNVDLNEFQTNLVPYPRIHFPLVAYAPVISAAKASHEANSVNEITMSCFEPNNQMVKCDPRNGKYMATCLLYRGDVVPKETHAAVATLKTKRTIQFVDWCPTGFKIGICYQPPKQVPNGDLANLSRAVCMLSNTTAISEAWSALDHKFDLMYSKRAFVHWYVGEGMEEGEFSEAREDLAALERDYEEVASDSLEEEVEAEY

SEQ ID NO: 246 - DNA, 5509 bp, synthetic construct
  gene 1970..2868: note = pyrG
  regulatory 3260..4009: note = promoter - agsA; regulatory_class = promoter
  regulatory 4004..4009: note = promoter - TIS; regulatory_class = promoter
  gene 4010..5474: note = essential gene ERG10
  exon 4010..4074
  exon 4142..4191
  exon 4249..4324
  exon 4398..4887
  exon 4956..5474
  source 1..5509: mol_type = other DNA
aacgcgacgttgcccttttctgggcttagtcgccatttttcgccttgtccaggcaagacactttcaatgacagtgttgacttcttcaatcacatgcttgagcttgaaataggcgtcgttgggtggtatcttcagttcgaggatcagccgatccattttgttcacaaccatggtcaacggaaggccctctagcacagcgtgcttgatgatttgctccgtattcgattgtactccttccacaatatcaacgactatcaccacgccatcaaccagtcgtgatgaggcagctacttcatcaacaaaattcacatgccccggggtatcaagaatgttgaacaaatgagacttccccttagtgccctgaagaactaggctcataggagctgatttaatagaaaggcctcgctctctctctagaaagtgaatatcggtataacgaagctgtacttcctttcttttacctgctcgcttctctagccgttcagtcaaatcatgcgtttgcataacaagcatatccatgaacgcggtcttcccatgatgtaaatgaccgacaagcgcaatgttccttatttgttcaggaaaattcagaagatcagtcatgaattcccttgaaaagtgaacccgggggagctcagtctcttcaatggcgaacttcttttgctgtacgggggcaacgataggctcggagagaggctgtgcgtcttcttcttgaactaatgtctctacctctgtcccgtagacctgttgtgcgctagggtagtattgtttgtcttcgtgtagaatgacggcatttgagggggcttctgtaatgagatatatcagcaccgactaaatccaggggctttaagagagtgcccaccgtccacctccatagactgctgcccatataattcttcatcttcttcctccccaaacgcctcgttgaaggcgaaagcccgaggcttcacttcgtcgtgttggccgtcttcatctgaatctgcagcctctccaatgtagttgccaaacctgtgtggtcttgattagaatttgcacaattagagcgttagattggatgcaacttactcatcgtagagatcatccatcgtgcggtgggaaaggtggttcaagctgccaaaaacttgtcaccagtgagctgtcgcatctctttttgtcccaagctgctgaagtccgtctaatggactcaccgagtggtccgtgcgtcgaccctaccattttggtagccccgccgcgcggatgccacgtccaggaggggtccttaaatgactttgatttctcaccacctcttctcgtgtcagagatacctcactgctatcgtcggctgaataactcaactctttcttgtttcccttcttcccccctttaatcccaccaatcttctttattaccggcaagttctgtctacacgggtgccctttaccacacaccgaacccctttaaacgcttcacgctacagaagcttttggtttacgactcttctaatcagctatcgtaggccgccgatagctgattaaatattctagacccaagccgctgctggaattgacattattatggccgatagggttgggcttattgctatgtccctgaaaggatatcaaaagcaggcaaaaagccaggcataatccccgcgtggacggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggatgccacttacatgtgatctagtaatggttaatggtggattatataacaggactcggtccgtacattgccgtgatcaaaactcacatcgatatcctctccgatttcagcgaagaaaccatcaccggtctgaaggcccttgcagagaagcacaatttcctcatcttcgaagatcgcaagttcatcgatatcggaaacacagtccaaaagcagtaccatggcggcactctgcgtatctctgagtgggcccacatcatcaactgcagtattctgcccggtgagggtatcgtcgaggctctggcccagactgcttcggccgaggacttcccctacggctccgagaggggccttttgatccttgcggagatgacctccaagggatctttggctaccggtcaatatactacttcttctgttgactatgctcggaagtataagaagtttgtgatgggattcgtctcgacacgtcaccttggcgaggttcagtctgaagttagctcgccttcggaggaggaagattttgtcgtcttcacgacaggtgtcaacctctcctcgaagggtgacaagctgggacagcagtaccaaactcctgagtcggctgttggacgcggtgccgactttattattgctggccgtggaatttatgctgctcctgatcccgtggaggcggcgaagcagtaccagaaggagggatgggatgcatacctgaagcgtgttggtgcgcaataagtagtggtggatacgtactccttttatggcagtatgtcgcaagtatgatgcgatttataaattcagcactcgaaatgactactactatgtgtctacgacagataccctctccgtacgaataagacacctgcctcgatatatggacaaattcaaaatcagggtcaagggtcatgtttcaaagtcacaacaatctccaacatagacgagaatttgtaccggagtgtctgaaggtgcagctggagattggtctattttcttagagtggggtatcactaatgtacagtcggtcactatcgtacaaacaatcacaattatatacaagatttcccaccaccccctactctaacacggcacaattatccatcgagtcagagcctagccaccatttggtgttgtttcttaatataggcacacggatcccgccatcgatcggcgccctgttcgagtatggccatcctcacacattgtcgggctccgctggcgctgcctcaacatatttttaaatggggaaaccataagcaaagagactccatcgggcgcactacacatctcgcctcttctggaccctgtggatcgggactggggtggatggcaaaccgtcaagttgccgccgcttcttcgcgctgttgcggcgtcacgccatataggcgactctcccaatcatggaaagcctgcgttccagcgatattcaacgacgtggtggcttggattgcagcttcgaccagatcacagcaatagaatagcgatgattccagccgtgggcctttagggtagaaaccatactgggctaggaaacatgccgtatcaagtgagaaacaccgctaccccaccacggctgaaaagtagatagtggatggttccatgtcatctatcagcaggcgaggagtgggtctatagtagaggttgtaatggtcgtggtagaggtccctggcccatttttatccccatccttcccggcctgatggttccaagccttctttacgaaaagttttgcagctgctttttcgagtcgacaggtcgctcgttccttcgtttgatttccctcgcttttgaccacgcccagggcatgtaggatttattccgtttgagattatcgagagcctaagtcgcgcgcagttgtcggtagcaggccactgccgggatgtcttctcttccgccggtctatattgtttcctctgcccgcaccccagtcggctctttcttggggtgagattggccttgtgccttgtctatacgtacaatattgacttacactgactatccgcttccacaggtcgctctcaagtctcactgccccgcagttaggctctcatgctattaaaggtatgacagctctcgagtggcggtacgtgttgatatactaacaaactcgaaattcagctgcgctcagcaaagcggatggaatcaagccgtctgatatccaggaggtcttctttggcaatgtcatctccgcaaagtgagtcaacgtccacccttataccttatagccaacatcgaaggcacacttacattctcgttctatggatcagcgttggacaaaatcctgctagacagtgtgctctcggcgctggtctcaatgaatcaactgtctgtactacggttaataaggtgtgcgcgtctggcttgaaagcggttattctcggtgcacagaccatcatgactggcaatgcggatattgtcgtagcaggcggtgctgaatccatgtctaacgcccctcattaccttccaaaccttcgcgtcggtgcgaaatacggcaaccagagtctggtggacggtattatgaaggatggcttgacagacgcaggaaagcaggaactcatgggcttgcaagccgaggagtgtgctcaggatcatggctttagcagggaacaacaggatgattatgccattcgcacttacgaaaaagcacaggcggctcaaaaggctggcctttttgacgaagaaattgcgcctattgaacttcctggctttaggggcaagccaggtgtgactgtgtcacaagacgaagaaccaaagaatgtgaggaagatattactttctacagattttttaagtcgtcggccactgctaatatactcattgcgtagcttaacccggataagcttcgagctatcaagcctgcatttatccccggatccggcacggtcacagccccgaattcctcacctcttaacgacggtgctgctgctgttatcctcgtctcagaagctaaactgaaagagcttaacctaaagcctgttgcaaagattcttggctggggagatgccgcccagcagccaagcaaattcacaactgccccagctctagcaattcccaaggccctcagccatgcaggtgtggctcaggatgctgttgatgcgttcgagattaacgaagcgttcagcgtagttgctctggccaatatgaaactcctggggttggctgaagataaagtcaacatccatggtggtgcagtggctatcggtcatcctatcggcgccagcggtgctcgtatcttgactacattgctcggtgtattgaaagcgagaaagggtaagattggttgtgccgggatttgtaatggaggaggtggtgctagcgctattgttgtcgaatctctcgtctgagtcatgccgagtcgcccttgatcttggtatggaat

SEQ ID NO: 247 - DNA, 5509 bp, synthetic construct
  gene 1970..2868: note = pyrG
  regulatory 3260..4009: note = promoter - agsA promoter; regulatory_class = promoter
  regulatory 4004..4009: note = promoter - TIS; regulatory_class = promoter
  gene 4010..5509: note = essential gene FKS2
  exon 4010..4014
  exon 4072..4142
  exon 4229..4460
  exon 4534..5509
  source 1..5509: mol_type = other DNA
ataaaaatttcgccaccgaagaagtatcaaggaaactgtctagcgagagggactggactggaacacttgagtattcggtatccagttaaggtgatttaagccaccccttctcccttcaatccctaccccccttcaaaaaggggaagaacagtagctaaaggtagttgccatcaagaactcatacttaaaaacacgaggtaagaggcccgtaatagtaattggttgcaagagaaaaggggggttatccttctgagcatcacagttttgattacgggaagaaccagaatatgaacgtggcaacagcggctgtcggtaccagaccaggcctttattggataccggaggagtccctctttcgtgtgtttcctaccccttttacagtacaattacaacctacataatgtcactgatagatatttatacgtgtgccagaagatacttatgggttagtcaatgtaaatagcttctaattttgattcatgattgaggggtctaaaattggactgaacctaatcctactcaagtaggagttagatgacgatggtgagtgtgaaatgccaggattaagggtagcatatcagccgcacaggaactaccgagggggtggaggctaacagccctctttttttctttccctttttcctcattttcctctctcttctcccgatgatgacgatttgtagattcataagttatagaaaaatatttgttctatgcacacgctaataggacatagtactgcttattgtatgtatatgcctggtgtttaaagaagtagacttcttgagttccgtacggagcactattttacaatacgaataaaaaaaatccatggagcagggaagggaaggatatagctagctaggtccctaatagtcgatatgttatttttgatctgttagtggagccactgacctgttgaatttttggtaggtaaacttagcaacatgtccatgccataaaaggtctccaaccgagagtcacgagtgaaacataccagtaggtccaacaaactgtgtaaggtgcagaaaaggcacattattggccagctccatgttgaatgaatggctgcgccctggagccggtccatagtagcttccctggtctctgtgtgggccgatctcacaacataaaacatagctcttccccctccttccaacagattgtcgatctactcaatttcccctttgctatcaacttctccccctcctcgcttccttctctcactcgccacaccgccctttaagaatatcacaagtaatattctagctggattatccaaagtgtgttttatgttgtccttccatggtatcttccaattcgtttgttgacagtatattcccttccagagtgcactttgaagaaaccccactaccgtttacgtgacttgctttcaatatccggaggcaacatttgttatcgaagcattacaacgcctagtcatctcattctacacctttccacattttgactcgctctctctcttgacataccttaaccagctgattaaatattctagacccaagccgctgctggaattgacattattatggccgatagggttgggcttattgctatgtccctgaaaggatatcaaaagcaggcaaaaagccaggcataatccccgcgtggacggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggatgccacttacatgtgatctagtaatggttaatggtggattatataacaggactcggtccgtacattgccgtgatcaaaactcacatcgatatcctctccgatttcagcgaagaaaccatcaccggtctgaaggcccttgcagagaagcacaatttcctcatcttcgaagatcgcaagttcatcgatatcggaaacacagtccaaaagcagtaccatggcggcactctgcgtatctctgagtgggcccacatcatcaactgcagtattctgcccggtgagggtatcgtcgaggctctggcccagactgcttcggccgaggacttcccctacggctccgagaggggccttttgatccttgcggagatgacctccaagggatctttggctaccggtcaatatactacttcttctgttgactatgctcggaagtataagaagtttgtgatgggattcgtctcgacacgtcaccttggcgaggttcagtctgaagttagctcgccttcggaggaggaagattttgtcgtcttcacgacaggtgtcaacctctcctcgaagggtgacaagctgggacagcagtaccaaactcctgagtcggctgttggacgcggtgccgactttattattgctggccgtggaatttatgctgctcctgatcccgtggaggcggcgaagcagtaccagaaggagggatgggatgcatacctgaagcgtgttggtgcgcaataagtagtggtggatacgtactccttttatggcagtatgtcgcaagtatgatgcgatttataaattcagcactcgaaatgactactactatgtgtctacgacagataccctctccgtacgaataagacacctgcctcgatatatggacaaattcaaaatcagggtcaagggtcatgtttcaaagtcacaacaatctccaacatagacgagaatttgtaccggagtgtctgaaggtgcagctggagattggtctattttcttagagtggggtatcactaatgtacagtcggtcactatcgtacaaacaatcacaattatatacaagatttcccaccaccccctactctaacacggcacaattatccatcgagtcagagcctagccaccatttggtgttgtttcttaatataggcacacggatcccgccatcgatcggcgccctgttcgagtatggccatcctcacacattgtcgggctccgctggcgctgcctcaacatatttttaaatggggaaaccataagcaaagagactccatcgggcgcactacacatctcgcctcttctggaccctgtggatcgggactggggtggatggcaaaccgtcaagttgccgccgcttcttcgcgctgttgcggcgtcacgccatataggcgactctcccaatcatggaaagcctgcgttccagcgatattcaacgacgtggtggcttggattgcagcttcgaccagatcacagcaatagaatagcgatgattccagccgtgggcctttagggtagaaaccatactgggctaggaaacatgccgtatcaagtgagaaacaccgctaccccaccacggctgaaaagtagatagtggatggttccatgtcatctatcagcaggcgaggagtgggtctatagtagaggttgtaatggtcgtggtagaggtccctggcccatttttatccccatccttcccggcctgatggttccaagccttctttacgaaaagttttgcagctgctttttcgagtcgacaggtcgctcgttccttcgtttgatttccctcgcttttgaccacgcccagggcatgtaggatttattccgtttgagattatcgagagcctaagtcgcgcgcagttgtcggtagcaggccactgccgggatgtggtacgttcccgaagacccaggcttactgaatgtttgatgtaactaattgacccctagcggcatcttcggctacattaactacctcgtcgagagggaccgcaagtttattcttgacactcttctcaatggttggcattcctgttgttttcaccctcactggaccttgtttactctggttttgggagtgtttgtgtattcctctgacatattgaaggactctcccgtcttgaataccgaggctacgactctgcgggtcttgccatagatggcgataagaagaatgaagtttgtgctttcaaggaagtgggcaaggttgctaagcttcgagagcttattgatgagtgcaagcctgacttgaccaaatcctttgaatctcatgctggtatttctcacactcgttgggccactcatgggacgccttcccgcttgaactgccacccccacaggtgagtccgatgttccacggtttaatcagctcgactaggatcaggcttgtttgctaattttcctggcacgcaggtccgatctgaactgggaattttcagttgtccacaatggtatcattaccaactacaaagagctcaaggctttgttggagagcaagggattccgctttgagactgacacagacaccgaatgtattgcaaagcttaccaaatatctctatgaccaacagcccgatatcgaattcacagttttggctaaggctgttgtgaaggagcttgagggcgcgtttgggcttctcatcaaatccgtacattatccccatgaggtcattgcggctcgcaaggggtcgccgcttgttattggcgtgagaacgtcgaagaagatgaaggtggatttcgtggacgtcgaatactcagaagatggagcccttcctgcagagcaggcctcccagaacgtggccatcaagaagtctgcgaccggcctccttgccccacctgacaaatcccttttgcataggtcacagtctcgcgctttcctttccgatgatggcatcccccagccagctgagtttttcttgtcttctgacccatctgcaattgtcgagcatacgaagaaggtcctctatctcgaagatgatgatatcgctcatgtgcatgagggacagttaaacattcatcgcctcacaaaagatgatggtacttctaatgttcgcgctattcagacaattgaactcgaattgcaggagattatgaagggcaagtttgaccacttcatgcaaaaggaaattttcgagcaacccgagtctgtgatcaataccatgagaggacgactggatgttgcaaacaagcaagtcacactcggtggcctgcggcagtacatttctactatccgccgctgcagaagaatcatatttgttgcctgtggaactagttaccattcatgcatggctgtgcgtggagtctttgaagagcttactgagattcctatttctgttgaacttgcttccgatttcctggatagacaggctccagtattccgtgatgacacg

SEQ ID NO: 248 - DNA, 5509 bp, synthetic construct
  gene 1970..2868: note = pyrG
  regulatory 3260..4009: note = promoter - agsA; regulatory_class = promoter
  regulatory 4004..4009: note = promoter - TIS; regulatory_class = promoter
  gene 4010..5509: note = TUB1 essential gene
  exon 4010..4034
  exon 4124..4164
  exon 4239..4265
  exon 4337..4412
  exon 4473..4527
  exon 5481..5509
  source 1..5509: mol_type = other DNA
cgggaagatgttccatagaaagggatcaaggatgatatccgctgcaaccgggggaacattctccattagtgctttgttaagagaaaagtagtccgccgccgcagtaacacgaaggaaaagcaccgaatcagacgtaggtgcttgatcatgaaaagaggaagatttgcgaggaacagcgttagactgaggggaggcgagatgggcttgggaatagaggctaatcgaggagaagagtgcgggatcatcaatggcttgtggaagagtatgcgttgtcagggtaaaggcggtcggttgctttgactacttgtcagctaattttctttggtcgccttgctgggcactggtagagggtatatgaggacgtacagttgccaaccaacacacccgcacctcataccgctggccgggggtcaagttctccagaaaataccatgagtctgtaccagggaactcgttggtcggaaatgtagcgttaattctggttcgaagaacgggactagaggaggagaggcgatctagacctaggtcatcgagggtaggatcgaccgtggggatgatgaggggttgcggagcgatgaagatcgttttctcgacattggcgatagcggggaatgcgagggtgcagagtaagagtagatattggagtagcatcatgacagagagttcgctatgttcaattgacgctgctgctagacggtcatggttctagtccacacgcagttgagtatggtaggctatatgtatgtatgtagtaaatgaggtagatctgatactaactgttagattcctaagtgaagtccttacatgaaattgattcaatgtcaatacgatcttaccataaattaggccattgtgcgcagcaatgttggtgactagaaatagagcactgtgtgtccgagacttgttctatgtgagtattttcaatagtacttactatcgcaagctgtacggatagtcaaatacaatgcagctgcacgaagagatgccgtcattgacacgtgatcgacccttgccgtgaactaagcccaaataagtggctgagtcagcggtgggctggaagggcggtcacggcatcggacaagccaatcagaaatgggattgtgggcgacgcgacccaacggatggatcaacaatgaaaaggtggaagtcctgcttttgggttgacagcgcatcgcaagaccacaacaaattgaccgatcattcgggtcttgggcaattgtttaattgatccccttagtctatttttctcctcgtcaatcaccccaagggtttgttctcctcccccatttcctcccaccggttcccctttccctttcttttggttcttttcccaggtggtgtttttttcgtttacttacccctgaaacttccttacagatcagggcatttttccccctctaaagtccttccacattccacccactccgtttcacacacccccctccttccaatcttaattcctttgtcactccgacattcgtcccttttagactcgacaagagctgattaaatattctagacccaagccgctgctggaattgacattattatggccgatagggttgggcttattgctatgtccctgaaaggatatcaaaagcaggcaaaaagccaggcataatccccgcgtggacggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggatgccacttacatgtgatctagtaatggttaatggtggattatataacaggactcggtccgtacattgccgtgatcaaaactcacatcgatatcctctccgatttcagcgaagaaaccatcaccggtctgaaggcccttgcagagaagcacaatttcctcatcttcgaagatcgcaagttcatcgatatcggaaacacagtccaaaagcagtaccatggcggcactctgcgtatctctgagtgggcccacatcatcaactgcagtattctgcccggtgagggtatcgtcgaggctctggcccagactgcttcggccgaggacttcccctacggctccgagaggggccttttgatccttgcggagatgacctccaagggatctttggctaccggtcaatatactacttcttctgttgactatgctcggaagtataagaagtttgtgatgggattcgtctcgacacgtcaccttggcgaggttcagtctgaagttagctcgccttcggaggaggaagattttgtcgtcttcacgacaggtgtcaacctctcctcgaagggtgacaagctgggacagcagtaccaaactcctgagtcggctgttggacgcggtgccgactttattattgctggccgtggaatttatgctgctcctgatcccgtggaggcggcgaagcagtaccagaaggagggatgggatgcatacctgaagcgtgttggtgcgcaataagtagtggtggatacgtactccttttatggcagtatgtcgcaagtatgatgcgatttataaattcagcactcgaaatgactactactatgtgtctacgacagataccctctccgtacgaataagacacctgcctcgatatatggacaaattcaaaatcagggtcaagggtcatgtttcaaagtcacaacaatctccaacatagacgagaatttgtaccggagtgtctgaaggtgcagctggagattggtctattttcttagagtggggtatcactaatgtacagtcggtcactatcgtacaaacaatcacaattatatacaagatttcccaccaccccctactctaacacggcacaattatccatcgagtcagagcctagccaccatttggtgttgtttcttaatataggcacacggatcccgccatcgatcggcgccctgttcgagtatggccatcctcacacattgtcgggctccgctggcgctgcctcaacatatttttaaatggggaaaccataagcaaagagactccatcgggcgcactacacatctcgcctcttctggaccctgtggatcgggactggggtggatggcaaaccgtcaagttgccgccgcttcttcgcgctgttgcggcgtcacgccatataggcgactctcccaatcatggaaagcctgcgttccagcgatattcaacgacgtggtggcttggattgcagcttcgaccagatcacagcaatagaatagcgatgattccagccgtgggcctttagggtagaaaccatactgggctaggaaacatgccgtatcaagtgagaaacaccgctaccccaccacggctgaaaagtagatagtggatggttccatgtcatctatcagcaggcgaggagtgggtctatagtagaggttgtaatggtcgtggtagaggtccctggcccatttttatccccatccttcccggcctgatggttccaagccttctttacgaaaagttttgcagctgctttttcgagtcgacaggtcgctcgttccttcgtttgatttccctcgcttttgaccacgcccagggcatgtaggatttattccgtttgagattatcgagagcctaagtcgcgcgcagttgtcggtagcaggccactgccgggatgagagaagttattagtttgaacggtatgtgttttctcaatcttttcctcacaccgcctccttcaccggagtcggtctgttgaccgttactaattgtttttttacgtgtgcagttggtcaggctggttgccagatcgccaattcttgctgggaggtacggagatggaactatactagaagatagaggtgtttggaatgtgactattgaaactgacattatggttctagctctactgtcttgagcacggcatccaggtacgtggccacgatgacaatccatccatcttcatctccatcgggcacacagactaaccggtatacgatagcccgatggttacttgaccgaggaacgcaagaaggaagaccctgaccatggtttcagcaccttcttctccgaaactggtacgtcgtattgacttgttatcttgccgccgagccctctaacaggctatggtgtcttaggccagggcaagtatgttcctcgtaccatctacgccgatctggagcccaatgttgtcgatgaggtccgcactggcacctaccgtacccttttccaccccgagaacatgatcaccggcaaggaggatgcctcgaacaactatgcccgtggtcactacaccgttggcaaggagatgatcgaccaggtcctcgacaaggttcgccgtgtggccgacaactgcgctggtctccagggcttcctcgtcttccactctttcggtggtggtactggttccggtttcggtgctctcctgatggagcgtctgtctgtggactacggcaagaagtccaagctggagttctgcgtctaccctgccccccagaatgccacctccgtcgttgagccctacaactccatcctgactacccacaccacccttgagcactccgactgcagtttcatggttgacaacgaagccatctacgacatctgccgccgcaaccttggcatcgagcgtcccagctatgagaacctgaaccgcctgattgctcaggttgtctcctccatcaccgcctccctgcgtttcgatggttccctgaacgtggatctcaacgagttccagaccaacctggttccctacccccgtattcacttccctctcgttgcctatgctcccgtcatctccgcggccaaggcctcccacgaggccaactccgtcaacgagatcaccatgtcttgcttcgagcccaacaaccagatggtcaagtgtgacccccgcaatggcaagtacatggctacttgcttgctgtaccgtggtgatgttgtgcccaaggagacccacgccgccgttgctaccctcaagaccaagcgtaccatccagttcgtcgactggtgccctactggtttcaagatcggtatctgctaccagccccctaagcaggttcccaacggtgaccttgccaacctcagccgtgctgtgtaagttccctatctaccactcgtgcaagagataaaaagctgaccagttatagctgcatgctgtctaacaccaccgccatct

SEQ ID NO: 249 - DNA, 5509 bp, synthetic construct
  gene 1970..2868: note = pyrG
  regulatory 3260..4009: note = promoter - bipA promoter; regulatory_class = promoter
  regulatory 4004..4009: note = promoter - TIS; regulatory_class = promoter
  gene 4010..5474: note = ERG10 essential gene
  exon 4010..4074
  exon 4142..4191
  exon 4249..4324
  exon 4398..4887
  exon 4956..5474
  source 1..5509: mol_type = other DNA
aacgcgacgttgcccttttctgggcttagtcgccatttttcgccttgtccaggcaagacactttcaatgacagtgttgacttcttcaatcacatgcttgagcttgaaataggcgtcgttgggtggtatcttcagttcgaggatcagccgatccattttgttcacaaccatggtcaacggaaggccctctagcacagcgtgcttgatgatttgctccgtattcgattgtactccttccacaatatcaacgactatcaccacgccatcaaccagtcgtgatgaggcagctacttcatcaacaaaattcacatgccccggggtatcaagaatgttgaacaaatgagacttccccttagtgccctgaagaactaggctcataggagctgatttaatagaaaggcctcgctctctctctagaaagtgaatatcggtataacgaagctgtacttcctttcttttacctgctcgcttctctagccgttcagtcaaatcatgcgtttgcataacaagcatatccatgaacgcggtcttcccatgatgtaaatgaccgacaagcgcaatgttccttatttgttcaggaaaattcagaagatcagtcatgaattcccttgaaaagtgaacccgggggagctcagtctcttcaatggcgaacttcttttgctgtacgggggcaacgataggctcggagagaggctgtgcgtcttcttcttgaactaatgtctctacctctgtcccgtagacctgttgtgcgctagggtagtattgtttgtcttcgtgtagaatgacggcatttgagggggcttctgtaatgagatatatcagcaccgactaaatccaggggctttaagagagtgcccaccgtccacctccatagactgctgcccatataattcttcatcttcttcctccccaaacgcctcgttgaaggcgaaagcccgaggcttcacttcgtcgtgttggccgtcttcatctgaatctgcagcctctccaatgtagttgccaaacctgtgtggtcttgattagaatttgcacaattagagcgttagattggatgcaacttactcatcgtagagatcatccatcgtgcggtgggaaaggtggttcaagctgccaaaaacttgtcaccagtgagctgtcgcatctctttttgtcccaagctgctgaagtccgtctaatggactcaccgagtggtccgtgcgtcgaccctaccattttggtagccccgccgcgcggatgccacgtccaggaggggtccttaaatgactttgatttctcaccacctcttctcgtgtcagagatacctcactgctatcgtcggctgaataactcaactctttcttgtttcccttcttcccccctttaatcccaccaatcttctttattaccggcaagttctgtctacacgggtgccctttaccacacaccgaacccctttaaacgcttcacgctacagaagcttttggtttacgactcttctaatcagctatcgtaggccgccgatagctgattaaatattctagacccaagccgctgctggaattgacattattatggccgatagggttgggcttattgctatgtccctgaaaggatatcaaaagcaggcaaaaagccaggcataatccccgcgtggacggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggatgccacttacatgtgatctagtaatggttaatggtggattatataacaggactcggtccgtacattgccgtgatcaaaactcacatcgatatcctctccgatttcagcgaagaaaccatcaccggtctgaaggcccttgcagagaagcacaatttcctcatcttcgaagatcgcaagttcatcgatatcggaaacacagtccaaaagcagtaccatggcggcactctgcgtatctctgagtgggcccacatcatcaactgcagtattctgcccggtgagggtatcgtcgaggctctggcccagactgcttcggccgaggacttcccctacggctccgagaggggccttttgatccttgcggagatgacctccaagggatctttggctaccggtcaatatactacttcttctgttgactatgctcggaagtataagaagtttgtgatgggattcgtctcgacacgtcaccttggcgaggttcagtctgaagttagctcgccttcggaggaggaagattttgtcgtcttcacgacaggtgtcaacctctcctcgaagggtgacaagctgggacagcagtaccaaactcctgagtcggctgttggacgcggtgccgactttattattgctggccgtggaatttatgctgctcctgatcccgtggaggcggcgaagcagtaccagaaggagggatgggatgcatacctgaagcgtgttggtgcgcaataagtagtggtggatacgtactccttttatggcagtatgtcgcaagtatgatgcgatttataaattcagcactcgaaatgactactactatgtgtctacgacagataccctctccgtacgaataagacacctgcctcgatatatggacaaattcaaaatcagggtcaagggtcatgtttcaaagtcacaacaatctccaacatagacgagaatttgtaccggagtgtctgaaggtgcagctggagattggtctattttcttagagtggggtatcactaatgtacagtcggtcactatcgtacaaacaatcacaattatatacaagatttcccaccaccccctactctaacacggcacaattatccatcgagtcagagcctagccaccatttggtctatgtagaatagatagtagtaggggtgtcatcctccattatctgacaagcagataccagcatgtggccacgtgtagtgatgttgtgcaagtcattaagctgcagaggtcatgacttcacagtgactagagttgagtaacttgagatatggaaagtacacggtgtactcaaggtacagtacaattggaggttgacgacgtcctttgtccacgtatcctaaaggagcatatggccggggggctgaaaaatagggcaacttcaagaaagattacctaacaggggcttctagccaatgaaacacatttctagtatgttcggtggaccgcttgcagaagatccatgaacactttggggcaatttctacggcagaatacagggcggtggccaaggcggatgggtcattctggtccctgaaaggcgatgcaccaatcacaacgcgaggagtaacctgcagagcgatggccgtaccagccatgctgtccttttctctggcctcaagcattttaaaaaagctgatctcttcctctcacgtccttcgttcagcaacttctctcttttgcctcaactttcccttccttcccccttccattccgtcgcctgtggttgggttctctttttcttccttttgcttctctttcttgaatacaggaaactgtattgaagcacaagggatttactaccaacacgtctcccgataatcacacgcgtgccccttggcgaagaccaccactcgatatacacataggcacaatgtcttctcttccgccggtctatattgtttcctctgcccgcaccccagtcggctctttcttggggtgagattggccttgtgccttgtctatacgtacaatattgacttacactgactatccgcttccacaggtcgctctcaagtctcactgccccgcagttaggctctcatgctattaaaggtatgacagctctcgagtggcggtacgtgttgatatactaacaaactcgaaattcagctgcgctcagcaaagcggatggaatcaagccgtctgatatccaggaggtcttctttggcaatgtcatctccgcaaagtgagtcaacgtccacccttataccttatagccaacatcgaaggcacacttacattctcgttctatggatcagcgttggacaaaatcctgctagacagtgtgctctcggcgctggtctcaatgaatcaactgtctgtactacggttaataaggtgtgcgcgtctggcttgaaagcggttattctcggtgcacagaccatcatgactggcaatgcggatattgtcgtagcaggcggtgctgaatccatgtctaacgcccctcattaccttccaaaccttcgcgtcggtgcgaaatacggcaaccagagtctggtggacggtattatgaaggatggcttgacagacgcaggaaagcaggaactcatgggcttgcaagccgaggagtgtgctcaggatcatggctttagcagggaacaacaggatgattatgccattcgcacttacgaaaaagcacaggcggctcaaaaggctggcctttttgacgaagaaattgcgcctattgaacttcctggctttaggggcaagccaggtgtgactgtgtcacaagacgaagaaccaaagaatgtgaggaagatattactttctacagattttttaagtcgtcggccactgctaatatactcattgcgtagcttaacccggataagcttcgagctatcaagcctgcatttatccccggatccggcacggtcacagccccgaattcctcacctcttaacgacggtgctgctgctgttatcctcgtctcagaagctaaactgaaagagcttaacctaaagcctgttgcaaagattcttggctggggagatgccgcccagcagccaagcaaattcacaactgccccagctctagcaattcccaaggccctcagccatgcaggtgtggctcaggatgctgttgatgcgttcgagattaacgaagcgttcagcgtagttgctctggccaatatgaaactcctggggttggctgaagataaagtcaacatccatggtggtgcagtggctatcggtcatcctatcggcgccagcggtgctcgtatcttgactacattgctcggtgtattgaaagcgagaaagggtaagattggttgtgccgggatttgtaatggaggaggtggtgctagcgctattgttgtcgaatctctcgtctgagtcatgccgagtcgcccttgatcttggtatggaat

SEQ ID NO: 250 - DNA, 5509 bp, synthetic construct
  gene 1970..2868: note = pyrG
  regulatory 3260..4009: note = promoter - bipA promoter; regulatory_class = promoter
  regulatory 4004..4009: note = promoter - TIS; regulatory_class = promoter
  gene 4010..5509: note = PFS2 essential gene
  exon 4010..4014
  exon 4072..4142
  exon 4229..4460
  exon 4534..5509
  source 1..5509: mol_type = other DNA
ataaaaatttcgccaccgaagaagtatcaaggaaactgtctagcgagagggactggactggaacacttgagtattcggtatccagttaaggtgatttaagccaccccttctcccttcaatccctaccccccttcaaaaaggggaagaacagtagctaaaggtagttgccatcaagaactcatacttaaaaacacgaggtaagaggcccgtaatagtaattggttgcaagagaaaaggggggttatccttctgagcatcacagttttgattacgggaagaaccagaatatgaacgtggcaacagcggctgtcggtaccagaccaggcctttattggataccggaggagtccctctttcgtgtgtttcctaccccttttacagtacaattacaacctacataatgtcactgatagatatttatacgtgtgccagaagatacttatgggttagtcaatgtaaatagcttctaattttgattcatgattgaggggtctaaaattggactgaacctaatcctactcaagtaggagttagatgacgatggtgagtgtgaaatgccaggattaagggtagcatatcagccgcacaggaactaccgagggggtggaggctaacagccctctttttttctttccctttttcctcattttcctctctcttctcccgatgatgacgatttgtagattcataagttatagaaaaatatttgttctatgcacacgctaataggacatagtactgcttattgtatgtatatgcctggtgtttaaagaagtagacttcttgagttccgtacggagcactattttacaatacgaataaaaaaaatccatggagcagggaagggaaggatatagctagctaggtccctaatagtcgatatgttatttttgatctgttagtggagccactgacctgttgaatttttggtaggtaaacttagcaacatgtccatgccataaaaggtctccaaccgagagtcacgagtgaaacataccagtaggtccaacaaactgtgtaaggtgcagaaaaggcacattattggccagctccatgttgaatgaatggctgcgccctggagccggtccatagtagcttccctggtctctgtgtgggccgatctcacaacataaaacatagctcttccccctccttccaacagattgtcgatctactcaatttcccctttgctatcaacttctccccctcctcgcttccttctctcactcgccacaccgccctttaagaatatcacaagtaatattctagctggattatccaaagtgtgttttatgttgtccttccatggtatcttccaattcgtttgttgacagtatattcccttccagagtgcactttgaagaaaccccactaccgtttacgtgacttgctttcaatatccggaggcaacatttgttatcgaagcattacaacgcctagtcatctcattctacacctttccacattttgactcgctctctctcttgacataccttaaccagctgattaaatattctagacccaagccgctgctggaattgacattattatggccgatagggttgggcttattgctatgtccctgaaaggatatcaaaagcaggcaaaaagccaggcataatccccgcgtggacggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggatgccacttacatgtgatctagtaatggttaatggtggattatataacaggactcggtccgtacattgccgtgatcaaaactcacatcgatatcctctccgatttcagcgaagaaaccatcaccggtctgaaggcccttgcagagaagcacaatttcctcatcttcgaagatcgcaagttcatcgatatcggaaacacagtccaaaagcagtaccatggcggcactctgcgtatctctgagtgggcccacatcatcaactgcagtattctgcccggtgagggtatcgtcgaggctctggcccagactgcttcggccgaggacttcccctacggctccgagaggggccttttgatccttgcggagatgacctccaagggatctttggctaccggtcaatatactacttcttctgttgactatgctcggaagtataagaagtttgtgatgggattcgtctcgacacgtcaccttggcgaggttcagtctgaagttagctcgccttcggaggaggaagattttgtcgtcttcacgacaggtgtcaacctctcctcgaagggtgacaagctgggacagcagtaccaaactcctgagtcggctgttggacgcggtgccgactttattattgctggccgtggaatttatgctgctcctgatcccgtggaggcggcgaagcagtaccagaaggagggatgggatgcatacctgaagcgtgttggtgcgcaataagtagtggtggatacgtactccttttatggcagtatgtcgcaagtatgatgcgatttataaattcagcactcgaaatgactactactatgtgtctacgacagataccctctccgtacgaataagacacctgcctcgatatatggacaaattcaaaatcagggtcaagggtcatgtttcaaagtcacaacaatctccaacatagacgagaatttgtaccggagtgtctgaaggtgcagctggagattggtctattttcttagagtggggtatcactaatgtacagtcggtcactatcgtacaaacaatcacaattatatacaagatttcccaccaccccctactctaacacggcacaattatccatcgagtcagagcctagccaccatttggtctatgtagaatagatagtagtaggggtgtcatcctccattatctgacaagcagataccagcatgtggccacgtgtagtgatgttgtgcaagtcattaagctgcagaggtcatgacttcacagtgactagagttgagtaacttgagatatggaaagtacacggtgtactcaaggtacagtacaattggaggttgacgacgtcctttgtccacgtatcctaaaggagcatatggccggggggctgaaaaatagggcaacttcaagaaagattacctaacaggggcttctagccaatgaaacacatttctagtatgttcggtggaccgcttgcagaagatccatgaacactttggggcaatttctacggcagaatacagggcggtggccaaggcggatgggtcattctggtccctgaaaggcgatgcaccaatcacaacgcgaggagtaacctgcagagcgatggccgtaccagccatgctgtccttttctctggcctcaagcattttaaaaaagctgatctcttcctctcacgtccttcgttcagcaacttctctcttttgcctcaactttcccttccttcccccttccattccgtcgcctgtggttgggttctctttttcttccttttgcttctctttcttgaatacaggaaactgtattgaagcacaagggatttactaccaacacgtctcccgataatcacacgcgtgccccttggcgaagaccaccactcgatatacacataggcacaatgtggtacgttcccgaagacccaggcttactgaatgtttgatgtaactaattgacccctagcggcatcttcggctacattaactacctcgtcgagagggaccgcaagtttattcttgacactcttctcaatggttggcattcctgttgttttcaccctcactggaccttgtttactctggttttgggagtgtttgtgtattcctctgacatattgaaggactctcccgtcttgaataccgaggctacgactctgcgggtcttgccatagatggcgataagaagaatgaagtttgtgctttcaaggaagtgggcaaggttgctaagcttcgagagcttattgatgagtgcaagcctgacttgaccaaatcctttgaatctcatgctggtatttctcacactcgttgggccactcatgggacgccttcccgcttgaactgccacccccacaggtgagtccgatgttccacggtttaatcagctcgactaggatcaggcttgtttgctaattttcctggcacgcaggtccgatctgaactgggaattttcagttgtccacaatggtatcattaccaactacaaagagctcaaggctttgttggagagcaagggattccgctttgagactgacacagacaccgaatgtattgcaaagcttaccaaatatctctatgaccaacagcccgatatcgaattcacagttttggctaaggctgttgtgaaggagcttgagggcgcgtttgggcttctcatcaaatccgtacattatccccatgaggtcattgcggctcgcaaggggtcgccgcttgttattggcgtgagaacgtcgaagaagatgaaggtggatttcgtggacgtcgaatactcagaagatggagcccttcctgcagagcaggcctcccagaacgtggccatcaagaagtctgcgaccggcctccttgccccacctgacaaatcccttttgcataggtcacagtctcgcgctttcctttccgatgatggcatcccccagccagctgagtttttcttgtcttctgacccatctgcaattgtcgagcatacgaagaaggtcctctatctcgaagatgatgatatcgctcatgtgcatgagggacagttaaacattcatcgcctcacaaaagatgatggtacttctaatgttcgcgctattcagacaattgaactcgaattgcaggagattatgaagggcaagtttgaccacttcatgcaaaaggaaattttcgagcaacccgagtctgtgatcaataccatgagaggacgactggatgttgcaaacaagcaagtcacactcggtggcctgcggcagtacatttctactatccgccgctgcagaagaatcatatttgttgcctgtggaactagttaccattcatgcatggctgtgcgtggagtctttgaagagcttactgagattcctatttctgttgaacttgcttccgatttcctggatagacaggctccagtattccgtgatgacacg

SEQ ID NO: 251 - DNA, 5509 bp, synthetic construct
  gene 1970..2868: note = pyrG
  regulatory 3260..4009: note = promoter - bipA promoter; regulatory_class = promoter
  regulatory 4004..4009: note = promoter - TIS; regulatory_class = promoter
  gene 4010..5509: note = TUB1 essential gene
  exon 4010..4034
  exon 4124..4164
  exon 4239..4265
  exon 4337..4412
  exon 4473..5427
  exon 5481..5509
  source 1..5509: mol_type = other DNA
cgggaagatgttccatagaaagggatcaaggatgatatccgctgcaaccgggggaacattctccattagtgctttgttaagagaaaagtagtccgccgccgcagtaacacgaaggaaaagcaccgaatcagacgtaggtgcttgatcatgaaaagaggaagatttgcgaggaacagcgttagactgaggggaggcgagatgggcttgggaatagaggctaatcgaggagaagagtgcgggatcatcaatggcttgtggaagagtatgcgttgtcagggtaaaggcggtcggttgctttgactacttgtcagctaattttctttggtcgccttgctgggcactggtagagggtatatgaggacgtacagttgccaaccaacacacccgcacctcataccgctggccgggggtcaagttctccagaaaataccatgagtctgtaccagggaactcgttggtcggaaatgtagcgttaattctggttcgaagaacgggactagaggaggagaggcgatctagacctaggtcatcgagggtaggatcgaccgtggggatgatgaggggttgcggagcgatgaagatcgttttctcgacattggcgatagcggggaatgcgagggtgcagagtaagagtagatattggagtagcatcatgacagagagttcgctatgttcaattgacgctgctgctagacggtcatggttctagtccacacgcagttgagtatggtaggctatatgtatgtatgtagtaaatgaggtagatctgatactaactgttagattcctaagtgaagtccttacatgaaattgattcaatgtcaatacgatcttaccataaattaggccattgtgcgcagcaatgttggtgactagaaatagagcactgtgtgtccgagacttgttctatgtgagtattttcaatagtacttactatcgcaagctgtacggatagtcaaatacaatgcagctgcacgaagagatgccgtcattgacacgtgatcgacccttgccgtgaactaagcccaaataagtggctgagtcagcggtgggctggaagggcggtcacggcatcggacaagccaatcagaaatgggattgtgggcgacgcgacccaacggatggatcaacaatgaaaaggtggaagtcctgcttttgggttgacagcgcatcgcaagaccacaacaaattgaccgatcattcgggtcttgggcaattgtttaattgatccccttagtctatttttctcctcgtcaatcaccccaagggtttgttctcctcccccatttcctcccaccggttcccctttccctttcttttggttcttttcccaggtggtgtttttttcgtttacttacccctgaaacttccttacagatcagggcatttttccccctctaaagtccttccacattccacccactccgtttcacacacccccctccttccaatcttaattcctttgtcactccgacattcgtcccttttagactcgacaagagctgattaaatattctagacccaagccgctgctggaattgacattattatggccgatagggttgggcttattgctatgtccctgaaaggatatcaaaagcaggcaaaaagccaggcataatccccgcgtggacggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggatgccacttacatgtgatctagtaatggttaatggtggattatataacaggactcggtccgtacattgccgtgatcaaaactcacatcgatatcctctccgatttcagcgaagaaaccatcaccggtctgaaggcccttgcagagaagcacaatttcctcatcttcgaagatcgcaagttcatcgatatcggaaacacagtccaaaagcagtaccatggcggcactctgcgtatctctgagtgggcccacatcatcaactgcagtattctgcccggtgagggtatcgtcgaggctctggcccagactgcttcggccgaggacttcccctacggctccgagaggggccttttgatccttgcggagatgacctccaagggatctttggctaccggtcaatatactacttcttctgttgactatgctcggaagtataagaagtttgtgatgggattcgtctcgacacgtcaccttggcgaggttcagtctgaagttagctcgccttcggaggaggaagattttgtcgtcttcacgacaggtgtcaacctctcctcgaagggtgacaagctgggacagcagtaccaaactcctgagtcggctgttggacgcggtgccgactttattattgctggccgtggaatttatgctgctcctgatcccgtggaggcggcgaagcagtaccagaaggagggatgggatgcatacctgaagcgtgttggtgcgcaataagtagtggtggatacgtactccttttatggcagtatgtcgcaagtatgatgcgatttataaattcagcactcgaaatgactactactatgtgtctacgacagataccctctccgtacgaataagacacctgcctcgatatatggacaaattcaaaatcagggtcaagggtcatgtttcaaagtcacaacaatctccaacatagacgagaatttgtaccggagtgtctgaaggtgcagctggagattggtctattttcttagagtggggtatcactaatgtacagtcggtcactatcgtacaaacaatcacaattatatacaagatttcccaccaccccctactctaacacggcacaattatccatcgagtcagagcctagccaccatttggtctatgtagaatagatagtagtaggggtgtcatcctccattatctgacaagcagataccagcatgtggccacgtgtagtgatgttgtgcaagtcattaagctgcagaggtcatgacttcacagtgactagagttgagtaacttgagatatggaaagtacacggtgtactcaaggtacagtacaattggaggttgacgacgtcctttgtccacgtatcctaaaggagcatatggccggggggctgaaaaatagggcaacttcaagaaagattacctaacaggggcttctagccaatgaaacacatttctagtatgttcggtggaccgcttgcagaagatccatgaacactttggggcaatttctacggcagaatacagggcggtggccaaggcggatgggtcattctggtccctgaaaggcgatgcaccaatcacaacgcgaggagtaacctgcagagcgatggccgtaccagccatgctgtccttttctctggcctcaagcattttaaaaaagctgatctcttcctctcacgtccttcgttcagcaacttctctcttttgcctcaactttcccttccttcccccttccattccgtcgcctgtggttgggttctctttttcttccttttgcttctctttcttgaatacaggaaactgtattgaagcacaagggatttactaccaacacgtctcccgataatcacacgcgtgccccttggcgaagaccaccactcgatatacacataggcacaatgagagaagttattagtttgaacggtatgtgttttctcaatcttttcctcacaccgcctccttcaccggagtcggtctgttgaccgttactaattgtttttttacgtgtgcagttggtcaggctggttgccagatcgccaattcttgctgggaggtacggagatggaactatactagaagatagaggtgtttggaatgtgactattgaaactgacattatggttctagctctactgtcttgagcacggcatccaggtacgtggccacgatgacaatccatccatcttcatctccatcgggcacacagactaaccggtatacgatagcccgatggttacttgaccgaggaacgcaagaaggaagaccctgaccatggtttcagcaccttcttctccgaaactggtacgtcgtattgacttgttatcttgccgccgagccctctaacaggctatggtgtcttaggccagggcaagtatgttcctcgtaccatctacgccgatctggagcccaatgttgtcgatgaggtccgcactggcacctaccgtacccttttccaccccgagaacatgatcaccggcaaggaggatgcctcgaacaactatgcccgtggtcactacaccgttggcaaggagatgatcgaccaggtcctcgacaaggttcgccgtgtggccgacaactgcgctggtctccagggcttcctcgtcttccactctttcggtggtggtactggttccggtttcggtgctctcctgatggagcgtctgtctgtggactacggcaagaagtccaagctggagttctgcgtctaccctgccccccagaatgccacctccgtcgttgagccctacaactccatcctgactacccacaccacccttgagcactccgactgcagtttcatggttgacaacgaagccatctacgacatctgccgccgcaaccttggcatcgagcgtcccagctatgagaacctgaaccgcctgattgctcaggttgtctcctccatcaccgcctccctgcgtttcgatggttccctgaacgtggatctcaacgagttccagaccaacctggttccctacccccgtattcacttccctctcgttgcctatgctcccgtcatctccgcggccaaggcctcccacgaggccaactccgtcaacgagatcaccatgtcttgcttcgagcccaacaaccagatggtcaagtgtgacccccgcaatggcaagtacatggctacttgcttgctgtaccgtggtgatgttgtgcccaaggagacccacgccgccgttgctaccctcaagaccaagcgtaccatccagttcgtcgactggtgccctactggtttcaagatcggtatctgctaccagccccctaagcaggttcccaacggtgaccttgccaacctcagccgtgctgtgtaagttccctatctaccactcgtgcaagagataaaaagctgaccagttatagctgcatgctgtctaacaccaccgccatct

SEQ ID NO: 252 - DNA, 5509 bp, synthetic construct
  gene 1970..2868: note = pyrG
  regulatory 3260..4009: note = promoter - clxA promoter; regulatory_class = promoter
  regulatory 4004..4009: note = promoter - TIS; regulatory_class = promoter
  gene 4010..5474: note = ERG10 essential gene
  exon 4010..4074
  exon 4142..4191
  exon 4249..4324
  exon 4398..4887
  exon 4956..5474
  source 1..5509: mol_type = other DNA
aacgcgacgttgcccttttctgggcttagtcgccatttttcgccttgtccaggcaagacactttcaatgacagtgttgacttcttcaatcacatgcttgagcttgaaataggcgtcgttgggtggtatcttcagttcgaggatcagccgatccattttgttcacaaccatggtcaacggaaggccctctagcacagcgtgcttgatgatttgctccgtattcgattgtactccttccacaatatcaacgactatcaccacgccatcaaccagtcgtgatgaggcagctacttcatcaacaaaattcacatgccccggggtatcaagaatgttgaacaaatgagacttccccttagtgccctgaagaactaggctcataggagctgatttaatagaaaggcctcgctctctctctagaaagtgaatatcggtataacgaagctgtacttcctttcttttacctgctcgcttctctagccgttcagtcaaatcatgcgtttgcataacaagcatatccatgaacgcggtcttcccatgatgtaaatgaccgacaagcgcaatgttccttatttgttcaggaaaattcagaagatcagtcatgaattcccttgaaaagtgaacccgggggagctcagtctcttcaatggcgaacttcttttgctgtacgggggcaacgataggctcggagagaggctgtgcgtcttcttcttgaactaatgtctctacctctgtcccgtagacctgttgtgcgctagggtagtattgtttgtcttcgtgtagaatgacggcatttgagggggcttctgtaatgagatatatcagcaccgactaaatccaggggctttaagagagtgcccaccgtccacctccatagactgctgcccatataattcttcatcttcttcctccccaaacgcctcgttgaaggcgaaagcccgaggcttcacttcgtcgtgttggccgtcttcatctgaatctgcagcctctccaatgtagttgccaaacctgtgtggtcttgattagaatttgcacaattagagcgttagattggatgcaacttactcatcgtagagatcatccatcgtgcggtgggaaaggtggttcaagctgccaaaaacttgtcaccagtgagctgtcgcatctctttttgtcccaagctgctgaagtccgtctaatggactcaccgagtggtccgtgcgtcgaccctaccattttggtagccccgccgcgcggatgccacgtccaggaggggtccttaaatgactttgatttctcaccacctcttctcgtgtcagagatacctcactgctatcgtcggctgaataactcaactctttcttgtttcccttcttcccccctttaatcccaccaatcttctttattaccggcaagttctgtctacacgggtgccctttaccacacaccgaacccctttaaacgcttcacgctacagaagcttttggtttacgactcttctaatcagctatcgtaggccgccgatagctgattaaatattctagacccaagccgctgctggaattgacattattatggccgatagggttgggcttattgctatgtccctgaaaggatatcaaaagcaggcaaaaagccaggcataatccccgcgtggacggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggatgccacttacatgtgatctagtaatggttaatggtggattatataacaggactcggtccgtacattgccgtgatcaaaactcacatcgatatcctctccgatttcagcgaagaaaccatcaccggtctgaaggcccttgcagagaagcacaatttcctcatcttcgaagatcgcaagttcatcgatatcggaaacacagtccaaaagcagtaccatggcggcactctgcgtatctctgagtgggcccacatcatcaactgcagtattctgcccggtgagggtatcgtcgaggctctggcccagactgcttcggccgaggacttcccctacggctccgagaggggccttttgatccttgcggagatgacctccaagggatctttggctaccggtcaatatactacttcttctgttgactatgctcggaagtataagaagtttgtgatgggattcgtctcgacacgtcaccttggcgaggttcagtctgaagttagctcgccttcggaggaggaagattttgtcgtcttcacgacaggtgtcaacctctcctcgaagggtgacaagctgggacagcagtaccaaactcctgagtcggctgttggacgcggtgccgactttattattgctggccgtggaatttatgctgctcctgatcccgtggaggcggcgaagcagtaccagaaggagggatgggatgcatacctgaagcgtgttggtgcgcaataagtagtggtggatacgtactccttttatggcagtatgtcgcaagtatgatgcgatttataaattcagcactcgaaatgactactactatgtgtctacgacagataccctctccgtacgaataagacacctgcctcgatatatggacaaattcaaaatcagggtcaagggtcatgtttcaaagtcacaacaatctccaacatagacgagaatttgtaccggagtgtctgaaggtgcagctggagattggtctattttcttagagtggggtatcactaatgtacagtcggtcactatcgtacaaacaatcacaattatatacaagatttcccaccaccccctactctaacacggcacaattatccatcgagtcagagcctagccaccatttggtagtgtggtctgaagttttgcaaagtgagtgaggaggggaaaagaacgtcggcggggagaggggcaacgtgaagaactccattttttttttttctggatatggataattaactagtagcgggaagtagatcgcgcaatcggtatttgcaaggatagtatgcaattacttcgaagattaagaaaggaacaagaggaaaaatagaaaagaatttatttatattcatttgaaatttacattgactggattgaattgtcttatttgaattgggtgactagcatgtttgggagaagtgtactaatatgaatgaatggtatggagtactacaaataacatacagggacgctaagaccgtgacaaccaaagagagaaaacaaaaaaagcaataaaacggttctttagaatcaatggacactggacagaacattggagaatctgccactaatcctgtttgggtcggtggaactttaccccaaggggcaagcggcagcggaagaggccagattgaacttaagctaccttcccagcttcatataatcaacctccccaacactcgtcttctctttctcctctcctctctcttcttccttctgtcgtgcggttagcttgtggtacgctctcaccttgacactcttttccccctcgaacctctacggaggaccaattgcttgatcgtttgcttatttgtttctttctttcctaaagctactaccgagtcgggcttcttagtttgagagcccgttatcattcatcatgtcttctcttccgccggtctatattgtttcctctgcccgcaccccagtcggctctttcttggggtgagattggccttgtgccttgtctatacgtacaatattgacttacactgactatccgcttccacaggtcgctctcaagtctcactgccccgcagttaggctctcatgctattaaaggtatgacagctctcgagtggcggtacgtgttgatatactaacaaactcgaaattcagctgcgctcagcaaagcggatggaatcaagccgtctgatatccaggaggtcttctttggcaatgtcatctccgcaaagtgagtcaacgtccacccttataccttatagccaacatcgaaggcacacttacattctcgttctatggatcagcgttggacaaaatcctgctagacagtgtgctctcggcgctggtctcaatgaatcaactgtctgtactacggttaataaggtgtgcgcgtctggcttgaaagcggttattctcggtgcacagaccatcatgactggcaatgcggatattgtcgtagcaggcggtgctgaatccatgtctaacgcccctcattaccttccaaaccttcgcgtcggtgcgaaatacggcaaccagagtctggtggacggtattatgaaggatggcttgacagacgcaggaaagcaggaactcatgggcttgcaagccgaggagtgtgctcaggatcatggctttagcagggaacaacaggatgattatgccattcgcacttacgaaaaagcacaggcggctcaaaaggctggcctttttgacgaagaaattgcgcctattgaacttcctggctttaggggcaagccaggtgtgactgtgtcacaagacgaagaaccaaagaatgtgaggaagatattactttctacagattttttaagtcgtcggccactgctaatatactcattgcgtagcttaacccggataagcttcgagctatcaagcctgcatttatccccggatccggcacggtcacagccccgaattcctcacctcttaacgacggtgctgctgctgttatcctcgtctcagaagctaaactgaaagagcttaacctaaagcctgttgcaaagattcttggctggggagatgccgcccagcagccaagcaaattcacaactgccccagctctagcaattcccaaggccctcagccatgcaggtgtggctcaggatgctgttgatgcgttcgagattaacgaagcgttcagcgtagttgctctggccaatatgaaactcctggggttggctgaagataaagtcaacatccatggtggtgcagtggctatcggtcatcctatcggcgccagcggtgctcgtatcttgactacattgctcggtgtattgaaagcgagaaagggtaagattggttgtgccgggatttgtaatggaggaggtggtgctagcgctattgttgtcgaatctctcgtctgagtcatgccgagtcgcccttgatcttggtatggaat

SEQ ID NO: 253 - DNA, 5509 bp, synthetic construct
  gene 1970..2868: note = pyrG
  regulatory 3260..4009: note = promoter - clxA promoter; regulatory_class = promoter
  regulatory 4004..4009: note = promoter - TIS; regulatory_class = promoter
  gene 4010..5509: note = FKS2 essential gene
  exon 4010..4014
  exon 4072..4142
  exon 4229..4460
  exon 4534..5509
  source 1..5509: mol_type = other DNA
ataaaaatttcgccaccgaagaagtatcaaggaaactgtctagcgagagggactggactggaacacttgagtattcggtatccagttaaggtgatttaagccaccccttctcccttcaatccctaccccccttcaaaaaggggaagaacagtagctaaaggtagttgccatcaagaactcatacttaaaaacacgaggtaagaggcccgtaatagtaattggttgcaagagaaaaggggggttatccttctgagcatcacagttttgattacgggaagaaccagaatatgaacgtggcaacagcggctgtcggtaccagaccaggcctttattggataccggaggagtccctctttcgtgtgtttcctaccccttttacagtacaattacaacctacataatgtcactgatagatatttatacgtgtgccagaagatacttatgggttagtcaatgtaaatagcttctaattttgattcatgattgaggggtctaaaattggactgaacctaatcctactcaagtaggagttagatgacgatggtgagtgtgaaatgccaggattaagggtagcatatcagccgcacaggaactaccgagggggtggaggctaacagccctctttttttctttccctttttcctcattttcctctctcttctcccgatgatgacgatttgtagattcataagttatagaaaaatatttgttctatgcacacgctaataggacatagtactgcttattgtatgtatatgcctggtgtttaaagaagtagacttcttgagttccgtacggagcactattttacaatacgaataaaaaaaatccatggagcagggaagggaaggatatagctagctaggtccctaatagtcgatatgttatttttgatctgttagtggagccactgacctgttgaatttttggtaggtaaacttagcaacatgtccatgccataaaaggtctccaaccgagagtcacgagtgaaacataccagtaggtccaacaaactgtgtaaggtgcagaaaaggcacattattggccagctccatgttgaatgaatggctgcgccctggagccggtccatagtagcttccctggtctctgtgtgggccgatctcacaacataaaacatagctcttccccctccttccaacagattgtcgatctactcaatttcccctttgctatcaacttctccccctcctcgcttccttctctcactcgccacaccgccctttaagaatatcacaagtaatattctagctggattatccaaagtgtgttttatgttgtccttccatggtatcttccaattcgtttgttgacagtatattcccttccagagtgcactttgaagaaaccccactaccgtttacgtgacttgctttcaatatccggaggcaacatttgttatcgaagcattacaacgcctagtcatctcattctacacctttccacattttgactcgctctctctcttgacataccttaaccagctgattaaatattctagacccaagccgctgctggaattgacattattatggccgatagggttgggcttattgctatgtccctgaaaggatatcaaaagcaggcaaaaagccaggcataatccccgcgtggacggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggatgccacttacatgtgatctagtaatggttaatggtggattatataacaggactcggtccgtacattgccgtgatcaaaactcacatcgatatcctctccgatttcagcgaagaaaccatcaccggtctgaaggcccttgcagagaagcacaatttcctcatcttcgaagatcgcaagttcatcgatatcggaaacacagtccaaaagcagtaccatggcggcactctgcgtatctctgagtgggcccacatcatcaactgcagtattctgcccggtgagggtatcgtcgaggctctggcccagactgcttcggccgaggacttcccctacggctccgagaggggccttttgatccttgcggagatgacctccaagggatctttggctaccggtcaatatactacttcttctgttgactatgctcggaagtataagaagtttgtgatgggattcgtctcgacacgtcaccttggcgaggttcagtctgaagttagctcgccttcggaggaggaagattttgtcgtcttcacgacaggtgtcaacctctcctcgaagggtgacaagctgggacagcagtaccaaactcctgagtcggctgttggacgcggtgccgactttattattgctggccgtggaatttatgctgctcctgatcccgtggaggcggcgaagcagtaccagaaggagggatgggatgcatacctgaagcgtgttggtgcgcaataagtagtggtggatacgtactccttttatggcagtatgtcgcaagtatgatgcgatttataaattcagcactcgaaatgactactactatgtgtctacgacagataccctctccgtacgaataagacacctgcctcgatatatggacaaattcaaaatcagggtcaagggtcatgtttcaaagtcacaacaatctccaacatagacgagaatttgtaccggagtgtctgaaggtgcagctggagattggtctattttcttagagtggggtatcactaatgtacagtcggtcactatcgtacaaacaatcacaattatatacaagatttcccaccaccccctactctaacacggcacaattatccatcgagtcagagcctagccaccatttggtagtgtggtctgaagttttgcaaagtgagtgaggaggggaaaagaacgtcggcggggagaggggcaacgtgaagaactccattttttttttttctggatatggataattaactagtagcgggaagtagatcgcgcaatcggtatttgcaaggatagtatgcaattacttcgaagattaagaaaggaacaagaggaaaaatagaaaagaatttatttatattcatttgaaatttacattgactggattgaattgtcttatttgaattgggtgactagcatgtttgggagaagtgtactaatatgaatgaatggtatggagtactacaaataacatacagggacgctaagaccgtgacaaccaaagagagaaaacaaaaaaagcaataaaacggttctttagaatcaatggacactggacagaacattggagaatctgccactaatcctgtttgggtcggtggaactttaccccaaggggcaagcggcagcggaagaggccagattgaacttaagctaccttcccagcttcatataatcaacctccccaacactcgtcttctctttctcctctcctctctcttcttccttctgtcgtgcggttagcttgtggtacgctctcaccttgacactcttttccccctcgaacctctacggaggaccaattgcttgatcgtttgcttatttgtttctttctttcctaaagctactaccgagtcgggcttcttagtttgagagcccgttatcattcatcatgtggtacgttcccgaagacccaggcttactgaatgtttgatgtaactaattgacccctagcggcatcttcggctacattaactacctcgtcgagagggaccgcaagtttattcttgacactcttctcaatggttggcattcctgttgttttcaccctcactggaccttgtttactctggttttgggagtgtttgtgtattcctctgacatattgaaggactctcccgtcttgaataccgaggctacgactctgcgggtcttgccatagatggcgataagaagaatgaagtttgtgctttcaaggaagtgggcaaggttgctaagcttcgagagcttattgatgagtgcaagcctgacttgaccaaatcctttgaatctcatgctggtatttctcacactcgttgggccactcatgggacgccttcccgcttgaactgccacccccacaggtgagtccgatgttccacggtttaatcagctcgactaggatcaggcttgtttgctaattttcctggcacgcaggtccgatctgaactgggaattttcagttgtccacaatggtatcattaccaactacaaagagctcaaggctttgttggagagcaagggattccgctttgagactgacacagacaccgaatgtattgcaaagcttaccaaatatctctatgaccaacagcccgatatcgaattcacagttttggctaaggctgttgtgaaggagcttgagggcgcgtttgggcttctcatcaaatccgtacattatccccatgaggtcattgcggctcgcaaggggtcgccgcttgttattggcgtgagaacgtcgaagaagatgaaggtggatttcgtggacgtcgaatactcagaagatggagcccttcctgcagagcaggcctcccagaacgtggccatcaagaagtctgcgaccggcctccttgccccacctgacaaatcccttttgcataggtcacagtctcgcgctttcctttccgatgatggcatcccccagccagctgagtttttcttgtcttctgacccatctgcaattgtcgagcatacgaagaaggtcctctatctcgaagatgatgatatcgctcatgtgcatgagggacagttaaacattcatcgcctcacaaaagatgatggtacttctaatgttcgcgctattcagacaattgaactcgaattgcaggagattatgaagggcaagtttgaccacttcatgcaaaaggaaattttcgagcaacccgagtctgtgatcaataccatgagaggacgactggatgttgcaaacaagcaagtcacactcggtggcctgcggcagtacatttctactatccgccgctgcagaagaatcatatttgttgcctgtggaactagttaccattcatgcatggctgtgcgtggagtctttgaagagcttactgagattcctatttctgttgaacttgcttccgatttcctggatagacaggctccagtattccgtgatgacacg

SEQ ID NO: 254 - DNA, 5509 bp, synthetic construct
  gene 1970..2868: note = pyrG
  regulatory 3260..4009: note = promoter - clxA promoter; regulatory_class = promoter
  regulatory 4004..4009: note = promoter - TIS; regulatory_class = promoter
  gene 4010..5509: note = TUB1 essential gene
  exon 4010..4034
  exon 4124..4164
  exon 4239..4265
  exon 4337..4412
  exon 4473..5427
  exon 5481..5509
  source 1..5509: mol_type = other DNA
cgggaagatgttccatagaaagggatcaaggatgatatccgctgcaaccgggggaacattctccattagtgctttgttaagagaaaagtagtccgccgccgcagtaacacgaaggaaaagcaccgaatcagacgtaggtgcttgatcatgaaaagaggaagatttgcgaggaacagcgttagactgaggggaggcgagatgggcttgggaatagaggctaatcgaggagaagagtgcgggatcatcaatggcttgtggaagagtatgcgttgtcagggtaaaggcggtcggttgctttgactacttgtcagctaattttctttggtcgccttgctgggcactggtagagggtatatgaggacgtacagttgccaaccaacacacccgcacctcataccgctggccgggggtcaagttctccagaaaataccatgagtctgtaccagggaactcgttggtcggaaatgtagcgttaattctggttcgaagaacgggactagaggaggagaggcgatctagacctaggtcatcgagggtaggatcgaccgtggggatgatgaggggttgcggagcgatgaagatcgttttctcgacattggcgatagcggggaatgcgagggtgcagagtaagagtagatattggagtagcatcatgacagagagttcgctatgttcaattgacgctgctgctagacggtcatggttctagtccacacgcagttgagtatggtaggctatatgtatgtatgtagtaaatgaggtagatctgatactaactgttagattcctaagtgaagtccttacatgaaattgattcaatgtcaatacgatcttaccataaattaggccattgtgcgcagcaatgttggtgactagaaatagagcactgtgtgtccgagacttgttctatgtgagtattttcaatagtacttactatcgcaagctgtacggatagtcaaatacaatgcagctgcacgaagagatgccgtcattgacacgtgatcgacccttgccgtgaactaagcccaaataagtggctgagtcagcggtgggctggaagggcggtcacggcatcggacaagccaatcagaaatgggattgtgggcgacgcgacccaacggatggatcaacaatgaaaaggtggaagtcctgcttttgggttgacagcgcatcgcaagaccacaacaaattgaccgatcattcgggtcttgggcaattgtttaattgatccccttagtctatttttctcctcgtcaatcaccccaagggtttgttctcctcccccatttcctcccaccggttcccctttccctttcttttggttcttttcccaggtggtgtttttttcgtttacttacccctgaaacttccttacagatcagggcatttttccccctctaaagtccttccacattccacccactccgtttcacacacccccctccttccaatcttaattcctttgtcactccgacattcgtcccttttagactcgacaagagctgattaaatattctagacccaagccgctgctggaattgacattattatggccgatagggttgggcttattgctatgtccctgaaaggatatcaaaagcaggcaaaaagccaggcataatccccgcgtggacggtaccctaaggataggccctaatcttatctacatgtgactgcatcgatgtgtttggtcaaaatgaggcatgtggctcaccccacaggcggagaaacgtgtggctagtgcatgacagtcccctccatagattcaatttaatttttcgcggcaattgtcgtgcagtttgtatctacatttcattccatatatcaagagttagtagttggacatcctgattattttgtctaattactgaaaactcgaagtactaacctactaataagccagtttcaaccactaagtgctcatttatacaatatttgcagaaccccgcgctacccctccatcgccaacatgtcttccaagtcgcaattgacctacagcgcacgcgctagcaagcaccccaatgcgctcgtaaagaagctcttcgaggttgccgaggccaagaaaaccaatgtcaccgtttccgccgacgtgacaaccaccaaagagctgctggatttggctgaccgtatgcgcaccggggatgccacttacatgtgatctagtaatggttaatggtggattatataacaggactcggtccgtacattgccgtgatcaaaactcacatcgatatcctctccgatttcagcgaagaaaccatcaccggtctgaaggcccttgcagagaagcacaatttcctcatcttcgaagatcgcaagttcatcgatatcggaaacacagtccaaaagcagtaccatggcggcactctgcgtatctctgagtgggcccacatcatcaactgcagtattctgcccggtgagggtatcgtcgaggctctggcccagactgcttcggccgaggacttcccctacggctccgagaggggccttttgatccttgcggagatgacctccaagggatctttggctaccggtcaatatactacttcttctgttgactatgctcggaagtataagaagtttgtgatgggattcgtctcgacacgtcaccttggcgaggttcagtctgaagttagctcgccttcggaggaggaagattttgtcgtcttcacgacaggtgtcaacctctcctcgaagggtgacaagctgggacagcagtaccaaactcctgagtcggctgttggacgcggtgccgactttattattgctggccgtggaatttatgctgctcctgatcccgtggaggcggcgaagcagtaccagaaggagggatgggatgcatacctgaagcgtgttggtgcgcaataagtagtggtggatacgtactccttttatggcagtatgtcgcaagtatgatgcgatttataaattcagcactcgaaatgactactactatgtgtctacgacagataccctctccgtacgaataagacacctgcctcgatatatggacaaattcaaaatcagggtcaagggtcatgtttcaaagtcacaacaatctccaacatagacgagaatttgtaccggagtgtctgaaggtgcagctggagattggtctattttcttagagtggggtatcactaatgtacagtcggtcactatcgtacaaacaatcacaattatatacaagatttcccaccaccccctactctaacacggcacaattatccatcgagtcagagcctagccaccatttggtagtgtggtctgaagttttgcaaagtgagtgaggaggggaaaagaacgtcggcggggagaggggcaacgtgaagaactccattttttttttttctggatatggataattaactagtagcgggaagtagatcgcgcaatcggtatttgcaaggatagtatgcaattacttcgaagattaagaaaggaacaagaggaaaaatagaaaagaatttatttatattcatttgaaatttacattgactggattgaattgtcttatttgaattgggtgactagcatgtttgggagaagtgtactaatatgaatgaatggtatggagtactacaaataacatacagggacgctaagaccgtgacaaccaaagagagaaaacaaaaaaagcaataaaacggttctttagaatcaatggacactggacagaacattggagaatctgccactaatcctgtttgggtcggtggaactttaccccaaggggcaagcggcagcggaagaggccagattgaacttaagctaccttcccagcttcatataatcaacctccccaacactcgtcttctctttctcctctcctctctcttcttccttctgtcgtgcggttagcttgtggtacgctctcaccttgacactcttttccccctcgaacctctacggaggaccaattgcttgatcgtttgcttatttgtttctttctttcctaaagctactaccgagtcgggcttcttagtttgagagcccgttatcattcatcatgagagaagttattagtttgaacggtatgtgttttctcaatcttttcctcacaccgcctccttcaccggagtcggtctgttgaccgttactaattgtttttttacgtgtgcagttggtcaggctggttgccagatcgccaattcttgctgggaggtacggagatggaactatactagaagatagaggtgtttggaatgtgactattgaaactgacattatggttctagctctactgtcttgagcacggcatccaggtacgtggccacgatgacaatccatccatcttcatctccatcgggcacacagactaaccggtatacgatagcccgatggttacttgaccgaggaacgcaagaaggaagaccctgaccatggtttcagcaccttcttctccgaaactggtacgtcgtattgacttgttatcttgccgccgagccctctaacaggctatggtgtcttaggccagggcaagtatgttcctcgtaccatctacgccgatctggagcccaatgttgtcgatgaggtccgcactggcacctaccgtacccttttccaccccgagaacatgatcaccggcaaggaggatgcctcgaacaactatgcccgtggtcactacaccgttggcaaggagatgatcgaccaggtcctcgacaaggttcgccgtgtggccgacaactgcgctggtctccagggcttcctcgtcttccactctttcggtggtggtactggttccggtttcggtgctctcctgatggagcgtctgtctgtggactacggcaagaagtccaagctggagttctgcgtctaccctgccccccagaatgccacctccgtcgttgagccctacaactccatcctgactacccacaccacccttgagcactccgactgcagtttcatggttgacaacgaagccatctacgacatctgccgccgcaaccttggcatcgagcgtcccagctatgagaacctgaaccgcctgattgctcaggttgtctcctccatcaccgcctccctgcgtttcgatggttccctgaacgtggatctcaacgagttccagaccaacctggttccctacccccgtattcacttccctctcgttgcctatgctcccgtcatctccgcggccaaggcctcccacgaggccaactccgtcaacgagatcaccatgtcttgcttcgagcccaacaaccagatggtcaagtgtgacccccgcaatggcaagtacatggctacttgcttgctgtaccgtggtgatgttgtgcccaaggagacccacgccgccgttgctaccctcaagaccaagcgtaccatccagttcgtcgactggtgccctactggtttcaagatcggtatctgctaccagccccctaagcaggttcccaacggtgaccttgccaacctcagccgtgctgtgtaagttccctatctaccactcgtgcaagagataaaaagctgaccagttatagctgcatgctgtctaacaccaccgccatct

(19) United States
(12) Patent Application Publication
(10) Pub. No.: US 2024/0368535 A1
(43) Pub. Date: Nov. 7, 2024

(54) BURDEN-ADDICTED PRODUCTION STRAINS

(71) Applicant: Enduro Genetics ApS, Copenhagen N (DK)

(72) Inventors: Peter RUGBJERG, Copenhagen N (DK); Kristoffer Bach FALKENBERG, Copenhagen N (DK); Johannes KÜHN, Copenhagen N (DK); Christian MUNCK, Copenhagen N (DK); Martin TARP LOGIS, Copenhagen N (DK)

(21) Appl. No.: 18/683,429

(22) PCT Filed: Aug. 18, 2022
(86) PCT No.: PCT/EP2022/073110

§ 371 Date: Feb. 13, 2024

(30) Foreign Application Priority Data
Aug. 18, 2021 (EP) 21191995.6

(51) Int. Cl. C12N 1/20; C12N 1/16

(52) U.S. Cl. CPC C12N 1/205; C12N 1/165; C12N 2510/02; C12N 2511/00

(57) ABSTRACT

The invention provides a microbial production cell for synthesis of a product, further comprising a burden-addiction genetic circuit whose expression confers a selective growth and/or survival advantage on those cells that synthesize the product; while limiting proliferation of low- or non-productive escaper cells.

### FIELD OF THE INVENTION

The invention provides a microbial production cell for synthesis of a product, further comprising a burden-addiction genetic circuit whose expression confers a selective growth and/or survival advantage on those cells that synthesize the product; while limiting proliferation of non/low-productive escaper cells.

### BACKGROUND OF THE INVENTION

An increasing share of the world's chemical production relies on microorganisms that are genetically engineered to function as cell factories, and tailor-made for the biosynthesis of a given molecule. Production processes, employing these cell-factories, are typically initiated from a starter culture of a small number of cells of a production organism, which go through a phase of growth and expansion of cell numbers in large fermentation tanks (up to 1,000,000 L volume). In some setups, production of a given molecule proceeds both during the growth phase and during a subsequent period (batch and fed-batch cultures); and alternatively, production may be continuous. A chemostat fermenter allows a production organism to be grown in a fermentation broth that is constantly diluted, thus tapping product and cells from the culture, while replenishing with fresh nutrient medium. On an industrial scale, such production processes may continue operation for 1-2 months before starting a new culture in a clean tank. The fermentation processes and equipment used in this industry are very similar, both for the production of a wide range of commodity small molecules and for therapeutic proteins, and consequently these processes are subject to similar problems. In other applications, microbial cells may be engineered to produce a non-native molecule that confers a commercial advantage in the environment in which the cell is cultured.

Cell factories engineered to allocate finite metabolic resources to the biosynthesis of a given molecule are subject of an unnatural load; commonly reflected in slower growth. This load places a selective pressure for cells unable to synthesize the product molecule (non-producing or lower-producing), this problem particularly arising when the production run is for extended periods of time (e.g. in chemostats). Such non-producing cells within an industrial fermentation are highly undesirable, since they consume nutrients, oxygen and space. Since lower- and non-producing cells grow faster, they have a strong selective advantage over producing cells. In a growing cell culture, such improvements in fitness can lead to significant out-competition of the producing cells over time. This drift from the optimal production state is an eventual reason for discarding the fermentation broth and spending resources on cleaning, sterilization, not to mention nutrients, to replenish the fermentation tank with new, producing organisms. Such non-producing cells originate from genetic mutations that arise in the cells of an original producing organism undergoing many growth divisions.

Since the occurrence of genetic mutations and non-genetic adaptation in cells of a production organism that lead to a decline in product formation by the cells during a production run, cannot be avoided, there is a need for methods for eliminating or slowing the growth of non-producing cells in the production. Preferably, such methods of elimination are sufficiently effective that they prevent the observed drift from the production state, and thereby prolong the life-time of an industrial fermentation.

The present invention addresses the problem of how to deprive non-producing members of a cell factory with a fitness advantage; such as to delay their proliferation amongst members of a productive cell factory over time, and thereby improve the productivity of the cell factory.

### SUMMARY OF THE INVENTION

A first aspect of the invention provides a microbial production cell genetically engineered to synthesize a product, wherein said cell further comprises:
a first essential gene operably linked to a first burden-sensing promoter, and
a second essential gene operably linked to a second burden-sensing promoter
   wherein said first burden-sensing promoter is heterologous with respect to said first essential gene, and said second burden-sensing promoter is heterologous with respect to said second essential gene; wherein synthesis of the product confers a burden and/or fitness cost on said cell; and wherein expression of said first essential gene is up-regulated when said first burden-sensing promoter is induced by said burden and/or fitness cost relative to a basal level expression of said essential gene when said first burden-sensing promoter is not induced, and expression of said second essential genes is up-regulated when said second burden-sensing promoter is induced by said burden and/or fitness cost relative to a basal level expression of said second essential gene when said second burden-sensing promoter is not induced.

A second aspect of the invention provides a method of product biosynthesis comprising the steps of:
providing at least one microbial production cell of the invention,
introducing the at least one cell into a cultivation medium comprising substrate for production of said product,
recovering said product.

A third aspect of the invention provides for a use of a first and a second essential gene operably linked to a first and a second burden-sensing promoter, respectively, to enhance product yield of a cultured population of microbial production cells arising from a single cell following at least 50 generations; wherein said first burden-sensing promoter is heterologous with respect to said first essential gene, and said second burden-sensing promoter is heterologous with respect to said second essential gene; wherein production of the product confers a burden on said cell; and wherein expression of said first essential gene is up-regulated when said first burden-sensing promoter is induced by said burden relative to a basal level expression of said first essential gene when said first burden-sensing promoter is not induced, and expression of said second essential gene is up-regulated when said second burden-sensing promoter is induced by said burden relative to a basal level expression of said second essential gene when said second burden-sensing promoter is not induced.

A fourth aspect of the invention provides for a use of a microbial production cell of the invention for producing a biosynthetic product.

A further aspect of the invention provides a microbial production cell genetically engineered to synthesize a product, wherein said cell further comprises:
an essential gene operably linked to a burden-sensing promoter
   wherein said burden-sensing promoter is heterologous with respect to said essential gene; wherein synthesis of the product confers a burden and/or fitness cost on said cell; wherein expression of said essential gene is up-regulated when said burden-sensing promoter is induced by said burden and/or fitness cost relative to a basal level expression of said essential gene when said burden-sensing promoter is not induced; and wherein said microbial production cell belongs to a genus selected from among Bacillus, Corynebacterium, and Aspergillus.

### DESCRIPTION OF THE INVENTION

### Brief Description of the Figures

FIG. 1: Graph showing growth (measured as optical density: OD 630ₙₘ) over time of cells of the parent strain E. coli BL21(DE3) (grey line) and cells of the parent strain transformed with a hGH-GFP production plasmid pEG34 (black line; pEG34). Growth of four replicate cultures of each strain was measured under hGH-GFP expression inducing conditions; error bars depict standard error of the mean (n=4).

FIG. 2: Graph showing growth (measured as optical density: OD 630ₙₘ) over time of cells of the parent strain E. coli BL21(DE3) (black line) and cells of the non-producing burden-addicted E. coli strains s5.0 #3, s7.0 #8 and s9.0 #8 derived from the parent strain (as indicated; wherein essential genes folP-glmM are controlled by p_{yccV}, p_{ycjX} and p_{mutM} respectively). The measured growth rate is based on an average of four replicate cultures (n=4).

FIG. 3: Graph showing growth (measured as optical density: OD 630ₙₘ) over time of cells of the burden-addicted strain s9.0 #8 harboring either the hGH-GFP production plasmid pEG34 (black line) or the control plasmid pEGO (grey line). Growth of four replicate cultures of each strain was measured under hGH-GFP expression inducing conditions; error bars depict standard error of the mean (n=4).

FIG. 4: Graph showing growth (measured as optical density: OD 630ₙₘ) over time of cells of the parent strain E. coli BL21(DE3) (black line), and cells of three burden-addicted strains: s5.0 #3, s7.0 #8 and s9.0 #8 harboring and expressing the hGH-GFP production plasmid pEG34 (as indicated). The measured growth rate, measured under hGH-GFP expression inducing conditions, is based on an average of four replicate cultures (n=4).

FIG. 5: Histogram of GFP/OD values (Y-axis) measured during the first and sixth culture transfer (seed 1 and 6) of cultures of the hGH-GFP producing strains: s3.6 (p_{ibpA}), s6.6 (p_{grpE}), s7.6 (p_{ycjX}), and s10.6 (p_{ybbN}) comprising the respective burden-sensing promoters and one of four variant RBS coding sequences, driving expression of the essential gene, folP-glmM. Cultures of non-burden addicted E. coli strain BEG34 and the parent strain E. coli BL21(DE3), BEG0, are included as controls. Measurements are performed on cultures grown under hGH-GFP expression inducing conditions. Error bars depict standard error of the mean (n=4).

FIG. 6: Histogram of GFP/OD values (Y-axis) measured after serial culture transfers (seed 1 to 12) of cultures of the hGH-GFP producing strains: s3.6 #2 (p_{ibpA}), s6.6 #6 (p_{grpE}), s7.6 #8 (p_{ycjX}), and s10.6 #7 (p_{ybbN}) comprising the respective burden-sensing promoters and one of four variant RBS coding sequences, driving expression of the essential gene, folP-glmM. Cultures of non-burden addicted E. coli strain BEG34 and the parent strain E. coli BL21(DE3) are included as controls. Measurements are performed on cultures grown under hGH-GFP expression inducing conditions. Error bars depict standard error of the mean (n=3).

FIG. 7: Histogram of OD/min as a measure of the growth rate of burden-addicted hGH-GFP producing strains s3.6 #2, s6.6 #6, s7.6 #8 and s10.6 #7 compared to non-burden addicted hGH-GFP producing E. coli strain BEG34 and the parent strain E. coli BL21-DE3 (BEG0). The growth rate of the respective strains is based on 7 replicate cultures measured under hGH-GFP expression inducing conditions at seed 0; error bars denote standard error of the mean (n=7).

FIG. 8: Graphs showing GFP/OD values (Y-axis) measured after serial culture transfers (seed 1, 4 to 9) of cultures of hGH-GFP producing strains, minus the values measured for an empty plasmid control BEG0-empty strain. The strains shown in: (A) s7.6 #8 (p_{ycjX}), (B) s13.6 #2 (p_{fsxA}), and (C) s13.6 #2evo (p_{fsxA}), each comprise the respective burden-sensing promoter driving expression of the essential gene, folP-glmM (gray line) and a non-burden addicted hGH-GFP producing E. coli strain BEG34 (black line). Measurements are performed on cultures grown under hGH-GFP expression inducing conditions. Error bars depict standard error of the mean (n=5).

FIG. 9: Graphs showing GFP/OD values (Y-axis) measured after serial culture transfers (seed 1, 4 to 9) of cultures of hGH-GFP producing strains, minus the values measured for an empty plasmid control BEG0-empty strain. The strains shown in: (A) s15.6.7 (p_{rrnB}) and (B) s16.6.6 (p_{rrnE}), each comprise the respective burden-sensing promoter driving expression of the essential gene, folP-glmM (gray line) and a non-burden addicted hGH-GFP producing E. coli strain BEG34 (black line). Measurements are performed on cultures grown under hGH-GFP expression inducing conditions. Error bars depict standard error of the mean (n=5).

FIG. 10: Graph showing relative hGH-GFP production (Y-axis) measured after serial culture transfers (seed 2 to 5, and 7) of cultures of a hGH-GFP producing strain, s29.6 #3, minus the values measured for a parallel empty plasmid control BEG0 strain. Strain s29.6 #3 comprises the osmotic stress sensing promoter p_{poxB} driving expression of the essential gene, folP-glmM (black squares), compared to non-burden addicted hGH-GFP producing E. coli strain BEG34 (gray dots). Serial passage 7 was analyzed after 72 h. Measurements are performed on cultures grown under hGH-GFP expression inducing conditions. Error bars depict standard error of the mean (n=5 for s29.6 #3 and n=8 for BEG34).

FIG. 11: Histogram showing S. aureus lysis rate per minute, normalized to E. coli culture OD, as a measure of the secreted lysostaphin production rate of cultures of lysostaphin-producing strains s6.4 #5 comprising the burden sensing promoter p_{grpE} and one of four variant RBS coding sequences driving expression of the essential gene, folP-glmM, compared to non-burden addicted lysostaphin producing E. coli strain BENDU5cam. The rate was measured on cultures grown under lysostaphin expression inducing conditions after serial culture transfers (seed 1 and 7). Error bars depict standard error of the mean (n=3).

FIG. 12: Graph showing mevalonic acid titer (g/L) synthesized by cultures of the burden-addicted mevalonic acid-producing strains s19.1 #1 (p_{cspD}) and s9.1.4 (p_{mutM}), measured after serial culture transfers (seed 2, 5 and 6) compared to the parent non-burden addicted mevalonic acid-producing E. coli strain BL21(DE3)-pMevT. The burden sensing promoters, p_{cspD} and p_{mutM}, are each combined with one of four variant RBS coding sequences driving expression of the essential gene, folP-glmM. Measurements are performed on cultures grown under MevT expression inducing conditions. Error bars depict standard error of the mean (n=3).

FIG. 13: Bar plot showing human serum albumin (hSA) production titers of cultures of Pichia pastoris strain EGS31 comprising a genomically-integrated copy of a cDNA version of the hSA coding ALB1 gene under control of the AOX1 promoter, and a PDI1 promoter operably linked to the kanMX selection gene. The cultures were grown under either selecting conditions (addiction activated by 750 μg/mL G418) or non-selecting conditions (addiction not activated). The hSA titer of the ‘addiction not activated’ culture is set to 100%, and the titer of the ‘addiction activated culture is shown as a percent thereof.

FIGS. 14A and B: Bar plot showing human serum albumin (hSA) production titers of cultures of burden-addicted versions of the Pichia pastoris strain EGS31 each comprising a genomically-integrated copy of a cDNA version of the hSA coding ALB1 gene under control of the AOX1 promoter and a kanMX selection gene operably fused to a burden responsive promoter FPR2, or RPL3 or RPL6A. The cultures were grown under either non-selecting conditions (addiction not activated) or under selecting conditions (addiction activated with either 150 or 750 μg/mL G418) for a period of about 30 cell divisions ([A] Seed 1), or after a further 10 cell divisions ([B] Seed 2). Average hSA titers are shown in percent of the titer in the seed 1 ‘EGS31’ culture. Error bars depict standard error of the mean (n=4).

FIG. 15: Comparison of IgA-GFP production of burden-addicted EGS340 and parental EGS84 in long-term cultivation. Demonstration of burden addiction in Bacillus to sustain production using the IgA production burden-sensing perR promoter to regulate transcription of the essential iscU gene operon (sufC-sufD-sufS-sufU-sufB). Comparison in EGS340 and EGS84 during serial-passaging experiment at 30 deg. C. in which a 500× dilution seed is transferred to new culture medium every 24 hours. Points are averages and error bars show s.e.m. (n=7).

FIG. 16: Comparison of EGS343 and EGS84 in long-term cultivation. Demonstration of burden addiction in Bacillus subtilis to sustain production using the perR promoter to regulate transcription of the essential accC gene operon (accB-accC-yhqY). Comparison of IgA-GFP production in EGS343 and EGS84 during serial-passaging experiment at 30 deg. C. in which a 500× dilution seed is transferred to new culture medium every 24 hours. Points are averages and error bars show s.e.m. (n=7).

FIG. 17: Use of several burden-sensors to simultaneously regulate different essential genes and thereby sustain production of a heterologous protein in Bacillus subtilis. Comparison of IgA-GFP production strains following five serial passages (500× dilution seeds) corresponding to approximately 75 generations. The single burden-sensor strain EGS340 carries only the p_{perR}-based burden sensor controlling transcription of the iscU essential gene operon (EGS340). EGS340 is compared to derived strains in which transcription of the accC essential gene operon is also regulated by respectively a p_{ctsR}-based burden sensor (EGS460), a p_{dnaK}-based burden sensor (EGS462), and a p_{hrcA}-based burden sensor (EGS466). Bars show averages and error bars show s.e.m. (n=4).

FIG. 18: Comparison of relative IgA-GFP levels measured by IgA specific ELISA following approx. 75 generations of cell cultivations in the non-burden addicted control strain EGS084 and the double-burden addicted derived strain EGS466 (based on a p_{perR} controlling the iscU essential gene operon and a p_{hrcA} controlling the accC essential gene operon).

FIG. 19: Use of TIS variation on an essential gene to regulate growth rate. Two biological replicates of the parent strain (EGS621) and the strains with the TIS sequence variations of the essential gene CIA1 (EGS1100, EGS1101, EGS1102, EGS1104) grown in YPD. The Pichia strains exhibited growth correlated to the relative strength of the four TIS sequences.

### Abbreviations, Terms and Definitions

Burden refers to the cellular state of a microbial cell (such as a cell genetically engineered to synthesize a product) during production of a product under production conditions, and which results in a fitness cost attributable to the synthesis of said product, in particular in a cell that exhibits high level efficient synthesis of the product. Fitness cost is a measure of said burden that can be quantified by measuring the percent reduction in the maximum exponential growth rate of the cell (measured along the growth curve) during synthesis of the product under production conditions as compared to a parent microbial cell incapable of said synthesis when grown under comparable production conditions.

Burden-sensing promoter refers to a promoter that is induced by said ‘burden’, and when induced, said burden-sensing promoter can upregulate the expression of a gene to which it is operably linked in a microbial production cell, when cultured under production conditions as compared to a mutant derivative of said microbial production cell that synthesizes essentially none, or at least 50% less, of the intended product of the production cell. Such mutant derivative includes a non-productive escape mutant isolated following long-term cultivation of a population of cells derived from said production cell. Non-limiting examples (see Table 2 for details) of burden-sensing promoters include promoters of the E. coli genes htpG, ibpA, clpB, yccV, grpE, ycjx, ldhA, mutM, ybbN, prlC, groES, fxsA, htpX, rrnB, rrnE, cspD, katE, xthA, uspE, gadB, ahpC, katG, grxA, oxyS, poxB, trxC and their homolog versions in other gram-positive and gram-negative bacteria; for S. cerevisiae non-limiting examples of burden-sensing promoters include promoter of the genes KAR2, PDI1, SAA1, FPR2, RPL3, RPL6A, RPL28, OGG1, RAD51, RAD54; for Bacillus non-limiting examples of burden-sensing promoters include promoter of the genes groES, ctsR, dnak, perR, hrcA, spx, sigB, yflT and their homolog versions; for Corynebacterium non-limiting examples of burden-sensing promoters include promoter of the genes groES, kata, cplX, mutM and their homolog versions; for Aspergillus non-limiting examples of burden-sensing promoters include promoter of the genes bipA, clxA and their homolog versions. Burden-sensing promoters may also be hybrids, scrambled or truncated versions of such natural promoters as long as such promoters still maintain the response to burden.

Burden-addicted microbial cell refers to a microbial cell engineered to comprise a genetic circuit comprising at least one ‘burden-sensing promoter’ operably linked to an essential gene of the cell. Preferably said cell has a genetic circuit comprising two, namely a first essential gene operably linked to a first burden-sensing promoter, and a second essential gene operably linked to a second burden-sensing promoter wherein said first burden-sensing promoter is heterologous with respect to said first essential gene, and said second burden-sensing promoter is heterologous with respect to said second essential gene

Burden-addicted microbial production cell refers to microbial cell genetically engineered to synthesize a desired product, said cell being further genetically engineered to comprise a genetic circuit comprising said at least one, preferably two, ‘burden-sensing promoter’ operably linked to an ‘essential gene’ of the cell, as defined above. The desired product may be a protein encoded by a nucleic acid molecule in said engineered microbial cell, or a product of a heterologous pathway encoded by one or more nucleic acid molecules to be expressed by the cell.

Non-burden addicted microbial production cell is considered to be a “parent” cell of the burden addicted microbial production cell. It is a microbial production cell comprising and expressing one or more genes encoding the product or encoding a metabolic pathway for biosynthesis of the product, but lacking the genetic circuit comprising said at least one, preferably two, burden-sensing promoter operably linked to an essential gene as disclosed in the present invention. The at least one, preferably two, essential genes in this non-burden addicted microbial production cell wherein each is operably linked to its native promoter.

Desired products of the invention include—but are not limited to—a product that, when synthesized by the genetically engineered microbial production cell, conveys a fitness cost (burden) upon the production cell. Non-limiting examples of such product include an organic acid, terpenoid, isoprenoid, polyketide, alcohol, sugar, vitamin, aldehyde, carboxylic acid, fatty acid, amino acid, peptide, enzyme, therapeutic protein and precursors thereof, such as human growth hormone, insulin, glucagon-like peptide-1, a monoclonal or polyclonal antibody, a single-fragment antibody, and a nanobody. Examples further include proteins naturally found in eggs such as ovalbumin, or milk such as casein, lactadherin, alpha-lact lactoferrin, secreted immunoglobulin A and G, a secretory component. Examples of an enzyme includes an amylase, lipase, protease, barnase, β-galactosidase, crystal protein, cutinase, PETase, and laccase as well as a carbohydrate active enzyme such as a xylanase, lichenase, cellulase, lytic polysaccharide monooxoygenase, and a pectase.

Essential gene (or essential gene operon) refers to gene(s) in the microbial production cell which, if down-regulated, lead to reduction in the growth rate of the microbial production cell under production conditions. Preferably an essential gene is essential for growth irrespective of the nutrient composition of these production conditions, whereby sufficient expression of such essential genes to support cell growth would not be dependent on the presence or absence of specific inhibitors or nutrients provided under the production conditions. Non-limiting examples of E. coli essential genes during standard laboratory conditions include folP-glmM, gimM, murI, asd, thyA, rpoD, nusG, rpsU, accD, degS, fldA, ftsN, hflB, lolA, mraY, mreD, murA, murB, murF, nadD, rplV, rpsG, and homologues thereof. Non-limiting examples for Bacillus include iscU operon (sufC-sufD-sufS-sufU-sufB), accC operon (accB-accC-yhqY), glmM, ylaN, infA, dapA and homologues thereof. Non-limiting examples for Saccharomyces include, FOL1, MED7, RRP40, NOP8, PGI1, NEP1, URA3 LEU2, TRP1, HIS3 and homologues thereof. Non-limiting examples for Aspergillus include ARG3, adeA, and homologues thereof: Non-limiting examples for Corynebacterium include alr, glmM, and homologues thereof: For example, suitable genes encode enzymes responsible for synthesizing cell wall constituents. Further, an essential gene used in the context of this invention preferably neither encodes a desired product to be expressed by the production strain; nor encodes a protein that facilitates synthesis of a desired product or intermediate thereof in a heterologous pathway to be expressed by the production strain.

Conditional essential gene is one that allows the burden addiction system in the microbial production cell to be “activated” by culturing the cells under the condition of essentiality such as added antibiotic or removed nutrient.

Essential gene basal expression level is the level of transcription of each ‘essential gene’ (as defined above) that is operably linked to a ‘burden-sensing promoter’ in a burden-addicted microbial production cell, when the ‘burden-sensing promoter’ is not induced. The burden sensing promoter to which the essential gene is operably linked is heterologous with respect to said essential gene, in the sense that the promoter is not the native promoter of said essential gene (even though the promoter may be present in the same genome), and is thus not found operably linked to said essential gene in nature. Said basal (i.e. un-induced) level expression of an essential gene is a level sufficient to support growth of a cell under production conditions (or during an exponential growth phase) at a level equal to or less than 10, 20, 50, 90 or 95% of the growth rate of a corresponding cell wherein each said essential gene is operably linked to its native promoter. A lowered growth rate due to basal level essential gene expression in those cells where the burden sensing promoter is not activated constitutes a selective disadvantage for non-productive cells, for example non-productive mutants arising during product production.

Fitness cost: is quantified by comparing the maximum exponential growth rate (when measured along the growth curve) of a non-burden addicted microbial production cell or cell culture thereof (i.e. lacking burden-sensing promoters operably linked to the at least one, preferably two essential genes), but comprising and expressing one or more genes encoding the product or encoding a metabolic pathway for biosynthesis of the product) relative to the maximum exponential growth rate of (i) a parent microbial cell (or cell culture thereof) either devoid or incapable of expressing gene(s) encoding the product or metabolic pathway for biosynthesis of the product, from which the microbial production cell was derived (or (ii) an escape cell derived from the non-burden addicted microbial production cell, wherein said escape cell produces less than 50% of the product compared to the non-burdened addicted microbial production cell), when grown under comparable production conditions. The maximum exponential growth rate of the respective cells or cell cultures thereof is to be measured at the start of production following introduction of the respective cells or cell cultures thereof into a cultivation medium comprising substrate for production of said product.

Production conditions refers to specific cultivation conditions to be used during production and can relate to medium and/or other culture conditions (e.g. temperature, pH, stirring, aeration, etc) for production of a desired product by the microbial production cell.

Ribosomal binding site (RBS), translation initiation region, or translational strength element refers to the genetic region of the 5′ untranslated region that control the translation strength of a particular messenger RNA.

### DETAILED DESCRIPTION OF THE INVENTION

### I. A Burden-addicted Microbial Production Cell

A first aspect of the present invention provides a microbial production cell genetically engineered to synthesize a product, wherein said cell further comprises:
a. an essential gene operably linked to a burden-sensing promoter,
   wherein said promoter is heterologous with respect to said essential gene; wherein production of the product confers a burden and/or fitness cost on said cell, and wherein expression of said essential gene is up-regulated when said burden-sensing promoter is induced by said burden and/or fitness cost relative to a basal level expression of said essential gene when said burden-sensing promoter is not induced.

In a further aspect of the present invention, the microbial production cell genetically engineered to synthesize a product comprises:
a. a first essential gene operably linked to a first burden-sensing promoter, and
b. a second essential gene operatively linked to a second burden-sensing promoter,
   wherein said first burden-sensing promoters is heterologous with respect to said first essential genes, and said second burden-sensing promoters is heterologous with respect to said second essential genes;
   wherein synthesis of the product confers a burden on said cell, and
   wherein expression of said first essential genes is up-regulated when said first burden-sensing promoter is induced by said burden relative to a basal level expression of said first essential gene when said first burden-sensing promoter is not induced, and expression of said second essential genes is up-regulated when said second burden-sensing promoter is induced by said burden relative to a basal level expression of said second essential gene when said second burden-sensing promoter is not induced.

In one embodiment, the first and second essential genes are different. In another embodiment, the first and second essential genes are identical. In a specific embodiment, the first and second essential genes are identical while the first and second burden-sensing promoters are different.

In one embodiment, the first and second burden-sensing promoters are different. In another embodiment, the first and second burden-sensing promoters are identical. In a specific embodiment, the first and second burden-sensing promoters are identical while the first and second essential genes are different.

In one embodiment, the microbial production cell comprises more than two essential gene operably each linked to a burden-sensing promoter. In one embodiment, the invention provides a microbial production cell genetically engineered to synthesize a product, said microbial cell comprising two, three, four, five, six or more burden sensing promoters to control essential genes in the cell.

In one embodiment, the microbial cell comprises three essential genes, each operably linked to a burden-sensing promoter. In one embodiment, the microbial cell comprises four essential genes, each operably linked to a burden-sensing promoters. In one embodiment, all the burden-sensing promoters are different. In another embodiment, some of the burden-sensing promoters are identical, but they are linked to different essential genes. In one embodiment, all the essential genes are different. In another embodiment, some of the essential genes are identical, but they are linked to different burden-sensing promoters.

A microbial production cell genetically engineered to synthesize a product is one that comprises one or more genes encoding the product or encoding a metabolic pathway for synthesis of the product, wherein optionally said one or more genes are operably linked to a constitutive or inducible promoter. In one embodiment, the one or more genes encoding the product or encoding a metabolic pathway for synthesis of the product may be recombinant.

According to one embodiment, said burden and/or fitness cost on said microbial production cell of the invention, is quantified by comparing the maximum exponential growth rate of the microbial production cell comprising one or more genes encoding the product or encoding a metabolic pathway for synthesis of the product, (but lacking burden-sensing promoters operably linked to the at least one preferably two essential gene), relative to the maximum exponential growth rate of a parent microbial cell lacking or incapable of expressing one or more genes encoding the product or encoding a metabolic pathway for synthesis of the product, and from which said microbial production cell was derived, where the respective cells are cultured under essentially identical production conditions. The burden and/or fitness cost on said microbial production cell preferably corresponds to a percent reduction in the quantified maximum exponential growth rate selected from among ≥5%, ≥10%, ≥15%, ≥20%, ≥25%, ≥35% and ≥45%.

The burden-addicted microbial production cell according to the invention may be any prokaryotic or eukaryotic microorganism such as a bacterium, yeast, and filamentous fungus.

In one embodiment, the microbial production cell of the invention is a prokaryote. A non-exhaustive list of suitable bacteria is given as follows: a species belonging to the genus selected from among Escherichia, Lactobacillus, Lactococcus, Corynebacterium, Bacillus, Acetobacter, Acinetobacter, Pseudomonas; Proprionibacterium, Bacteroides and Bifidobacterium.

In one preferred embodiment, the invention provides a microbial production cell genetically engineered to synthesize a product, said cell comprising an essential gene operably linked to a burden-sensing promoter; wherein said microbial production cell belongs to the genus Bacillus. In another embodiment, the microbial production cell of the invention is a eukaryote, such as a yeast or fungus. In certain embodiments, the eukaryote can be a member of the genus Saccharomyces, Komagataella or Aspergillus.

A non-exhaustive list of suitable yeasts is given as follows: a yeast belonging to the genus of Saccharomyces, e.g. S. cerevisiae, S. kluyveri, S. bayanus, S. exiguus, S. sevazzi, S. uvarum; a yeast belonging to the genus Kluyveromyces, e.g. K. lactis K. marxianus var. marxianus, K. thermotolerans; a yeast belonging to the genus Candida, e.g. C. utilis C. tropicalis, C. albicans, C. lipolytica, C. versatilis; a yeast belonging to the genus Pichia, e.g. P. stipidis, P. pastoris, P. sorbitophila, or other yeast genera, e.g. Cryptococcus, Debaromyces, Hansenula, Yarrowia, Zygosaccharomyces or Schizosaccharomyces.

A non-exhaustive list of suitable filamentous fungi is given as follows: a filamentous fungus belonging to the genus of Penicillium, Rhizopus, Fusarium, Fusidium, Gibberella, Mucor, Mortierella, Trichoderma Thermomyces, Streptomyces and Aspergillus. More specifically, the filamentous fungus may be selected from Fusarium oxysporum, A. niger, A. awamori, A. oryzae, and A. nidulans.

The product(s) synthesized by the microbial production cell of the invention is one that incurs a burden on the cell and fitness cost reflected in a reduction in growth rate, such product(s) including amino acids, organic acids, terpenoids, isoprenoids, polyketides, alcohols, sugars, vitamins, aldehydes, carboxylic acids, fatty acids, peptides, enzymes, therapeutic proteins and their precursors such as human growth hormone, insulin, glucagon-like peptide-1, monoclonal and polyclonal antibodies, single-fragment antibodies and nanobodies. Examples further include proteins naturally found in eggs such as ovalbumin, or milk such as caseins, lactadherins, alpha-lactalbumin, beta-lactoglobulin, osteopontin, lactoferrin, secreted immunoglobulin A and G, secretory components. Further examples of enzymes are amylases, lipases, proteases, barnases, β-galactosidases, crystal proteins, cutinases, PETases, and laccases as well as carbohydrate-active enzymes such as xylanases, lichenases, cellulases, lytic polysaccharide monooxoygenases, and pectases. In one embodiment the product is not a native product of the microbial production cell of the invention, and thus not produced by a parent cell from which the microbial production cell was derived. In one embodiment, the one or more genes encoding the product or encoding a metabolic pathway for synthesis of the product are heterologous with respect to the microbial production cell of the invention; where said one or more genes may be transgenes.

### II. Features of the Microbial Production Cell of the Invention

### II.i Fitness Cost—Production Burden

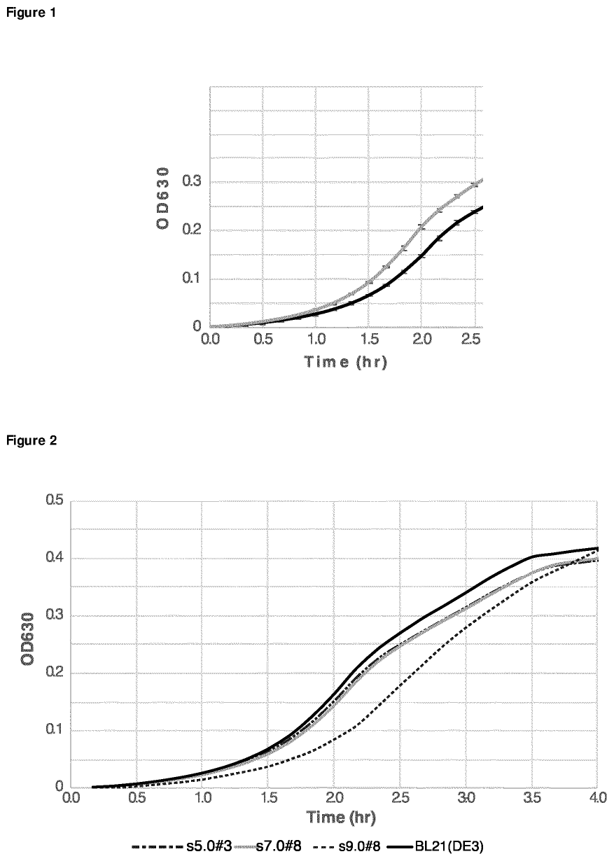

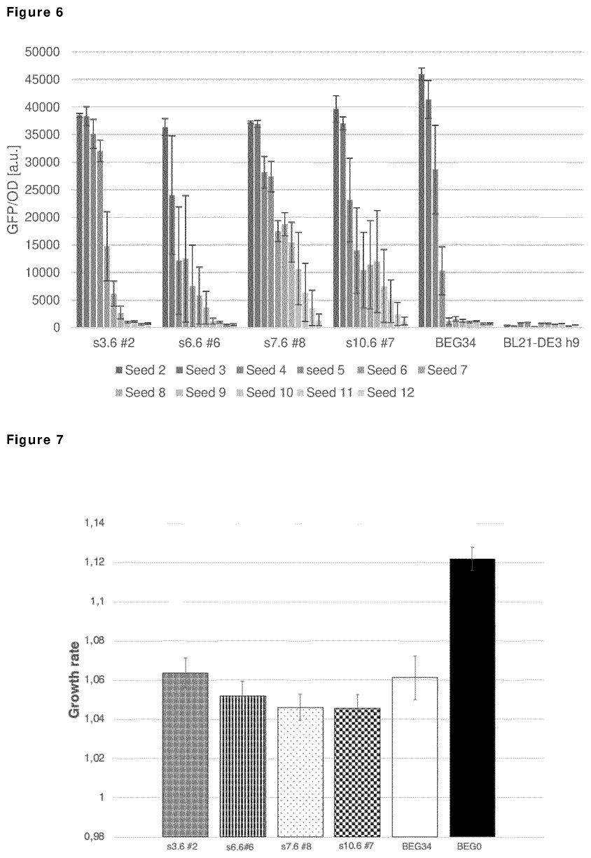

Heterologous expression of a desired product or pathways leading to a desired product in a microbial cell imposes a burden on the cell. Burden can also result from overproduction of a native product, e.g. in cells of microbial strains selected following mutagenesis. The burden may be described as a fitness cost which results in slower growth of the cell (Example 1; FIG. 1). This may be due to general and/or product-specific metabolic toxicities, increased use and/or depletion of potentially finite cell resources and metabolites, which a microbial cell experiences during the biosynthesis of a product of the invention under production conditions. Particularly during industrial scale fermentation, a combination of fitness cost and slower growth places a selective pressure on the population of cells in the fermenter, which over time leads to a decline in product yield due to an accumulation of non-productive cells resulting from genetic mutations or non-genetic variation (example 2, BEG34 in FIG. 6).

In the present invention, the presence and expression of one or more genes encoding a product or encoding a metabolic pathway for biosynthesis of a product in a microbial production cell confers a fitness cost (burden).

The fitness cost of producing a product on a microbial production cell is quantified by comparing the growth rate of a non-burden addicted microbial production cell (i.e. lacking at least one, preferably two, burden-sensing promoters operably linked to the essential genes, but comprising and expressing one or more genes encoding the product or encoding a metabolic pathway for biosynthesis of the product) relative to the growth rate of (i) a parent microbial cell from which the non-burden addicted microbial production cell was derived, wherein said parent microbial cells is devoid of gene(s) encoding the product or metabolic pathway for biosynthesis of the product, or (ii) an escape cell derived from the non-burden addicted microbial production cell, wherein said escape cell produces less than 50% of the product compared to the non-burdened addicted microbial production cell. The burden and/or fitness cost on said microbial production cell preferably corresponds to a percent reduction in the quantified growth rate selected from among ≥5%, ≥10%, ≥15%, ≥20%, ≥25%, ≥35% and ≥45%, more preferably at least 5%.

Preferably quantification of the relative growth rates is performed on the respective microbial cells by measuring their maximum exponential growth rate when cultured under essentially identical conditions, these conditions being chosen to closely mimic those in which eventual large-scale fermentation is to take place. All uses of growth rate refers to the term specific growth rate.

### II.ii Essential Gene

A microbial production cell of the invention comprises at least one preferably two essential gene(s) encoding at least one preferably two protein(s), respectively, whose expression is required for cell growth and/or survival.

In one embodiment, the microbial production cell comprises a first and a second essential gene encoding a first and a second protein (where the first and second protein may be the same or different), respectively, wherein the expression of both said first and said second protein is required for cell growth and/or survival.

Preferably, the at least one preferably two essential genes and their expression do not indirectly cause a fall in production of the desired product by the production cell. Further, the essential gene(s) used in the context of this invention do not encode or lead to synthesis of a desired product or intermediate of a heterologous pathway to be expressed in the microbial production cell.

The at least one preferably two essential gene(s) are preferably non-conditional essential gene(s), such that expression of the gene(s) is essential for cell growth and/or survival irrespective of the composition of the growth medium or conditions in which the cell is cultured.

In one embodiment of the invention, when the production cell is a prokaryote, such as E. coli, the at least one preferably two non-conditional essential gene(s) are selected from folP-glmM, glmM, murI, asd, thyA, usA, rpoD, nusG, rpsU, accD, degS, fldA, ftsN, hflB, lolA, mraY, mreD, murA, murB, murF, nadD, rplV and rpsG, and homologues thereof.

In a further embodiment of the invention, when the production cell is a prokaryote, such as a Bacillus strain, the at least one preferably two non-conditional essential gene is selected from iscU operon, accC operon, glmM, ylaN, infA, and dapA, and homologues thereof.

In one embodiment of the invention, when the production cell is a prokaryote, such as a Corynebacterium strain, the at least one preferably two non-conditional essential gene is selected from alr, glmM, and homologues thereof

In one embodiment of the invention, when the production cell is a eukaryote, the essential gene when non-conditional is selected from S. cerevisiae FOL1, MED7, RRP40, NOP8, PGI1, NEP1 and homologues thereof; and when conditional is selected from S. cerevisiae URA3, LEU2, TRP1, HIS3 and homologues thereof.

In one embodiment of the invention, when the production cell is a filamentous fungi, such as Aspergillus, the essential gene is selected from ARG3, adeA, ERG10, PFS2 and TUB1 and homologues thereof.

In one embodiment of the invention, the at least one preferably two essential genes are conditionally essential genes leading to the synthesis of a product required for auxotrophic growth, or a protein product required for resistance to growth inhibitors such as an antibiotic, specific toxin, protoxin, or the like.

When the at least one preferably two essential genes are conditionally essential then the composition of the production cell's growth medium/growth conditions must be adjusted, such as using growth medium lacking specific nutrients or supplemented with growth inhibitors.

In one further embodiment, the production cell may comprise a combination of a non-conditional and a conditional essential gene.

TABLE 1
Non-exhaustive list of essential genes

| SEQ ID | Gene^{#1} | | Ecocyc/Biocyc |
|---|---|---|---|
| NO. | E. coli | Gene encoded product(s) | accession ID^{#2} |
| 1 | folP-glmM | Operon of genes encoding | EG50011 |
| | | dihydropteroate synthase | |
| | | and phosphoglucosamine | |
| | | mutase | |
| 3 | glmM | phosphoglucosamine | EG11553 |
| | | mutase | |
| 5 | murI | glutamate racemase | EG11204 |
| 7 | asd | aspartate-semialdehyde | EG10088 |
| | | dehydrogenase | |
| 9 | thyA | thymidylate synthase | EG11002 |
| 11 | rpoD | RNA polymerase, sigma70 | EG10896 |
| 13 | nusG | transcription termination | EG10667 |
| | | factor NusG | |
| 15 | rpsU | 30S ribosomal subunit | EG10920 |
| | | protein S21 | |
| 17 | accD | acetyl-CoA carboxyl- | EG10217 |
| | | transferase subunit-β | |
| 19 | degS | serine endoprotease | EG11652 |
| 21 | fldA | flavodoxin 1 | EG10318 |
| 23 | ftsN | cell division protein FtsN | EG11529 |
| 25 | hflB | ATP-dependent zinc | EG11506 |
| | | metalloprotease FtsH | |
| 27 | lolA | outer membrane lipoprotein | G6465 |
| | | carrier protein | |
| 29 | mraY | 18hosphor-N-acetylmuramoyl- | EG10604 |
| | | pentapeptide-transferase | |
| 31 | mreD | cell shape determining protein | EG10610 |
| | | MreD | |
| 33 | murA | UDP-N-acetylglucosamine | EG11358 |
| | | 1-carboxyvinyltransferase | |
| 35 | murB | UDP-N-acetylenolpyruvoyl- | G11205 |
| | | glucosamine reductase | |
| 37 | murF | D-alanyl-D-alanine-adding | EG10622 |
| | | enzyme | |
| 39 | nadD | nicotinate-mononucleotide | G6350 |
| | | adenylyltransferase | |
| 41 | rplV | 50S ribosomal subunit | EG10882 |
| | | protein L22 | |
| 43 | rpsG | 30S ribosomal subunit | EG10906 |
| | | protein S7 | |
| | S. | | SGD |
| | cerevisiae | | ID^{#3} |
| 45 | FOL1 | dihydroneopterin aldolase | SGD: S000005200 |
| 47 | MED7 | mediator complex subunit | SGD: S000005495 |
| | | MED7 | |
| 49 | RRP40 | exosome non-catalytic core | SGD: S000005502 |
| | | subunit RRP40 | |
| 51 | NOP8 | Nucleolar protein required | SGD: S000005504 |
| | | for 60S ribosomal subunit | |
| | | biogenesis | |
| 53 | PGI1 | glucose-6-phosphate | SGD: S000000400 |
| | | isomerase | |
| 55 | NEP1 | 18S rRNA pseudouridine | SGD: S000004176 |
| | | methyltransferase | |
| 57 | URA3 | orotidine-5′-phosphate | SGD: S000000747 |
| | | decarboxylase | |
| 59 | LEU2 | 3-isopropylmalate | SGD: S000000523 |
| | | dehydrogenase | |
| 61 | TRP1 | phosphoribosylanthranilate | SGD: S000002414 |
| | | isomerase | |
| 63 | HIS3 | imidazoleglycerol-phosphate | SGD: S000005728 |
| | | dehydratase | |
| | Bacillus | | BL lotus tag^{#4} |
| 147 | glmM | Phosphoglucosamine mutase | NL02703 |
| 149 | ylaN | Protein controlling cell shape | BL02974 |
| 151 | infA | Translation initiation factor | BL01028 |
| 153 | dapA | Dihydrodipicolinate synthase | BL01208 |
| 165 | sufC (in | | |
| | iscU | | |
| | operon) | | |
| 167 | sufD (in | | |
| | iscU | | |
| | operon) | | |
| 169 | sufS (in | | |
| | iscU | | |
| | operon) | | |
| 171 | sufU (in | | |
| | iscU | | |
| | operon) | | |
| 173 | sufB (in | | |
| | iscU | | |
| | operon) | | |
| 175 | accB (in | | |
| | accC | | |
| | operon) | | |
| 177 | accC (in | | |
| | accC | | |
| | operon) | | |
| 179 | yghY (in | | |
| | accC | | |
| | operon) | | |
| | Corynebacterium^{#3} | | |
| 181 | glmM | phosphoglucosamine mutase | G18NG-10145 |
| 183 | alr | Alanine racemase | G18NG-10150 |
| | Aspergillus^{#5} | | |
| 185 | ARG3/argB | Ornithine carbamoyltransferase | AO090023000856 |
| 187 | adeA | Phosphoribosylaminoimidazole- | AO090020000395 |
| | | succinocarboxamidesynthase | |
| 240 | ERG10 | Acetyl-CoA acetyl-transferase | AO090103000012 |
| 242 | PFS2 | Glucosamine 6-phosphate | AO090003000003 |
| | | synthetases | |
| 244 | TUB1 | Alpha-1 tubulin | AO090005000840 |

^{#1}An essential gene according to the invention is one encoding protein that has at least 70%, 80%, 90%, 95% or even 100% amino acid homology with the protein encoded by the respective essential gene listed in the Table.
^{#2}BioCyc/EcoCyc Database Collection is a large online collection of Pathway/Genome Databases-accessed through https://biocyc.org/
^{#3}SGD is the “Saccharomyces Genome Database”-accessed through https://yeastgenome.org/
^{#4}BL: accessed through https://www.genome.jp/kegg-bin/show_organism?org=bli
^{#5}Aspergillusgenome.org

The essentiality of a gene can be determined by creating cells in which the respective gene is knocked-out, where a non-conditional gene knockout will result in a loss of cell viability, or failure to grow, irrespective of growth conditions; while a conditional gene knockout will result in a failure to grow or loss of cell viability depending on the composition medium/conditions of cultivation.

A suitable essential gene can be identified by Performing a test assay that swaps the native promoter of the assayed essential gene for an inducible promoter, such as the L-arabinose inducible p_{BAD} (in E. coli), xylose-inducible p_{XYL} (in Bacillus subtilis or Bacillus licheniformis) or galactose inducible p_{GAL} (in S. cerevisiae or Pichia pastoris), or plac (in E. coli or Lactobacillus) or p_{thiA} (in Aspergillus). In such an essential gene assay, an integration DNA construct comprising the inducible promoter is chromosomally integrated into a host microbial cell using standard methods. In the case of prokaryotic organisms, the integration DNA preferably harbors an RBS catalog (e.g., Table 3) to direct translation of the essential gene at different rates to account for different baseline expression levels of different promoters and different baseline required expression levels required of essential genes to support growth at the corresponding wildtype specific growth rate. After targeted integration of the integration DNA, integrant cells are plated on plates supplemented with permissive conditions (temperature or concentration of inducers: >0.9% L-arabinose, >0.8% xylose, >1% IPTG, >2% galactose). Cells are subsequently tested for their synthetic addiction to the inducer condition in standard growth curve assays, and suitable essential genes can be identified as genes for which inducer-dependent growth rates can be identified. Inducer-dependent growth can be observed as a reduced growth rate of >5 percent in absence of inducing conditions. Suitable essential genes are characterized by conferring less than <5% reduction in exponential-phase specific growth rate during optimal inducer conditions in production-relevant cultivation medium.

Methods for identification of an essential gene are detailed in “Gene Essentiality—Method and Protocols” January 2015 [DOI 10.1007/978-1-4939-2398-4] which enables the skilled person to determine whether a gene is essential for microbial cell growth under specific growth conditions; including methods for mapping and identifying essential genes of Campylobacter jejuni; Streptococcus sanguinis; Porphyromonas gingivalis; Escherichia coli; Leptospirosis; Mycobacterium tuberculosis; Pseudomonas aeruginosa; and Candida albicans; primarily by screening transposon tagged libraries. Additionally, a variety of “computational tools” described therein enable the skilled person to directly predict and identify genes encoding essential proteins in a microbial genome, facilitated by the widespread availability of whole genome microbial sequences; and the structural features of the many known essential genes. Accordingly the skilled person is provided with both databases of essential genes; and various freely accessible on-line tools and algorithms for successfully and reproducibly identifying large numbers of essential genes in the genome of microbial cells without undue burden. It should further be noted that 82% of the essential genes in Saccharomyces cerevisiae encode essential proteins that are similar to a protein in another organism [Giaever G et al., 2002]; demonstrating that most essential genes, either known or identified in one micro-organism (e.g. yeast) will have a counterpart in other microorganisms—and could thus be identified in a microorganism of choice by a simple BLAST search.

### II.iii Burden-sensing Promoter

The microbial production cell of the invention comprises at least one preferably two burden-sensing promoter(s) operably linked to the at least one preferably two essential gene(s) encoding at least one preferably two protein(s), respectively, required for cell growth and/or survival, as described above. A burden-sensing promoter is one that is induced in a microbial production cell that is subject to a burden and/or fitness cost due to its production or over-production of a product. Typically, induction of the burden-sensing promoter will occur when a microbial cell's production of a product results in a reduction in the cell's growth rate that, in turn, is attributable to the fitness cost of production. Importantly, the at least one preferably two burden-sensing promoters, according to the invention, are induced by the “burden status” in the production cell resulting from production of a product, and not by the product per se. Since the use of the at least one preferably two burden-sensing promoter and essential gene to enhance productivity of a microbial production cell is largely independent of the product it is engineered to make, the burden-addiction conferred by the invention supports a wide range of microbial production cell applications.

Since the essential gene is operably linked to the burden-sensing promoter, its level of transcription and expression is only increased above a basal level when the burden-sensing promoter is induced. A suitable burden-sensing promoter is one that, when un-induced, permits a basal level of expression of the operably linked essential gene that is significantly limiting for, or prevents cell growth and/or survival. Basal level expression of essential gene is a level sufficient to support growth of a cell under production conditions (or during an exponential growth phase) at a level equal to or less than 10, 20, 50, 90 or 95% of the growth rate of a corresponding cell wherein said essential gene is operably linked to its native promoter. This basal level of expression of essential gene will be exerted in burden addicted cells when the burden-sensing promoters is not activated by the burden of efficient product synthesis, and thereby, the resulting lowered growth rate will constitute a selective disadvantage upon lack of product synthesis.

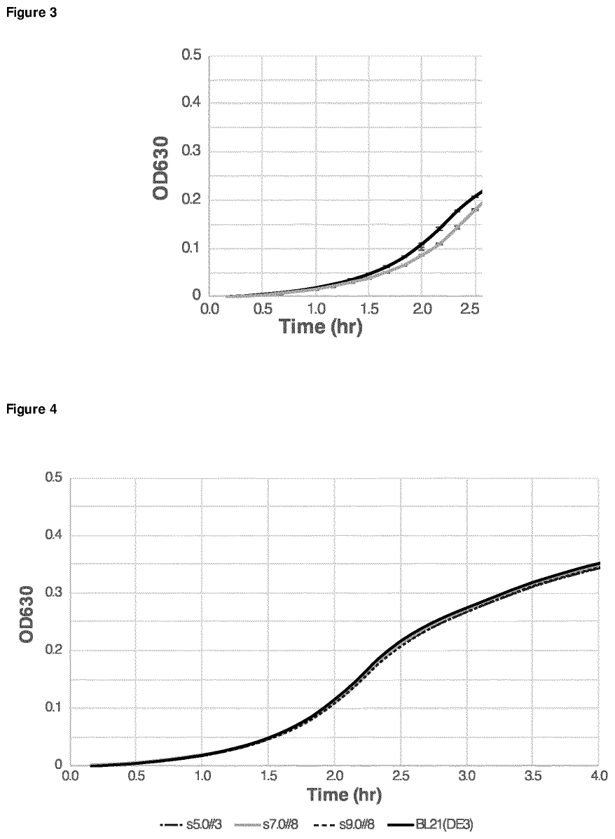

Cells comprising burden-sensing promoter in the un-induced state, will grow significantly slower than cells where the essential gene is operably linked to its native promoter (example 1, FIG. 2). By contrast, microbial production cells comprising the burden-sensing promoter linked to an essential gene exhibit a significantly increased growth rate when the cells synthesize the desired product as compared to cells not producing the product (example 1, FIG. 3). Furthermore, microbial production cells of the invention, despite the inherent burden of producing a product, can surprisingly achieve an initial growth rate that is indistinguishable from the growth rate of parent microbial cells, devoid of gene(s) encoding the product or metabolic pathway for biosynthesis of the product, and from which the microbial production cell was derived (example 1, FIG. 4). Hence under production conditions, a microbial production cell of the invention will have a selective advantage over an escape variant of the production cell that ceases to synthesize the product, and thereby depress the escape rate.

To increase production of desired products and further prolong the period during which the production cells are productive, more than one burden-sensing promoter may be suitable to use to promote growth of beneficial cell variants. By using two or more different burden-sensing promoters to addict a production cell, the highly productive cell variant can more uniquely be differentiated from less productive (genetic/non-genetic) cell variants that over time may arise from the same ancestral starting cell. For example, environmental factors e.g. resulting from outside stresses in a production culture may to some extent activate even less productive cells and thereby permit growth.

By using more than one burden-sensor to control essential genes, the transcriptional space that directs the imposed addiction-based selection regime can be further controlled. This may be important to limit potential cellular escape modes in which the burden addiction response of a single burden biosensor may permit sufficient growth to allow production declines.

Table 2 provides a non-exhaustive list of potential burden-sensing promoters.

In one embodiment, the burden-sensing promoter may be a native promoter, or a modified native promoter, with respect to the microbial production cell, while being heterologous with respect to the essential gene.

In one embodiment, the promoter contains native TF/sigma factor binding sites. Suitable burden-sensing promoters are those of the E. coli σ32 regulon since they are generally upregulated in response to over-expression of proteins and their intracellular aggregation. A non-exhaustive list of E. coli σ32 promoters is included herein in Table 2, while additional suitable promoters are identified by Nonaka et al, 2006.

In one embodiment, the burden-sensing promoter is a non-vegetative o factor regulated promoter. In a preferred embodiment, the burden-sensing promoter is activated via the σ³² regulon, and can be selected from among promoters of the E. coli genes htpG, ibpA, clpB, yccV, grpE, ycjx, ldhA, mutM, ybbN, priC, groES, fxsA, and htpX.

In one embodiment, the burden-sensing promoter is the promoter of the mutM gene. mutM encodes a functionally conserved DNA-glycosylase responsible for initiating repair of one of the most common oxidative stress induced DNA damage, i.e. oxidation of guanine to 7,8-dihydro-8-oxoguanine (8-oxoG) (Jain et al, 2007).

Other suitable burden-sensing promoters may be selected from among promoters of the E. coli genes rrnB, rrnE, cspD, katE, xthA, uspE, gadB, ahpC, katG, grxA, oxyS, poxB, trxC.

Burden-sensing promoters also include promoters related to a cell's carbon nutrient status and growth phase. Accordingly, in one embodiment, the burden-sensing promoter is the promoter of the toxin gene, cspD, which is activated by reduced growth rates and carbon starvation (Uppal et al., 2014; Yamanaka and Inouye, 1997) and hence predicted to be differentially up-regulated in cells subject to burden or fitness cost due to production of metabolites.

In one embodiment, the burden-sensing promoter is a ribosomal RNA promoter, in particular a promoter of rrnB and rrnE genes, that respond to the nutritional supply, are induced by the Fis protein, and are inhibited by ppGpp alarmone. Rrn promoter activity is induced in cells with high protein production, as seen in their early exponential growth phase (Nonaka et al, 2006).

In one embodiment, the burden-sensing promoter is one responsive to oxidative stress associated with metabolic burden and microbial heterologous production (Dragosits & Mattanovich, 2013). Oxidative stress response promoters include those promoters regulated by E. coli OxyR or its homologs such as Bacillus PerR, which activates several genes associated with amelioration and protection of the cell in response to oxidative damage; and promoters of genes belonging to rpoS (σ^{S}) that are upregulated during overexpression of proteins primarily during stationary growth phase.

For Bacillus, suitable burden-sensing promoters may be selected e.g. among HrcA regulated promoters including the promoter of groES. Other suitable burden-sensing promoters may be selected from among promoters of the B. subtilis genes ctsR, dnak, perR, hrcA, spx, sigB, yflT, mutM.

For Corynebacterium, such suitable burden-sensing promoters may be selected from among katA, cplX, mutM and groES.

For Saccharomyes, suitable burden-sensing promoters may be selected from among promoter of genes KAR2, PDI1, SAA1, FPR2, RPL3, RPL6A, RPL28, OGG1, RAD51, RAD54.

For Aspergillus, such suitable burden-sensing promoters may be selected from among bipA, PDI, clxA and the homologues of S. cerevisiae OGG1, RAD51 and RAD54.

TABLE 2
Non-exhaustive list of burden-sensing promoters

| | | | Ecocyc/ |
|---|---|---|---|
| | | | Biocyc |
| | | | accession |
| | | Encoded protein of gene regulated | ID**/AspGD |
| No.* | Promoter | by the promoter | ID*** |
| 2 | p(htpG) | Heat shock protein G (HtpG), a member | EG10461- |
| | (SEQ ID | of the Hsp90 family, is a molecular | MONOMER |
| | NO.: 65) | chaperone that aids the refolding of | |
| | | protein aggregates in the cell. htpG | |
| | | transcription is activated via the σ32 | |
| | | regulon. (Yeast homolog: Hsp90) | |
| 3 | p(ibpA) | Small heat shock proteins LbpA and | EG11534 |
| | (SEQ ID | LbpB are chaperone proteins associated | |
| | NO.: 66) | with herterologous protein expression | |
| | | and inclusion body formation that aids | |
| | | refolding of aggregated protein. ibpA | |
| | | transcription is activated via the σ32 | |
| | | regulon. | |
| 4 | p(clpB) | ClpB is a caseinolytic protease | EG10157 |
| | (SEQ ID | catalyzing protein disaagregation. clpB | |
| | NO.: 67) | transcription is activated via the | |
| | | σ32 and/or σ70 regulon. | |
| | | (Yeast homolog: Hsp104). | |
| 5 | p(yccV) | YccV also known as HspQ participates | G6500 |
| | (SEQ ID | in degradtion of denatured protein | |
| | NO.: 68) | during heat shock. yccV transcription | |
| | | is activated via the σ32 regulon. | |
| 6 | p(grpE) | GrpE is a nucleotide exchange factor | EG10416 |
| | (SEQ ID | in the DnaK-DnaJ-GrpE chaperone | |
| | NO.: 69) | system. grpE promoter is activated | |
| | | via the σ32 regulon. | |
| 7 | p(ycjX) | ycjX is activated via the σ32 regulon. | G6659 |
| | (SEQ ID | | |
| | NO.: 70) | | |
| 8 | p(ldhA) | D-lactate dehydrogenase and the ldhA | G592 |
| | (SEQ ID | promoter is activated via the σ32 regulon. | |
| | NO.: 71) | | |
| 9 | p(mutM) | DNA-formamidopyrimidine glycosylase | EG10329 |
| | (SEQ ID | is a component of the base excision repair | |
| | NO.: 72) | pathway repairing certain types of DNA | |
| | | lesions. mutM promoter is activated via | |
| | | the σ32 regulon, but is inhibited by DksA- | |
| | | ppGpp binding under amino acid | |
| | | starvation. | |
| 10 | p(ybbN) | YbbN also known as CnoX is a | G6268 |
| | (SEQ ID | chaperedoxin. The ybbN promoter is | |
| | NO.: 73) | activated via the σ32 regulon. | |
| 11 | p(prlC) | prlC also known as oligopeptidase A; | EG11441 |
| | (SEQ ID | and belongs to the σ32 regulon. | |
| | NO.: 74) | | |
| 12 | p(groES) | GroES is a chaperonin, and belongs to | EG10600 |
| | (SEQ ID | the σ32 and σ70 regulon. | |
| | NO.: 75) | | |
| 13 | p(fxsA) | FxsA is a polytopic membrane protein | G7832 |
| | (SEQ ID | belonging to the σ32 regulon. | |
| | NO.: 76) | | |
| 14 | p(htpX) | HtpX is a zinc-dependent endoprotease | EG10462 |
| | (SEQ ID | and heat shock protein that belongs to | |
| | NO.: 77) | the σ32 regulon. | |
| 15 | p(rrnB) | 16S rRNA gene, whose promoter | EG30085 |
| | (SEQ ID | responds to nutritional supply, and is | |
| | NO.: 78) | induced in cells with high protein | |
| | | production. | |
| 16 | p(rrnE) | 16S rRNA gene, whose promoter responds | EG30088 |
| | (SEQ ID | to nutritional supply, and is induced in | |
| | NO.: 79) | cells with high protein production. | |
| 19 | p(cspD) | CspD is a toxin. The cspD promoter | EG11111 |
| | (SEQ ID | responds inversely with growth rate. | |
| | NO.: 80) | Transcription of cspD is activated by | |
| | | cyclic-AMP-CRP, H-NS and ppGpp. | |
| 21 | p(katE) | Monofunctional catalase (HPII) | EG10509 |
| | (SEQ ID | responsible for decomposing the toxic | |
| | NO.: 81) | hydrogen peroxide into water and | |
| | | oxygen in the cell. katE transcription | |
| | | is dependent on σ^{S} and activated by | |
| | | Fur-Fe²⁺ and ppGpp. | |
| 22 | p(xthA) | Multifunctional endonuclease, | EG11073 |
| | (SEQ ID | Exodeoxyribonuclease III, that e.g. | |
| | NO.: 82) | removes DNA damaged due to hydrogen | |
| | | peroxide. xthA belongs to the σ³² regulon. | |
| 23 | p(uspE) | Universal stress protein E; which is | EG11246 |
| | (SEQ ID | induced in stationary phase as a result | |
| | NO.: 83) | of various stress conditions such as | |
| | | oxidative stress. uspE transcription is | |
| | | activated by GadX and ppGpp. | |
| 24 | p(gadB) | Glutamate decarboxylase B helps the | EG11490 |
| | (SEQ ID | cell maintain a near-neutral intracellular | |
| | NO.: 84) | pH upon exposure of acidic conditions | |
| | | by converting glutamate into gamma- | |
| | | aminobutyrate, accepting one proton in | |
| | | the reaction. gadB is a part of the the σ⁷⁰ | |
| | | and σ^{S} regulon, and is activated by AdiY, | |
| | | GadE, GadX, p^{asp56}-RcsB and ppGpp. | |
| 25 | p(ahpC) | Alkyl hydroperoxide reductase C plays | EG11384 |
| | (SEQ ID | a role in protecting cells from oxidative | |
| | NO.: 85) | stress by catalyzing the reduction of | |
| | | endogenously synthesizes hydroperoxide. | |
| | | ahpC is part of the σ⁷⁰ and transcription | |
| | | is activated by OxyR. | |
| 26 | p(katG) | Bifunctional hydroperoxidase I (HPI) | EG10511 |
| | (SEQ ID | that acts both as a catalase and a | |
| | NO.: 86) | peroxidase. | |
| 27 | p(grxA) | Glutaredoxin is mainly responsible for | EG10417 |
| | (SEQ ID | catalyzing the reduction of disulfides. | |
| | NO.: 87) | Transcription initiation of grxA is | |
| | | activated by OxyR. | |
| 28 | p(oxyS) | oxyS is a small RNA gene activated by | EG31116 |
| | (SEQ ID | oxidative stress via OxyR. | |
| | NO.: 88) | | |
| 29 | p(poxB) | Pyruvate oxidase that is upregulated by | EG10754 |
| | (SEQ ID | oxidative and osmotic stress via OxyR, | |
| | NO.: 89) | but also by Cra, MarA, fnr, ppGpp and σ^{S} | |
| 30 | p(trxC) | Thioredoxin that is induces during | EG11887 |
| | (SEQ ID | oxidative stress via OxyR | |
| | NO.: 90) | | |

Saccharomyces

| | |
|---|---|
| p(KAR2) | HAC-upregulated promoter comprising |
| (SEQ ID | and unfolded proteins response element, |
| NO.: 91) | positively responding to high production |
| p(PDI1) | HAC-upregulated promoter comprising an |
| (SEQ ID | unfolded proteins response element, |
| NO.: 92) | positively responding to high production |
| p(SAA1) | HAC-upregulated promoter, comprising |
| (SEQ ID | an unfolded proteins response element, |
| NO.: 93) | positively responding to high production |
| p(FPR2) | HAC-upregulated promoter comprising |
| (SEQ ID | an unfolded proteins response element |
| NO.: 94) | |
| p(RPL3) | RNA polymerase I upregulated promoter, |
| (SEQ ID | positively responding to high production |
| NO.: 95) | |
| p(RPL6A) | RNA polymerase I upregulated promoter, |
| (SEQ ID | positively responding to high production |
| NO.: 96) | |
| p(RPL28) | RNA polymerase I upregulated promoter, |
| (SEQ ID | positively responding to high production |
| NO.: 97) | |
| p(OGG1) | DNA damage response, resulting from |
| (SEQ ID | heterologous expression broadly induces |
| NO.: 98) | transcription of the DNA repair system. |
| p(RAD51) | DNA damage response, resulting from |
| (SEQ ID | heterologous expression broadly induces |
| NO.: 99) | transcription of the DNA repair system. |
| p(RAD54) | DNA damage response, resulting from |
| (SEQ ID | heterologous expression broadly induces |
| NO.: 100) | transcription of the DNA repair system. |

Bacillus

| | | |
|---|---|---|
| P_{groES} | GroES is a chaperonin and is regulated | BSUB:BUS |
| (SEQ ID | by SigA and HrcA. | 06020- |
| NO.: 212) | | MONOMER |
| P_{ctsR} | CtsR is a tramscription repressor of class | BSUB:BUS |
| (SEQ ID | III heat shock genes and is regulated by | 00830- |
| NO.: 213) | SigM, SigA, SigB, SigF, Spx and is also | MONOMER |
| | autoregulated. | |
| P_{dnaK} | DnaK is a molecular chaperone and is | BSUB:BUS |
| (SEQ ID | regulated by HrcA and SigA | 25470- |
| NO.: 214) | | MONOMER |
| P_{perR} | PerR is a transcriptional repressor of the | BSUB:BUS |
| (SEQ ID | peroxide regulon and a sensor of the | 08730- |
| NO.: 215) | intracellular Fe/Mn ratio, and is regulated | MONOMER |
| | by SigA and is also autoregulated. | |
| P_{hrcA} | HrcA is a transcriptional repressor of | BSUB:BUS |
| (SEQ ID | class I heat-shock genes and is regulated | 25490- |
| NO.: 216) | by SigA and is also autoregulated. | MONOMER |
| Pₛₚₓ | Transcriptional regulator involved in the | BSUB:BUS |
| (SEQ ID | prevention of protein aggregation and is | 11500- |
| NO.: 217) | regulated by PerR, SigW, SigX, SigM, | MONOMER |
| | and SigA | |
| P_{sigB} | RNA polymerase sigma factor σ^{B} involved | BSUB:BUS |
| (SEQ ID | in General stress response and is regulated | 04730- |
| NO.: 218) | by SigA and CcpA, and is also autoregulated | MONOMER |
| P_{yflT} | General stress protein 17M regulated by | BSUB:BUS |
| (SEQ ID | SigB | 07550- |
| NO.: 219) | | MONOMER |

Aspergillus***

| | | |
|---|---|---|
| PbipA | An ER localized chaperone (BiP) involved | AO090003000257 |
| (SEQ ID | in protein secretion | |
| No.: 189) | | |
| PclxA | A calnexin homologue involved in protein | AO090009000313 |
| (SEQ ID | secretion | |
| No.: 190) | | |
| PagsA | 1,3-alpha-D-glucan synthase | AO090010000106 |
| (SEQ ID | | |
| No.: 239) | | |

Corynebacterium**

| | | |
|---|---|---|
| pgroES | Chaperone | G18NG-10159 |
| (SEQ ID | | |
| No.: 191) | | |
| pkatA | Catalase | G18NG-9810 |
| (SEQ ID | | |
| No.: 192) | | |
| pcplX | | |
| (SEQ ID | | |
| No.: 193) | | |
| pmutM | DNA damage repair | G18NG-11665 |
| (SEQ ID | | |
| No.: 194) | | |

*No. corresponds to the numbers assigned to the different strains tested in the Examples section of the present application.
**BioCyc/EcoCyc Database Collection is a large online collection of Pathway/Genome Databases-accessed through https://biocyc.org/
***For Aspergillus, Aspergillusgenome.org is a database (AspGD) featuring the genomes of relevant Aspergillus species.

The basal expression level and specific response curve upon induction/activation of the different burden-sensing promoters (e.g. in Table 2) vary, which can be exploited when selecting a burden-sensing promoter having the best match (in terms of strength) to a specific microbial production cell and its product.

In some cases, the gene of interest is part of an operon, in which case the first gene of the operon is preferably used to define the relevant promoter sequence.

Suitable burden-sensing promoters that are induced by the burden and/or fitness cost in a microbial production cell during production can be selected from public databases known to the skilled person, for example https://ecocyc.org for E. coli promoters; https://bsubcyc.org for Bacillus promoters (which also works for Corynebacterium promoters), their respective prokaryotic homologues; https://yeastgenome.org for Saccharomyces cerevisiae promoters, and their fungal homologues; and https://aspergillusgenome.org is a database (AspGD) featuring the genomes of relevant Aspergillus species.

Suitable burden-sensing promoters can also be validated in a test assay in which a candidate burden-sensing promoter is operably linked to a gene encoding a fluorescent protein (e.g. green fluorescent protein) and integrated into a microbial production cell genetically engineered to synthesize a product, and integrated into a corresponding non-producing cell (control), which was isolated following a serial dilution cultivation experiment in production medium for between 50-150 cell generations or from the end of a large scale fermentation culture. Burden-sensing promoters induce transcription of the candidate burden-sensing promoter operably linked to fluorescent protein expression in producing cells compared to the non-producing cell (control) by at least 5%, 7.5%, 10%, 15%, 25%, 60%, 150%, 300% in the production cell compared to the activity in the isolated corresponding non-producing cell.

### II.iv Matching a Burden-sensing Promoter to a Microbial Production Cell

Microbial production cells, in response to the burden or fitness cost of producing a product, exhibit a transcriptional state characterized by the expression of certain genes, whose expression is not induced in a corresponding non-productive parent cell or a non-productive escape cell derived from a microbial production cell during production fermentation. The physiological nature of the burden or fitness cost will depend on the product synthesized by a given microbial production cell, and will be reflected in the types of genes whose expression is induced. The identity of the gene promoters induced in a cell producing a given product can be determined by techniques well-known in the art (e.g. transcriptomics, see example 7). Since many of the induced gene promoters will be related to, or correspond to those listed in Table 2, these provide a starting point for finding a matching burden-sensing promoter for a given microbial production cell. The choice of promoters can be extended by identifying 5 to 15 promoters of genes found to be specifically upregulated in the production strain of interest. As illustrated in the examples herein, the method used to match a burden-sensing promoter operably linked to an essential gene to a microbial production cell in a product-specific manner, is experimentally fast and can be conducted in simple laboratory setups.

### II.v Translation Control Elements

Regulating the growth of a microbial production cell of the invention by means of essential gene expression, requires that the response threshold and curve of the burden-sensing promoter and the expression level of the essential gene are balanced; such that basal level essential gene expression supports limited or no growth, while in highly productive cells the induced burden-sensing promoter drives sufficient essential gene expression to support a significantly increased growth rate, preferably a growth rate similar to that of productive cells lacking the burden-sensing promoter of the invention or ≤5% lower, when measured in the exponential growth phase.

One suitable approach to balancing the burden-sensing promoter's burden-response to the expression level of the essential gene is to modify the translational strength of the essential gene. In bacteria, translational strength is defined by the Shine-Dalgarno/ribosome binding site (RBS) sequence directly upstream of the start codon, while in eukaryotic cells translation initiation regions, translation initiation sites (TIS) or Kozak elements can be used to modify translational strength. RBSs in E. coli conferring a broad range of translational strengths are provided in Table 3.1, while further examples can be found in the literature (e.g. Bonde et al, 2016). A skilled person in the art can balance the translational strength of the regulated essential gene by constructing four variants of the ribosomal binding site for each burden-sensing promoter (Rugbjerg et al, 2018, PNAS), and testing which variant enables a selected essential gene to effectively regulate the growth rate of a cell. Exemplary RBSs for use in Bacillus licheniformis or Bacillus subtilis are provided in Table 3.2. Exemplary TISs for use in Pichia are provided in Table 3.3.

TABLE 3.1
List of suitable RBSs in E. coli generally
covering a broad expression range as defined in
assay by expression of red fluorescent protein
(Bonde et al, 2016).

| RBS | Log (Expression level) |
|---|---|
| ACTTGA | 2.80244 |
| ACTTGC | 2.73094 |
| ACTTGG | 3.14847 |
| ACTTGT | 2.77523 |

TABLE 3.2
List of suitable RBSs in B. subtilis or B.
licheniformis generally covering a broad
expression range.
RBS

ACGAGA
ACGAGC
ACGAGT
ACGAGA

TABLE 3.3
List of suitable TIS sequence variations in P.
pastoris to modulate essential gene expression.

| TIS | Sequence: |
|---|---|
| TIS1 | TGATAT |
| TIS3 | ACGTTC |
| TIS5 | TAGGTT |
| TIS9 | AAAACA |

### II.vi Engineering Burden-sensing Promoters and Translational Control Element

A native burden-sensing promoter of choice is generally encompassed by the −1 to −300 bp region (upstream) of the native regulated ORF in prokaryotic organisms and the −1 to −500 bp region in eukaryotic organisms. Core promoters that must be included and the sequence boundaries of their regulatory sites e.g. transcription factor binding sites, are common general knowledge, such as for σ32 (Nonaka et al, 2006). The translational control element/RBS can be added downstream of the promoter, to avoid alteration in the regulatory properties of the selected promoter sequence.

### II.vii Improved Production Levels

A surprising advantage of the microbial production cell of the invention, in addition to having an increased initial growth rate as compared to non-productive cells, is that the cells retain significantly improved productivity during a large scale fermentation (simulated in example 2; FIG. 6), over many cell divisions, compared to microbial production cells lacking engineered burden-addiction. The surprising advantages conferred by burden-addiction in a microbial production cell of the invention are obtained irrespective of the type of product synthesized—as illustrated by the range of products synthesizes by microbial production cells in examples 2-5.

According to one embodiment, the microbial production cell of the invention is characterized by improved product yield following at least 20, 25, 30, 35, 40, 45, 50, 55, 60, 70, 100, 150, 250 or 400 generations of cell division from a single cell, compared to a non-burden-addicted production cell following the same generations of cell division. Product yield is measured as moles or grams of product produced per unit substrate.

In one embodiment, production levels are increased by at least 10, 25, 50, or 80% following at least 50 generations of cell division from single cell, compared to a reference non-burden-addicted production cell following the same generations of cell division.

Universal practice in the engineering of microbial cell factories has aimed at minimizing the burden known to arise due to maintenance and expression of heterologous pathways. The present invention provides a counter-intuitive solution to improving cell factory yields, since it makes microbial cell growth and survival dependent on the cell producing its product while being subject to a constant burden or fitness cost. While not wishing to be bound by theory, it is speculated that the selection pressure placed on a microbial production cell of the invention and its descendants, where only productive cells in a state of burden can grow/survive, may serve to progressively select for highly-burdened cell sub-populations during long-term culture in a starting population of initially isogenic cells.

### III. Methods for Preparing and Identifying Cells of a Burden-addicted Microbial Production Strain

In a preferred method of performing the invention, a burden-addicted microbial production strain is prepared and identified by the following steps. To obtain the best working relation between burden sensing promoter and essential gene, it may be relevant to prepare and test different candidate burden-sensing promoters with different RBSs for regulating a selected essential gene.

### III.i Identification of Burden-sensing Promoter Candidate(s) in a Specific Production Strain

Candidate burden-sensing promoters listed in Table 2 may be tested. Alternatively, unique, positively differentiating gene transcripts detected in a specific production strain of interest may be identified; whose respective promoters provide a source of candidate burden sensing promoters. Preferably such differentiating gene transcripts are identified by comparing the transcript profile of a microbial production strain (during production) with an isolated genetic or non-genetic escape mutant variant(s) derived from the corresponding production strain, where such escape mutant variant is characterized by at most a 50% lower production rate. Positively differentiating gene transcripts can be identified by use of transcriptomics; for example by RNA sequencing of the transcribed RNA extracted from productive microbial production cell(s) compared to the non/low-producing escape mutant variant. Identified promoters can be tested in combination with a given essential gene in a microbial production cell.

A method for identifying at least one preferably two burden-sensing promoters is illustrated in example 7; where after said promoter(s) is operably linked to an essential gene in a chosen microbial production cell for testing.

### III.ii Introduction of Burden-sensing Promoter in Growth-regulating Processes

The at least one preferably two promoter(s) selected from the list of general candidate burden-sensing promoters (Table 2) or from identified specific burden-sensing promoter(s) (see IIIi), are tested by operably linking it to an essential gene in the microbial production cell. Different translation strengths of the essential gene can be simultaneously tested by providing alternative RBS sequences for the cognate essential gene; as well as testing different essential genes (as describe in section Iiii). The essential gene may be native or heterologous with respect to the microbial production cell.

When the essential gene is a native gene, its cognate native promoter may be disrupted (i.e. made non-functional) by e.g. mutation or deletion events, and the burden-sensing promoter (that is heterologous with respect to the essential gene) is then operably linked to the native essential gene by targeted introduction. In another embodiment, the native essential gene promoter is replaced with the burden-sensing promoter by targeted introduction.

Suitable methods for targeted introduction of a genetic sequence in microorganisms include recombineering in bacteria, e.g. Lambda Red recombineering in E. coli; while homologous recombination can be used in yeasts and filamentous fungi. Both concepts can optionally be used in combination with CRISPR to increase gene replacement efficiency.

### III.iii Screening for Balanced Burden-sensing and Growth-regulation

Microbial production cell clones comprising an inserted burden-sensing promoter operatively linked to an essential gene are then screened to identify clones where the burden-sensing promoter regulates an essential gene in a growth-controlling manner. For example, the growth of a number of such clones (e.g. 8-96 clones) is compared with cells of a corresponding non-burden-addicted microbial production strain, under conditions were product production is controlled (e.g. using microbial production strains where the production gene is inducible).

When cell growth is measured under conditions where product synthesis by both strains is low/absent; a suitable burden-addicted production clone is one that exhibits a significant, and preferably at least 5%, lower growth rate than the non-burden-addicted production strain (Example 1, FIG. 2). If instead production is high/present; the up-regulation of essential gene expression in a suitable burden-addicted production clone, means that the difference in growth rate is significantly reduced or eliminated when compared to the corresponding non-burden addicted production strain.

### III.iv Validation of Non-perturbed Central and Production Metabolism

Changes in transcriptional regulation or expression of essential genes can lead to unwanted, indirect perturbation of the production genes and central carbon or nitrogen metabolism, compromising product formation and in turn reduced burden in a burden-addicted microbial production cell. In order to exclude such clones, cell growth is measured under conditions where both strains synthesize product; where a suitable burden-addicted production clone is one that exhibits a growth rate equal to or lower than the non-burden-addicted production strain (as seen in example 2, FIG. 7).

### III.v Screening for Growth-rate Stability

Clones that fulfil the above criteria are tested for growth rate stability under production-mimicking conditions by cultivation for at least 20 generations of cell division, e.g. by serial passage and measurement of growth rates, or by taking samples from various steps in a scaled-up production process. Suitable burden-addicted microbial production cells maintain growth rates lower than the non-burden-addicted production strain over time (e.g. example 2, FIG. 7).

### IV. Method for Producing a Desired Product Using Cells of a Burden-addicted Microbial Production Strain

A second aspect of the present invention concerns a method for producing a desired product comprising the steps of:
a. providing at least one microbial production cell genetically engineered to synthesize a product, wherein said at least one cell further comprises at least one preferably two essential gene(s) operably linked to at least one preferably two burden-sensing promoter(s), respectively, and wherein said promoter(s) are heterologous with respect to said essential gene,
b. introducing the at least one genetically modified microbial cell into a cultivation medium comprising substrate for production of said product,
c. recovering said product synthesizes by said culture,
   wherein synthesis of the product confers a burden and/or fitness cost on said at least one cell, and wherein expression of said at least one preferably two essential gene(s) are up-regulated relative to basal level expression of said at least one preferably two essential gene(s) when said at least one preferably two burden-sensing promoter(s) are induced by said burden and/or fitness cost; and wherein a lack of said product synthesis in said at least one production cell or progeny cell thereof reduces growth rate of said at least one cell.

The method for producing the desired product may comprise the step of providing a cell culture of said at least one microbial production cell genetically engineered to synthesize a product; and introducing the cell culture of the at least one genetically modified microbial cell into a cultivation medium comprising substrate for production of said product. The cells are cultured in the cultivation medium to allow the cells to produce the desired product and to support growth and multiplication of the cells. The time of cultivation may be optimized depending on that the desired product is.

The method for producing the desired product may further comprise a step of isolating the product and/or formulating of the product into a composition, such as a nutritional, pharmaceutical, cosmetic, detergent, lubricant, or fuel composition

### V. Use of Cells of a Burden-addicted Microbial Production Strain for Producing a Desired Product

A third aspect of the present invention concerns the use of a burden-addicted microbial production cell of the invention for producing a desired product, wherein a lack of product synthesis in said a burden-addicted production strain or progeny cell thereof reduces growth rate of said strain even if product is present in the intracellular or extracellular environment.

### EXAMPLES

### Example 1: Engineering Burden-addicted E. coli Strains Producing Recombinant Human Growth Hormone

### 1.1 Maintenance and Expression of Heterologous Production Genes by a Cell Factory Constitutes an Unnatural Burden on its Cells

The growth rate of parent E. coli BL21(DE3) cells was compared with an engineered derivative transformed with the plasmid, pEG34, comprising a gene encoding recombinant human growth hormone (hGH) fused to Green Fluorescent Protein (GFP).

### 1.1.1 Materials and Methods

Cells of the host strain E. coli BL21(DE3) (E. coli Genetic Stock Center at Yale University), were made electrocompetent and transformed by standard electroporation methods (1800 V, 25 μF, 200 Ohms, 1 mm cuvette width) with the plasmid pEG34, and plated on LB agar plates containing chloramphenicol.

TABLE 4
Plasmids

| Plasmid | Genotype | Expressed proteins |
|---|---|---|
| pEG34 | hGH-GFP fusion | hGH fused to GFP |
| [SEQ ID No.: 101] | camR gene* | [SEQ ID No.: 102] |
| | [SEQ ID No.: 103] | Chloramphenicol |
| | | resistance protein |
| | | [SEQ ID No.: 104] |
| pEG0 | camR gene* | Chloramphenicol |
| [SEQ ID No.: 105] | [SEQ ID No.: 103] | resistance protein |
| | | [SEQ ID No.: 104] |
| pENDU5CAM | lss gene | Lysostaphin |
| [SEQ ID No.: 106] | camR gene* | [SEQ ID No.: 107] |
| | [SEQ ID No.: 103] | Chloramphenicol |
| | | resistance protein |
| | | [SEQ ID No.: 104] |
| pMevT | mevT operon | Acetoacetyl-CoA |
| [SEQ ID No.: 108] | camR gene* | synthase |
| | [SEQ ID No.: 103] | [SEQ ID No.: 109] |
| | | Hydroxymethyl- |
| | | glutaryl-CoA |
| | | synthase |
| | | [SEQ ID No.: 110] |
| | | Hydroxymethyl- |
| | | glutaryl-CoA |
| | | reductase |
| | | [SEQ ID No.: 111] |
| | | Chloramphenicol |
| | | resistance protein |
| | | [SEQ ID No.: 104] |
| pSIM5-test | | |
| [Koskiniemi et al | | |
| 2011] | | |

camR gene [SEQ ID No: 103] encoding chloramphenicol resistance protein [SEQ ID No: 104] is in pEG34 [SEQ ID No: 101] located at nucleotide positions 3768-4427 on the complementary strand; is in pEG0 [SEQ ID No: 105] located at nucleotide positions 2339-2995 on the complementary strand; is in pENDU5CAM {SEQ ID No: 106] located at nucleotide positions 1617-2276 on the complementary strand; and is in pMevT [SEQ ID No: 108] located at nucleotide positions 6585-7244 on the complementary strand.

Pre-cultures of single colony transformants were cultured in 96-well microtiter plates on 200 μL 2xYT medium (16 g/L tryptone, 5 g/L NaCl, 10 g/L yeast extract) containing 500 μM IPTG and 30 mg/L chloramphenicol at 37° C. with fast horizontal shaking in an Elx808 plate reader (Biotek) with reads at OD630 every ten minutes. OD630 values were background-subtracted using the OD630 value of the first read.

### 1.1.2 Results

E. coli cells comprising the hGH-GFP expression plasmid, pEG34, grow slower than cells of the parent E. coli host from which is was derived (FIG. 1); illustrating the burden or load placed on cells as a result of allocating cellular resources to maintenance and expression of a synthetic construct.

### 1.2 Engineering a Burden-addicted Cell Factory

Burden-addiction strains of the parent E. coli host were genetically engineered to incorporate a genetic circuit designed to confer a selective fitness advantage on productive cells in a cell factory. Specifically, a burden-sensing promoter (p_{mutM}, p_{yccV} or p_{ycjX}), was substituted for the native promoter of the essential gene operon folP-glmM in the E. coli BL21(DE3) chromosome. The genetic circuit further included an RBS, between the promoter and essential gene, where four different RBSs (Table 3) were tested for modulating the expression level of the essential gene. The growth rate of strains comprising the genetic circuit was compared with the parent host strain E. coli BL21(DE3).

### 1.2.1 Materials and Methods

Burden-sensing promoters: The promoters, p_{mutM}, p_{yccV} or p_{ycjX}, were generated by PCR amplifying a 0.3 kb region immediately upstream of the respective gene (see Table 3) using primers specified in Table 5 and genomic DNA derived from lysed E. coli BL21(DE3) cells as template. The PCR mix: 10 μl MQ water, 2 μl forward primer (10 μM), 2 μl revers primer (10 μM), 1 μl DNA template, 15 μl Phusion U MasterMix (Thermo Scientific). The PCR reaction protocol: 95° C. for 180 sec (1×); 95° C. for 20 sec, 68-58° C. (touchdown) for 30 sec, 72° C. for 60 sec (35×); 72° C. for 300 sec (1×); leave at 15° C.

TABLE 5
Primers

| Promoter of | Primer | | SEQ ID |
|---|---|---|---|
| E. coli gene | name | Primer sequence (5′-3′)* | No.: |
| yccV | yccV for | ATTCATATGUGGTTATAAAGACATCAACATGC | 112 |
| | yccV rev | ATCCCCATTACGTGAGGATGGCTAAGGTCCAG | 113 |
| | | TGAAGTACCCTGGGCAAAGAGTTTCATaccttN | |
| | | CAAGTAGTCACCTCCCGGGAAATCT | |
| ycjX | ycjX for | ATTCATATGUTTGGCTGATACTGTAATTCTTC | 114 |
| | ycjX rev | ATCCCCATTACGTGAGGATGGCTAAGGTCCAG | 115 |
| | | TGAAGTACCCTGGGCAAAGAGTTTCATaccttN | |
| | | CAAGTTGCTGTCCTGTGCTGCTCTG | |
| mutM | mutM for | ATTCATATGUGGCGAAAAGTACTAAGTACTTA | 116 |
| | mutM rev | ATCCCCATTACGTGAGGATGGCTAAGGTCCAG | 117 |
| | | TGAAGTACCCTGGGCAAAGAGTTTCATaccttN | |
| | | CAAGTAGCATCTCCAGGAATGAACA | |
| | P492 | TGCAACTCTTCTTCAACGC | 118 |
| | P493 | AAGAACCAGGCGCTTCTAA | 119 |
| | P525 | ATTGATGACCTGATGGCAC | 120 |
| | P526 | GATCCAGACTTCGAAGCG | 121 |

*RBS complementary coding sequences are in bold font; wherein N is either A, G, T or C. The RBS is encoded on the opposite strand of the oligo sequence shown.

USER cloning was used to generate integration sequences comprising the amplified promoter region fused to a linear 1.5 kb DNA fragment containing a kanR gene for selection of correct recombineering products and a 221 bp targeting sequence identical to 221 bp directly upstream of the folP gene. The amplified promoter region and kanR-folP fragment were fused by mixing them in equimolar amounts and adding 1 μl 10× T4 ligation buffer (Thermo Scientific) and 0.75 μl USER enzyme (New England Biolabs), in a total 10 μl reaction volume. The USER reaction was placed at 37° C. for 30 minutes. The reaction was then placed at room temperature for 15 minutes followed by the addition of 0.75 μl T4 DNA ligase (Thermo Scientific) and incubation at room temperature for 30 minutes. An example of such integration sequence comprising promoter, KanR resistance gene, and folP targeting sequence can be found in the sequence listing SEQ ID NO. 140 (s9_pmutM_folP). The ligated product was then amplified using primers ‘rev’ (according to specific promoter in Table 5) and P493 (Table 5) to approximately 250 ng/μl using the PCR reaction protocol: 98° C. for 180 sec (1×); 85° C. for 20 sec, 72-68° C. (touchdown) for 30 sec, 72° C. for 60 sec (35×); 72° C. for 5 min (1×); leave at 15° C. Primer overhang provided 50 bp folP identical targeting sequence to direct recombineering to the folP locus.

Chromosomal integration of burden-addiction promoters: promoters were integrated upstream of the folP gene in the genome of E. coli BL21(DE3) cells as follows: 100 ml of 2xYT medium containing tetracycline were inoculated with 600 μl of BL21(DE3) overnight culture pre-transformed with pSIM5-tet (Koskiniemi et al, 2011). The cells were cultured at 30° C., and upon reaching OD600=0.20, the culture was transferred to a 42° C. shaking bath for 15 minutes to allow for the expression of the recombineering enzymes located on pSIM5-tet. The culture was then transferred to 2× cold 50 ml centrifuge tubes; centrifuged at 4000 g for 10 min; the supernatant was discarded; and the remaining cell pellets were washed with 20 ml ice-cold 10% glycerol. The partially re-suspended cells were then centrifuged at 4000 g for 6 min; the supernatant was discarded; and the cell pellets washed with 20 ml ice-cold 10% glycerol. The partially re-suspended cells were centrifuged at 4000 g for 6 min; each cell pellet was carefully re-suspended in 495 μl ice-cold 10% glycerol and pooled. 90 μl of re-suspended cells were added to electroporation cuvettes each containing 1 μl (>250 ng) of one of the burden-sensing promoter integration sequences. The cells were electroporated with the following settings: 1800 V, 25 μF, 200 Ohms, 1 mm cuvette width. 900 ml 2xYT-media was added to the cuvette straight after electroporation and the cells were left to recover for 1.5 hour at 37° C. in 1.5 ml Eppendorf tubes. The electroporated cells were incubated at room temperature overnight to allow time for recombination; and subsequently plated on LB agar plates containing 50 mg/L kanamycin and cultured at 37° C. overnight to ensure curing for pSIM5-tet. Correct targeting to the essential gene locus was validated using primers P525 and P526. Generally, single kan^{R} colonies were picked having a size average or smaller than the population of colonies.

Burden-addiction promoter integration in the genome of the kanR selected colonies was validated by colony PCR using Taq DNA polymerase and primers targeted to the folP promoter region. Additionally, the identity of the RBS in the selected colonies was determined by Sanger sequencing.

Subsequently, the growth rate of strains comprising the genetic circuit was compared with the parent host strain E. coli BL21(DE3) by cultivation in 200 μl 2xYT supplemented with 500 μM IPTG at 37° C. with horizontal shaking.

### 1.2.2 Results

The growth rate of the selected burden-addicted E. coli strains were slower, to various degrees, compared to parent E. coli host from which they were derived (FIG. 2; showing growth of burden-addicted strains s5.0 #3, s7.0 #8, and s9.0 #8 wherein essential genes folP-glmM are controlled by burden-sensing promoters p_{yccV}, p_{ycjX}, and p_{mutM}, respectively). Since cellular growth is dependent on the level of expression of the essential folP-glmM genes, it may be concluded that the level of essential gene expression driven by the respective burden-sensing promoter in a non-productive, burden-addicted strain is insufficient to support growth at the level of a wildtype parent strain.

The observed reduction in growth rate in these burden-addicted strains provides a measure of the penalty that may be exerted on a non-producing cell that evolves spontaneously during cultivation of a cell factory population comprising the burden-addiction genetic circuit. The degree of penalty is determined by the choice of the burden-sensing promoter combined with the strength of the chosen RBS. The larger the penalty-the wider the window for “negative selection” of any low-or non-producing variants (e.g. resulting from mutation in production genes) spontaneously arising during cultivation.

### 1.3 Production Increases Growth Rate of a Burden-addicted Cell Factory

The burden-addicted E. coli strains, which conferred a growth penalty for non-production (FIG. 2), were used as host strains for demonstrating the selective fitness advantage for burden-addicted cells that are productive.

### 1.3.1 Materials and Methods

The slowest-growing clones of the generated burden-addicted E. coli strains (s9.0 #8; s5.0 #3; and s7.0 #8) were made electrocompetent and transformed by standard electroporation methods (1800 V, 25 μF, 200 Ohms, 1 mm cuvette width) with respectively pEG34 or pEGO (Table 4), and plated on LB agar plates containing chloramphenicol and kanamycin. Single colony transformants were cultured over-night on 2xYT medium containing chloramphenicol, and then used to inoculate 96-well microtiter plates comprising 200 μL 2xYT medium (500 μM IPTG, chloramphenicol) and cultured at 37° C. with fast horizontal shaking in an Elx808 plate reader (Biotek) with reads at OD630 every ten minutes. OD630 values were background-subtracted using the OD630 value of the first read. E. coli strains were each transformed with the plasmid pEG34 or pEGO (Table 4), and their growth rate properties were measured when the cells were induced to synthesize the recombinant protein, hGH fused to GFP.

### 1.3.2 Results

The burden-addicted strain, E. coli strain s9.0 #8, comprising the burden sensing promoter p_{mutM} controlling of the folP-glmM essential genes expression, exhibited the slowest growth of the strains tested when compared to the parent E. coli BL21(DE3) strain (FIG. 2). This reduction of exponential growth rate is reversed in cells of the E. coli strain s9.0 #8 transformed with the pEG34 plasmid and induced to express the recombinant protein, hGH-GFP, in contrast to cells of E. coli strain s9.0 #8 transformed with the empty plasmid, BEG0 (FIG. 3). This demonstrates that the burden-sensing promoter p_{mutM} in E. coli strain s9.0 #8 is induced by the burden or load on the cells brought about by synthesis of the recombinant protein hGH-GFP. This, in turn, leads to up-regulated expression of the folP-glmM genes and enhanced exponential growth.

The synthesis of hGH-GFP in each of the burden-addicted strains, E. coli strain s5.0 #3, s7.0 #8, and s9.0 #8 harboring the pEG34 plasmid, not only led to a reversal of the reduction of exponential growth rate; but additionally the growth rate of these strains was indistinguishable from growth of the parent E. coli BL21(DE3) (FIG. 4).

In summary, this example illustrates that the burden-addiction genetic circuit can be used to confer a selective fitness advantage on those cells of a cell factory whose synthesis of proteins or metabolites is sufficiently high to constitute a burden or load; and that this burden or load is detectable by a burden-sensing promoter operably linked to an essential gene in the cells. By contrast, non-productive variant cells (e.g. resulting from production gene mutations), that spontaneously appear during cultivation of the burden-addicted cell factory, will be subject to a negative selection pressure, since their growth will be slowed to a rate supported by the basal expression of the essential gene. A reduced growth rate is, of itself, sufficient to slow the increase in frequency of such variant cells in the cell factory and thereby delay the decline in cell factory productivity over time.

### Example 2: Burden Addicted E. coli Strains Producing Human Growth Hormone Show Enhanced Long-term Production Stability

Burden-sensing promoters are shown to be promoters that can sense and be activated by a burden-induced state in a cell resulting from the cells synthesis of a recombinant protein, hGH-GFP. Once activated, burden-sensing promoters are shown to elevate expression of an essential gene, folP-glmM, to a level sufficient to confer a selective growth advantage on a cell when compared to a non-productive cell. In order to maximize the dynamic range of essential gene expression in response to its cognate burden sensing promoter, the burden-sensing promoter is randomly combined with variant RBS coding sequences (Table 3) conferring different translational strengths.

Promoters having burden-sensing properties suitable for use in a burden-addiction genetic circuit are shown to include heat-shock-, DNA damage-, oxidative stress response-promoters and rRNA promoters, as illustrated by the following engineered production strains cultured under simulated large-scale production conditions.

Firstly, a number of clones harboring random variant RBS coding sequences were selected for each of the burden-sensing promoters, and their hGH-GFP synthesis was followed over many cell divisions in order to demonstrate their relative ability to elevate/preserve recombinant hGH-GFP synthesis over time.

### 2.1 Materials and Methods

Burden-sensing promoters: The promoters, p_{yccV}, p_{ycjX}, p_{ibpA}, p_{grpE}, p_{ldhA} and p_{ybbN}, and their respective integration sequences were generated by PCR and USER cloning as described for Example 1.2.1, using the primers specified in Table 6; while the ligated products were amplified using primers ‘rev’ (according to specific promoter in Table 6) and P493 (Table 5).

TABLE 6
Primers

| Promoter | | | |
|---|---|---|---|
| of E. coli | | | SEQ ID |
| gene | Primer name | Primer sequence (5′-3′)* | No.: |
| ibpA | ibpA fwd | ATCCCCATTACGTGAGGATGGCTAAGGTCCAGT | 122 |
| | | GAAGTACCCTGGGCAAAGAGTTTCATaccttNCA | |
| | | AGTAATCAATAGCTCCTGAAATC | |
| | ibpA rev | ATTCATATGTTTGGCCTGATGAGTTATAGCG | 123 |
| grpE | grpE fwd | ATTCATATGUTCTCCGCGAGCGTGCCAGTTTT | 124 |
| | grpE rev | ATCCCCATTACGTGAGGATGGCTAAGGTCCAGT | 125 |
| | | GAAGTACCCTGGGCAAAGAGTTTCATaccttNCA | |
| | | AGTGAATTTCTCCGCGTTTTTTT | |
| ldhA | ldhA fwd | ATTCATATGUGGGAACCCACAGCCCGAGCGTC | 126 |
| | ldhA rev | ATCCCCATTACGTGAGGATGGCTAAGGTCCAGT | 127 |
| | | GAAGTACCCTGGGCAAAGAGTTTCATaccttNCA | |
| | | AGTAAGACTTTCTCCAGTGATGT | |
| ybbN | ybbN fwd | ATTCATATGUCCCATTCGTACTCGCTTCACCGAT | 128 |
| | | CCCCATTACGTGAGGATGGCTAAGGTCCAGTGA | |
| | | AGTACCCTGGGCAAAGAGTTTCATaccttNCAAG | |
| | | TGGAGTCGCTCTCTGTTGTCG | |
| | ybbN rev | ATCCCCATTACGTGAGGATGGCTAAGGTCCAGT | 129 |
| | | GAAGTACCCTGGGCAAAGAGTTTCATaccttNCA | |
| | | AGTGGAGTCGCTCTCTGTTGTCG | |
| fxsA | fxsA fwd | ATTCATATGUTAAACACACAGAATATATGTGG | 130 |
| | fxsA rev | ATCCCCATTACGTGAGGATGGCTAAGGTCCAGT | 131 |
| | | GAAGTACCCTGGGCAAAGAGTTTCATaccttNCA | |
| | | AGTAGGTTTCTCCTGTAATAGCA | |
| rrn B | rrnB fwd | ATTCATATGUGCAAACACGCCGCCGGGTCAG | 132 |
| | rrnB rev | ATCCCCATTACGTGAGGATGGCTAAGGTCCAGT | 133 |
| | | GAAGTACCCTGGGCAAAGAGTTTCATaccttNCA | |
| | | AGTAAAAGTTTGACGCTCAAAGA | |
| rrnE | rrnE fwd | ATTCATATGUAATGCGCCTCCGTTGAGACGACA | 134 |
| | | AC | |
| | rrnE rev | ATCCCCATTACGTGAGGATGGCTAAGGTCCAGT | 135 |
| | | GAAGTACCCTGGGCAAAGAGTTTCATaccttNCA | |
| | | AGTAAAAGTTTGATGCTCAAAGA | |
| poxB | poxB fwd | ATTCATATGUTAGGTTGTCGCTGCCTGCCGTG | 136 |
| | poxB rev | ATCCCCATTACGTGAGGATGGCTAAGGTCCAGT | 137 |
| | | GAAGTACCCTGGGCAAAGAGTTTCATaccttNCA | |
| | | AGTGGTTCTCCATCTCCTGAATG | |

*RBS complementary coding sequences are in bold font; wherein N is either A, G, T or C. The RBS is encoded on the opposite strand of the oligo sequence shown.

Chromosomal integration of burden-addiction promoters: each of the promoters was integrated upstream of the folP gene in the genome of E. coli strain BEG34 (corresponding to E. coli BL21(DE3) harboring plasmid pEG34), as follows. Cells of the E. coli strain BEG34, pre-transformed with the recombineering plasmid pSIM5-tet, were prepared and transformed by electroporation with each of the promoter integration sequences, as described in example 1.2.1. Following the described steps of recombineering and curing of pSIM5-tet, eight colonies were picked from each plate, corresponding to eight clones with the same promoter integration but having a random variant RBS sequence (see Table 6). Each clone was then transferred to a well of a 96-well plate containing 200 μl 2xYT supplemented with chloramphenicol for maintenance of the plasmid, pEG34. 2 μl of each cultured clone was used to validate promoter integration via colony PCR as described in example 1.2.1; prior to freezing the 96-well plate.

Short term hGH-GFP production screening assay: The frozen 96-well plate was thawed and a pin replicator used to transfer cells into a new 96-well plate containing 200 μL 2xYT supplemented with chloramphenicol. This plate was sealed with Breathe-Easy sealing membrane (Sigma-Aldrich) and placed in a SynergyH1 plate reader (Biotek) overnight at 37° C. and 754 rpm linear shaking for 20 hours; and the OD (600 nm) and GFP fluorescence (ex/em 485 nm/528 nm) of each well was measured every 10 minutes over a 20 hour period. The plate was then placed in a regular tabletop plate shaker at room temperature for 4 hours followed by the transfer of 2 μL culture from each well to a new 96-well plate containing 200 μL 2xYT supplemented with chloramphenicol and 0.5 mM IPTG. The new 96-well plate was likewise sealed with Breathe-Easy sealing membrane (Sigma-Aldrich) and placed in a SyngergyH1 plate reader overnight at 37° C. and 754 rpm linear shaking for 20 hours; and the OD and GFP fluorescence of each well was measured at 600 nm and ex/em 485 nm/528 nm every 10 minutes for 20 hours. The following day 2 μL culture was transferred to a new 96-well plate with 200 μl 2xYT supplemented with chloramphenicol and 0.5 mM IPTG and the process was repeated every day for 6 days in total.

Long term hGH-GFP production assay (FIG. 6): A single selected colony from each burden-addicted hGH-GFP producing strain was used to inoculate a 15 mL Greiner culture tube containing 4 mL 2xYT supplemented with chloramphenicol and 0.5 mM IPTG. The cultures were grown for 23 hours at 37° C. on a 250 rpm shaking table. 2 μL of each culture were then seeded in 2000× dilution (corresponding to approx. 11 generations of cell division) into a new Greiner culture tube under identical conditions, where these method steps were repeated for a total of 12 seeds. After each passage, 200 μl samples of the culture were taken to determine hGH-GFP synthesis by measuring OD600 and GFP fluorescence (ex/em 485 nm/528 nm) in a SynergyH1 plate reader (Biotek).

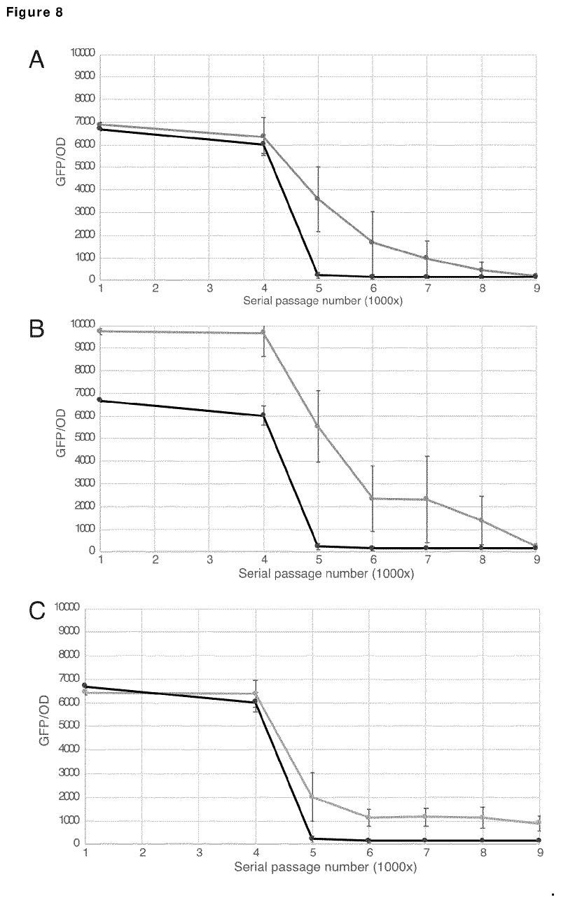

Long term hGH-GFP production assay (FIG. 8): A single selected colony from each burden-addicted hGH-GFP producing strain was used to inoculate a 24 deep-well plate carrying 1.8 mL 2xYT supplemented with chloramphenicol and 0.5 mM IPTG. The cultures were grown for 23 hours at 30° C. on a 200 rpm shaking table. 2 μL of each culture were then seeded in 1000× dilution (corresponding to approx. 10 generations of cell division) into a new deep well plate under identical conditions, where these method steps were repeated for a total of 9 seeds. After each passage, 200 μl samples of the culture were taken to determine hGH-GFP synthesis by measuring OD600 and GFP fluorescence (ex/em 485 nm/528 nm) in a SynergyH1 plate reader (Biotek).

Growth rate measurements of chosen strains: Selected high-hGH producing strains, s3.6 #2, s6.6 #6, s7.6 #8 and s10.6 #7, were streaked on LB agar plates containing chloramphenicol and kanamycin. BEG34 and BEG0 strains were streaked on LB agar plates containing chloramphenicol. 7 colonies from each plate were used to inoculate a 96-well plate containing 200 μl 2xYT supplemented with chloramphenicol; which were sealed with Breathe-Easy sealing membrane (Sigma-Aldrich). Their growth was measured in a SynergyH1 plate reader (Biotek) overnight at 37° C. and 754 rpm linear shaking. 2 μl samples from each well were transferred to wells in a new 96-well plate containing 200 μl 2xYT supplemented with chloramphenicol and 0.5 mM IPTG. The 96-well plate was sealed with breathe-easy film and incubated for 20 hours at 37° C. and 754 rpm linear shaking and growth (OD600 nm) was measured every 10 minutes using a Synergy H1 plate reader.

Strain catalogue:
BEG34=E. coli BL21(DE3) carrying pEG34;
BEG0=E. coli BL21(DE3) carrying empty plasmid pEG0
sX.Z #Y=E. coli BL21(DE3) carrying pEG34, having wt folP promoter swapped for burden addiction-promoter X, wherein X refers to the promoter IDs in Table 2. The number Y following #indicates the selected clone from the pool of four RBS variants introduced by degenerate primer (Table 7). Z refers to the production genes for hGH-GFP (0 or 6), mevalonic acid (1), lysostaphin (4), respectively.

TABLE 7
Identity of the “N” nucleotide of the RBS of the respective E. coli strains

| Strain | | RBS “N” | Strain | | RBS “N” | Strain | | RBS “N” |
|---|---|---|---|---|---|---|---|---|
| identifier | | identity | identifier | | identity | identifier | | identity |
| s5.0 | #3 | C | s16.6 | #6 | A | s19.6 | #2 | A |
| s7.0 | #8 | A | s18.6 | #8 | T | s21.6 | #8 | C |
| s9.0 | #8 | NGS | s9.1 | #1 | C | s23.6 | #4 | T |
| s6.4 | #5 | C | s15.1 | #1 | A | s24.6 | #2 | T |
| s3.6 | #2 | A | s19.1 | #1 | T | s25.6 | #1 | C |
| s6.6 | #6 | C | s14.1 | #5 | T | s29.6 | #3 | T |
| s7.6 | #8 | T | s15.1 | #5 | A | s22.6 | #8 | T |
| s10.6 | #7 | A | s19.1 | #7 | A | s26.6 | #3 | A |
| s13.6 | #2 | C | s15.6 | #1 | A | s29.6 | #4 | T |
| s15.6 | #7 | A | s16.6 | #7 | T | s30.6 | #6 | T |

### 2.2 Results

2.2.1 Short Term hGH-GFP Productivity Screening

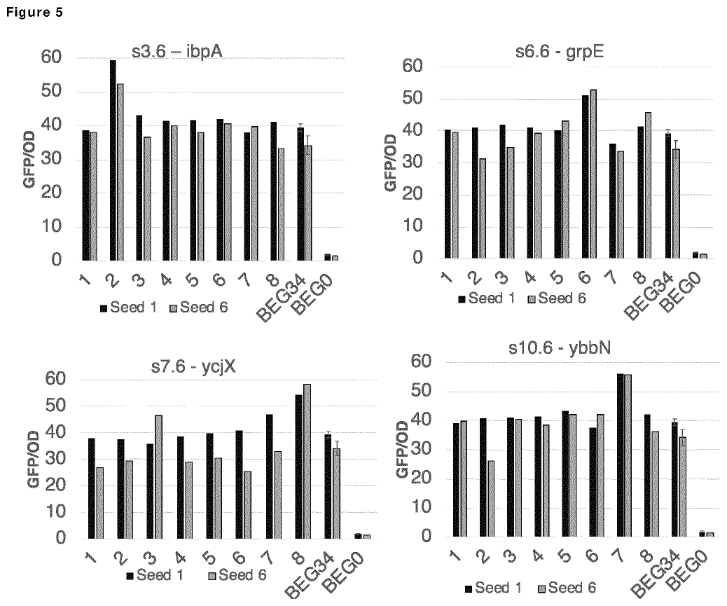

The productivity of four hGH-GFP producing strains comprising burden-sensing promoters selected from a group of heat shock promoters (p_{ibpA}, p_{orpE}, p_{ycjX}, and p_{ybbN}) and having one of four RBS coding sequence variants was tested under simulated large-scale production conditions, as follows. The strains were serially passaged by 100× back-dilution every day for 6 consecutive days corresponding to ca. 6 generation per seed. By day 6 (seed 6), several strains showed an elevated hGH-GFP synthesis compared to the non-burden addicted production strain BEG34, both during seed 1 and seed 6. The strains having high short term hGH-GFP productivity relative to strain BEG34 are s3.6 #2 (p_{ibpA}), s6.6 #6 (p_{grpE}), s7.6 #8 (p_{ycjX}), and s10.6 #7 (p_{ybbN})-see FIG. 5.

2.2.2 Enhanced Long Term hGH-GFP Productivity

hGH-GFP productivity of burden-addicted strains, s3.6 #2, s6.6 #6, s7.6 #8, s10.6 #7, and non-burden addicted strains BEG34 and non-producing strain BEG0 was monitored under simulated large-scale fermentation by serial transfer. Following seed 2, burden-addicted strains s3.6 #2, s6.6 #6, s7.6 #8, s10.6 #7 and the non-burden addicted control strain BEG34 performed equally well in terms of hGH-GFP synthesis (FIG. 6. However, after an additional ca. 20 generations (seed 4), hGH-GFP productivity by the control strain, BEG34, had declined considerably; and by seed 6, the strain essentially ceased production. Surprisingly, the burden-addicted strain s7.6 #8 (with p_{ycjX} controlling folP-glmM) still retained a productivity of around 50% of seed 2 level. Additionally, burden-addicted strains s3.6 #2 (with p_{ibpA} controlling folP-glmM), s10.6 #7 (with p_{ybbN} controlling folP-glmM), and s6.6 #6 (with p_{grpE} controlling folP-glmM) also showed significantly better long-term productivity as compared to the non-burden addicted strain BEG34.

### 2.2.3 Growth Rate of Burden Addicted Strains

The growth rates of hGH-GFP producing burden-addicted strains s3.6 #2, s6.6 #6, s7.6 #8 and s10.6 #7 is no higher than the non-burden addicted hGH-GFP production strain BEG34; and hence their improved hGH-GFP production levels over time does not to simply stem from a reduced in initial production level and thus inherently lower burden, (FIG. 7).

### 2.2.4 Dependence of Burden Addiction Genetic Circuit on Selected Essential Gene

A hGH-GFP producing burden-addicted strain (s13.6 #2 (fxsA)) comprising the heat shock promoter p_{fxsA} controlling the essential gene folP-glmM was compared with a mutant derivative comprising a frame-shift folP abolishing folP expression, strain s13.6 #2evo (fxsA), whose growth was solely dependent on glmM expression. Although the loss of folP expression led to a lower growth rate (data not shown) in complex 2xYT medium, the essential gene glmM, alone is shown to be sufficient to confer enhanced long-term hGH-GFP production stability in the burden-addicted strain s13.6 #2evo (fxsA) at levels comparable to the strain s7.6 #8 (pycjX), when compared to the non-burden addicted hGH-GFP producing strain BEG34 (FIG. 8).

### 2.2.5 Use of Ribosomal RNA Promoters in the Burden Addiction Genetic Circuit

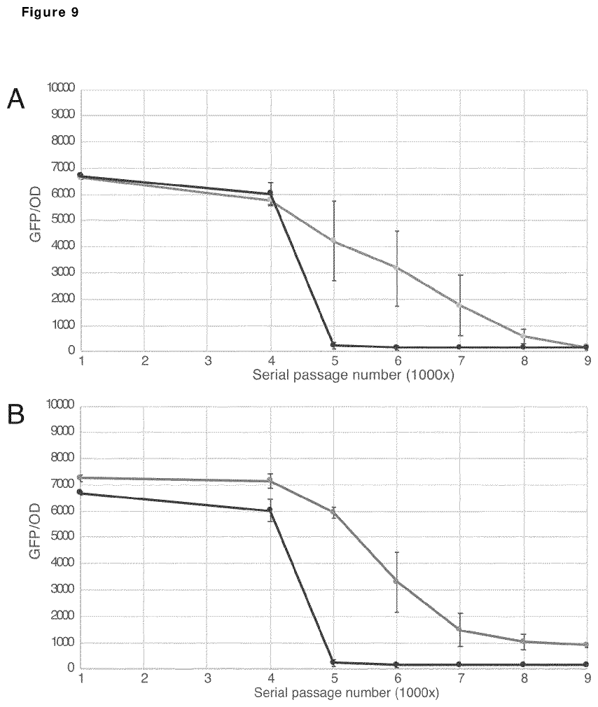

In addition to heat-shock promoters, ribosomal RNA promoters such as p_{rrnB} and p_{rrnE} are shown to be capable of sensing a burden-addicted state in a cell and, in response, to control essential gene expression such as to enhance long-term productivity in a hGH-GFP production strain. As seen in FIG. 9, the long-term stability of hGH-GFP production in strains s15.6.7 (p_{rrnB}) and s16.6.6 (p_{rrnE}), was significantly improved when compared to a non-burden addicted hGH-GFP producing E. coli strain BEG34, under simulated large scale production corresponding to around 110 generations of cell division.

### 2.2.6 Use of Oxidative Stress Sensing Promoters in the Burden Addiction Genetic Circuit

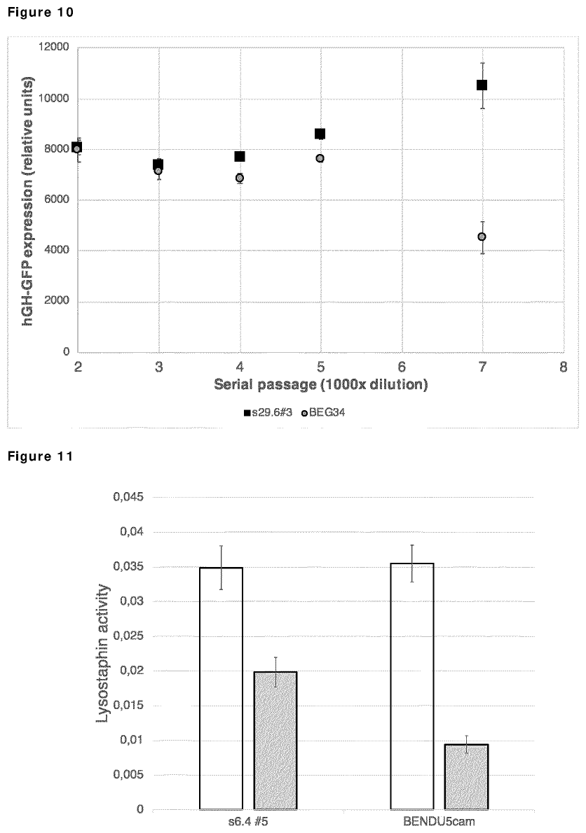

Oxidative stress-sensing promoters such as p_{poxB} are shown to be capable of sensing a burden-addicted state in a cell and, in response, to control essential gene expression such as to enhance long-term productivity in a hGH-GFP production strain. As seen in FIG. 10, both production levels and long-term stability of hGH-GFP production in strain s29.6 #3 (p_{poxB}), was significantly increased when compared to a non-burden addicted hGH-GFP producing E. coli strain BEG34, under simulated large scale production of seven serial passages corresponding to approximately 90 generations and time-extended cultivation for the final seed. As seen in FIG. 10, production using the burden addiction genetic circuit based on p_{poxB} surprisingly resulted in elevated production over time, indicative of the enrichment of non-genetically high performing variants in the population.

In summary, this example illustrates that burden addiction enhances long-term stability and production in E. coli strains engineered to synthesize human growth hormone fused to GFP by coupling transcription of essential genes folP-glmM to any one of a E. coli heat shock, oxidative stress and DNA damage responsive promoter as well as an rRNA promoter.

### Example 3: Burden Addicted E. coli Strains Producing Lysostaphin Show Enhanced Long-term Production Stability

Lysostaphin is a 27 kDa endopeptidase that cleaves crosslinking pentaglycine bridges in the cell wall peptidoglycan of Staphylococcus aureus resulting in cell lysis. This antibacterial agent can be synthesized recombinantly in E. coli. The burden-addiction genetic circuit comprising a promoter having burden-sensing properties is shown to enhance the production stability of such E. coli strains engineered to synthesize lysostaphin, as illustrated by the following burden-addicted E. coli strains producing lysostaphin under simulated large-scale production conditions. The productivity of the burden-addiction production strains is further shown to be optimized by selecting a strain where the burden-sensing promoter is combined with an RBS sequence (Table 6) conferring an optimized translational strength of the cognate essential gene. These advantages are exemplified by burden-addicted strain s6.4 #5 (p_{grpE}), described below, that is shown to be more resilient to loss of production due to addiction to the burden or fitness cost resulting from lysostaphin expression.

### 3.1 Materials and Methods

Burden-sensing promoters: A promoter integration sequence comprising the promoter p_{grpE} was generated as described for example 1.2.1, by amplifying the p_{grpE} promoter by PCR, using the p_{grpE}-specific primers specified in Table 6.

Chromosomal integration of burden-addiction promoters: the grpE promoter was integrated upstream of the folP-glmM gene in the genome of E. coli strain BENDU5cam (corresponding to E. coli BL21(DE3) harboring a lysostaphin producing plasmid pENDU5cam), as follows. Cells of the E. coli strain BENDU5cam, pre-transformed with the recombineering plasmid pSIM5-tet, was prepared and transformed by electroporation with the p_{grpE} promoter integration sequence, as described in example 1.2.1. Following the described steps of recombineering and curing of pSIM5-tet, 5 colonies were picked, corresponding to clones with the same promoter integration but having a random variant RBS sequence (see Table 6). Each clone was then transferred to a 15 mL Greiner culture tube containing 4 mL 2xYT supplemented with chloramphenicol for maintenance of the plasmid, pENDU5cam, and cultured. 2 μl of each cultured clone was used to validate promotor integration as described in example 1.2.1; prior to freezing.

Lysostaphin production screening: The E. coli strain s6.4 #5 (p_{grpE}) having the highest lysostaphin-productivity was identified using a screening assay in which the three other potential combinations of RBS and p_{grE} were cultured in 2xYT supplemented with chloramphenicol in 96-well format over six serial passages of 1000× dilution (corresponding to 60 generations) as compared to the non-burden addicted production strain BENDU5cam. The strains were grown in 3 ml 2xYT media containing 30 mg/L chloramphenicol in 15 mL culture tubes at 37° C. for 21 hours. 150 μL of the cultures were mixed with 50 μl 50% glycerol and stored in a 96-well plate at −80° C. Additionally, 30 μL of the cultures were transferred to 3 ml fresh 2xYT media supplemented with chloramphenicol and grown for another 21 hours at identical conditions; and repeated for a total of 5 transfers where freeze-stocks were made of the overnight cultures with each transfer.

Lysostaphin expression and detection assay: The 96-well plate containing all freeze-stocks from the 5 transfers were thawed on ice. 20 μL from each well were transferred to a 96-well plate containing 180 μL 2xYT supplemented with chloramphenicol, and cultured grown at 37° C. in a plate reader until most wells had reached OD630=0.35; and then 10 μl 2xYT supplemented 30 mg/L chloramphenicol and 20 mM IPTG was added to each well to a final concentration of 1 mM IPTG, sufficient to induce lysostaphin gene expression. The induced cultures were grown at 37° C. for 4 hours, and the plate was then centrifuged at 4000 RPM for 10 minutes. 100 μl supernatant was transferred to a new 96-well plate containing 100 μl overnight S. aureus culture. The S. aureus lysis in each well was monitored at OD630 on a plate reader at 37° C. for 2 hours every 10 minutes. The S. aureus lysis rate was quantified by dividing the change in S. aureus OD by the timeframe of 60 minutes that the change occurred in. The equation can be seen below:

$${Rate}_{lysis} = \frac{{OD}_{{t}_{0}} - {OD}_{{t}_{1}}}{{t}_{1} - {t}_{0}}$$

The specific lysostaphin synthesis rate was determined by normalizing the measured rate to the final OD630 measurement of the respective production E. coli culture.

### 3.2 Results

The burden-addicted lysostaphin-producing strains having a p_{grpE} promoter and one of four RBS coding sequence variants controlling expression of the essential genes, folP-glmM, were cultivated under simulated large-scale production conditions, achieved by serial passaging. As seen in FIG. 11, strain s6.4 #5 (p_{grpE}) maintained significantly higher levels of lysostaphin production as compared to the steep reduction in production seen in the non-burden addicted lysostaphin-production BENDU5cam strain. Since the initial lysostaphin production rates for s6.4 #5 (p_{grpE}) and BENDU5cam strains at seed 0 were the same; the smaller decline in production rates for s6.4 #5 (p_{grpE}) is not due to an inherently lower initial burden from lysostaphin production.

In summary: Burden-addiction was demonstrated to enhance long-term stability and production in E. coli engineered to synthesize secreted lysostaphin by coupling essential genes folP-glmM transcription to the E. coli heat shock promoter p_{grpE}.

### Example 4: Burden Addicted E. coli Strains Producing Mevalonic Acid Show Enhanced Long-term Production Stability

E. coli BL21(DE3) cells were engineered to synthesize mevalonic acid by introducing a plasmid pMevT expressing a heterologous three-step enzymatic pathway (Martin et al., 2003) that converts glucose to mevalonic acid via the acetyl-CoA pool. A burden-addiction genetic circuit comprising a promoter having burden-sensing properties and controlling transcription of an essential gene is shown to enhance the production stability of such mevalonic acid-producing E. coli strains under simulated large-scale production conditions. The productivity of the burden-addiction production strains is shown to be further optimized by selecting a strain where the burden-sensing promoter in combined with a RBS coding sequence (Table 5) conferring an optimized translational strength of the cognate essential gene. Specifically, mevalonic acid-production strains comprising the burden-addiction promoter, p_{cspD} (an oxidative stress and glucose starvation sensing promoter), or p_{mutM} (heat shock/DNA damage-sensing promoter) controlling folP-glmM transcription, are both shown to be more resilient to loss of production than the non-burden addicted strain due to their addiction to transcriptional signals resulting from mevalonic acid production.

### 4.1 Materials and Methods

Burden-sensing promoters: Promoter integration sequences comprising the promoters p_{mutM} and p_{cspD} were generated as described for example 1.2.1, by amplifying the p_{mutM} and p_{cspD} promoters by PCR, using the p_{mutM}-and p_{cspD}-specific primers specified in Table 8.

TABLE 8
Primers

| Promoter | | | |
|---|---|---|---|
| of E. coli | | | SEQ ID |
| gene | Primer name | Primer sequence (5′-3′)* | No.: |
| mutM | mutM for | ATTCATATGUGGCGAAAAGTACTAAGTACTTA | 116 |
| | mutM rev | ATCCCCATTACGTGAGGATGGCTAAGGTCCAGTGAA | 117 |
| | | GTACCCTGGGCAAAGAGTTTCATaccttNCAAGTAGC | |
| | | ATCTCCAGGAATGAACA | |
| cspD | cspD fwd | ATTCATATGUCGGTTATCGGCAGAACGCCCTG | 138 |
| | cspD rev | ATCCCCATTACGTGAGGATGGCTAAGGTCCAGTGAA | 139 |
| | | GTACCCTGGGCAAAGAGTTTCATaccttNCAAGTGCT | |
| | | TCGACATCCTTCGCAAA | |

*RBS complementary coding sequences are in bold font; wherein N is either A, G, T or C. The RBS is encoded on the opposite strand of the oligo sequence shown.

Chromosomal integration of burden-addiction promoters: the p_{mutM} and p_{cspD} were individually integrated upstream of the folP-glmM gene in the genome of cells of the E. coli strain BL21(DE3) pMevT, harboring a lysostaphin producing plasmid pMevT, as follows. Cells of the E. coli strain BL21(DE3) pMevT, pre-transformed with the recombineering plasmid pSIM5-tet, were prepared and transformed by electroporation with each of the promoter integration sequences, as described in example 1.2.1. Following the described steps of recombineering and curing of pSIM5-tet, 5 colonies were picked, corresponding to clones with the same promoter integration but having a random variant RBS sequence (see Table 8). Each clone was then transferred to a well of a 96-well plate containing 200 μl 2xYT supplemented with chloramphenicol for maintenance of the plasmid, pMevT, and cultured. 2 μl of each cultured clone was used to validate promotor integration as described in example 1.2.1; prior to freezing the 96-well plate.

Mevalonic acid production screening: The E. coli strains s19.1.1 (p_{cspD}) and s9. 1.4 (p_{mutM}) having the highest mevalonic acid productivity were identified using a screening assay in which strains comprising the four other potential combinations of RBS and promoter were cultured in 96-well plates over six serial passages of 1000× dilution (corresponding to 60 generations) in parallel with the non-burden addicted production E. coli strain BL21(DE3) pMevT, as follows. The strains were grown in 200 μL 2xYT media containing 30 mg/L chloramphenicol and 0.5 mM IPTG in microtiter plate sealed with breathe-easy seal at 37° C. for 21 hours with horizontal shaking. 150 μL of the cultures were mixed with 50 μl 50% glycerol and stored in a 96-well plate at −80° C. Additionally, 2 μl of a 10-fold diluted culture was transferred to 200 μL fresh 2xYT media supplemented with chloramphenicol and 0.5 mM IPTG and grown for another 21 hours under identical conditions. In total, 5 transfers identical to the one described above were made and freeze-stocks were made of the overnight cultures with each transfer.

Mevalonic acid synthesis and detection assay: The 96-well plate containing freeze-stocks from the second, fifth and sixth transfers were thawed on ice and used to inoculate 10 mL 2xYT with 0.5 mM IPTG and 30 mg/L chloramphenicol and cultivated at 37° C. with horizontal shaking (250 rpm) for 54 hours. 300 μL aliquots from each culture were treated with 23 μL 20% sulfuric acid; vigorously shaken and then spun down at 13 000× g for 2 min. Supernatant (medium) samples were injected into an Ultimate 3000 high-performance liquid chromatography running a 5 mM sulfuric acid mobile phase (0.6 mL/min) on an Aminex HPX-87H ion exclusion column (300 mm ×7.8 mm, Bio-Rad Laboratories) at 50° C. A refractive index detector was used for detection. A standard curve for mevalonic acid was generated with mevalonolactone (Sigma-Aldrich) dissolved in 2xYT medium supernatant of a non-producing E. coli strain incubated under the same conditions.

### 4.2 Results

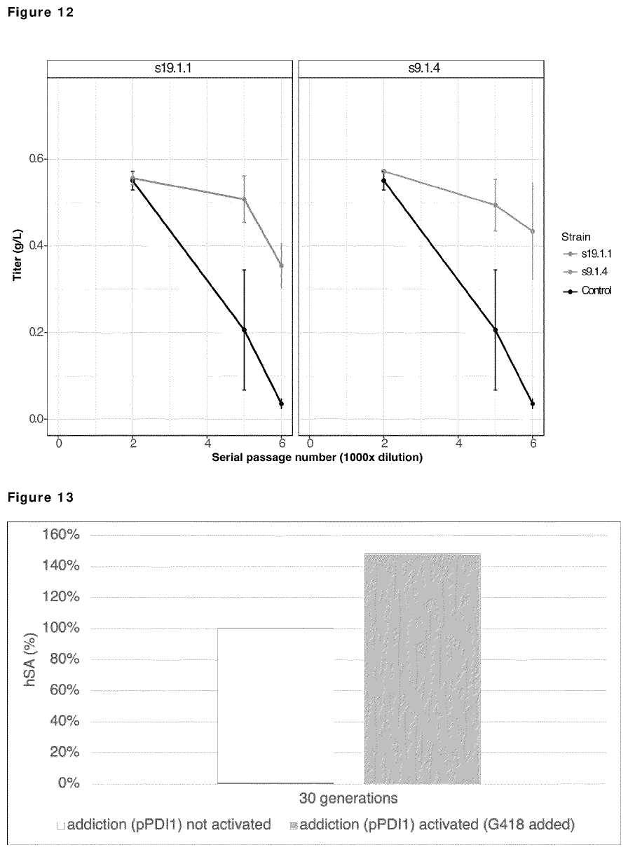

The burden-addicted mevalonic acid-producing strains having the p_{cspD} or p_{mutM} promoters and one of four RBS coding sequence variants controlling expression of the essential genes, folP-glmM, were cultivated under simulated large-scale production conditions, achieved by serial passaging. As seen in FIG. 12, both selected burden-addicted strains s19.1.1 (p_{cspD}) and s9.1.4 (p_{mutM}) retained significantly higher levels of mevalonic acid production as compared to the steep reduction in production seen in the non-burden addicted mevalonic acid-production strain BL21(DE3) pMevT. Since the initial mevalonic acid production rates for each burden-addicted strain was the same and the control BL21(DE3) pMevT strain at seed 0; the smaller decline in production rates for strains s19.1.1 (p_{cspD}) and s9.1.4 (p_{mutM}) is not due to an inherently lower initial burden from mevalonic acid production.

In summary: Burden-addiction is demonstrated to enhance long-term stability and production in E. coli engineered to synthesize mevalonic acid by coupling essential genes folP-glmM transcription to the E. coli oxidative stress and glucose starvation sensing promoter p_{cspD}, and the heat shock/DNA damage-sensing promoters p_{mutM}.

### Example 5: Evaluation of Promoters as Burden Sensors to Render Yeast Cells Addicted to the Burden of Engineered Recombinant Protein Production

Promoters capable of sensing the burden or load on a cell brought about by recombinant expression of a protein or a biosynthetic pathway, and that then induce the expression of an essential gene, can be used to create a burden addiction genetic circuit tailored for use in yeast. A method for evaluating candidate promoters is illustrated in yeast cells genetically engineered to synthesize recombinant human serum albumin (hSA) or insulin precursor (IP), optionally translationally fused to green fluorescent protein (GFP).

To render growth responsive to the activity of a candidate burden-sensing promoter, the native promoter of an essential gene in the yeast Saccharomyces cerevisiae is genetically replaced with a candidate promoter using homologous recombination of linear DNA constructs transformed into the yeast cells using a standard selectable marker. By way of example, candidate promoters may be selected from upregulated promoters of ribosomal RNA genes, such as those transcribed by RNA polymerase I (Laferté et al., 2006), DNA damage sensing promoters (e.g. pOGG1), and unfolded protein response (UPR) promoters upregulated by the HAC1 transcription factor (Kimata et al., 2006). In order to ensure that the burden-sensing promoter, once activated, confers the cell with a selective growth advantage compared to a non-sensing promoter, it may be necessary to fine-tune the expression level of essential gene. A range of translational strengths can be engineered by varying the translation initiation region introduced with the burden-sensing promoter.

Clones with potentially different combinations of candidate burden-sensing promoters are selected and evaluated for maintained protein production over 30-100 generations of cell division.

### 5.1 Materials and Methods

Growth medium: YPD medium comprises 1% yeast extract, 2% peptone, 2% glucose. SC medium comprises 6.7 g/L yeast nitrogen base without amino acids and with ammonium sulfate, but lacks uracil.

Chromosomal integration and validation of promoter constructs: Constructs for chromosomal integration will contain 300-600 bp upstream of the natively regulated gene. Chromosomal integration of the promoter construct is performed by transformation and homologous recombination using standard electroporation procedures for S. cerevisiae. Correct chromosomal integration of the promoter was validated using colony PCR.

Long-term cultivation and production: A single colony from each strain is transferred to 24-deep well plates and cultivated in 1.8 mL YPD medium under conditions inducing recombinant protein production, at 250 rpm and 30° C. Following 48 hours of cultivation, cells are passaged to a new deep-well plate under identical conditions by 1000× back-dilution. Samples are analyzed for production using recombinant protein specific assays (e.g. GFP detection) and the cell density is monitored by OD600.

Growth rate measurements of selected strains: Growth rates of individual strains are compared to non-burden addicted production strains. The 96-well plate is sealed with breathe-easy film and growth is measured in a Synergy H1 plate reader for 20 hours at 37° C. and 754 rpm linear shaking. OD600 is measured every 10 minutes.

### 5.2 Results

5.2.1 HAC1-upregulated Promoters

The HAC1-upregulated promoters comprising an unfolded protein response (UPR) element, e.g. KAR2 (SEQ ID NO.: 91), PDI1 (SEQ ID NO.: 92), SSA1 (SEQ ID NO.: 93) or FPR2 (SEQ ID NO.: 94), are demonstrated to be useful in regulating growth when introduced in front of a native growth regulating gene (e.g. the conditionally essential gene URA3 encoding orotidine 5′-phosphate decarboxylase essential for pyrimidine biosynthesis) in a recombinant protein production Saccharomyces strain producing human insulin precursor or human serum albumin, optionally coupled to GFP. Stability of production is followed under simulated long-term production via serial passages corresponding to 60-80 generations of cell division. In Saccharomyces strains comprising the HAC1-upregulated promoter controlling transcription of the growth-regulating gene (URA3), production is expected to be more stable than the corresponding parent recombinant protein production Saccharomyces strain.

### 5.3.2 RNA Polymerase I Upregulated Promoters

RNA polymerase I transcribe ribosomal RNA genes in yeast. RNA polymerase I upregulated promoters such as promoters of the genes: RPL3 (SEQ ID NO.: 95), RPL6A (SEQ ID NO.: 96) and RPL28 (SEQ ID NO.: 97) are useful in regulating growth of yeast essential genes. Such upregulated promoters are introduced in front of native growth regulating gene (e.g. the conditionally essential gene URA3) in a recombinant protein overproduction strain producing human insulin precursor or human serum albumin, potentially coupled to GFP. Stability of production is followed by experimentally simulated long-term production via serial passages corresponding to 60-80 generations of cell division. In Saccharomyces strains comprising an RNA polymerase I upregulated promoter controlling transcription of the conditionally essential gene, URA3, production will be more stable than the corresponding parent recombinant protein production Saccharomyces strain.

### 5.3.3 DNA Damage Responsive Promoters

DNA damage response in yeast, resulting from heterologous expression broadly induces transcription of the DNA repair system, including OGG1 (SEQ ID NO.: 98), RAD51 (SEQ ID NO.: 99) and RAD54 (SEQ ID NO.: 100). Promoters of the genes encoding OGG1, RAD51 or RAD54, when operatively linked to an essential gene, are useful for regulating growth of a yeast production cell of the invention. Such promoters are introduced in front of native essential gene (e.g. the growth regulating gene encoding URA3) in cells of a yeast protein production strain producing human insulin precursor or human serum albumin, optionally fused to GFP.

Stability of production is followed by under simulated long-term production via serial passages corresponding to 60-80 generations of cell division. In strains with a p_{RAD51} and/or p_{RAD54}-upregulated essential gene, production will be more stable.

In summary: Burden addiction enhances long-term stability and production in cells of budding yeast engineered to synthesize human serum albumin production or insulin precursor by coupling essential gene transcription to the burden-sensing promoters selected from promoters activated during burden of recombinant protein production or associated with ribosomal RNA promoters (Table 2).

### Example 6 Examples of Burden-addicted Yeast Strains Producing Human Serum Albumin Showing Enhanced Long-term Production Stability

Burden-addiction systems for use in yeast strains include promoters derived from genes encoding: 1) Protein isomerase PDI1 encoding a chaperone belonging to the unfolded protein response of yeasts such as S. cerevisiae and P. pastoris, and whose abundance is frequently upregulated in response to overexpression of a recombinant protein; 2) Ribosomal subunits encoded by the RPL6A and RPL3 genes and 3) FPR2 encoding a peptidyl-prolyl cis-trans isomerase which is known to be activated upon DNA replication stress. Burden-addiction systems based on these promoters were introduced into a strain of Pichia pastoris engineered to express and secrete human serum albumin in order to determine their effect on long term human serum albumin (hSA) production stability.

### 6.1 Materials and Methods:

The Pichia pastoris (Komagataella phaffii) strain EGS31 is a derivative of the CBS7435 strain (NRRL-Y11430 or ATCC 76273), engineered to secrete hSA by a genomically integrated cDNA version of the hSA coding ALB1 gene under control of the AOX1 promoter.

Strain construction: Burden-addicted versions of the EGS31 strain were generated by genetically integrating a construct comprising a kanMX conditionally selectable G418 resistance gene operably linked to one of the burden-responsive promoters: pPDI1, pFPR2, pRPL3 and pRPL6A. These burden-addiction constructs were integrated into the KU70 genomic locus of the P. pastoris strain by transforming EGS31 cells with linear integration DNA (sequences N1-N3 respectively) flanked by >750 bp homology arms. Transformation was performed using a standard electroporation procedure on exponentially grown cells pretreated with lithium acetate and dithiothreitol (Wu & Letchworth, 2004).

TABLE 9
Recombinant genes and burden-addiction constructs introduced into
P. pastoris

| | Construct nucleotide | Encoded protein |
|---|---|---|
| Gene/construct name | SEQ ID NO: | SEQ ID NO .: |
| pAOX1-ALB construct | SEQ ID NO: 155 | hSA protein |
| | | SEQ ID NO: 156 |
| KU70(up)-pPDI1-kanMX- | SEQ ID NO: 157 | kanMX protein |
| terminator-KU70(dw) | | SEQ ID NO: 158 |
| KU70(up)-pFPR2-kanMX- | SEQ ID NO: 159 | kanMX protein |
| terminator-KU70(dw) | | SEQ ID NO: 160 |
| KU70(up)-pRPL3-kanMX- | SEQ ID NO: 161 | kanMX protein |
| terminator-KU70(dw) | | SEQ ID NO: 162 |
| KU70(up)-pRPL6A-kanMX- | SEQ ID NO: 163 | kanMX protein |
| terminator-KU70(dw) | | SEQ ID NO: 164 |

Growth Media: BMGY and BMMY liquid media (1L) was prepared as follows: 10 g yeast extract and 20 g peptone was added to 700 mL H₂O; mixed with a magnetic stirrer and then autoclaved. After cooling to room temperature; the following was added to the solution: 100 ml 1 M potassium phosphate buffer (pH 6.0); 100 ml 13.4% (w/v) Yeast Nitrogen Base with Ammonium Sulfate without amino acids; 2 ml 0.02% (w/v) biotin, and in the case of BMGY, 100 ml 10% (v/v) glycerol was added; and in the case of BMMY, 100 mL 5% (v/v) methanol was added.

Cultivation: EGS31 and burden-addicted EGS31 strains were streaked on YPD (1% yeast extract, 2% peptone, 2% D-glucose) agar plates and incubated overnight at 30 deg. C. Single colonies were picked and pre-cultured in 2 mL BMGY where cultures of burden-addicted strains were supplemented 50 μg/mL G418 and cultured at 30 deg. C. with 300 rpm horizontal shaking overnight. Expression cultures were seeded using 1 μl pre-culture into 500 μL BMMY medium containing different concentrations (0 μg/mL, 750 μg/mL) of G418 in a 96-well deep well plate with aerating lid to generate “seed 1”.

The cultures were incubated at 30 deg. C. for 72 hours (300 rpm horizontal shaking). To generate the next seed, four additional times, grown cultures were serially passaged (500× dilution) to new 500 μL BMMY medium containing different concentrations (0 μg/mL, 750 μg/mL) of G418 in a 96-well deep well plate with aerating lids. At each serial passage, glycerol stocks (20% glycerol) were stored at −80 deg. C. from the grown cultures.

To quantify production of secreted hSA, quantification cultures were re-grown from glycerol stocks in 500 μL BMMY medium containing different concentrations (0 μg/mL and 750 μg/mL) of G418 in a 96-well deep well plate with aerating lid for 72 hours.

Cultures were centrifuged at 3000 g for 15 minutes and the concentration in 50 μL supernatants was quantified using a hSA-specific ELISA kit (Abcam catalog no: ab179887: Human Albumin SimpleStep ELISA® Kit) following manufacturer's instructions.

### 6.2 Results

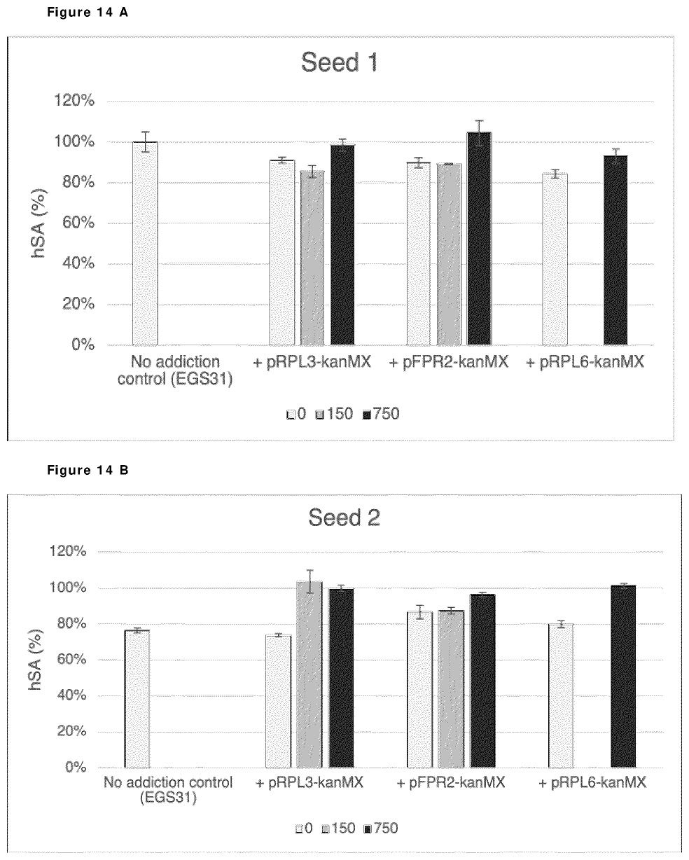

Strains with a genomically integrated burden addiction promoter, PDI1, operably linked to the selectable kanMX gene displayed higher production of secreted hSA when strain's burden-addiction system was activated by addition of G418 (FIG. 13). Further, improved hSA production was seen with higher selection (750 μg/mL G418), for each of the other tested burden-addiction promoters after approximately 30 cell divisions (FIG. 14A-Seed 1). When further cultivated for about an additional 10 cell divisions (FIG. 14B-Seed 2) the strains show enhanced levels of hAS production when burden addiction is activated (150-750 μg/mL G418) compared to the non burden-addicted control and strains without burden addiction activated (0 μg/mL G418).

In conclusion, the cultivation of yeast cells comprising the exemplified burden-addiction systems of the invention, under conditions that activate their respective burden-addiction system, is believed to enrich for high-producing yeast variants within the cultured population. While an increase in hSA production may be detectable after relatively short cultivation (30 cell divisions), enrichment for high-producing yeast variants is both maintained and further enhanced over longer cultivation periods when traditional cultures commonly exhibit a significant decline in productivity.

### Example 7 Identification of Suitable Burden Sensing Promoter Candidates

Different engineered production genes and pathways elicit different transcriptional responses indicative of the production process. In order to identify suitable promoter candidates for use as burden sensors, the following experiment was conducted.

Methods: Typical genetic escaper cells were isolated from long-term cultivation with the genetically engineered microbial production cells of interest in the intended fermentation medium. Suitable genetic escaper cells are characterized by having at least 5% higher exponential-phase growth rate and at least 30% lower production rate or product yield than the original genetically engineered production cell.

Production cells and corresponding escaper cells were cultured under intended fermentation conditions, scaled down conditions or shake flask conditions mimicking intended fermentation conditions. At time points corresponding to the highest rate of production in the production cells, samples were taken for RNA sequencing. Total RNA was purified using Purelink RNA Mini kit (Thermo Fischer) and prepared using TruSeq

Stranded mRNA kit (Illumina) following the kit manufacturer's instructions. Reads were mapped and analyzed to the reference genome of the strain and next analyzed for differential expression between the production cells and corresponding escaper cells.

Results: Candidate suitable promoters were identified as those driving expression of genes that showed a differential expression of >3 fold higher expression in the production organism relative to at least one isolated genetic escape strain.

### Example 8: Burden Addicted Bacillus subtilis Strains Producing IgA Fragment Show Enhanced Long-term Production Stability

Burden-sensing promoters are shown to be promoters that can sense and be activated by a burden-induced state in a cell resulting from the cell's synthesis of a recombinant protein, an IgA fragment C-terminally fused to GFP (IgA-GFP). Once activated, burden-sensing promoters are shown to elevate expression of essential gene (operons), iscU or accC, to a level sufficient to confer a selective growth advantage on a cell when compared to a non-productive cell. In order to maximize the dynamic range of essential gene expression in response to its cognate burden sensing promoter, the burden-sensing promoter is randomly combined with variant RBS coding sequences (Table 3.2) conferring different translational strengths.

Promoters having burden-sensing properties suitable for use in a burden-addiction genetic circuit are shown to include heat-shock-, DNA damage-, and oxidative stress response-promoters, as Mustrated by the following engineered production strains cultured under simulated large-scale production conditions.

### 8.1 Materials and Methods

Burden-sensing promoters: The promoters, p_{hrcA} p_{perR} and their respective integration sequences to target essential genes were generated by PCR and USER cloning resulting in integration vectors (Table 10).

TABLE 10
Bacillus chromosomal integration constructs for inserting burden-sensing
promoter to control essential

| | Promoter- | Resistance gene used to select |
|---|---|---|
| Plasmid | essential gene | for chromosomal integration |
| pEG151 (SEQ ID NO. 195) | P_{groES}-iscU | specR (SEQ ID No. 211) |
| pEG152 (SEQ ID NO. 196) | P_{ctsR}-iscU | specR (SEQ ID No. 211) |
| pEG153 (SEQ ID NO. 197) | P_{dnaK}-iscU | specR (SEQ ID No. 211) |
| pEG154 (SEQ ID NO. 198) | P_{perR}-iscU | specR (SEQ ID No. 211) |
| pEG155 (SEQ ID NO. 199) | P_{hrcA}-iscU | specR (SEQ ID No. 211) |
| pEG156 (SEQ ID NO. 200) | Pₛₚₓ-iscU | specR (SEQ ID No. 211) |
| pEG157 (SEQ ID NO. 201) | P_{sigB}-iscU | specR (SEQ ID No. 211) |
| pEG158 (SEQ ID NO. 202) | P_{yflT}-iscU | specR (SEQ ID No. 211) |
| pEG159 (SEQ ID NO. 203) | P_{groES}-accC | specR (SEQ ID No. 211) |
| pEG160 (SEQ ID NO. 204) | P_{ctsR}-accC | specR (SEQ ID No. 211) |
| pEG161 (SEQ ID NO. 205) | P_{dnaK}-accC | specR (SEQ ID No. 211) |
| pEG162 (SEQ ID NO. 206) | P_{perR}-accC | specR (SEQ ID No. 211) |
| pEG163 (SEQ ID NO. 207) | P_{hrcA}-accC | specR (SEQ ID No. 211) |
| pEG164 (SEQ ID NO. 208) | Psₚₓ-accC | specR (SEQ ID No. 211) |
| pEG165 (SEQ ID NO. 209) | P_{sigB}-accC | specR (SEQ ID No. 211) |
| pEG166 (SEQ ID NO. 210) | P_{yflT}-accC | specR (SEQ ID No. 211) |
| pEG204 | P_{groES}-accC | camR (SEQ ID No. 220) |
| pEG205 | P_{ctsR}-accC | camR (SEQ ID No. 220) |
| PEG206 | P_{dnaK}-accC | camR (SEQ ID No. 220) |
| pEG207 | P_{perR}-accC | camR (SEQ ID No. 220) |
| pEG208 | P_{hrcA}-accC | camR (SEQ ID No. 220) |
| pEG209 | Pₛₚₓ-accC | camR (SEQ ID No. 220) |
| PEG210 | P_{sigB}-accC | camR (SEQ ID No. 220) |
| pEG211 | P_{yflT}-accC | camR (SEQ ID No. 220) |

TABLE 11
List of Bacillus strains investigated

| | | | Last | |
|---|---|---|---|---|
| | | | nucleotide | |
| | | | in RBS | |
| | | | of new | |
| Strain | New | Transformed | targeted | Parental |
| name | features | with | essential gene | strain |
| B. subtilis KO7 | — | — | — | — |
| EGS084 | IgA-GFP | pEG062 | — | B. subtilis |
| | expression | (SEQ ID NO. 222) | | KO7 |
| EGS340 | P_{perR}-iscU | pEG154 | T | EGS084 |
| EGS343 | P_{perR}-accC | pEG162 | T | EGS084 |
| EGS460 | P_{ctsR}-accC | pEG205 | C | EGS340 |
| EGS462 | P_{dnaK}-accC | pEG206 | G | EGS340 |
| EGS466 | P_{hrcA}-accC | pEG208 | T | EGS340 |

Chromosomal integration of burden-addiction promoters: To construct the candidate burden-addicted strains (Table 11), integration vectors (Table 10) featuring each of the promoters fused to RBS variants (Table 3.2) were integrated by homologous recombination upstream of the operons containing either iscU or accC in the genome of the IgA-GFP producing EGS084 strain (corresponding to B. subtilis KO7 containing the expression cassette of pEG062 inserted in the amyE locus) as follows. Competent cells were prepared according to standard Bacillus transformation methods following a described protocol (dx.doi.org/10.17504/protocols.io.bdmti46n) and previously transformed with pEG151 to pEG166. The transformants were selected on LB agar plates supplemented with spectinomycin (200 μg/mL) and confirmed by colony PCR using the primers E257/E258 (iscU) or E261/E262 (accC).

TABLE 12
Primers

| SEQ ID NO. | Oligo ID | Sequence (5′-) |
|---|---|---|
| 223 | E257 | CTGAGTCAATTTCATCAAGG |
| 224 | E258 | CAGAAGAAAACATTCTCGAAGC |
| 225 | E261 | GAGGTCGCAACTGAAAAAGC |
| 226 | E262 | ATGTTCAGTCTCCTTACTCC |

Long term IgA-GFP production assay: A single selected colony from each burden-addicted IgA-GFP producing strain was used to inoculate a 96 deep-well plate containing 500 μl/well Cal18-2 media (dx.doi.org/10.17504/protocols.io.bdmui46w) supplemented with kanamycin (10 μg/mL). The cultures were grown for 24 hours at 30° C./250 RPM horizontally shaking incubator (Innova, 2-inch amplitude). 1 μL of each culture were then transferred (as serial passage) in 499 μL fresh Cal18-2 media (corresponding to approx. 10 generations per transfer) into a new deep well plate under identical cultivation conditions. These method steps were repeated for a total of up to 13 times. At each serial passage, 100 μL of each strain were banked in equal amount of 50% glycerol for storage and follow-up examinations. To quantify IgA-GFP expression after each passage, cultures were grown for an additional 24 h at the previously described growth conditions. The cultures were spun down at 2000 g for 5 min and washed in 1 volume PBS twice. 20 μL samples of the culture were added to 180 μl PBS (10× dilution) in a 96-well plate and the cell density and IgA-GFP production was quantified by measuring OD600 and GFP fluorescence (λex/λex=485 nm/528 nm) in a Synergy H4 plate reader (Biotek). The specific production level of each culture was quantified as the GFP signal normalized to the OD600 value followed by subtraction of the same value measured in a non GFP-producing Bacillus subtilis KO7 strain.

### 8.1 Results

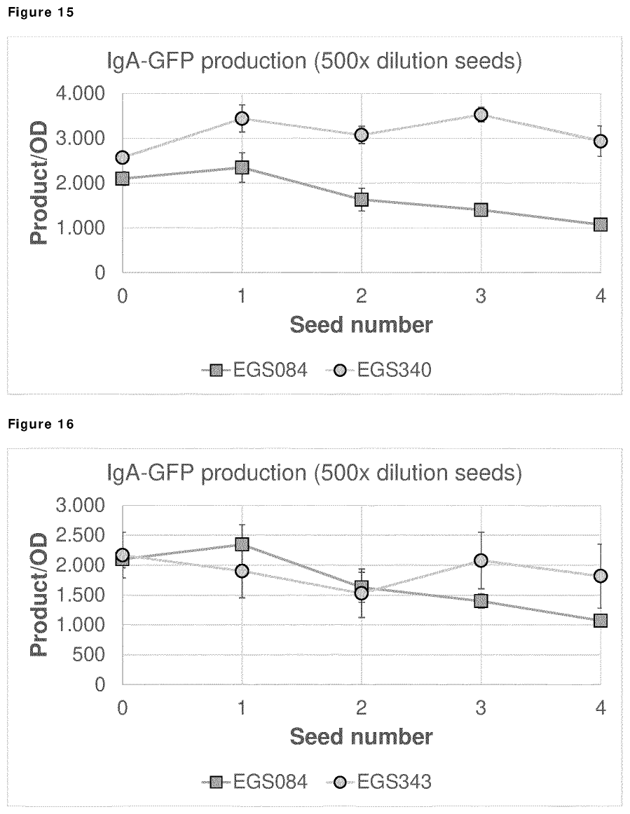

Following long-term cultivation of five 500-fold dilution serial passages (approx. 65 cell generations), IgA-GFP production was quantified and shown to be improved in the burden addicted strains (FIGS. 15 and 16).

Based on the methodology, similar burden-addicted strains can be constructed using other candidate burden sensors that may match the transcriptional burden response of other production strains. Their suitability can easily be assessed using the devised long-term production assays or for example in continuous small-scale (e.g. 400 mL) production cultures.

### Example 9: Burden Addiction Controlling Multiple Essential Genes Improves Production

It is here demonstrated how to introduce two (and by analogy further) burden-sensing promoters controlling different essential gene (operon) s in a single production cell to increase production of desired products and further prolong the period during which the production cells are productive Additionally, by using more than one burden-sensor to control essential genes, the transcriptional space that directs the imposed addiction-based selection regime can be further controlled.

The burden-addicted IgA-GFP production strain EGS340 (P_{hrcA} controlling the iscU essential gene operon) was genetically transformed to exchange the native promoter of the accC essential gene operon for the candidate burden sensors P_{ctsR}, P_{dnaK} and P_{hrcA} respectively.

### 9.1 Materials and Methods

Chromosomal integration of burden-addiction promoters: The spectinomycin resistance marker was exchanged for a chloramphenicol resistance marker in pEG159-pEG166 using the primers E372/E373 (for the vector backbones) and E374/E375 (for the chloramphenicol resistance marker in pDG1662, (Guerout-Fleury et al 1996) to create pEG204 to pEG211 (Table 10). Each of the promoters were integrated upstream of the essential gene operons containing accC in the genome of the EGS340 strain (corresponding to EGS084 containing P_{perR} regulating the iscU essential gene operon) as follows. Competent cells were prepared according to a previously described method (dx.doi.org/10.17504/protocols.io.bdmti46n) and transformed with pEG204 to pEG211. The transformants were selected on LB agar plates supplemented with chloramphenicol (5 μg/mL) and confirmed by colony PCR using the primers E261/E262.

TABLE 13
Primers

| SEQ ID NO | Oligo ID | Sequence (5′-) |
|---|---|---|
| 227 | E372 | ATTATAACAUGTATTCACGAACGAAAATCG |
| 228 | E373 | ATCAGATTTUAGAAAACAATAAACCCTTGCATAGG |
| 229 | E374 | AAAATCTGAUACGGGGCAGG |
| 230 | E375 | ATGTTATAAUACCCATTAGTTCAACAAACGAAAATTG |

Long term IgA-GFP production assay: A single selected colony from each burden-addicted IgA-GFP producing strain was used to inoculate a 96 deep-well plate containing 500 μL/well Cal18-2 media (dx.doi.org/10.17504/protocols.io.bdmui46w) supplemented with kanamycin (10 μg/mL). The cultures were grown for 24 hours at 30° C./250 RPM horizontally shaking incubator (Innova, 2-inch amplitude). 1 μL of each culture were then transferred (as serial passage) in 499 μL fresh Cal18-2 media (corresponding to approx. 10 generations per transfer) into a new deep well plate under identical cultivation conditions. These method steps were repeated for a total of up to 13 times. At each serial passage, 100 μL of each strain were banked in equal amount of 50% glycerol for storage and follow-up examinations. To quantify IgA-GFP expression after each passage, cultures were grown for an additional 24 h at the previously described growth conditions. The cultures were spun down at 2000 g for 5 min and washed in 1 volume PBS twice. 20 ul samples of the culture were added to 180 μl PBS (10× dilution) in a 96-well plate and the cell density and IgA-GFP production was quantified by measuring OD600 and GFP fluorescence (λex/λex=485 nm/528 nm) in a Synergy H4plate reader (Biotek). The specific production level of each culture was quantified as the GFP signal normalized to the OD600 value followed by subtraction of the same value measured in a non GFP-producing Bacillus subtilis KO7 strain.

Cell disruption and ELISA: Following washing in PBS, the cultures were spun down at 2000 g for 5 min. The pellets were resuspended in 1 volume of lysis buffer (10 mM Tris, pH 7.5, 150 mM NaCl, 500 μM EDTA), spun down again at 6000 g for 5 min, and resuspended in 1 volume of lysis buffer supplemented with lysozyme (10 mg/mL). The cells were disrupted by incubating the reactions for 30 min at 37° C. Afterwards, the reactions were briefly vortexed and spun down at 12000 g for 30 min at 4° C. The supernatants were subjected to ELISA using the Pig IgA ELISA Kit from Abcam (ab190536) according to manufacturer's protocol, except that after adding the chromogenic substrate, the absorbance of each well was read at 600 nm every 40 s for 10 min. The relative IgA concentrations were calculated as the slopes of values per OD600 of the original cultures.

### 9.2 Results

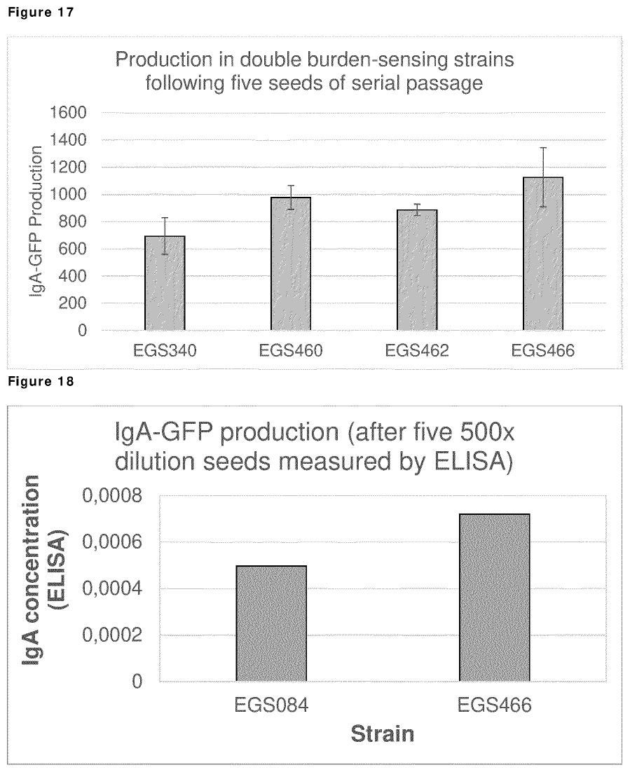

The single burden-sensor strain EGS340 carries only the p_{perR}-based burden sensor controlling transcription of the iscU essential gene operon (EGS340). EGS340 is compared to derived strains in which transcription of the accC essential gene operon is also regulated by respectively a p_{ctsR}-based burden sensor (EGS460), a p_{dnaK}-based burden sensor (EGS462), and a p_{hrcA}-based burden sensor (EGS466). Following long-term cultivation for approx. 75 cell generations, IgA-GFP production was quantified and shown to be improved in the burden addicted strains (FIGS. 17 and 18).

Based on the methodology, similar burden-addicted strains can easily be constructed using other candidate burden sensors that may match the transcriptional burden response of other production strains. Their suitability can easily be assessed using the devised long-term production assays or in continuous small-scale (e.g. 400 mL) production cultures.

### Example 10: Additional Method for Identification of Suitable Burden-sensing Promoters

In this example, suitable candidate burden-sensing promoters are identified for use in burden addiction to sustain long-term heterologous production in particular production strains.

The method is divided in an optional track 1 for de novo discovery (RNA-sequencing) and a track 2 for confirmation of putative burden-sensing promoters (q-PCR).

In track 1, using RNA-sequencing the transcriptional activity of coding genes in the production organism during typical production conditions (in terms of e.g. temperature, stirring, product/by-product/substrate concentration/growth phase) is compared to that of a an isolated non/low-producing organism during the same typical production conditions. The culture of the isolated non/low-producing organism may optionally be fed with product to the concentration approximately found in a corresponding culture of the production organism at a given time. The non/low-producing isolate can typically be isolated following culture for >50-100 divisions (preferred), but a suitable isolate strain can also be genetically engineered by genetically inactivating one or several key heterologous production genes.

RNA-sequencing is carried out, preferably on at least three replicates, preferably using Illumina short-read sequencing according to standard methods known in the art.

Using standard bioinformatical analysis for differential gene expression e.g. incorporating the edgeR work-flow (DOI: 10.18129/B9.bioc.edgeR), suitable candidate burden-sensing promoters are identified from genes up-regulated by at least 5-10 times in the high-producing culture compared to the non/low-producing culture under the same typical production conditions. By identifying candidate burden-sensing promoters using this methodology, promoters activated preferentially by the burden of product formation (compared to merely the stress of production) can be found.

Next, in track 2, putative burden-sensing promoters (from track 1 or other lists) are confirmed using q-PCR on samples, similarly comparing one non/low-producer isolates to high-producer isolates during the same typical production conditions (in terms of e.g. temperature, stirring, product/by-product/substrate concentration/growth phase). Suitable candidate burden-sensing promoters are identified from genes up-regulated by at least 5-10 times in the high-producing culture compared to the non/low-producing culture under the same typical production conditions.

### Example 11: Burden Addiction Controlling Multiple Essential Genes Improves Production of Recombinant Human Growth Hormone in Escherichia coli

The long-term production stability of the burden addicted E. coli strain s7.6 #8 was improved by integration of one additional, different burden-sensing promoter controlling transcription of a second essential gene selected e.g. from the list and screened as suggested.

### 11.1 Materials and Methods

s7.6 #8 single colonies were transformed with a recombineering plasmid such as pKD46 using standard electroporation. Using recombineering, candidate burden-sensing promoters (Table 2) fused to variable RBSs (Table 3.1) were chromosomally integrated directly upstream of the essential gene murI or a similar essential gene, and selected for using spectinomycin resistance gene present in the integration constructs.

Following the described steps of recombineering and curing of the recombineering plasmid, five colonies were picked, corresponding to clones with the same promoter integration but having a random variant RBS sequence. Each clone was then transferred to a well of a 96-well plate containing 200 μl 2xYT supplemented with chloramphenicol for maintenance of the plasmid, pMevT, and cultured. 2 μl of each cultured clone was used to validate promotor integration as described in example 1.2.1; prior to freezing the 96-well plate.

### 11.2 Results

The different resulting clones were assessed for long-term production stability by serial passaging of 200× dilutions. Following long-term cultivation for approx. 75 cell generations, IgA-GFP production was quantified and shown to be improved in the new strains compared to the single burden addicted strains only featuring one burden-regulated essential gene.

### Example 12: Expression-tuning of Essential Gene CIA1 With TIS Sequence Variations Shows Responsive Growth in P. pastoris Strains

This example demonstrates regulation of growth by variations in the TIS (translation initiation site) sequence of the essential gene is demonstrated. The translational strength of the essential gene is modified by such variations in the TIS sequence, which can be used in eukaryotic organisms to titrate the burden-sensing promoter's burden-response to the expression level of the essential gene similar to the use of ribosomal binding sites in prokaryotic organisms.

### 12.1 Materials and Methods

Burden-responsive promoter with TIS (translation initiation site) sequence variations: Variations of promoter, P_{gsh2} (promoter of P. pastoris homolog CDS: chr1-4_0496) with different TIS sequences of increasing strength and the cia1 (P. pastoris homolog CDS: chr1-3_0207) essential gene integration sequence were generated by PCR and USER cloning resulting in integration fragments (Table 14).

TABLE 14
Pichia chromosomal integration construct for inserting
promoter P_{gsh2} with TIS variations to control essential gene cia1

| | | | Resistance gene used to |
|---|---|---|---|
| Integration | Promoter- | TIS variation | select for chromosomal |
| fragment | essential gene | (see Table 3.3) | integration |
| INT1 (SEQ ID | P_{gsh2}-cia1 | TIS1 | Sh ble (SEQ ID NO 233) |
| NO 232) | | | |
| INT3 (SEQ ID | P_{gsh2}-cia1 | TIS3 | Sh ble (SEQ ID NO 233) |
| NO 234) | | | |
| INT5 (SEQ ID | P_{gsh2}-cia1 | TIS5 | Sh ble (SEQ ID NO 233) |
| NO 235) | | | |
| INT9 (SEQ ID | P_{gsh2}-cia1 | TIS9 | Sh ble (SEQ ID NO 233) |
| NO 236) | | | |

Chromosomal integration of burden-responsive promoter with TIS sequence variations: 5 To construct the strains with modulated expression of essential gene cia1, integration fragments (Table 14) featuring the promoter, P_{gsh2} with TIS sequence variations were integrated by homologous recombination upstream of the cia1 CDS in the IgAL-NanoLuc producing EGS621 strain as follows. Electro-competent cells were prepared according to standard Pichia transformation methods (Wu an Letchworth 2018) and transformed with integration fragments INT1, INT3, INT5, and INT9. Transformants were selected on YPD agar plates supplemented with Zeocin® (50 μg/mL) and confirmed by colony PCR using primers E521 (SEQ ID NO. 237) and E627 (SEQ ID NO. 238).

TABLE 15
List of Pichia strains investigated

| | | | TIS variation of the | |
|---|---|---|---|---|
| Strain | | Transformed | promoter targeting | Parental |
| name | New features | with | essential gene cia1 | strain |
| EGS621 | IgAL-NanoLuc | — | — | EGS75 |
| | expression | | | |
| EGS1100 | P_{gsh2}-cia1 | INT1 | TIS1 | EGS621 |
| EGS1101 | P_{gsh2}-cia1 | INT3 | TIS3 | EGS621 |
| EGS1102 | P_{gsh2}-cia1 | INT5 | TIS5 | EGS621 |
| EGS1104 | P_{gsh2}-cia1 | INT9 | TIS9 | EGS621 |

Pichia TIS sequence growth response assay: Single colonies of the parent strain, EGS621 and the TIS sequence variants EGS1100, EGS1101, EGS1102, and EGS1104 were diluted in 30 μL MilliQ H₂O, respectively. 5 μL of diluted colonies were inoculated in 95 μL YPD media to make pre-cultures. EGS621 colonies were supplemented with G418 (50 μg/mL) and the remaining colonies with Zeocin® (50 μg/mL). The pre-cultures were grown overnight at 30° C. and 300 RPM shake. Overnight pre-cultures were then diluted 1000 times in YPD media supplemented with appropriate antibiotic. 1 μL of diluted cultures were inoculated in 99 μL YPD supplemented with appropriate antibiotic in a 96-well culture plate and sealed with a Breathe-Easy® sealing membrane (Sigma-Aldrich, Darmstadt, Germany). The 96-well plate was placed in Synergy 4 plate reader (BioTek, Vermont, USA) and cultures were grown using the following settings: 30° C., high shaking speed, and OD₆₀₀ measurement every 10 minutes for 48 hours. 12.2 Results

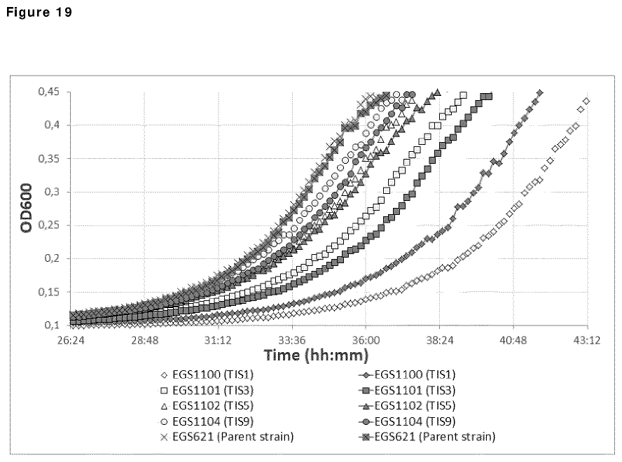

A growth difference between all strains is displayed—see FIG. 19. The parent strain (EGS621) with the native promoter, P_{cia1} exhibited the fastest growth. The strains with the TIS sequence variations of the P_{gsh2} promoter integrated just upstream of the cia1 CDS (EGS1100, EGS1101, EGS1102, and EGS1104) display growth in accordance with their relative TIS strength. The strain with the strongest TIS sequence, TIS9 (EGS1104) exhibited slightly slower growth than the parent strain (EGS621), and the fastest growth of the TIS strains (EGS1100, EGS1101, EGS1102, and EGS1104). The strain with the weakest TIS sequence, TIS1 (EGS1100) exhibited the slowest growth of all strains. The TIS3 (EGS1101) and TIS5 (EGS1102) strains exhibited growth in accordance with the relative strength between the four TIS sequences.

In summary: It is demonstrated that the growth of Pichia strains is responsive to the TIS sequence strength of P_{gsh2}, which was integrated just upstream of the cia1 CDS, confirming cia1 as being an essential gene for the growth of Pichia strains.

### Example 13: Implementation of Burden Addiction in an A. oryzae Production Strain

The burden addiction system of the present invention may also be implemented in other eukaryotes such as fungi. Examples of implementation of burden addiction in an A. oryzae production strain is provided herein.

### 13.1 Burden Sensing Promoter Element

Examples of burden sensing promoters include the promoter sequences (750 bp upstream of start codon) from the gene homologs of bipA (e.g. SEQ ID NO. 189), clxA (e.g. SEQ ID NO. 190) and agsA (e.g.SEQ ID NO. 239). To construct a burden-addicted A. oryzae production strain, the burden promoters are integrated in front of an essential gene, such as the ERG10 (e.g. SEQ ID NO. 240), PFS2 (e.g. SEQ ID NO. 242) or TUB1 (e.g. SEQ ID NO. 244) homologs. To tune the expression of the essential gene, the burden sensing promoter is integrated with a translation initiation sequence (TIS) library of 4 different variants as presented in table 3.3. These variants replaced the last 6 nucleotides of the promoter sequence, i.e. −6 to −1 relative to the start codon. Examples of burden-addiction integration fragments are presented in table 16.

TABLE 16
Aspergillus oryzae burden-addiction integration constructs

| | Burden sensing | |
|---|---|---|
| Integration fragment | promoter | Essential gene |
| agsA_ERG10_pyrG_NC_036442 | agsA | ERG10 |
| (SEQ ID NO. 246) | | |
| agsA_PFS2_pyrG_ NC_036436 | agsA | PFS2 |
| (SEQ ID NO. 247) | | |
| agsA_TUB1_pyrG_NC_036435 | agsA | TUB1 |
| (SEQ ID NO. 248) | | |
| bipA_ERG10_pyrG_NC_036442 | bipA | ERG10 |
| (SEQ ID NO. 249) | | |
| bipA_PFS2_pyrG_NC_036436 | bipA | PFS2 |
| (SEQ ID NO. 250) | | |
| bipA_TUB1_pyrG_NC_036435 | bipA | TUB1 |
| (SEQ ID NO. 251) | | |
| clxA_ERG10_pyrG_NC_036442 | clxA | ERG10 |
| (SEQ ID NO. 252) | | |
| clxA_PFS2_pyrG_NC_036436 | clxA | PFS2 |
| (SEQ ID NO. 253) | | |
| clxA_TUB1_pyrG_NC_036435 | clxA | TUB1 |
| (SEQ ID NO. 254) | | |

### 13.2 Construction of the Burden Sensing Integration Construct.

To swap the native essential gene promoters for the burden-sensing promoters at three essential gene targeting DNA fragments are prepared by standard molecular cloning. Approximately 1.5 kbp regions up- and down-stream of each essential gene start codons, (respectively ERG10, PFS and TUB1) are amplified from A. oryzae RIB40 genomic DNA. Next, a 750 bp promoter region immediately up-stream of the start codon of respectively bipA, clxA and agsA is likewise amplified from A. oryzae genomic DNA.

Lastly, a synthetic gene fragment containing a selection marker e.g. amdS or pyrG is obtained. All fragments are assembled using e.g. Gibson Assembly technique. From the assembly reaction full length knockout constructs are amplified using PCR.

A. oryzae protoplast are prepared according to standard protocols, such as Christensen et al. 1988, and transformed with the burden sensing integration constructs and selected on appropriate media.

Expression is assayed using shake flasks containing 10 ml YPM medium (2 g/l yeast extract, 2 g/l peptone, and 2% maltose) inoculated with spores from a transformant strain and a reference production strain without the burden sensing promoter element and incubated at 30° C., 200 rpm for 4 days followed by sampling for product.

Further assaying in fed batch fermentation is also performed: Tank medium (sucrose 24 g/L, yeast extract 10 g/L, (NH₄)₂SO₄ 5 g/L, MgSO₄-7H₂O₂ g/L, K₂SO 2 g/L, citric acid 1 g/L, KH₂PO 2 g/L trace metal solution 0.5 ml/L), temperature 34° C., aeration 1 vvm, and pH is controlled at 6.0 using 10% NH4OH. Medium is inoculated a seed cultivation (spores pre-grown in to shake flask (glycerol 20 g/L, yeast extract 18 g/L) for 1 day at 30° C. and 250 rpm) of a transformant strain comprising the burden sensing integration construct and a reference strain. When pH >6.4, feeding (400 g/L maltose sirup, 1 g/L citric acid) is started at a rate of 3.33 g/L/h. Stirrer speed is controlled to avoid too low (<20%) oxygen tension.

### REFERENCES

Bonde, M. T., Pedersen, M., Klausen, M. S., Jensen, S. I., Wulff, T., Harrison, S., et al. (2016). Predictable tuning of protein expression in bacteria. Nat. Methods 13. doi: 10.1038/nmeth.3727.
Christensen, T., Woeldike, H., Boel, E. et al. (1988) High Level Expression of Recombinant Genes in Aspergillus oryzae. Nat Biotechnol 6, 1419-1422. doi.org/10.1038/nbt1288-1419
Falkenberg et al (2022). Protein expression in Bacillus subtilis. Oct. 27, 2022. Website: protocols.io. dx.doi.org/10.17504/protocols.io.bdmti46n
Giaever G et al., Nature. 2002 Jul. 25;418(6896):387-91. DOI: 10.1038/nature00935
Jain, R., Kumar, P. & Varshney, U. A distinct role of formamidopyrimidine DNA glycosylase (MutM) in down-regulation of accumulation of G, C mutations and protection against oxidative stress in mycobacteria. DNA Repair (Amst). 6, 1774-1785 (2007).
Guerout-Fleury AM, Frandson N, Stragier P. 1996. Plasmids for ectopic integration in Bacillus subtilis. Gene 180:57-61
Koskiniemi, S., Pränting, M., Gullberg, E., Näsvall, J., & Andersson, D. I. (2011). Activation of cryptic aminoglycoside resistance in Salmonella enterica. Molecular Microbiology, Vol. 80, pp. 1464-1478. https://doi.org/10.1111/j.1365-2958.2011.07657.x
Kimata, Y., Ishiwata-Kimata, Y., Yamada, S., and Kohno, K. (2006). Yeast unfolded protein response pathway regulates expression of genes for anti-oxidative stress and for cell surface proteins. Genes to Cells 11, 59-69. doi: 10.1111/j.1365-2443.2005.00921.x.
Laferté, A., Favry, E., Sentenac, A., Riva, M., Carles, C., and Chédin, S. (2006). The transcriptional activity of RNA polymerase I is a key determinant for the level of all ribosome components. Genes Dev. 20, 2030-2040. doi: 10.1101/gad.386106.
Maeda, M., Shimada, T., and Ishihama, A. (2015). Strength and Regulation of Seven rRNA Promoters in Escherichia coli. PLoS One 10, 1-19. doi: 10.1371/journal.pone.0144697.
Nonaka, G., Blankschien, M., Herman, C., Gross, C. a, and Rhodius, V. a (2006). Regulon and promoter analysis of the E. coli heat-shock factor, sigma32, reveals a multifaceted cellular response to heat stress. Genes Dev. 20, 1776-89. doi: 10.1101/gad.1428206.
Pitera, D. J., Paddon, C. J., Newman, J. D., and Keasling, J. D. (2007). Balancing a heterologous mevalonate pathway for improved isoprenoid production in Escherichia coli. Metab. Eng. 9, 193-207. doi: 10.1016/j.ymben.2006.11.002.
Rugbjerg, P., Sarup-Lytzen, K., Nagy, M., and Sommer, M. O. A. (2018). Synthetic addiction extends the productive life time of engineered Escherichia coli populations. Proc. Natl. Acad. Sci. 115, 2347-2352. doi: 10.1073/pnas.1718622115.
Wu, S. and Letchworth, G.J. (2004) High efficiency transformation by electroporation of Pichia pastoris pretreated with lithium acetate and dithiothreitol. Biotechniques 36, 152-154
Wu an Letchworth (2018) High efficiency transformation by electroporation of Pichia pastoris pretreated with lithium acetate and dithiothreitol. BIOTECHNIQUESVOL. 36, NO. 1DRUG DISCOVERY AND GENOMIC TECHNOLOGIES. doi.org/10.2144/04361DD02.
Yoon, S. H., Han, M. J., Lee, S. Y., Jeong, K. J., and Yoo, J. S. (2003). Combined transcriptome and proteome analysis of Escherichia coli during high cell density culture. Biotechnol. Bioeng. 81, 753-767. doi: 10.1002/bit. 10626.
Uppal, S., Shetty, D. M., & Jawali, N. (2014). Cyclic AMP receptor protein regulates cspd, a bacterial toxin gene, in Escherichia coli. Journal of Bacteriology, 196(8), 1569-1577. https://doi.org/10.1128/JB.01476-13
1 Yamanaka, K., & Inouye, M. (1997). Growth-phase-dependent expression of cspD, encoding a member of the CspA family in Escherichia coli. Journal of Bacteriology, 179(16), 5126-5130. https://doi.org/10.1128/jb.179.16.5126-5130.1997

1. A microbial production cell genetically engineered to synthesize a product, said microbial cell further comprising:
a. a first essential gene operably linked to a first burden-sensing promoter, and
b. a second essential gene operatively linked to a second burden-sensing promoter,
wherein said first burden-sensing promoter is heterologous with respect to said first essential gene, and said second burden-sensing promoter is heterologous with respect to said second essential gene;
wherein synthesis of the product confers a burden on said cell, and
wherein expression of said first essential genes is up-regulated when said first burden-sensing promoter is induced by said burden relative to a basal level expression of said first essential gene when said first burden-sensing promoter is not induced, and expression of said second essential gene is up-regulated when said second burden-sensing promoter is induced by said burden relative to a basal level expression of said second essential gene when said second burden-sensing promoter is not induced.

2. The microbial production cell according to claim 1, wherein said burden conferred by synthesis of said product, when said first and second essential gene in the cell are operably linked to their native promoters, has a fitness cost measured as a percent reduction in the maximum exponential phase growth rate of the microbial production cell selected from among ≥5%, ≥10%, ≥15%, ≥20%, ≥25%, ≥35% and ≥45% measured relative to a corresponding non-producing microbial cell.

3. The microbial production cell according to claim 1, wherein the cell is:
i. a bacterium belonging to a genus selected from among Escherichia, Lactobacillus, Lactococcus, Corynebacterium, Bacillus, Acetobacter, Acinetobacter, Pseudomonas; Proprionibacterium, Bacteroides, and Bifidobacterium; or
ii. a yeast belonging to a genus selected from among Saccharomyces, Kluyveromyces, Candida, Pichia, Komagataella, Cryptococcus, Debaromyces, Hansenula, Yarrowia, Zygosaccharomyces and Schizosaccharomyces; or
iii. a filamentous fungus selected from among Penicillium, Rhizopus, Fusarium, Fusidium, Gibberella, Mucor, Mortierella, Trichoderma Thermomyces, Streptomyces and Aspergillus.

4. The microbial production cell according to claim 1, wherein the first and second essential genes are identical.

5. The microbial production cell according to claim 1, wherein the first and second burden-sensing promoters are different.

6. The microbial production cell according to claim 1, wherein the first and/or second essential gene is a non-conditional essential gene.

7. The microbial production cell according to claim 1, wherein the first and/or second essential gene is operably linked to a synthetic RBS whose sequence is selected to modify the translational strength of the first and/or second essential gene independent of induction of said first and/or second burden-sensing promoter, respectively.

8. The microbial production cell according to claim 1, wherein the first and second burden-sensing promoters are selected from among:
i. a ribosomal RNA promoter,
ii. a promoter upregulated by oxyR or a homolog thereof,
iii. a promoter comprising a UPR element upregulated by HAC1 or a homolog thereof, and
iv. a DNA damage-sensing promoter.

9. The microbial production cell according to claim 1, wherein the cell is characterized by an increased product yield after at least 25, 30, 35, 40, 45, 50, 60, 70, 80, 90 or 100 generations of cell division from a single cell, as compared to a parent microbial production cell lacking said first and second essential genes operably linked to said first and second burden-sensing promoters, respectively.

10. The microbial production cell according to claim 1, wherein the cell is characterized by an increased product yield of at least 10, 25, 50, or 80% following at least 50 generations of cell division from a single cell as compared to a parent microbial production cell lacking said first and second essential genes operably linked to said first and second burden-sensing promoters, respectively.

11. A method of product biosynthesis comprising the steps of:
i. providing at least one microbial production cell according to claim 1,
ii. introducing the at least one cell into a cultivation medium comprising substrate for production of said product,
iii. recovering said product.

12. The method according to claim 11, further comprising a step of isolating the product and/or formulating the product into a composition, such as a nutritional, pharmaceutical, cosmetic, detergent, lubricant, or fuel composition.

13. A method of product biosynthesis according to claim 11, wherein the product is selected from among: an organic acid, terpenoid, isoprenoid, polyketide, alcohol, sugar, vitamin, aldehyde, carboxylic acid, fatty acid, amino acid, peptide, enzyme (such as an amylase, lipase, protease, barnase, β-galactosidase, crystal protein, cutinase, PETase, laccase and carbohydrate active enzyme (such as a xylanase, lichenase, cellulase, lytic polysaccharide monooxoygenase, and pectase)), a therapeutic protein and a precursor thereof (such as human growth hormone, insulin, glucagon-like peptide-1, monoclonal- and polyclonal-antibody, single-fragment antibody and nanobody), a protein naturally found in eggs (such as ovalbumin), a milk protein (such as casein, lactadherin, lactoferrin), secreted immunoglobulin A and G, secretory components.

14. Use of a first and a second essential gene operably linked to a first and a second burden-sensing promoter, respectively, to enhance product yield of a cultured population of microbial production cells arising from a single cell following at least 50 generations;
wherein said first burden-sensing promoter is heterologous with respect to said first essential gene, and said second burden-sensing promoter is heterologous with respect to said second essential gene;
wherein synthesis of the product confers a burden on said cell; and
wherein expression of said first essential gene is up-regulated when said first burden-sensing promoter is induced by said burden relative to a basal level expression of said first essential gene when said first burden-sensing promoter is not induced, and expression of said second essential gene is up-regulated when said second burden-sensing promoter is induced by said burden relative to a basal level expression of said second essential gene when said second burden-sensing promoter is not induced.

15. Use of said first and second essential gene operably liked to said first and second burden-sensing promoter, respectively, according to claim 14, wherein product yield is enhanced by at least 10, 25, 50, or 80% following at least 50 generations of cell division from a single cell as compared to a parent microbial production cell lacking said first and second essential genes operably linked to said first and second burden-sensing promoters, respectively.

16. Use of a microbial production cell according to claim 1 for producing a biosynthetic product, such as organic acids, terpenoids, isoprenoids, polyketides, alcohols, sugars, vitamins, aldehydes, carboxylic acids, fatty acids, amino acids, peptides, enzymes (such as amylases, lipases, proteases, barnases, β-galactosidases, crystal proteins, cutinases, PETases, laccases and carbohydrate active enzymes (such as xylanases, lichenases, cellulases, lytic polysaccharide monooxoygenases, and pectases)), therapeutic proteins and precursors thereof (such as human growth hormone, insulin, glucagon-like peptide-1, monoclonal and polyclonal antibodies, single-fragment antibodies), proteins naturally found in eggs (such as ovalbumin), milk proteins (such as caseins, lactadherins, lactoferrin), secreted immunoglobulin A and G, secretory components, nanobodies.
